SEQUENCE LISTING (6 sequences)

SEQ ID NO: 1 - DNA, 81001 bp, Homo sapiens
  misc_feature 10946..12946: 5′regulatory region
  exon 12947..12958: exon 1
  exon 13470..13526: exon 2
  exon 13641..13752: exon 3
  exon 14271..15968: exon 4
  misc_feature 15969..17969: 3′regulatory region
  allele 1239: 20-828-311  : polymorphic base C or T
  allele 12347: 17-42-319   : polymorphic base C or T
  allele 15241: 17-41-250   : polymorphic base C or T
  allele 42218: 20-841-149  : polymorphic base A or G
  allele 45442: 20-842-115  : polymorphic base A or G
  allele 77058: 20-853-415  : polymorphic base C or T
  primer_bind 929..949: 20-828.pu
  primer_bind 1357..1377: 20-828.rp  complement
  primer_bind 12029..12050: 17-42.pu
  primer_bind 12581..12603: 17-42.rp complement
  primer_bind 14992..15012: 17-41.pu
  primer_bind 15460..15482: 17-41.rp complement
  primer_bind 42070..42090: 20-841.pu
  primer_bind 42572..42591: 20-841.rp  complement
  primer_bind 45328..45347: 20-842.pu
  primer_bind 45863..45883: 20-842.rp  complement
  primer_bind 76644..76664: 20-853.pu
  primer_bind 77166..77185: 20-853.rp  complement
  primer_bind 1220..1238: 20-828-311.mis
  primer_bind 1240..1258: 20-828-311.mis complement
  primer_bind 12328..12346: 17-42-319.mis
  primer_bind 12348..12366: 17-42-319.mis complement
  primer_bind 15222..15240: 17-41-250.mis
  primer_bind 15242..15260: 17-41-250.mis complement
  primer_bind 42199..42217: 20-841-149.mis
  primer_bind 42219..42237: 20-841-149.mis complement
  primer_bind 45423..45441: 20-842-115.mis
  primer_bind 45443..45461: 20-842-115.mis complement
  primer_bind 77039..77057: 20-853-415.mis
  primer_bind 77059..77077: 20-853-415.mis complement
  misc_binding 1227..1251: 20-828-311.probe
  misc_binding 12335..12359: 17-42-319.probe
  misc_binding 15229..15253: 17-41-250.probe
  misc_binding 42206..42230: 20-841-149.probe
  misc_binding 45430..45454: 20-842-115.probe
  misc_binding 77046..77070: 20-853-415.probe
cctcctgatatgacatcatgtgaacttctcacttcacctttaagtattcttagccaggcacagtggcacatgcttgaaatcccagcactttgggaggctgaggcagggggatcgcttgaggccaggatttcaagaccagcctgagcaacatgtcaagacccccacctctacaaaaaattaaaaagttagccaggtgtggtggtcatgcctgtagccccagctacttaggaggctgaggtgggaggatcacttgagcccaggagtttgaggctgcagtgagctatgatcacaccactgcactccagctggatgacagagcaagaccctgtctcaaaaaacaaaacaaaacaaaaaacaccgaaaaaaccccacagtaaattagaagaaagctgcataaattagaagaagctgatgtgaaatcctgagtccatggttcaatttaggagggaccatctggagagcagggaacccccaaaagtcagtttctttaggcttatttccttgggccagccaaatttctcagggagaatgttgttccattttcaatatggggaaagatggtgaactgaggagtcttttaaaaaaatttatttttgagatggggtctcattctgtcgcccaggctagagagcagtagcatgatcatggctcactgcggtctctaactcgtgggctcaagtaatccttctgcctcagcctcctgagtagctaggaccacaggtgccaccatacctggctaatttttgtattctttgcagagacagggtctcgctacattgcccaggctggtatcgaactcctgggctcaagcgatcctcctgccttgccttctcaaagtgttgggattacaggcatgagccactgtgcccagcctcaaaatttaatgtataaagttttccttaatttttcttagcacaaaaaccctggcccccaacaatacctagttttctccaggccggagtcccactcttttacccttttcagagagaataagcatctggttttctgctgctttgggggtacccagccaagtagagttgaagagaacagctgcttctcaaacagactctcgaccaactgccatatttctagtcccactgccacccactcttccagaagaatgttgacactaatgtcagagcatttggagagtttagtagtgaaaatcaggggccttcttggctttctccactgctgcttcaaaattcatgtcaggtgtgcctgtcaccaccgtttgaycatttggaagctttccagcttcccaaatgttgttatttttgtctccttttctattttccctttgggtttatgcattttgtaaaaagtgcacttcaatgccacgttattgagatttcagagaacagcagaggctaatgcatgcaattaatccaccgtccgttactagaagtcaatcggatgctctttagtctctcttccccatatactagtttaaaagttatccattctttctattcgttttatgggttatccttaaaattttaatattcttgtctgacctaacaaagtctatagataatcaatatccctatctttctcccgaataatgcaaaggctgctgaatgctttcactttgatctctcctttcccatttccaggttgcttcggtctgatattttagttcctcattacttttaacacctcctccaaagtagtcccttcatcaatagatgtttttgagccctccctaccatgtgataagcactggtctaggcactgggagtacagtaggaaatgagataaacttggccaggtgtagggtggcttacacctgtaatcccaacacttttgaggccgaggcgggcagctcgcttgagcccatgagttcgagaccagcctgggaaacatagcgagacccccgtccctacaaaaaaatataaaaattagctgggcatggtggtgtatgcctgtggtcctagctactcagaagactgaggtgggaggatcatctgagcccagggtggtcgaggctgcagtgattacaccactgcactccatcctgggcaacggtgagaccctgtctcaaaaaacaaacaaacaaacaagcaaacaaaacccccacaaactaaactatgtgtaaatacatttttgttaggtagaactatatgaaattgccactatttgaccaatttttagtgaaaactagtctcataagtgtgtgtgtgtgtattttcactaatgttttttggatttacctaaacgtttactaatttcattgctccccatgtctccttctatcctattccttttttctgggttctgtttccttttcagatttttagtagttcttttcagtgaggatctgtgagtggtaaactctctttctctgaaattaacttcttcctctcaaataatagttcacctgagtataagtcttggttggccattaatttcctttcagtctttagaaggtacattgatgataaatcagttgccggtttaatcatgcttcgtgtgtagatcattagtctttctctttggttgattttaagatatccattgccttcagtgttctgcagattcctgtgatgtgtcctcatttggttgtgtgttaatttttcctaccaagactcaggatgcttcctgtacctgaggattccggtcacatcttgcttcaatgtttgaaatttctcagccatcatcttttgaatattgcctcttccacagtccctgtgttctcttcgtggaaatcctacaggcatatattggacctcccattctgtcctccatgtctcttaccgtctattcataccctcctttttatatttaatttttttgagacagagttttgctctgtggcccaggacggagtgcaattgcatgatcttggctcacggcaaactctgcctcccaggttcaagctattctcctgcctcagtctcccaagtagctgggattacaggcatgcaccaccacgcccggctaatttttgtatttttagtagagacggggtttcaccatattggccaggctggtctgaaactcctgacctcaggtgatccacccacctcggcctcccaaagtgctgggattataggcatgagccaccatgcctggccaatatcacccgcctcggcctcccaaagtgctgggattacaggcatgagccaccgtgcctggtcaatatcctcctttttatttctgtgattctttctgtgtgattttctcagatctaccttctagcttactaattctctctccaactgtagctaaatgtgttttatattataatgactatatttttttcacttatagatgttctatttaattctctttctttttgacaaatagaacttatttcaaaacaaacaaaaggccaggcatggtggttcatgcctgtaatcccagtgctttgggaggctgaggcaggaggattgcttgagcccaggagttcaaggccagcctgggcaacatagtgagatcctttctctaccaaaaaaataaaaaatcagctgggtgttgtgggacactcctgtagtcccagaaactacagattcttgggaggcagaggctggaggattgcttgaggcagaggctggaggattgcttgagcctgggaggttgaggctgcagtgaactgtgatcatgccactgcactctagcctgggtgacaaagtgagatcctgtctcaaaaacaaataaacaaacaagcaaagaaacaaaaaaatgcttacacaggttactactttcttgctgggatagttttaacactgcgttaagcataaacacctttctttctgaaatgtatttgagatgtatattgatttttaaaaaacccacacctccattaaggtctggtgatagcagtagaaacaatgtagagtggctccacaatcatatagatgtttttggtgcgttctgagatggagtccaggaacaccaagtaaagactgctacctcacagtttacatctgagttcttagaagacaagactgaaggagaacaatttgtaacaagatttacttggcccgggtgtggtggctcacacctgtaatcccagtactttgggagctttgggagtccgaggtgggtggatcacctgaggtcaggagttcaagaccagcctggccaacatggaataacccccatctctactaaaaatacaaaaattagccaggcacggtggcacacgcctgtaatcccagctactcaggaggctgaggcaggagaatcgcttgaacccaagtgactgggttgccgagagccgagatcacgccactgtactccagccaccctgggtgacagagtgagactctgccaaaaaaaaaaaaaaaaaaaaaaaaatacttactgttcagaaggagaagtcataatgttgctttaaagaacaggtcacaaaagaaagactctagaagatcttctcacttggtgcatatcaagtgtctatttgagacccatacacttgcttaatccatgtgtttaaggcaaaagtgctgctgctgagcagtaaggaataaggtacctgctaacctttaccaatctacattttaaaatccttcttactacacatccagaatgagtcagcaattcttgtgtattaaaaaacaaaaacacaaaacaaagtagaggggcaactctcttaaaaatgcagctatccgcaaacactgtgatacaaaacgacagtcaaggaaagggcagcacaaacaagttcacctggaaggaatctgttcaaagtctctggatttaagaacaagttccctaaaagctcttacttacagaagaaatcggataataaatgtagctggaatgatggaattctttaagttttcattttgttttgggcaactctgtggcccaggctggagtgcagtggcttgatcacggctcactgcagcctcaacctcccaggctctggtgatcctcccacctcagcctcctgagtggctgggactacaagcatgtgtgccaccatgctaggctaatttttgtattttatttttagtttttttttttttttttttttttttgtagagacaggttttgccatgttgccaaggctggtctcaaattcctgggttcaagtgatcctcccatctcagccttccaaagagctgggattacaggcatgagctactgcacctggcctagaattttttaaaaatcactatctggcaactctcaggataatattcgattcaggcaaggatcatcaatgaatgctaaaaccattgggtgaaaaattgttgcagaatgggatgctcacatggcttcaaagtattgctccacaaattacttatcaataacgtaaaaaaccaaactttactagccatggagaaatctggttgttatcactttaatggagtgatcaaacttaacatcactaaatagagtgcaacctccagctaggatacagtaagaagggccagatatcacctagtatttttgccaaaaatgtttaaccttaatctaatcatgagaaagtaatcactcaaatccagaatgtgggacattttacaagatgtcctccttgcactcttccaaaaaaaaatcaatgtcatgaaaacaaacaaatggtggggttggggagaacggttctaaatttaaaaactaaagtgggataacaaccagatgagatgtgttagagcttgaatttacagagagagaaaaacaactatgaaagcattttggggaaaatctgaatatgtaggatatgttagatgatattaaggaattgtgttaattttcaaaggtatgataatgtttttttcttttgtaaaagagtccttatttttcacaatgtatgttgaagtattcagagtgaagtgtcatgttggctataattatttcaaatggttccacacacaaagcacataccacatacacatatacatatacctccaaccaactcaaaacatgttcaacactgaaactataagatgccaccaaacagggaagcatgagtgtgtgttgcatctacccattgtatcaatccaggttcagtcagaaaaacaaaagccattccacgtatttcaagcatgaaaggctttaaaacaaaaaattaaaggtttatccaactcttggaagggctggaggagtgaccaccttggtttgcagttcagaaggagtgactctcaaacgctcattagtaagtggctacaaatgggaagctcgccttattatgcctgcaatatcaatgcatgtgattcctgggaaggtcacccagaagctgctttaaactccaagcctgtccatgcttctgtctgcaaccggcattgaaacataatggcctctcctcttccgtctcacgctggctgactctaacctaggctcatatagagaagggattctagaaaatgtattaatagttccaagtgtcccctctgcatctcataaaagaccttagaaagggcactgataatgctatttgcaaaaagacaatccagcgcagttgtattttacagcacaggctctttaagtttgggttatcagcaaaaaaccattagagtatgagaaattcctttttaaattgtggcaaaatatacataacataaaaattaccattgtagctattgtacattgtagctaagtatatagcccagtagcactaaatacatttacactgttgtgcaccactatctagctccagaaacttttaatcttcccaaacagaaacttgtacctattaaccaataccttctccttcctcacttctcctagaaaccagaattatactttctgtctctacaaatctgactattctggatacctcagaatcacagtatgtgtcgttctacgactggcttgtttcacttagcatcatgtcttcaagggtcatccacgttatagcatgtgtcagatttccttttcttttcttttctttttttttttttttgagacacagtctcgctctgtcacccaggctggagggcagtagcacaatctcagttcactgcagcctctgccttccaggttcaagcaattctcgtgcctcagcctcccaagtagctggaatgacaggcatgcaccaccacacctggctaatttttgtatttttagtagagacggggttttaccatattggccaggctggtcttgcacttccggcctcaagtgatctgcccgtctcggcctcccaaagtgctgggattacaggcgtgagccactatgcctggcccccgattgtcattatttaaggctaagtgatattttgttgtctgtatataccacaatttgtttattcattcatctgtcaatggacatttgggttgtttccaccccctggttattgtggataatactactaggaacacgagcatacaaatatctgctccagtccctgcttttatcttttggatatatgcccagaggtggaattgctgggtcataaggtaattctagattaaattttttgagggactgccgtattgttctccaccatagctgcaccattttacctttccagcagcagcgtacaagcggtccagcttctccacatcttcacccacacttgctatttttggcttttattttattttttaaaataacattctaatgggtgttaagtggtcagaaatggttcttttaggagtagagatagaggccagggggatggctcacacctgtaatcccagcactttgggaggcctaggtgggcggatcacttggggtcaggagtttgagatcagcctggccaacatggtgaaactccatctctattaaaaatacaaaaattcgctgggtgtggtggtgtgcactcccagctacttgggaggctgagggaagagaatcgcttgaacccgggaggcggtagttgcagtgagccgagatcacaccactgtatggcctggtgacagagcaaggctctgtctcaaaaaaaaaaaaaaaaaaaaaagagtagagatagaaaagcattgaaaacacagcctcagctcagctcagtctgccatggtgggaagccattaattcttcactcttgaaaccttttcgtccttggtgtggcagaggctgcaagtctcctctgcaactttattcttcccttctttctcagttataaaatccctgattttagaaatatctttattgagatataattcacataccataacattcactacaattgaatggtttttagtatattcacagatttgtacaactatcaccacaaactaagtttagaacttttttcatcatcccacaaagaaaccccacacccattagcagttattcactatttctccccaatcaacctcccctcccctcaatagccctaggcagccaccagtctactttctgtctctacctatttgtcttttctggacattttatacaaatgagattttacaacatgtagtcttttgtgactggcttttttcccctagcataatgttttccaggttcatctgtggtgtagcaggtatcagtacttcaaccctttttattgccaaataatattccactatatggataggtaacattttgtttatccattcatcaattgatggacatttgggttgtttccattttcttgactgttatgaataatgttgccatgaacattaatgcacaagtttttgtgcagatgtgtattttcatgtgtcttggttttatacctaggaatagaattgctgagtcataggagaactcctccatgtttaaccattaatgaactgccgaactgttttccaaagaaattgcaccattgtacaatcccaccagcaatgtatgagggtagaatccctgatttttaactgatcattgaactcaggcccattcaaaacaaagatgacatttcctaaccttccttacaagtagttctgaccagtgagatgggagaagaagttaggttttgtccttaaaagaaaggagagtggctgggtgcagtggctcacgcctgtaatcccagcactttgggaggccgaggtgggcggatcacctgaggccgggagttcaagactagcctgaccaacatggagaaacctcgtctctactaaaaatacaaaattagctgggcgtcgtggcgcatgcctgtaatcccagctactcaggaggctgaggcaggagaatcgcttgaacctgggaggtggaggttgtgatgagccgagattgtgccatcgcactctagcctgggcaagaagagcgaaactccatctcaaaaaaaaaaagaaagaaaggagagtacattctacactctcctctcctccaccctgccccctttccagtggctggatgtggacatggtggtaagctatcttagatcatgtggacaagggaaacacatagggatattagaacctccagacagaaggaacttaggaccctggacagctttgtggagcagtgctcccataccagcgtgaagttttgtatgggagaaaacatacatttccagcttttgtaagccactgttattttgggtctctctcagagcagccaaatatataatttaactaatatattttctttctgtgattcttctttattttgattatacttctacttctctgcccctcttttaggtgggaggtgctgctccaagcactaactcagaatatagaccctctccctcttgtaatagtgccagcttggagttctttgcttccactgtaggggaaggaaggaaaaaatatggagaactcacatccactcttcattgtcctcaaacagaagtaacccattttgttctgctcacagcccattggccagaactaactgtatggccccaatctaattgcaaaggagtctgggaagtacagcacagcacatggatctttggaaagcgttaattttctctgccaaaggcttcttttgttgttgttgttgtttgaaatggagtcccgctccgtcacccgggctggagtgcagtggccctatctcagctcactgcaacctccacctcccaggttcaagcactcctccagccttggcctcccaagtagctaggattacaggcgtgtgccatcatacctggctattttttttgtatttttagtagagacagggttttaccgtgttggccaggctggtctcaaactcttggcttcaagtgatccgcctgcttcggtctcccaaagtgttgggattacaggcgtgagccactgtacccggccaatactggtgttttctgacctcagcgtttttcttttctcctgccatgtactctccctagagatttcatctactcccaagtcttccactgctcctatctgctgatgactcccaaaactcagtctccagccgagacttctctcctgggcttgagacatatgtatccaactgccagaacatctccagtggacagcctttgggcacaaggccacactagcttgtgggtacaagtaatcacccaaagtcaatttcagtggctctccactcccacattttttcaacccctggaaatgttcccttcccaaatactctgagtctctcttctcttttgatgactgtggttttgtgactggatggtagctcctgttgctttttttcctttcaaatgaattttctcttaggaggcttcttagccattaagcaaatagacctcaactgggcttgccctatgcctatctgaaacccagcttaggttcgagttaggactcctgttaatctgagctctcacttcctgtcccaacctgcttattttttttggtgaaagaaaatatcatccccctagttgctcagaaacctgggaatcattgggattttttcctctccttcaccttcctcatccaatcagtcaccaagtgctatcaactctgctgccttagtagccctcaatatatacttacctatcaaccatcactgctatgccatacttcaggccttcatttctcacttggattattaaaatatccctaaatagttcctctgccttctctctggccaccctcctgagtgatctctccatcatgtacctttcagtgactgcttgcacaagcccctttgtgacttggtcatagtctgctctcttgaaccaccagagccaagcacctgggttctgattctggttgcaactcttactgcgtgatgatggacaggccacttgatctcctcaaacctcagttcctaaatcaaatgaatgattgaattcagtcacttaactttgtatgtagtaggtaggcactgtgcaaaacatactagtggatatagagatgaataagaaaaagcccctgcactcaaagagctctcggattcatcaacaaattattgtgcagttagatagtaagtgctataatccaggaatatacagtgttgtgtgaataatgtggaatcagtttatctccagagcagaaaaaggtgaaggccgaagaaggcattcagagtgatactggagctgtgtagcaggggctactactgttccctcaaatcctttcttctcttcttcctgagtaatagagtcctttttcagctaggcatacagccatccaaaataaaaactatatttcccagcctccttttcagctaagtgtagccatgtgactaagttgtggccaatgggatgtcagtacaagtggtaatggcaatatctgggatgtgtctttaaaaggaaggaacatgtccttctccttttcctccttcctgttccctgggaggtgaacttggtagctggaggtgaagcagctggaacttggatatgagcgtcttgttgatgataatagtgcaacaagataaaagcagcccgagctggcctacatttacatgagaaggaaataagactccattttgtttaagctattctcttgcatatatatatacacatatatatcagctaagtgtagccatgtgactaacttgtggccaatgggatgtcagtatgagtggtaagagcagtatcttgtatatatatatgtgtatatatacacacacacatatatatacatatatgtatgcaagaatataaaatatacatgtgtgtgtgtatatatatacacacatatatatgtcccttgcagccaagtctaattctaactaatacaaactaactctaaaaaatgaatatatattcaccaggggataggctatttcaagcagagggaagcctgtataaaggctcagggaatgctgtggttttatgtggcagcagatgagactggaaatgagtcaggatgagccacagtggaggatgaattaaatgggcaggagtgtggtagaaagacctgttggaggctatgaatgcaatcaaggtgacagacaactggtgcaatgatggtagtggaaatggaggagaggggattgattcaagatgcatttaggaccaagaatcgggagcttgtgaacgtgtgtatgagtactgtagacggagtgggtgtgtcatcagagaagatctgagcatttgggcttgctctcctcagaggccctgcgagtggagttcagcttttcctcatggggcaaatctyactttcgctccagttcctggggctcagagtccctggcccagatgcctcttgccatctcatcttcaccctgcctggcttcccttgcttgttccaggattgtttcataaagagggatgtggttggtctttaaccctatgaatgctggctgaggatgcctgcggaacctgtagtgaagctttcaggggctgctcgggttctggctggtaggtgaacactgtccatcttgccggctgggacacagtgactctgggtagttgtgtaagagaggggcccttggcagacaaacaggttcttctctgttggtgggccagccagcaggtcagtgggaaggttaaaggtcatggggtttgggagaaactgggtgaggagttcagccccatcccccgtaaagctcctgggaagcacttctctactggggcagcccctgataccagggcactcattaaccctctgggtgccagggaaagggcaggaggtgagtgctgggaggcagctgaggtcaacttcttttgaacttccacgtggtatttactcagagcaattggtgccagaggctcagggccctggagtataaagcagaatgtctgctctctgtgcccagacgtgagcaggtgagcagctggggcagagggatgggggtcacagtcctaagggagggcattgcaggtggcctcaggggagagcctggggtggcccctaagacgtcctcttggaacattttggcagagttgcctcttcgccctcattatggctcagtttttccaccatgaaatgggagggagggagacaggtgggcaggggagaggtggtagaagtggcctagagaactgttcctggggtctgggacctttgcgaaggggttagagcaccacgctccctgctatgtgactgaggtagcaagagcacgccctcttcccatgtttgaggaagacaccctagcctccttgactcacctaggtcagtcctcttgagccccaacagctctgtgctccccagcccaaggaaggggtaacaggatttcgggcagttgcccctgcagaggccccctgggcaagtcccctgcgccatgtcccttcgtctccttcttcccctaaccaggcctccctccacctgtcttctcagagcaggtaatggcaagcatggctgccgtgctcacctgggctctggctcttctttcaggtgggtctccgaccctgacttcaacgtgggggtgtgggtggaggctggccagagggccctgtccaccctgggggaggagagcccaggccctgattacctagtccctctccacagcgttttcggccacccaggcacggaaaggcttctgggactacttcagccagaccagcggggacaaaggcagggtggagcagatccatcagcagaagatggctcgcgagcccgcgtgagtgcccaggggaaggggtgtaggcgaagggaggagacagctgggccatgccatgatgacctgcctctgctgcctcaacctctgtggccgctgctgggacagaggaaaggagcggtgctagctctgtctgcagatcccggccatcctgggctctttagcgccctctgcctgcagcccccgccttgacaactccgtagctgttgcccccttgctcactgaggcgcgggacctgggatcaatcgggaggacgcccgctgcagtccccagaatcaaaggatgatgtggcgcatctatgtttctttggagagtgttgtaggtctggatttgtatgggcaatgtgtttgtgcttcgtgcgtgagttgttactggccagggctaggacaagagccctcgaccctggggccaacgccctgcgtccttggttcccccagaggatcagtgcgcgatgacttggggacaaaggagatgatgggggctagcagtctgacggcctggatatctgtccccttctccaggaccctgaaagacagccttgagcaagacctcaacaatatgaacaagttcctggaaaagctgaggcctctgagtgggagcgaggctcctcggctcccacaggacccggtgggcatgcggcggcagctgcaggaggagttggaggaggtgaaggctcgcctccagccctacatggcagaggcgcacgagctggtgggctggaatttggagggcttgcggcagcaactgaagccctacacgatggatctgatggagcaggtggccctgcgcgtgcaggagctgcaggagcagttgcgcgtggtgggggaagacaccaaggcccagttgctggggggcgtggacgaggcttgggctttgctgcagggactgcagagccgcgtggtgcaccacaccggccgcttcaaagagctcttccacccatacgccgagagcctggtgagcggcatcgggcgccacgtgcaggagctgcaccgcagtgtggctccgcacgcccccgccagccccgcgcgcctcagtcgctgcgtgcaggtgctctcccggaagctcacgctcaaggccaaggccctgcacgcacgcatccagcagaacctggaccagctgcgcgaagagctcagcagagcctttgcaggcactgggactgaggaaggggccggcccggacccccagatgctctccgaggaggtgcgccagcgacttcaggctttccgccaggacacctacctgcagatagctgccttcactcgcgccatcgaccaggagactgaggaggtccagcagcagctggcgccacctccaccaggccacagtgccttcgccccagagtttcaacaaacagacagtggcaaggttctgagcaagctgcaggcccgtctggatgacctgtgggaagacatcactcacagccttcatgaccagggccacagccatctgggggacccctgaggatctacctgcccaggcccattcccagctycttgtctggggagccttggctctgagcctctagcatggttcagtccttgaaagtggcctgttgggtggagggtggaaggtcctgtgcaggacagggaggccaccaaaggggctgctgtctcctgcatatccagcctcctgcgactccccaatctggatgcattacattcaccaggctttgcaaacccagcctcccagtgctcatttgggaatgctcatgagttactccattcaagggtgagggagtagggagggagaggcaccatgcatgtgggtgattatctgcaagcctgtttgccgtgatgctggaagcctgtgccactacatcctggagtttggctctagtcacttctggctgcctggtggccactgctacagctggtccacagagaggagcacttgtctccccagggctgccatggcagctatcaggggaatagaagggagaaagagaatatcatggggagaacatgtgatggtgtgtgaatatccctgctggctctgatgctggtgggtacgaaaggtgtgggctgtgataggagagggcagagcccatgtttcctgacatagctctacacctaaataagggactgaaccctcccaactgtgggagctccttaaaccctctggggagcatactgtgtgctctccccatctccagcccctccctctgggttcccaagttgaagcctagacttctggctcaaatgaaatagatgtttatgatagaagtttgcctggcgtgactctcatttggaccatgtctgaaagcagtggcctcaccactatccccaaagcacacccatcacccactccattcccttgctgctctttctcatccacccactcccagtccaggtctgtcaaagggggtctggctgggctctgcttcagggatcctggctagacaacggctgtctgtcacacctggcaggagggcctgggttacgggcccttcctctgcacctgcactgttcactagcctgctcccccacaggacactgtgcatggaatgcaggctgtgtctggaagagctgtggccctggtggacctaagattcctgaggtgggctgcctcctttgttcctgctgttctagagtttgaatggcctctttttatgccggactctcttctggggactcccctcactcaggggcaccaatgctccctatagatcccctgggaactgaaactggggtgtggtggaggacgtggaaagggtaaacacagctccttgtctttggacttccctgtccggccccctttcctcccagctcagcctactgtccccgggttctcagcacctgcctgctccccaaccccatagcacagaccccacacatatgtaggctcatcatgcctgcaggctggtcttccctgacaccgtggattttgacaatgttggcaacagaactgggttgtggacccagcacctggagagaggaagtgctagaaaggtagaaataataaaaggtgtttttgttgttgttaggaaactggaaaagcataggtcaagggctatgatggggatgaggaggtaggagtgaaaatgagggctgtgtacttgaggctgggattggggaaggtagtgatgaggacagaatagggagtgggaagaacagaaagggacagagggattcagggattgtgagagaggggaagaggctgagccacccggaggggcgacctagcacgcaagcagtatgtggcccaacactggaaccaagcagcccggctccgggcgcaccttctcagggattcctcagggacaagtccagccccttgtcgtcaaggctcttgtagaccgacgtagggaccaatagaaccccgtgcggtggagctattgtgaaggagcaaaaaagtgccctggttctaagaggacgtcttaggggaagtgacggctgagttgaggtggatccggctggcgatgtaaggttcgagccatataaacccgggaaccgggagcccttgacgacattgttccccgagtgcccggagtctgcggctttttttggggtggtggcagctggcggaagtgacgggagaggggtggggccgcgagagcggcggaagtaggaagccgaggtctgaattgcgcgtggtggccatggcggccagcggggctgtggaaccagggcccccgggggctgccgtcgccccgtcgcccgccccggccccgccgcctgcccctgatcacctgttccggcccatcagcgccgaggacgaggagcagcagcccaccgagatcgagtcgctatgcatgaactgttactgcaatgtgaggcgggggcgcggccgcgagcggcgggtgggtagggcttgtgccgggacgagccgggggtcagacccagccaggtgggcccggccggggaccccgaggagcgcccggagatagtagggcggggaaagggagtttcattgatttttttccttagctactttctacttcaccagggcatgacgcgcctcctgctcaccaagattcccttcttcagagaaataatagtgagctccttttcctgcgagcactgtggctggaacaacacggagatccagtcggcaggcaggatccaggaccagggagtgcgctacactttgtctgtcagggctctggaggtgagaggacctcagagcaggtacctcagtattctagagagagattcggtactggggtcagagctctggtccaggcggtcggatcttaaataaatccccctatgtctcctagccttagtttcttttgtaaaatggtactggtgttgtcatcagctcaagtgagataagatttgtgaaaatgctttgtaaatgttgtctgagggtatgaaatgagcagtagttgttatgaatagtattctcaccatttggacagcctggctcacaccacctgtactattcgaaaggagcagtctggaccctgtggagtggcgagagggagagacagctgggctgcggtctgatggaaggggacggtctggttattacttgaggagaagctgttctgcagtctctttgtactgaacgtattcttttcttcccaggacatgaacagagaagtggtgaagactgactctgctgccacaaggattcctgagctagattttgaaattcctgcctttagccagaaaggaggtaagtttaagatcagagtatttgggctgttcgcatgtagctagaatcttaccctctttcttgcaaaaccccctggtgccaggtctagtggtttgtgtatgggttctaccatgtaatcattgtaatcttcattagttaatcatgagtggaaagcttaggtaaaacagctcgagatggtagaaagtaatggtaataccatagtgtgtttattgtagaattttagcatcactattatcagacatgaatggaaccttgcacctggccttctcattttacagatgaggaaattaagacccagagagttcaagtgaattgcccagggtcatacagggacttgatagcagagctgaggctaagtcacacttgtcttttgttcttgttcacaagctctgaccactgttgaaggattgatcacccgtgctatctctggcctggagcaggaccagcctgcacgaagggtaagctggggttttctgattgtagcagctcaaggaataattgcttatatagagtgtcccatgtgtctgaagagtctctgactaagtgttggaaacttgctgggtaagcatatggtggtgctggagctgctagaatgttgcatatgttataattattgtcttttccacttccctccctcagtatatattcaggcctcatcagacaatataacattacctctgaagagcctttcctaatccccttgggttatctgtgtttcctgagtattccactgcatcttatatttattatgacagaagtaatatattatagctgcatctttagttatttgtctccttaattggttggggagtcccatgagaggactgatatatatactattatatctctaacgtggcatgagtgcctgggctaaatggttgaatgagtgagaaaaatgaggctcacctgtcaccacccgtcaacttcaggcttgtttgctacatctcattgtgaagcctgagtcactctgtttccactcacactgagataggtggtgacttcctaatggaagtaacatgggcatgacaaggggcagggcataaggtacaccaccagggctgatgctttactctctgttcatggcaggcaaacaaagatgctacagctgaaagaattgatgagttcattgtcaaactgaaggagctaaagcaagtagcctcccctttcactctggtgagtattgagaggctggagttgcccttgattaggggaggagggaccattaagactgctgggattttccactgtgatgcctgcgtggtctgatagtaggtgatgtaggattgtttatgattttcttttgcagtgactctctcttctcccatttgttagatcattgatgatccctcagggaacagttttgtggaaaacccacatgctcctcagaaagatgatgccctggtgatcacacactacaaccggacccgacagcaggaagagatgctggggcttcaagtaagtggactgaaggatccagaagaatgctctggaattatcgcgcactctggattgcttggagtcagtggaacttattttgctgctgctgctctgtgtgccctatctcagcttgtttgtgcttctcatcttgcttatgtccaggacttgggtaaacttcacagaaaggatggcagtggctgtgatcctatcttaagtcctgttcttcctttggttggggctttactttgtggtttctctaactgatgaggtggcacctgacataactgatgaggtggcacctgagcatttctcccactccagggcagggtgttggggacactgaggtatacctgccaacttgcgacctgcatctttgctattttaggaagaagcaccagcagagaagccagaagaggaagatctcagaaatgaagtgagtcacattggctactggtgaggaaggagcagagttgtgggctgagactgatgctgttcagcatctgtcttccttgtcaccaacatagatgaccactagcataccaactgagtgtctttagcctgaacatcccatctgggagggctcaccctttgtcctcatgttgtgttccaggtgctccagttcagcacaaactgcccagaatgcaatgcccccgctcagaccaacatgaagctagtacgtatcttttgccaagcagttgggttgctcttcatctgactcaggcatgaagattataatcaggctgggagcatccctttcaaggaaaacacatgctttatacccagtgataaggaagatattatctttgattagactgtgtaccttgggttttgtgttcaaagagaaaattaggccatggtcactaggctgtgtgtgtgtgacctgagccatgccttcaaatgtgtgaacagggattgccatgggttgggaacttgccctcttctggaggactctgtgggctgcctcttgtgaatatttcctgcagttctgggaccagcatatgtttgtgtggggtttggctttgtttttccccagatggtggtcttgttcgcctggaagtagatttccttaactccgttttccagaaatccctcactttaaggaggttatcatcatggctaccaactgcgagaactgtgggcatcggaccaatgaggtgggtgggcgccattatttgggaagaagactagcttaagggtaacccccttttaactatcattcttcctaagtacttcagtactcgggggcagggggtgtagagtagcttgggtctttttcttaggtctagtgcagggtgatgagcccttgaaggaatgggaattagcctagagaagaaaagttagagattctataacttgtttaagacatattaaaggttgatgatatggaagagagaagtcttttgacccactcctcagaggtgtggaagttcagggaggcagactttgacttcacaatgaagaactcaatagagctgtctgaaaatagaatgagctgccttagagaattataaattcccttcccctgagtgtatctgagtataagctaattaaattctttaaccagaatattataaaggagacccacacattggatgcagtggatgaaaggactgcaataaatgcttaaaagtctctttcaaccccaaaagtctttttttttttttttttttttaagggaaattcagagtttattggcaatttgggataagttttggcacgttaccagggttgggcactgttcctggccagctcttggtgtttttggtttgggcgactgctgtttagaaggctctttctttggtagctattaatgaccctaactgattggatgtccaagcctacactccaggtctcctgggtaccaagtgaggctcaggtcttctctctgttgctcctgactgttatcgatgcaggtgaaatctggaggagcagtagaacccttgggcaccaggatcaccctccacatcacagatgcctcagatatgaccagagacctcctcaaggtaacagtctgcttgggaaagtcactgttgtcctcataagttagataaactgtctctatctaggggaagttgtcttttagaaataatgttcagggacatgaggtatctcctacttccctggctttgaggacccctgagcagagggagttaatctaagattggaattgcaggtttagaaggtgaccctgttagctctttctgatgacctggtagctttttggcctccagtgtctatctgggtcctcttctctgtctgtcccctagtgacaggatacctgacagctcgaagctatgactgagtttccctttattaagttggatctattcccgtggttcagatggacaggagtgagtctcaagctttaacaagggagtcagagaaaactgagacagctgcccttgtccgcagtctatcactgctggtattaattgggcaggaggcctctggttggctttctttgcttctgacttaaagcttaaactgacctctcatttcacagtctgagacttgcagtgtggaaatcccagagctagaatttgaactgggaatggcagtcctcgggggcaagttcaccacactggaagggctgctgaaagacatccgggaactggtgagtcacttgagtagtgtgtaaaccacctttgagcaatttggctgtgtgaagagggactggaagacttcagacatcctaattttcggtgctggtaactgtgggtttctttgccttaaagctggtttaagagacttaaatattactttggatggtgaagacaagtttatgggaaatgttgaatggaaattccaggcctaactttgggactttatagttattttgtggtcttggacaagtcatttattttactgtaaagttaggggatgagggtccttctagcacctaaaacttcccaacataaaaaacatacatgtatgggttgacttagcccaaatatttatctccttgatgaattatttcacttgattctctgttgggcattttgtatttgtaaattgtactttttctttgcatttcttctccaaattagtatcagagatcatattctaagaacatgtttcgattttgttttacattgccagttttgttatcttggcacattatcccatagactcctaggagtgactgccttggtcagaaaggtggtgtgggctaaatggaaaccaagtttccctgacagggcacccactgtcaccccctgcctagtttatggtcaggcctaacaaatataaagccagtattcacatagtttggattatcatttattgcaggtgaccaaaaatcctttcacactgggcgacagttccaatcctggacagacggagagactacaggagtttagccagaagatggaccaggtaagaggtcactggccagtcagtacttagaatcctggggctccagtgaacaggaggctgcatgctcatgtcttgccactttttgaacatctaaccatcatgttggggattcttcttctggttttcttttcttttttttttttttggagacagagtctcgttctgttgcccaggctggagtgcagtggcgtgatctcggctcactgcaacctctgcctcccgggttcatgcgattctgctgcctcagcctcccgagtagctgggattacaggcgcacaccaccacgcctggctaattttttgtatttttagtagagacagggtttcactatgttggccagactggtctcgaactcctgacctcgtgatctgcctgcctgggcctcccaaagtgctgggattacaggcgtgagccaccgcacctggccctcttctggttttctatattccatgtcttctttcttgtttttctcccttattttgttagagcatcttctccagtagcttcctgggaaaaagtgcatgggaaataaaatttctgggaccttccacatctgaacatgtctttattctgttatcacactagagtgattattggctgggaatacgattctaggatagaaataattttttatcagaatttcgaagactgcactatggtcttccagtttggtaaagacattgtactatagtcttccactttggctattgagaagtctgaagccatttgattcctggtcttttatatgtgacctagtcttgtgatctcttcgtttcctttgatgtgaaatttcagggtgtgccttggtgtgggtcagttttcatccactgggctaagcacttggtagatgtattttttcagtctggaaaactcatgtggttcagttctaggaagctttcatgaattattctattgaagattatcttttttctgattttttttttctgttctttctgaaaactcctgggttttagatattagacttacttgtctggtcttctataatttactctttttttggtcctatttttggtttcttggtttttttgttcttctttgggagattccctcaactttattttccaacccttcaattgaggttttcatttccactaaaacttttattttccaaggagctttttaaaaaatagcatcctgtcttaaggatgtagtattttttcttcttgaggattattattttttaaattttttcagcatgcagtttatatagcacttattgatactgatgttggatcttttttgacattttattctccttgcatcctttttttccccatttgatttcccatctttcagtgttaagggctttgctcagatgtctgataatcattgattgtcttcttacaagtagcgggctaaagacctgagtagggggtgagccttgttgacatggggcttattacaggggaatttaacttagctgttttgctgaggtggcctccaatgccagaattgttaggtctttcctcttgggctagtcagacaaagaggctatgcctgccagacaagaaccaacccaatttatattacagaatcctccaaagttttaagaattggtgaaattaggtagctctgaaagtagaggggaaaggatctaaaataaggactgttagaaaagccaacatcagcaagctgtggaccagggggtgctggcccagttgaaggtagagggaaaggactcaagaacaaggaggttaaatgactgtggctgctccatttcctggcctgagggtaaagacttggctggcagtgttctgagagctgtgggaagggggctggggtgtgtatgtattctgtttcccataagcttacattcatttaacctacttgttcttgattcctttccctctaccttcaactgggccagcaccccctagtccacagcttgctgatgttggcttttctaacagtccttattttagaacctttcccctctactttcagagctacctaatttcaccaattcttaaaacttttggggattctgtaatataaattgggttggttcttgtctggcaggcatagcctcttttttttaaaaatttttctttactttcatttatttcatttctttttttatttatatatatatttattatacttaagttctagggtacatgtgcacaacgtgcaggtttgttacatatgtatacatgtgccatgttggtgtgctgtacccattaactagtcatttacattaggtatatctcctaatgctatccctcccccctcccccgacatagcctctttttaccaggggttatgtaaaatttacttgctgtgttctctctctcctttagatcatcgaaggtaacatgaaggcccactttattatggatgatccagcaggaaacagttacttgcaggtatagtagaccttccctgatgtttcatagagatacattttctgatcttccttgaatatgtatcatttccctagaaactgaaagacttgattctaaatggactgctttttaccaaggaggcagaagttagaagatacataagggagaaaagaggggggtgtgggaggggattttttttttaagtgtatgtaaagaaggaaaaaaaaaggatactctgaggccccttttttttctgtagtcaagctcttgtcccagttctggagttttcattttgcaaatctgattatggattgaggagaaatggtaccttgtccggaggccaagataggaaactgataggctttaagttgactgaggagagagtctggtgattgatgtacctttaccatttattggatacttaccatgggtcaggcaaaactcttctaagttctttccaagattaagaccatttgtcatacttttcataagaaggtgcagatataacaaaataggtggattgttccctgattctggggctggggtcttctacccctggcccatgtgggctctgttgctacccactagttcagtctagccatgggaaattggaggccaataaaaagaaaaaaacccaaaactcattctgttcttgggaaggtgggaattggaagctgcctgcagagagggggagtggcacagcggcactgattgtctctccttgaccttgcagaatgtgtatgcgcctgaagatgatcctgagatgaaggtggagcgttacaagcgcacctttgaccaaaatgaggagctagggctcaatgacatgaagacagagggctatgaggcaggcctggctccgcaacggtagcagtgggtggctcaagggccagcctccagcgctgctctttctgtaggttatttattagtattggatgaaggcgaaggctgggagtgtctttcccaccagcccttgcccatggtggggaggacatctggtctgagtcagagatctgtgcacactttctaaacagcttgtgatgcaagtgtgagcctattgtgttacttgaccttattttggaagttttgaattggcctaggaggaaacccagaaatgaaccaggggtatgtcatcacttttttcatatcaagtcctcaccctccttccacataatgctctatcctctaaggttggaactctgaagttggagaaggtggaataaagttacacctggagtttgttgttggttttgagtatataaaatactgactttgaacaggaaaggagtctcctgaggggagaccgagtccagcacaagtgaaggagttaccattgaaatgatgactttcaaacagcactggcctctgtattgaccctatccttgtttgatatcatgctgttagaattcagcctcctaaagaaaattttcccgtgtatctactgtaatttggggattgcagctggcatttaattcattccttcagcagatgcttgcttatctactttatgccaagcacaccacagaacagaggacaaaataaatctctttcatggaacttgaagtctagtggggaaaaatgacaataaacagatgaggaaaacagtacatcggatggtgattagtgtcatggaggaatgtaaagcagggaatggggctgggttgtgcaaggaaatggggagtagggaagatcttctgaacaagaaggtgataatttgacatttgggcaaagctgaggaggaggtcagggagtgagctgtgtgggtatctgggggaagagatttcctggcagagggaactgcaaatgctagagctgtgaggttagagccagtgggggtacctgatcagttagccctactctcagttagaaatgttgaaacacattcatccagagagaaaggaggctctctgcagttcttctgaaagttaaggaccagtgaacactctgctgcttgggccctggacttcccagccagtgttctttgcactgtagtctcctgcctctcataaacaaaaggtgactcctgtggtgcagactgcagggtatttgttcaataaagaccagttcattgattaagatgttatcacaaaccttattgctttgtgttgcaaacttttttttttttttttttggtagagacagagtctcgcgctgttgcccaggctggagtgcagtggtgtgatcttggcttattgcaacctttgcctcccgggtttaagcaattctgcctcagcctcccgagtagcggggactacaggcgcacgccgccacacccagctaatttcttttgtattttagtagagacggggtttcaccgtgttgcccaggctggtctcgaactcctgagctcaggcaatccgccagccttggcctcccaaagtgctaggattacaggcgtgagctaccgcgtccggctgtgttgcaaacttaactctgaagaatgagttagagatgatttagcaatcagacatctactctcgttgctctgttaaactactctcaaaggtcacaagtgattttccacttggaaagtgttatctatttctgtcacatttgatagtggaccatctcttcttaacccttcttttggctctcgagatactgtacccttggttttcccttcactttgaatatttcttatttggacactttgcctcctcccctcttactaaatttgtttctgagagttctgctttcagacatctctccatatcagattctttggcatcttactatcatgtgggactaacttccatacctgtatctctaatttctgtaagtgagggcgagagtaaggtgtttggagactcaggttagcaaatgactcattcaaactataaagcttcagtactttgggcaaacttctcacaggcaactattctaccctaagagattatctctagatatgtttccagctgacttgggaatctaagtttaccagacaaccctctgatctatcatttggtctcttaagtagacttccccatagtgtaggtgtgctctgttaagataaactgcagtatcaggttcattttccattgtgaaggaaaaaaaatggcactctggcttattactgaatctcttgtgagtttggttggtgtgaggcaatctcccagaatccacccagttcctaggaaatgatgacctgagtttctgaacttgaagactgaggaccaggtgccagactgggataacccagctaaggtacttcctgccctttgatgacaaggcattcttcagtttcatacccttctcttgaattatctttcagctcctttctcatttcataaagtgtgtcaggatgggagcttacatatagttaactgagtcttgctccacagcacccagcatatgacacatggttaataaaatctgttaaaatcctccatcatagattaaaccctattaggacactagggataacatctataggtaaaaagatagggaaagggtctcgctttaaggccagggtaggtgagtcaaagtgccaggaacatgtggattcagtttcacagccatgagtaacatttaagtggaaaaggtttgtgattgtgttaaaaaaggaaagtggttcatgcatgagggcacgcctcctactttcatgggtcaagtatgtgaaatgcagccatgttcggtcattagacgtgtggacgaacatacaggtaggtactgaatgtacatctgtgtcatgatgagtggaggaggggaggtgagtgttgggagcaagtatgaagggcagttgacttggccaactgaaaactttaatgatggcaacactttcattctaacatcagtatatttccttctttgtacacctgtggtatgtgcctgatgcacactgcagaaaatggtgtctctgcttacatttagggcagtggtggctaagactacttggctttattcctattaaaaaatgttttttacacacttctttttaaggcaaatggcagtgtttccaactgtggagagaaaaagctgcttattttgtgcgttcacctcagtttagtattggcacttccaatgcttgttttcctagtctgtaaaatggggataattaggataaacaaaattatccccattttacaggtgacaaaacagcctcagagaggttacttccagaaggaagcagagctttttttttttttttttttttttttttttttttgagacggagtctcactctgttgcccagactggagtgcagtggcgtgatcttggctcaccacaacctctgcctcccaggttcaagcgattctcttgcctcagccttctgagtagctgggactacaggtgcacgccaccatgcctggctaattttttttttttttttttttttggagacagtcttgctctgtcacccaggatggagtgcagtggtgcgatcttggctcactgcagccttgcctcctgggttcaagcaattctcctgcctcagcctcctgagtagctgggattacagacgcccaccactgcacccagctaatttttgtatttttagtagagatggggtttcaccatgttggtcaggctggtctcaaacccctgacctcgtgatccacctgccttggcctcccaaagtgcagggattacaggcgtgagccaccgtgcctggccagaagtagagcttctaagaggcaggtgtcaaacccatgccagtcagactctgaaacctgaggtctgaatattttaagctcagctccacaattactgtatttgttctattagatacaactgcagagagaggccaaagctcagagcaggccattggcacggcatctgatcctgcttaactgccctgcagcacagggtggagacacttgggccttgtcaggctaagggactggggtaggatgggtagcagtggaattgaagacaaagaaaacaaatgggcagaagatgggtgggtggaagttcagagggctatcctggcagtgttcactggagcagaagtgtgaggctttctgaatgcctgctgacaccagggtggggcagggctgtgtagtgtgagatgcaccaaattggacgagaagccaaggcaggcttgtgtatggggtaccctggggccctccgcctagtgaagccctatcttctcagcccagcgtcctcgtctcacaagccctcagagccccagcctgctttcctttctgggcctgtgctgcaaccccctcaacatggcccactgggtaaaaattcatgcttcaagactgatccccaagcctgttttgtattatataagtaaagaattttttaaacacttatccccagataatgtgtatttatttataaagtatagccctgtctgcttgactaatatattaaaaaagtgacctttttgactcttggtccaagaaactattgtttgcacttttgtgtagttgtgtatagcatcctgcaaattctgtcagaagtctgtgatttcagtaacactaaaacttctctctcaaatggtacaagttttcagttttaagacaagttctgggaatctaatgtacaacatgttgactatagctaataattctgtattgtttacttgaagtttgctaagacaaatttgcttaagtgccccccccccgaactactacataaattagtgaaggatgttttacttaatttgattttggtaatcattacacaatgtataattatatccagtcatcacattgtacaccttgaatatatataattttttgtcaaataataaagctgggaagaaaacgataaaactgacaatctaacatatatgatgaggtaaaaagatctggtatttcaagaaatttggatgacctcttgcaccccacttcgctagcctctactccctagagtcatgttctgactatgtgaagttgagcaacttccttaatcttgccacgcttcagttcttatctgtaaaatatgataatgaaacgatttgagaattgtgagggttaaatgagataattcaggtaaagctctaaaccccctgcaaagaataaagtcttttctttccccccaaaacttgagtgttagaattcattctctctggtttctctcttttactatgctaaaaaatgatccaggtccgtccttctcattggcaagattgttttacagtttccttcaagagataactgggggctacttgtaatagaacactagaagagttacacaggtcttagaaactacgtttttcatagtaattatgttggtttgcacttcctggcttcctttcttggacggtaggtacctggaagcctagtggagcagattccccccacttaaagatacttataaaaataacgaacatagttccgatgaccatgtgcccaacaccgctctgcgcacttcacacagtaatatcatctcagcctgtgaccgttccgagttaggtgccgggattctccctgttttgtgataacttgcccgaggtcgtacagctagccagctatggaaccagtattcaaactcggctacgctgcagcccgtggccctccactttcttgctcgtgctctggagtttaagcggccgatgggggcggggtcctttgggtcaggttgtggcctcatagaacagcgcttgctgcggttggatggatgggtgggtgggtgggtgagtgaatgaagagaagaaacctggtggcgtcgtcccagggctgtggagcgccccgaaggtgcgcacgtccctggctagcctgcgagcgcgtcccggtggccgcacctgccagccgcgcgattcttagcactctccgccacttccggccgtggccccgccctgtcgggtggctggcgtccgttacgcgttgaggcattttgtccccagcgccgacccgtctctctgcccccgccgctgccatggcggcagctccgccgctttccaaggccgagtatctgaagcgttacttgtccggggcagatgccggcgtcgaccggggatctgagtccggtcgcaagcgtcgcaaaaagcggccgaagcctggcggggccggcggcaaggggtgagttggtaccggccggggcggggcctgcgacgtctagccaccccttcccaactccgccctcggccgcccggcagttccttccctcttggcgggtctctaggcttctccgccgacccctgctcatgctcagggccccactcacatcgcttgaggccccccgccttgcctctgccgccccgcttacatctcgagcctctgagcagtagcgtcagtgcgttaaagccggggtgtaactttgattactcttttcccgccttggtagacttccgtcctctcccatagagaatgaatattgtgcctcgagaataatggagcggattcaaagcctctgtgcttggatctttagggcccggtacctgcctgtggggattcccagatcccaaggacttggttctgaatcagagacctttgatcacctcctgttactcttagtaatagtcctaacgtgagaagctaacatttatatagttatacttttcttgccaggcaccattataagaaatttaaacatatataagtttatttaagcctcaccacactaccatgaagtgggtattattttcatatgggacaactttgtggcccagtgtagtgggccctaccaaatcgttttccacccagaaccacagaatgtgacatttagaaatagggtctttggagatgtaatcagttaagatgaggtcatacttgattaaggtgggtgccaaatccaagatgactgtttgccttttaaaaagagggaagtatggatacagacacagcaacaaaggaaaaacgaccatgtctagatgaaggctgagattggattggatttatgttgccaaaagccaaggtatgcttggggctaccagaagctgggagaggcaaggaaagattctctgctagagactttccagggaggatggccttgcagacacctggatttcagattgctagcttccagaactgcgagagagtttctgttgttttatgccacgcaatttgtggtactttgttacagtaaccctaaggcactgagatacccagtgaagtcacattgttctaaagaactagtccaagttcttttctggtttggtgcttgccttattattgtcttcgtctttctgcctcgctgacttgtgtggatcctttggaggcagtgccatgtgatgaaagtagcttgagtgttggagcagcactggtcagagttcgaatcctagctgtgccactttataggctgtgtacccttgggcaaggtgtttcatctcactgagctgcagtttccttatctttaaaatgagaataataataataatggttttattaagtggtggttgtgattagagggattgcattaagtgaccctggctttttgaataacttcacatcatggcagtcattgttattatttgggctcaactatatagtttatccacgtaacaaacatttattgaatatttcgtattccagttactgtagggtgttgaggatgcctctgtgaacaaaaaaaacatagtccctgttcttgtggagcttatagattaatgggaatacaggctttaagcaactaattacacaactaatcgatatattttctttatgataaatgcaatgaaagtattccaagagtttttaacaagggttcttagtcttgagtgagtgtgggatggtgagggaattgggtgaagcagtcaagaaaagtttgccaaaggaagttaaatttgtgttaggacctgaagaatgaggaagagtttcctggaatgagaaagagtttcctagcactttgggaggccaaggcgggtggattgcttgcatctaggagtttgagaccagtctgggcaacatggtgaaaccctgtttctacaaaaaatacaaaaaagtagtcaggcacagtggcacatgcctgtagtcccagctacttgggagactgagtagggagaataacttaaccccgggaagtcaaggctgcagggagccatgatggtgtccctgcactccagcctgggcagcagtgtgagaccctgtctcaaaaaagaaaaagactagggccgggcgcggtggctcacgcctataatcccagcactttgggaggccaaggcgggcggatcacgaggtcaggagatcgagaccatcctgactaacacggtgaaaccccgtctctactaaaaatacaaaaaattagccaggcgtggcagtggtcgcctgtagtcccagctactcaggaggctgaggcaggagaatggtgtgaacccgggaggcagagcttgcagtgagctgagatcgtgccactgcactccagcctgggcaacagagcgagactccgtctcaaaaaaaaaaaaaaagaaaaagattgaaaagagtagatcaggcagagggaaggaactgcatatgtgaaaactgatgtgataggaacttggctcctttctaagcaccaaaggatgaccagggaggtgttcaaccaaggaagtagcagtaggagatgaagttgaccttgtagacaaagactggaccatgcaggatcctgtagaattttgaactttattttaaaaccaatagataaatgtttttatgctggtatcctagaatgactctttttgatcttcttattctcatttagaatgcggattgtggatgatgatgtgagctggacagctatctccacaaccaaactagaaaaggaggaagaggaagatgatggagatttgcctgtggtatgtatctttggggctgtcaggattttgaaatgaagcaagcttctcaatttcctttttttttttttttttgagatggagtctcgctctgtcacccaagctggagtgcaatggcatgatcttggctcactgcaacctccgcctcccgggttcagatgattctcctgcctcagcctcctgagtagctgggattacaggcacccgccatcatgcctggctaattttttttttttaatttttgtagaaatggggtttcaccatgttggccaggctggtcttgaactcatgacctcaggtgatccacccaccttggcctcccaaagtgctgggattacaggcgtgagccattgtgccaggccaagcttctcaaatttatttttccagcttagggtagatttgtaggggggttagttgagaacagaaaagaggtgacagtttccaaagagctgtttaaaatagttttctttgctgatgcctatacaccaacttgtctctttatggagggtccatcagcctagaaatgagatggatcacagagagaggacttctcgacatcagtgttagagtaatgatgagacttagctgtggttcattttgttggaatcttccttaaaaacaatgctgaggttcagttatagggaactttttatttgctccttttttcagtgctttaactaagctttcaaattgtatattcttgcctaagtactggagatatgtagattaatagaacatagctgtattttagagattataccctagaatgaaaaactgaatttcagataaatttcaacagtatggtaaatgttggggatgtttcacatacgtaccaagtaccattagagcccggggaggtagcatactttgtattctgttgggagatatcctgaaagatgtaaatactgagttttgaagggtaagtaagagccaggtggacaaaatcataggcaaatgacctcccagtggttcatgaaaccattgctgagaatttctgagaaacaaagtggaaaggagagctgtagataggctgaattccactaaagttggtaaagatggattccagtgagtatttaatattaattcccagcaaaattctttttttttttttttttttgagacggagtctcaccgtgttgcccaggcttgagtgtagtggtatgatctcggctcactgcaagctctgcctcctgggttcacaccattctcctgcctcagcctcctgagtagctgggactacaggggcccaccaacacgcccggctaattttttgtgtttttttttgtatttttagtagagacagggtttcatcatgttagccaggttggtcttgatctcctgacctcgtgatccgcccacctcggcctcctgaagggctgggattacatgtgtgagccactgcacctggccaattcccagcaaaattctaaaatagattatttaaaagagggcttgctagtaattagaagggaaatagtgatcttagggagccaatttagttttggtcagaagaagtaaatattaacaatgtttgttgtttttttttaattttataaaagcaatgcatatttattacagaatatttgggaattaaaaaaagtttaaagtttttaaaagaataaacacttgcatgttaccatccaaagataactgctaatacttggatgcatatctcttcaggtttttctctttagagattatacacgcttgtattttatttttttgaacataattgggatcatactgtacatagggttttggagtctactcctctgctaaataatatgttgtggatactctggtaagaccttaaaatattctttgagtgtatgatttttaacggttatgtagtagttcaatatatatattttatctgttctgttactggatacatggattgtttccacttataaaatatcatacatcgttgtacataaatcatttgtagaaatcagtttatttctttattcccataaaatgaaatgagtggatcaaagggtatggttcgctttagttatattcccatgttgctttccaaaaggtgctaccaatttacactcccactagaggtgatgagaatgccagtttgcctacgtcgtcaatattagagcactattattattattattattattattattattatttttgagacagggtcttgctctgttgcccaggctgaaatacagtggtgtgatcatgccccattgtagcctctaccttccaggtgcaagcaatcctcctaccttagtcctgcacgtagctggtaccacaggtgcgcacctccatgcctggctaattttgggtattttttgtagagatgaggtctctgtgttgcccagtctggtcttgacctcctgggctcaagtgatcctcctgcctaggcctcctaaagtgttgggattacaggtgttagccacatgccccaccaatattgaaggattattaacaaaacaaaaccaatagcaaacctatgctgatttagcaagtgagtaatggtatccagtttctttttttttttttttttttttggtggagtctcactctgttgcccaggctggagtgcagtggcgtgatctcggatcactgcaagctccgcctcccgggttcacgccattctcctgcctcagcctcccgagtagctgggactacaggcacctgccaccatgcctggctaattttttgtatttttagtagagatgaagtttcactgtgttagccaggatgatcttgatctcctaacttcatgatctgcctgcctcggcctcccaaagtgctgggattacagacgtgagccaccgtgcctggcccagtttctttgaacactagtgaaatgaatcattttttatatggtcattaggcttatgttgtgggtgtattttatcatttgccttttcacttagttcataattgtttttattattttttttttttgagacatattctcacactgtcaccaacgctggagtgcagtggctcaatcttggctcactgcagcctctgcctcccaggttcaagtgaacctcctgcctcagcctcccaaatagctgggattacaggcgcctgccaccatgcccagctaatttttgtgtttttggtagagacggggtttcaccgtgttgcctaggctggtcatgaactcctgggctcaagcaatccacccgcctcagcctcccaaagtgctaggattacaggaatgagccaccgcacctggccattttcttttataatattgtcttgcttttatgatggcccaaaaacctaaacaaaggaggctttagtgccactgctctctgcattggttaaaacagatcttgattactgaattcatttttgtaactgcaagatcaccaccaccaccagtaataatggtaaaacgattatgatgattacgatagcagtggcagcacaggaataattaataggtgtcatttattgggcatttatcatgtaccaagcatggctaagcttacgtgaattatctcattcaatccttatagcaatcctttgaatcccacttttacaaatgaggaaattgaggcttagaataatttaccaaaggttacacaaatgctaagtggccgagctggggtttagatcttaattcatcttgcccgaaacctgtgcttttaacgctcagtgagagatctcaaaactgggttatatgaagaatgatggtaggattttgggagaagtagagggctattatgacctggatttaaattccagttctgccacttgttagcttgtgacattgagaaagttactgaacttctccgaatcagtttgctattcagtagtgtggagcctttctttacaggattattgtgagaatcgagttagattatatgagtaaaccacaaaacacagtaccttgcacatagttgctactctgtaactaggatttattatgccagcaatcttgaaagtttgtcatgtggacttacatcacaggtcagaactagaatgaattcaaagaagttataggaaggcagatacggtactagtaaaactaagaactttctaacatttttgaaaaatgcagtggactgtgttgtaagataatgagttccttgtcaggaagggattgggcgagtgaccacttattaggaatactatagagaagattctgatttgttcagaagtttagaagatatgaaatgtggttttagctcttaaagattaatgattctgtgtacatctcagtggctctttttggttgtattcaagaaaacctatcactgtgactacctacaacaagaaaggattcatagaaacccaaagtccttgattagtgctaggcatgatggggggagccacctatgtttccactgttaatgtagaagtaaaagtgtaaaaggaatttagttgttagtttacaagtggctgttgtgtacctagcaggttaataaaaaggtttgtaatgaaaaaaattaacctttgcagggtaattgattaattagagaaggatctacaattaaaccaatacagtgacaatcagatgtttatggagcaaacataactgaagacccctacttacttcttcatgaccattttggatatatgtacgcccttgtgttaggaatcggaatggttgtaacttcaggttcttgagacagtgcttcttggctggatcctgtggatttcctaagataggaaatctttactctcacaggtggcagagtttgtggatgagcggccagaagaggtaaagcagatggaggcctttcgttccagtgccaaatggaagcttctgggaggtgagttccaacaatagataaatctaaatttccattagtaggggagatcttttttccttttctatacttttctttagaggaatgcatagtttgttttagcatgtcattgatcctgaaaaataaagcaggaaaaaggaagttggacttcttcagaggtagtaagacataggaacttatagaccagatattcctctttctcttatattttggcattaggattactgaggttggtgactaatttggaggcctttgagcacccattcctctttgactcagaataggagagcacttggtgtataggcaagactttgggaggagtttacaagaaacaggagcagaatttggggactttagctaccactatcctcttccacaatgaagcaactcatttctggggggtcaaggaccagtgtattttgttctgtttgtgttagtaaatttctctcttctttccttaagtattggtgttttatagtgcattaccctccacttgcccactttggactcttcctaagctaataatcttaatttaactctctttaaaaaaatttgagtccttgagaattggataaaaagcatttgtttgtcacatctgtttgagcatagactagtcttatttgccatgatattcccctatctagcacaaggagctacctttaataaatatttgtagaattgtttaattgctagtaggtatttgatttttgtagcctaatcagaattcttttccaaagagaatcttttggtctccatgtctgtttgtctaggaagacacacagacacacacatacacacggtgccttttcttgtgtatctgaatgtttagaaggtcagtcacacacacacacagtcacactcacactcactcactccttttcttctgccttgctttcactcccccatcccaatttgttattggaagtacgcttaccaatctctgacccttgcttccttcagatttactgttaggccccccacagacatcatacttttctctgtggagtggattccaatcagatagggctgttccccaaagtaaaggagttgacttggtttaattcatccatttaacaagtattcatcagttcctacccagcttttatttgtatttgggctgtggagctcaatgacaagaaatagaggtagaggaagaactggattttgaaactgttccagttgctatagatatttcattctgtggtgcctttgaatatggaaggtctcaaactatgtattttaactttaaagctttacatattttaaagattgcacatggactctggagtcaaatagagctggatttaaatattcctctaccacttattagcaatatgactttggcaagttaatctttctgagtatcaacttcctcatctataaatgaaatgacagtatttatgtcctaggcttgttgtgagaattgaatcaggtaatctctatgaaacactggcacacagtaggtgattggtaattacttgctcctttcttttttgtggtgatttttcccctctctaagaaagagcacagtgaaatggattaaatagttaacagcattttattttgatattcaccgttttagtcaatttacctattgaaagaagagtgagtacaaactataaagtatctaggcaaaatatcagtaggagccactagctgggaactctgcacaaatgaggttttagaaagaacattggactgggagttagaagacttggattctagcctgtgtcttcattttaggagctgtgaccttaggtaagaaatcactcagctcctcagctgaaaacagaggctgtggcctctgttttcatgctgtaatacaagggtacctgccctgcttaccttaaagggtataatatgaaaaatgtgctgaaaatgctttgcaaatttatgacttggtaggcggaggggttattcttaagatattcatgagtggtggtttgctgcccctaagcaagaaaactgtgcaggccttttgggctttctgagcagttctgaggtatgtgctactggagcatcttttgaaactgggtgtgggggtggacagagggacagagcatttgtgggaatctgactctttgttatttgtgtttaggccacaacgaagacctaccctcaaacagacattttcgtcacgataccccggattcatctcctaggagggtccgtcatggtaccccagatccatctcctaggaaggaccgtcatgacaccccggatccatctcctaggagggcccgtcatgacaccccrgatccttctcccctcagaggggctcgtcatgactcagacacatctcctcccaggaggatccgtcatgactcctcagacacttcacccccaaggagggcccgtcatgattctccagatccttctcccccaaggaggcctcagcataattcttcaggtgcatctcctaggagagtccgtcatgattcaccagatccctctcctcctaggcgagcccgtcatggttcctcagatatctcttcccccagaagggtccataacaactcccctgacacatctaggaggactcttggctcttcagacacacagcaactcagaagggcccgtcatgactcccctgatttggctcctaatgtcacttattccctgcccagaaccaaaagtggtaaagccccagaaagagcctctagcaagacttctccacattggaaggagtcaggagcctcccatttgtcattcccaaagaacagcaaatatgagtatgaccctgacatctctcctccacgaaaaaagcaagcaaaatcccattttggagacaagaagcagcttgattccaaaggtgagcatatgactgtccatgagagggatgtatgtatccctgggagctttcttttggccgtttagttgatttattaggtataagaatggaatttctttgggaaagttttgaaaagtgagaggctgggtttgggagagttgaacaattatggtggcagggaggaaggttttcatatgtctttttatgtgctcgctccaccccatataaaagtacaatttatttgttgcagaagctttggaatgtacagaagtatataaagataaaatgtgacacaaaattctgtcattcagaggtaattattgttagtgttgtggccagggtcatcttagtcttttttggtttgcattttttatatagttgagctctggctgtgtagaacatttgtattgcatgtgcaatcttgcctgctgtcctttcttaacgtagtcttcctcaggccataaaacatctttatgttagtataatttttaatagtgtttaactacaaatttaaccattttcctaataatgaatgtttttggttatttctaagttttttcttctgtaattctataatgaacatccttgtacataaactgttgtccccattttggattatttccttatctacttctagatagatttcctcgaaattcaactacttctatataagtttcctagaaagctgagaagttgatgtactgggtcagaagatgtgggcgttttaaagactgtcgatacatattgcagaatgccctttgggaggtgacttgtgatgcttatacagtcgctagaacttaggcctgtgcagtgaaattttttactccctcttttgttcctaggaactattttaattttaaaattaaagtactttgttatcctttagtccaaggatatcagactatattccaggtatttctgacttttgccacactatggagagctgactacaagttattttgtctttccatgggtggggttactgacatagaggagtgtgtggagattttacacaccagttacagtcagtggtcacttgggaatagaatgccatattctacctgttggtcttaaaagttgtttctgactttgctcttctcctgtgccatccatacttatgtttaggttaatgataattcttcataggaaaactaaataaatatgtacattagcttggtaagttatctttttagattcttccaaggggattaaagaactctttggccaggcatggtggctcaagcctataatcccagcactttggaggccgaggtgggcagatcacctcaggtcaggagttcaagaccagcctggccaacatagcgaaaccccatctctactaaaaacacaaaaattagctgggcgtggtggcacacacctgtaattccagctacttgggaggctgaggcacaataattgcttgaacccaggaggcggaggttgcagtgagctgagatcatgccactgcactccagcctgagactgtctcaaaaaaaaaaaaataataataatgtaaggaattctttaatttctaggttcttctcccagccataggagatttcatgtaagttcttcacctaggggacatcagagtcacacagattctttgctcctatccaggtgactgccagaaagcaactgattcagacctttcttctccacggcataaacaaagtccagggcaccaggattctgattcagatctgtcacctccacggaatagacctagacaccggagctctgattctgacctctctccaccaaggaggagacagaggaccaaatcttctgattctgacctgtccccgcctcgaaggagtcagcctcctggaaagaaggtcaggattttcaggagccatgtccttcttttcgtgtgactcttctctctcagctttatatatgtcccagagaatcaagccaagaagaaggatgtagctctgagaagtgtagtaaatcttagttaaaaggacctttggaggtctttccttctgaaggcaaactggctataacccatcccagatggacttacagataccagatttcccaagtagcttctgcgtttatagcctcttaattctacaatctcattttaacaggtctagaaaaacttcctattttcttaggaataaaccgaggactgtaatttgcagagtgaagtttggagcacaagcattgtgtcaaacggatcacatacagaaattctccttctgccacttactgtctgtgagatttgggcaagatgcttaatatctctaaacctcagtttctttatctgtaaattgtggataatagtagtacctactgtgtaaggttgttatgataattaagccagtgtaagtaaaactcttagcacagtgtctgggacaaagtaagcatctagtgttagtgatttactagtataaattggactataggtctctgtatccagagcttaaggagaggcacttaaaatatgaaaccacttttagaaatcatgttgtctgagaagtcaatggtttgttttaaattcatgataaggcttgatgaattagccaaaaaaccccaaaatccatatgaaccttgagtaattatattgtaaagaacattggtagtagtaaaggaatcttatttgtaagtggttaagaaacagaatgatagagcctgtgatgtgcatttttctccccagtgcagacttatcaggtaaatcttrtctgtagaagactctgagaagactggtcctggcagggtagaccagcctgttctttacccaggaagtgagattcttcttttttagggaaaaaagaggtctctgtttcagctggacctcttggcattttatatatccatttagttcatggacaacttaatattattccaatttcgttgctgaaggatatcaagatatactgtgtgcttcattcgtgggctgatttgctatcttgatgctaagtggaaattagcaaaaagttttcatttgttagatcttctgttattaccatgaaatatatccacaatttaaggatcttaggtttgcttgagtttgtactgtaaatggaatattttatcagtatgggtctctaatggaaggcttagtgctttatactggtttctgatcagatgacctgagatagtcttcatgtgtgcagtttatatactgaaaagtcagaaatacaaatgcgtagccctccatttaatatattgttagtgtttctgcttattcttaaacttgatggttttgatgatggttttcttttatagtttttgaatcctatggtaattattgagaatataaagctagagttttaggtttaattattgctggtcattagacacaaatgcaactaactgtgtacccatggaacctactgtcacatcatctcattaatgttggatgctcccttttccgttgcataggctgcacacatgtattctggggctaaaactgggttggtgttaactgacatacagcgagaacagcaggagctcaaggaacaggatcaagaaaccatggcatttgaaggtaaaaatatgaaagtgcagagaccaatttaggctcatactgttttttttttttaatttagcttattgtacctgatattttgaacttttaattgctatcaaatttcagctctggttttatgcattgttgtaatttctcagtgaatcccagtgcttctttccttcttgaaaaatgccatttcgcccaggcgcggtggctcatgcttgtaatcccagcactttggtaggccgaggcgggtggatcagctgaggtctgtagttcaagaccagcctggctaacatgatgaaaccctgtctctaccaaaaatacaaaaaaaaactagccaggcatggtgttgcatgcctgtaatcccagctactcaggaggctgagacaggagaatcgcttgaacctgggaggtggaggttgcagtgagccaagatcgcgccactgcactccaacctgggcaacagagtgagactccatctcaaaaaaaaaaaaaaaaaaaaaaggaaaatgccatttcttgggcccagtgccaatatgcaccaagatgttggtaggaactactttggtctggctgcagaagttcttatctagcattagaatcccaagcggttgatttgatctcttagaatgttatttctgattttgatcctgatatttgagtataattttcctttgcagctgaatttcaatatgctgaaaccgtatttcgagataagtctggtcgtaagaggaatttgaaactcgaacgtttagagcaaaggaggaaagcagaaaaggactcagagagagatgagctgtatgcccagtggggaaaagggtaagggaaccactgaaagggtaaacaagatggcagtgactggaacaagtcatttctctgctcttctgatcactcactttcttattatgccttcagagctgttatcagtaatgggaaatttggtgtgctgaatcttcttcctaggatattgatatattccacgcttctagtgggtattctgggaattttaccctgctcagtatttgccctagggtactagaaagaggagattgtccaaacttagcagtatggtccatctcgtgtagaagtggaaatgtcatacaggatagcaaacactcttggttcctttttgcccaggcttgcccagagccggcaacagcaacaaaatgtggaggatgcaatgaaagagatgcaaaagcctctggcccgctatattgatgacgaagatctggataggatgctaagagaacaggaaagagagggggaccctatggccaacttcatcaagaagaataaggccaaggagaacaagaataaaaaaggtgggacttctgggaatcatcagctggaggtgacttgtgaagagagaatcattaggatgctgatacatagctatatgcaaagaaggatttcccaaataatttaaattcattgtatttgtgagtttagattcaactcaattggtctttctatataaaaatttttccaggccaggcgcagtggctcatgtttgtaatcctagcacttcgggaggccgaggtgggtggatcaccttagagttcgagaccagcctggccaacatggtgaaaccccatctctactaaaaatacaaaaaaaaaaaaaaaatagccggtcatggtggtgcacgcctgtaatcccggctagttgggaggctaaggcacgagaattgcttgaacccaggaggcagaggttgcagtgagctgagatcgtgccactgtactccagcctgggcaacagagtgagactctactaaaaaaaaaggaaaattccacattgccatccagctctgaattaaactatgtcattaactgaatacttttttcttactcctctcattagtgagacctcgctacagtggtccagcacctcctcccaacagatttaatatctggcctggatatcgctgggacggagtggacaggtaagcctgggtatttcttacattttctacctgactgtaaccttccctaaccactcgtaagttgctccacactttatttcattctctgctttaatcacaaggtgaaaaatatgcactttgcttgcttctttttctgaggtccacaaatcctatttctcatgcttggaacactcctccttctgtttccttttcataatactttttaaagctctggacaaacacctcaggtggttcagataagacaccctttgttttggacattggttacttttaatgatattttgtaaggtttagaggggttgagtgatgagttgtagtctggagccttctacctgatatatctttaaaatgtagcctctgaatctttattcactttatggttttaggaggcagtcacttttaatgtcttccagtcctctaccccacctcatctgtctacctaccaacatctgtacccacgtactctaacttcctgttatgatggatgaatatttgcttccatccaaagtcagtttgtatactcacatattagatcccatctcctcttgcctactaaaggacattgttttagtatctagcacaataattcttaaactttttaatctcaggacccttttacagtctttttttttttgagatggagcctcgccctgtcgcccaggctggagtgcagtggcgcagtctcggctcactgcaacctctgcctcctgggttcaagcgattctcctgccctagcctcccgagtagctgggattaccggcgcccgctgccacacccagctaattttttgtatctttaatagagacggagtttcaccatgttgccaggctggtctcaaactcctgacctcaggtgacccgcccacttcagcctcccaaagtgctggtattacaggcttacaggattacaggattacaggtgagccaccatgcccaaccactcttatttttttttgaggcagagtcttgctctgttgctcaggctggagtgcagtggtgcaatctccacttcccgggttcaagcaattctcatgcttcagtggcctgagtagctgggattacaggcatgtgtcaccatatccagctaatttttgcatttttagtagagacgaggttttgccacattggccaggctggtcttgaattcttgggctcatatgattgtctgcctcgatttcccaaagtgctgggattacacgcttgagccaccgtgcctggcctacgctcttaaaaattactgatgacctcaaaaagcttttgttgatgtggattatatatattgatatttaccacattataaattaaaactcagacatttaaaaagtatttattcattcaaaaaaaaataaactaattatgttaacataattactgtatatttaaatgaaaaataattacattttccaagtgaaaaaacagaaggatggcattgatttacatttttgcaaatctcattaatgtctgacttaataataggtaacttgactctgtatttgtctgttgtgatttgttcagttgaagtatagaaagaaaatttagcctcacacataggtagttagaaagagaaggcatattatatttcttaatgttacacaaaaactcaacaagtgctggtttcttaaaggtgagctatgtggaacctgaaaccagatcaaagaactttccttactgttatattaatttttttaatactttgagtggatcttttacccatgcatgattttataacatcatgcattggtcatttgtaaaatattggtttgctgagttgttcagaccttccaaatgttgacatatttccactatacaatatcagaaaatcacttttgttaacatcacttccggtcttatcacaaaactctttaaataatgggaagttaaagagttcacagtttttcagaattctaatttacacttgaaagtttaaatgttatcactggcatttccattgcttgagctatttccatttaatagcttaatttctctgctgagatttcccatctgttcattatgagatattttcctcttcttgaaaatatttgtgatatgtaattggtgctttgaagtccttcctgtctgttaattgcaacattggattcaccacaggattggtttctatattggcagctttttcttcagtgtgtattacattttcctgtttatttacctgtctggtcacttttttaaattgttactggacatcatgaatgatatactgtagagactttgagttctgttacactgtattgtcagttgttttccttcaggtaaagctcactttgtacaatagctttttatagattccatcagattttgtacctagagggtcttccatctttgcatacagtttaatttcttactttacaatctagatgctttttgtttcttttccttgccctgttgcattagctagcacttcagtgcagatgttgaatagaagtagtgagagcagacatctttgctttgttcctaatctcaggaagaaaacatttagtttttacaacacatttattaaatgtgttgtagctgtgtaggttttttgtagatatcctttatggagttaaagttgccttctattcctgttttgctgagagttttgttttcaggaatgtatgttggattttgttcaaaatattttctaattatccttttggtttattctttgctccatgggttatttaggagtgcattaattccaaatatttggacttttctagatatcttactgctatttacttataatttaattccattttggtcagataatatactgtaatattttgatcttttgaaatttgttggccaggcacagtggctcatgcctgtaatcccaacattttgggagcccaaggcagacggattgcttgagcctaggagtttgagaccagcctgggcaacatggcaaaaccctgtctctacaaaaaaatacaaaaattagtcgggcatggtggcacacgcctatagtcccagctactgtggaggctgaggtgggaggatcactgagcccagggaggtcgaggctgcaatgtgctgtgattgcaccactgcactccagcctggatgacagagtgagaccatgtatcaaaaaacaataataaaaatgaaatttgttaaggtgtgttttatggcccagtgcatagtctatcttctgaatgtttcctttgcacttgaagagaacgtatattctgtagctatataaggtagtgttttataaaagtcagtgaggtcaaggtgtttagatcttagatcgcctgtattctgggttttttgtgtttgtatttttctatcagttactatcaattgtggattttttgttttagctggtttattttgtttttcaactctaaaatttccatttaatagcttacatttctctgctgagatttcccatctgttcattatgagacattttcctcttcttgaaaatatttatgatatgtaattagtgctttgaagtcctgtctgctaattgcaacattggattcaccacaggattggtttctattgacagctttttcttcagtgtgtattacattttcctgtttatttacctgtctggtcactttttaaaattgttactacgcatcatgaatgatacactgtagagactttgagttctgttacactgtattcctcttaagtgtgttagtttttattctagcaggtagttaacatagctgaactccaaactgtctcctttgcagtggaccgcagctacaatctttgctcagttcttttagtttctagctgccatttttttaattggcctgatggtattttctctgcacatgtgtcatttaacagttagccaaggatttgactagggtttgtatgtagattttgaggttcatctcttttgtagttccttctcttccaagatttcccccctaatttcctagctgttctgcccactttgcactaattcctcaagccagtaagcctgtggctttctgccttcatgagccatgctgtttgggaagttgccctccagcaaatctcttttcacatatagacttcatccagttcatacttacttttggtcactcttccacaccttcaaatacctattttttttgttttgtctaggttttatagttgctaactgaggataggttagtctgttcgggttactctgcaatgtctagaatctgtctccccaccttatatgggcctaaggcgttgtcttctgtccctcggggtgccgtcaaaatccaacagccaggtgtggtggcgtgtgtctgtagtaccagctatttgggaggctgaggcaggaagatcgcttgaacccaggagtttaagtccaggctaggcaacatagtgagaccccaactccagaataaaaaaatttaaggctcttctttctgatattggcagatatctataagttgactgtagttcctcatcttggcagatatctttagggtagctgtggttacaggctagcttactagttccgtttgcagttcactgtatgtaggctttttggtagaaggaggatttttcagaacactcatttgccatacatacagccagaagtagatctttttatagcacaaccctacagatgttccctgctaccgagtttttgttttttgttttttaatttttggacccaaaacttagaattctccctgacctagttttttttactagtatttaatttgtcttcatttttctcagttgcttttttccacttcttgttctgattttatcctagatttcttctcttatttccctttgtcacccttgctttctttttgttattcatctttcatcatcttttctagttttgtctcttttcggcctgctgcaagggaatatttccggaacagatacatagtatctgttggaaaaacccataagataattacccagcctctcttaattgaaagagaaacggggccgggtgcggtggctcatgcctataatctcagcactttgggaggccgaggcgggcagatcacgaggtcaggagattgagaccatcctggctaacatggtgaaacctcgtctctactaaaaaaaatacaaaaaattagctgggcgtggtggcgggcgcctgtagtcccagctgctcggaaggctgaggcaggagaatggtgtgaacccgggaagcggagcttgcagtgagccgagatcgtgccactgcactccagcctgggcaacagagcaagactccgtctcaaaaaaaagagaaactgaggcccaataaataagcagtttgcctagagtcatgcaatttccctgagaaagctggaattagaactctgcgttcctgattctctggtccaaagctctttccactgtgagttctcctgcaatttgttttctgattctgcttaggatttggtgtttgttattcatatgtcctttgtattatcatattagtgtaactctcttaagaccttatttccaaggtaaaaaacagtggtttccttggtgctttggaataccatccatgcttctaaggttggagaggatgccatttataataagcttcccttcttttttttttgagacggaatttcgctcttgttgctcaggctggagtgcaaaatggcacgatcttggctcactggaacctccgcctcctaggttcaagcaattctcttgcctcagcctcccgagtagctgggattacaggcgtgagcaccacgcccagctaatttttgtatttttagtagagacagggtttcaccatgttggccaggctggtctcgaacttctcatctcaggtgattcacctgcctcggcctcccaaagtgctgggattacaggcgtgagccaccatgtccggccaagcttcccttcttaaagccctctgttactcactccactcatcccttaagggaaagtcttagcatacatgttataagtgtaagcagctaggtagtaggtactagggattccatgattaaagagagatagcccctgagcccaggagctcactttcaatctagaatagaagacagacagtttcaacgctgtttggttagtgttactatagaagattttcaagatgttttgggagcacaaaggaagagtaagttgagccttaggaggatgtgagtgatcaggaagtgcttcctaacggatgaaatgtgggagctgagttttaagggatgttggtaaccagccaagataagaaggaaaggaaggatattaaaggaagagggtcctatatgtgcagagtcataaggctatgagacaccatggtgttaccaggtggtgggggtccctaagtaatttgctgttgttggatagcataaagcgtgagatggatgagagaggttggcagaccgtgatcacagaaggccctggaagcctatctgaggagcttggtcttcaccctagaggtaaaggggagacacggaaggattaaaaacagctctgcattttagatagacaaatccattatagcagtaatatgaaggatttgaaaggtacaagattggaagtagaacagatacaaggcttttgcagtagctcaggctgaaagtaatgagtgtctgaactacgacttgcaggcagtggtagtaaggatggttagtaagagaataggaagtggggatgtggtcaggggtgagctggtgggacctgtttgctgatttggggaagaggaaggaggagtcattggatttggcaacaaggaatgtcagtgatgacctgaaagggctagttccgttgtgtagtgaaacaatggccaggttctaatgtattaaggagtgaatgaaaagtgaggaaacaaatagtgaatacaggctttttaagaagtttagataagaagaccaagagaatgcatggtaattaaagggatttgaggtccactcactacctccttgaaattcacccctgtattgttttccatgacaccctactcctggttcttttctctgatcattcttggccacctttgcaaactcctcttcctctgtgcacctcttaagtctttcccagggctccatccataatttttagctgttctcactctatgtgctccctctggctgattcttacctagtcatgttttcaactatgacctatatgtacatgatttccaaatcagtatttgcatcttggtctggtgtatttagctgtttgttggatttctctattgatttagacttgagagatttcaaatgcattgtattcaaagctgaactcatcagcttctactgtaagcctgctcctactcttgtgctctctaacttgcctcctccttctttgtccttgtgtacctaatcaattagtgctgctaattagtcttactaattttgcctcctgtgtcttctctcttccgtccctctttatcattgccttcatcatctgttgcctggaccattgcagtgattttcctgccccagatcccctccagagtgatctctttgaaatgcagtttaagttcttgctttaaacccatcgtttctggatgaaatctaagctttttaccatggcctacgcagctcattatgcattggcctctgcgcctttccaactttgactcatggctgttccctttgtcatacttcaggttccagccataccagtttgcctttggtttgctgcacataccaggccacttcttatttccgtgcctttgttcttgttgctcattctgttctgaaatgacctccagaccctttctggcccttccatccccagtgctgtagttaacacagagcctttactgatcattcttgcccaaccctcatgtcacctttttatcatacctcccccaagctgattaagatacccattctgtttccatgacaccccatgcgtatttctaccagagtttatgctgttcttttaatttatttgtttaaatgtctctctcttccactagtctgggagttcatcaggacaggtgctgtgtcatactcattttcatgtagtgtctaccatggtacctagtatataatgaaaattcaataaatgtttgtgtaatgaatggataaaatataagatgtggacatcagctgagagagaaccaggcttttagagtggcaaacgtttgaaatagctgtcagagtgggggaagggtgtgaacaaggactaagaaaaggatgaatgatatattttgtccctttgattctattgcatatgccttaaatattttcttttcttttttgtttctttctttcttttttttttttttttttttgagacagagtctctgtcacccaggctggagtgcagtggcacgatctccgctcactgcaacctctgctgtccaggttcaagcgattctcctgcgtcagcctcccgaatagctggattacaggcacctgccactgcacctggctaatttttgtattgttagtagagacagggtttcaccatattaggcaggctggtcttgaatgcctgacctcgtgatctacccgccttggcctcccaaagtgctgggattacaggcgtgagccgccgtgcccggccatgcctaaaatattttcatatgactccttactttccccccttttcattatattgtgaactcttccctttcttttgaaacaaatgcccctttcagggttctagctgaatagtatgtctcttttgattgcagatccaatggatttgaacagaagcgctttgccaggcttgccagcaagaaggcagtggaggaacttgcctacaaatggagtgttgaggatatgtaactttcctgaggctgtgggggtggctgggctgtggtagtgggcataggcagcgagatatccagtggtaacagttgtctgtgctaataattggagcccacacagaccagcaacttgttgaatgccagttttgaccacagaagaatattcgagacctgatgtttggactgaggtacctgtacttcttgggtgtgacagcaccggctgttgctggctttcagaggaagcattgatttctcattgaccagggtttgttcttggtagggtttttctttttcttttttaaataaacatgtatttatttttttaaaattatcttcttaactgggtattctgttttgggaagaatacaggctaatattgaacctgtggggatttggggggtggtggttgaatttttcactaatctagaaagcagtgtgagtaaaaactttcttagtgatagatccttcctctgagaaaacaggaattagtactaaactagactcaggaatagacacacagatatttggagacatggtgtatgataaagatagtgtcacaagttagtagggaagagatggataatagtgctcagaaaattggcttattatatggacaaaaataaagttgaaccctcataccatataaaacaaaaatcccaaagtgattaaagacctaaatgaaagataacgtgatatacagctagtaggaacaaatggcaaatgtttttgtgccttggggtaaagataaatttcttaaataagatcccaaagcacaatgcataagatttgatagctttgattactttgtaattaaaggtttctatttagcaaagcatcccatagtcaaagctaaagatggatacggattaggagaaaatatttgcagtgtctaaaactgacaaggattgcatatacaagggatagaatatagaaagagcttctgcaaatcatgaagaaaaagacagatgaaataaaaatatgcaaaaatatgaataggtaaaatctaaagagaaaaccagagtggctcataagtatattacctcactagtaattacagatacgctaattaaaacactgagtttctaccttacatcttagtttggcaaaaataagcaagagactgatctgatggggaatcagaactcatggaatattttctcttcttttcttttctcttttcctttctttctttttttttttttttttaaaaagacagagtcttactctgtcatccaggctggagtgcagtggtgcctctgcctcccaggttcaagcaatcctcccacctcagcctcctgagtagccaggactgtaggcatgcgctacacaatttttgtttttttagtagagatggggtttcaccatgttggccaggctgatctcgtactcctgacctcaggtgatccgcccgccttgtcctcccaaagtgctgggattacaggcatgaaccactgtgcccggctacatggaatatttcagctggtgtattcattttggagaacaaatgaatgaaacttactgaaatcaagtaatacctagggagcgatattgcagtttttaatagtgtggctctggagctatactgcctgaatttgaatcccagctatgtcgtttgctagctgtgtaacctttggtaagccagttaacttctctatgccttagttttttcgtctgtgaaatagacataatagtacctacctcataggtttattgtaagcttagaacaatgcctggcacacagtagtgtcacagaattattagctgttattattatcattgtcatcattatcatcaagtagggcagccagcttccacgatggcccctaatgatctctgccctctggtatataaacccttgtatagtcccctcccacaataaataaggttgacctgtgtaaccaataggatgtactagaaatgtgtgacttacgaaggtaggtcataaaaggtattaaaatatctgccttgtgctatcttggatccttgctctggaggatgccagcttccatatcatgaggacactcaagcagccctctggagaggcccaggtagagaagctctgaggcctctcaccaacagccagcaccaaattgccttctgtaggagtgaatcaccttggaagtgcatccgccagctctactcaagccctccctcaggcttgcagccctgatcgacctcgtgagagatcctgacccagaacatccagctccagattcttgaaccaaagaaactctgagataataaatgtttagtatttatttatttatttgaggtggagtcttactctgtttcccaggctggagtgcagtggtgtgatcttggctcaccacaacctctgcctcccgggttcaagcaattctcctgcctcagccacccgagtagctgggactacaggtgcccatgcccagctaatttttgtatttttagtagaggtggggtttcactatgttggccaggctggtcttgaactcctgacctcgtgatctgcccacctcggcctcccaaagtgctgggattacaggcatgagccactgcgcctggccatgtttagtattttttaaaaactgctttattgagatataatttacatactacacaattcacccacttaaggtgtatgattcagtggcttgtaatatatcacagagttatgcaaccatcaccacatttaattttaaaacatttttatcatcttgtgggtacaagaaaccttgtacccattagcagtcactccccactttcccctaacttctccagccttaggcaaacactaatctactttctgcctgtatagtttgcccgttctggactttcatatattaataagatatgagtgagactgtcatataatatttggccttttgtgtctggctttttgtgcttagcataatgttttcaaggttcatccatgttgtaatatgtatcagccctacattcctttttaaggctgaataatattcccttgtgcggatataccacattttatttatgcatcatttgatgggcattcgagttgtttccactgtttgggtattatgaataatgctgctgtgaacaattcatgtataagtttttgtatggatgtatattttcttttttatattcctaggggtggatatattcctaggagtgaaattgctgggtcatatgtaactccatgtttaactttttgaggaactgccaaacttattccaaagaaatgtatgcgcgttccaatttctccacatgcttgccaacacttattatttgtctttttgattatagccatcctgatgggtgggaggtggtatatcattatggttttgatttgcattttcctggtggctagtgatgttgagcatctttatatatcttttggagaaatctttattcaaatcctttgcccatttttcatttgggttattggttatatatacacatacacaggcactctcttacgtaaagggttctttatatatatcttggttataagtcctttactagatacatgatttgcaaatacttttctcattctatgggttgttattgttttaagctgctcaagcatttagggaaggaagtttgcatttagggtaggtaagcaacagcagttacagttataaccataacaattgttatacagcaacagataactaatactttatgtacatacttcaaagtagttccactcctggctatatccttcggagaaatttgcataaatccttgaggagtagatacaaggatgtttttctatgtatagtttgggtggcaagaatttggaaaaaacttagatgtccatcactaaggaaattgataagtaaaatgtggtatatgcatataatggagttttttttttaagtcaaatgcaagctgttaggaatttcgtgaaatggtagacttcaagtgaaaaaataaggaacagtgaaatttaaagcaaaaatcagctatataaacacatgaagtgatattattttataaggaggatatacaccttaatcagtagaataggtttgtgggagggaacagaatgagactagaatggagaatgaagcggggaaagggaagcaaagagggctttgcatggatgaaagtgatcgtgtatcatgaactcaggcctgtgataactcagttctgtgcacctgaagccctaagtgcagctccagaagcaaggtctggtttcatggaattgacatttagtacccatattagttagggtagactaaactgctaaagcagactccaaatagacatgtgtttcccccctgctcactcgtgtaacatgcaggtcgtttcaggttgagaaagtaggagtgagatactctatgaagttgtttagggatcaggctgatgccagcttcactatggcttccaaggttgtgttgggcattaccattttagtgagttgagcagaacgtgaaggattgcttggcagatgttaccggctagctctagaagcggcacacacttctactcatattctgttggtaagaacctagtcacaaggctccctcttaatgcaaaggagcccgaggcatataatatgcatcaggaagaagggataaatggatgttgttggacacataacatattatctttgccacattatatacttctcagacttgtaaagaaaggctgtccatcccccagccatttgagacatacatagatttttaaatagttttgagacctgaaaatcccgaagggagtatcagttggggtcactaccaagttagggatgtctcttccctaactctggccagtgattgtcagagaagggcttaggtagggagataaaatgtaaactggaattgtgtcgagaatcagcatttatgagatggaagtactttatttatttttacagctcagatcttgctgtgttgcccaggctggagtgctatggcatgcttgaaacttgtgggctgtgatcctactgtctcgccttcccaaggtggcagaggaggcattcaactcctagctcattgtcacaccccttctgcacagctaggttttgtttctgtatgggttcgggttttaacttcatttttgtgggaccctcacgctgtttcttaggaacacctcactcttaagaggctcagaggctttagaaatctgaaatcactttttttcccttaacaaaggaatgtattatttatttatttattcattttttatttttttgagaccgagtctcgctctgtcgcttaggctggagtgcagtggcacgatctcggctcactgcgacctccacctcctgggttcaagcaattctgcctcagtcttccgagtagctgagattacaggcatgcgcaggaatgtattatttatttttaagcagagattaaggtgtagggaaagcagttagattctgtttcaggtgagctctatgtttagtaggcattagtgaacatttaaaaactccaccccctccaatttctctgcctgtaagtgtgagaacacagcacatcttgtagggcagagatcgcaagcctgtccagtccctagctgactgtcatttcctaggctgtctactagagggcagtgtgatatgccttttctagactggcgaaagcggagccctgggtctatacacacttaataatagtaggccttagatctagaacataatcctgtttccaatatggaaaagattttcaagagagagacctagtatttaggttttgaccagaatctcccttacttgggctttggaaacctttctctacgtggaagattttttttttccttttaatcaaatctcctttcttgcgcctcctccactttcaagtttagacttaagaagccatgagcaatgtgaaaaacctctgaattgtacattttaaaagggtgaactttatggtatgataattatatctgaatttaaaaatattaaaacaggccgggaatggtagtgcattcctgtaattccagctactcaagaggtcaagtgggaggatcacttaagcacaggagttcaggaccagcctggacaatgagaccccatctcaatgaaataaataaaatttaaaaaatgtaaaaacaacaaaaaagaagaatcactgagcagacactctggctcagatagaatataatagtagtaatagacgtagtaatagactggaatgactcagtctctcctttatctctcctgaatgattcaaacttgatggtgttgatcttggccttatacagcttcaccctttcagaaaacaagggtacgtgggcgatcttggtggtaggttgatactcaggcagtacctgtgaacccacccccttgggaggggacagagagcagtctgctgaaaaaggagtcagtctttccctccgcatatcacatccagcgccctctgtcagctaaggaattacaaagtgtgtgactttatcctgtcacacgtaaaaatgcaatctctagtgcttagtaaacagggcctgttttctttattttccttttagtaggagaggaagcaattgtgcagagaggttaacttgctcaaaatcgctctgagttgggaaagagtcagaatttaaacaacagattcctctccctgcagttgtcacccccacctatcttcactccagtagcaaggtaagaggtgagggggcagtccaagggactgtttcctgtccagagcagttcttaccaggggttctcatagcctctgcaaggaggtcctagtgtacttgaagctgagttccccagtcctggaagaactgcttgctggtagtttcagtttggaactcggctttgaagatcctaaccaagtgtgttagaggggcacgtgcccttgactcgtgtgtgtgtgtgtgtgtgtgtgtgtgtgtgtgtgttttgagacagtttcattctgtcacccaggctggagtacagtggcgcgatctcagctcactgtaacctctgcctcctgggttcaggcaattcttgtgcctcagcctccccagtagctgggattacagatgtgcgccaccacacccagctgatttttgtattttcagtagagaccgggtttcaccatgttggtcaggctggtctcaaactcctgatctcaggtgatccgcccaccttgcctcccaaagtgttgggattataggcgtgagccactgcgcctggccccttgtctctttttgccaggggcttgggaggtggtagacaagacctctgggaagagaggaatgcctgtctgggaaaaaaattattgttttaattgctctctttcattaagtacttactgtgtgtcaaatgtgtatgttcagtgcgtacttacattacctctgttgtcagggagagatcagtctgtgaatggttgtagattagggagagccacatttctgggtctcccaagaaaaggggggttgggatatcccagcaggataaactcttccttgttttcccccatacatcctgaagttataggaccttcatgctgattacttgttgacagagagcttgggcactttaccctggtcactgtgtccccatcaaatttgacagtgctgttgccacccaggatggccgtctcctgctttggccagctcagctcttcatgcagattaagtagtgggctgccagccgggggcagtttccttctgcacatgctggatatgtttgctgcccggggtaaaaataatgctgcaggatggagccatgccaaggggcttcaggtggtatttaacatctcctggggctatttcctcctgggcttccaactgctgaaaagcagctcccttctgctgctccccccagtgggaagttcagtttgtggggggttgagggggggcgagggggggtccttctgaccttcccttcattaattgggcttagtaggggaagtcagtggtggtcagtctcctagagtgagaggtcatcaaaaagtagtccatgttacctcctatcaagttacttaacttctctgagccacagtttccttatttatttattttttgtttttttttttgatacagagtctcgctcttgtccctcaggctggagtgcagtggcgcaatctcagctcactgcaacctccgcctcccgggttcaagtgattctcctgcctcagcctcctgagtagctgggattacaggctcctgccaccatgcccggctaatttttgtatttttaatagagacggggtttcaccatgttggccaggctggtctcgaactcctgacctcaggtgatccacccaccttggcctcccaaagtgctgggattacaggcgtgagccactgcacctggccacacaatttccttatttgtaaaatcagggtaatacctccctttctggttattgagaggatctgaaataaagttatctagcatagtgtctggcatgtagtaggtgcttaataaatagtttcttaaaatagatacttaaagagcatgtctgctgcctcagtggcacatgtcccagtctctgttgatccttctcttgtcttattcctggctttacctgaacaccagggtcacagttctctggctgtgtttggagagccagtgtctcttgctgggtgggattgagggatgtactatttgaagaagagtagtcaaacaggtggggatcttctgtcccctggagagggggtgtctagaggcgggggagaagtgattggcttgttccagtgattcttgcctgtctcatggtagccttctattacccattgccccttgtcaggggagaggtgggtgctggtggtggtgtccagacaggacccagtttgaatttatttgaagctggggacataagaactagtggtgggggtggggaggggtgaggagttctctgcatttaacatttaagggcatgtggaatttcaaaagcagttttcaccagcatttcatgtttgtttggttgtttcttttgcctttgagcatagtgctggttgggggcagggtggcttatgatgaagatagtcagtggtggactcctgcataggtgggtggaactccatataggaaaacacattaccgctgggcttagtgcggttgctacggtgtctgtgtctcctaaggggcaggaaaaagaacagtctctaggagcaaaagaggaagagaattgagtcaggggttttgttctctctctgcttccaaagctgcaaggctgtgtccatgagaccttgaggaagcccctccagttcctcagtgggagggaagaaggttggccctctgcactggctgtgttttgacagaagtaaaactcttgactcagccttcaaagttcaagggtgggatattgggagaggatgagcagctttttggcttgttaagaaagtcacgtttttggccagtataaagtggaggaggcagagtggggtgaggtgtgtgggagtcctgtgggatggagaaagcagtctgccaagggccatgttacctgtgggtgatgcagagaggctgtggtcataatggggagcaggtagtgagaactctgtggggagggctgtgtttgaaccactgaggtctgagcagggcccagggcaggaagcgtcctgtgttctctgcctatccaccctgcattaaagggagggaagcagagagcaggagctctggggacagggaggggagtgagagggggatgccaggtagtgggtgactagaggactcttatccacagtctcagagcacacagagatggctgaaggatgccactttaagtgataccttctggtcatgctggcagtagcttctgtgctcagcaccatcttctgtcccccagcttttgagggggcacctatgctttggcaaatggactttggttctgaggtttggtgtcttaggttgcctcaagcctgcactggttttgggagaggaggtagaggcagctgtgtccccaacaatagatgactgtggccagcagctaagggaagggtccaactctggatctccaccgtgtgtgtgtgtgtgtgtgtgtgtgtgtgtgtgtgtgtgtgtgtgcccaagctctggccctgcagatgtctatcttttcctgccctcatctctgcttcccacacccaccccccacccacgtgcacacaccaggtgttgtcctgagacctgagacagaggggtgagctgcatcttgtcagctgtgtagatctgccagtagctctacagtggctccctttcagaatctggaatcacaaaataagaatctcagagttggaagataccttaaagggcttctagttattccaccttccccataccaattagtgtgtaagtatttccatcagttcactagctcaactgctgttcccctctagtaccatggaaatgcagtgttaattcgattaatcctagaatcaaagttggaaagagccttcgacaagagctaatcctcacactcaccccagtgccaggatcccctggcaaaacacatttatgtgatgtgccgttccactcacatttgaacatatgtattccattcctgagcaactctagcttttttccagttgtctttccccatggcttcaccatttgtctgaattatccaacttggggcttatataaaaaaaagactaactcctttctatctgacacaattttagatgtgttgggataaatttttctccctgtgccctacctgtttcccttcctgggcccacagcattaagctaataggaggagagtcttgcattaaaacttatacaggaaaggaagcagacaatcttttacataataaggtagtaggaaattaatcttcatacacggagttttatttttgaaattattatattcttatttgtataaatgtatggaatagaggtgcaattttgttccatgcacagcttgtgtagtggtgaattcagggcttttaggatatccatcacttgcataatgtacataaggcccatgaagtaatttctcatcattcacccccctcccaccctctcacccttccaagtctccattgtctatcattccatgctcttatgtccatgtgtacacattattattattttttgagacagagtcccacttagtcacccaggctggagtgcagtggcgcgatctcagctcactgcaccctccacctcctgggttcaagcgattctcttgcctcagcctcccgagtagctgggattacaagcaaacaccaccatgcctggctaattttttttttttttaatacttttattagagacagggttttgcgttgttggcctggctggtcttgaactcctgacctcaggtgacctacctgcctcagcctcccaaagtgttgggattacaggtatgagccaccgtgcccagggtgtacgcattatttagcccccacttacaagagaggacatgcaatatatgtctttctgtgtctgatttggtttcacttaagataatggcttccagttccatccatgttgctgcaaaagaccatgatttcattctttttttatggccgaatagtattccattgtgtatatatactacagtttctttatccaatcatccattgatggccacttagattgaggttgattctatatttttgctattgtgaacagtgctgtgatagacatatgagcacacacagaaggtttaatggtgttcatggacacctcccctagagggcctcatccttctcctctaccctcagttcaacagcagaaccaagcgtgacaacctgtgccagaggctgacaccagagggagaggcaggaggaaaaggatgcaggaccctagaagagccataatgttgccattagaagaaagctgctgggcccatctgactttttttttcccccagtttcttcaaccatcctttttaatgcatgatttcgagtctcacactgacttctctgaactcattccagttggagtgagctgggccataatgttcaaagtgtgctgatcagagtacaatgggtatcacttccctcagtgtagacattatgcgtgtgtgtatgtgtgtgtttatatgtatagtgtaacctcaaactcctgggctcaagagatccttcttgcctcagcctcctgagcagctgagactataggtgtgcaccaccatgcctggctaaattttaaatattttgtagagatgggggtcacgctatgttgcccaggctggtctcgaactcctgggctccctctcacctcagcctgccaaaatgttgggattacaggcatgagccaccacactcagctagatattctatttaattaactaattaattaatttttttagtgggttaaggagtggaacgtttaatagacagaagaaagcagagaggagagcagttccttgtgaaggagagagagatgtctgaaaaaaggcctagatagatattctattttaatcagtgtatttaagattggattagctttctttttttttttttttttttttttttttttttttttttgagatggagtctcactcttgttgcccaggctggagtgcaatggcacaatctcagctcactgcaacctctgcctcccaggttcaagccattatcttggctcagccccctgtgtagctgggattacaggcacctaccaccacgcctggctaatttttatattttttagtagagatggggtttcaccatgttggccaggctggtttcaaactcctgacctcaggtgatccacccgcctcggcttcccaaagtgctgggattacaggtgtgagctaccatggctggcctggattagctttcatggtgaccacatcacaccacggattcccactgcactatcgtcagaagctcccgtgtctcctttacacatatcgtgagaaaggagatggagccatgttgcatccatctctttatttgtgcagttggtgttttggactcagtatagttacctttaacctctagaaatcctactttcttgttcttctatgctttctcagcatgaaagtggtaggatgctaagctttgttctggtttttcttggtgcaggagaggaggaggccggaatgggggttacaacttgggcccggttatactcctgaggctcagatgagcccggccaacatggcctcatgctggagaggacttagcttctgagtcaatttgaaaagtcttttaatatgaattaaagagaaagtgtgggctgggggcagtggctcatgcatgtgatcctagtgctttgggaggctgaggtgggtaggttgcttgagcccaggagtttgagaccagcctgggcaacatagtgagaccttgtctctacaataagttaaaaaattagctgggcatggtggcacccacctgtagccccagctacttgagaggctgaggtggaaggatcacttgagtctgggaggttgaggctgcagtgagctgtgattgagccactgcactccagccagggtgacagagtaagactctgtctcaaaaaaaaaaaagttgttggcaaagacaaaactactattcaagcactcatctgaggtgctaggagacccccgggctttgtggcaggggctagaagggccctgtctgcccagttatacttaggaactgagaatttgaactcttgctactgctcccaggacaggatctgcttaccaattcagccattttctgagttttcatggcactttgaggacagcatttatcatactattttgtgcgttgggagttcctatggccagaaactatttcttattcattttcaaatccctagctccaaagccaaggtattacaccccagaactaatagctatcatttattaagtactaacttaggcactaaaattaggtactctccatatattaatctcacaacaacccaacaaggttaatgctaatatttccatgttgtaggtgaagaaactgaggctaaagaccacagctctaccaaagcctctgctctttcttcaaatgaatgagtgagtcgggggcggttgctctgagtgagagtgggccagagtgggttcagggcattcaggagccaccacagctcattcgtggaggtgtctaatcagctatgctaattgattgtcgtgtctgcaatatcagatgcatgcttaattagcacatgaactctcctgtcagatattcctggaggggctctgccagccaacctaggcccagccacaggagcctgctgctttcttggaaacagggtggggaagcatgatgatcccagtattgacaaggatcccctgagaacaggaaggtccactgctttgtcgtgtgggaagcagattcaggatgaagccctgtctacaagatgtgtccagtctcctccgcctcgaggcccttggttcatcgacctcttccctacagcctttggactttgcaccactctttgaggcctctgtgcttttttccaagctcctgcttctgcgctggtcttcctctccctagcaaatccttattcacccttaagggtccagctcaaatgtcacttcctttgtggagttccccctggcccccgtgctacccgcagagtggcagaatcaaatgcttcctcttcagacttgctcctattgtacttgtacctacctgactctaatgtgagtctcttcaaattacagtgtttttgacgtgtttttctcccccggccagacttagggcttattcaaggcagagaccacagtcttcagcatccggcacaaagcccggcctgtacaagacactcagtacatttaatttgttgaattaaactgaatgctaggttgaggttccactgcaggtaagaactgggcaagtgtaaacttccagaactcagcagctatttattagtgatatgcagatgtaggtagggcatctttgaaatcttcccaaggattctgtgatccttatttttccttttcacaataatcctctcctacttgggaaaaagggggataaaatcgcactttggccccagcctggggagaatcttggcattaggctggggggccatggtgggaggaagcatgggactggggcctaaaggccatcaaccagactgcaagctgcctgagtgcaggatgtgacttcccctccctcccacacatatggccattgcttcacctggagtgacttgggactggggaatcaggggtgggcagcagtgcaggaaaacccatccgcaccagcctggggctccctcttctcactacctcatgaggccctgtggagtgtggactgttgctgctgtcctgatcccccaaatgccataactgctgacttctgatgtaaatagtgtacatttgttttttactttgcagctaatttgcattaacttctctgaatgacagacatataggaattccctgatttctatgacctcacttccacaccacaaaaaaccacagtggtccctatacctctgtactcagggactcccaacccaggtgggcagataccagggggtttctgaggagccctgcatcaagcaggcaccagcccaggaggaagcgatccaggaatcccactaccccggagcccactgctctggcgtctctttcttgtctctcggggttgtgcggggtgagggcatgggggtttgtgaaatgtcttcgtgattttgtaattcaggctagggagtcattcggagaaaagcgaatgaaaagtagggtaaaggggaaagaggggctttgtgacttaggtgggctttgcagaaggacaggaagccacaccccgagccttacaaggcaagctggatggggctgcccggctgcaatggggatggaccgatacagaccgctcagtgtagccacctgctggacacatggagtcatagcgttctgcttccttcatgcttcctaccctccagctgcgcctgctcttctttcttccttctttctcctccttccgcctagttccgccccggcccgcatacctgctttccctcttccctcaggtgtgactacccagatgcatctgtcttggcccagggctggggactctgaatttcctcctttggtgatgactgaaagggagcagctatccctggttgaggccagagagaaggcttttcatctccatgttcagtctctccaccttctcgcttggcttgctgggatcctgcgcttggctgcgattagaggccactgaatttattgtccaaactgggatgcttctgagagtgaagggtgtgtgtgcttgctattagtaattatgcagagacaacagctgtagaccagaattgtcccgggccagcggggactcatggtaagcatagttccgagggtccgccggccttttcggccgtgcgtcaggtgtggaaggctaaggcggccagcagaggctgctgcggcacttgttatgtgcctcactatctaaaaagctccttttgccatgctgggactgtggtgggtgaagttggttgtcgtacagggctaaggtcccctgagtagtgattgtaccagatttgctcacctgggcctggcgcacaatgggataagaaagccgcctgcttcctctagtctgggtgggagacacagcccaaagtcccagggcctaccttctcagaaactccccatcatcctggattggactgtgcctttcctgaattctgacacttcttgttcctgccccttcaggcaaagactgggcaagcagtttcctagcactgccagctctgaggctggtgcctttctgggtggattattggccttcctggatacattggcaggaggactcatgggtcggccccaggatgccctggccagaggggacccaaggaatgcctgacagtttctctgcctgacttgcaggggggaattcagaccagccttggagtcctgtgaaggggaagggctcctggaatcttctagccctccttcctctcacccagttctaactctggcagaaagacctcatttcctcttccccatgtgggagactttccctcccttgacccctcttgcagttgggagaccttgatccttgtgggaggaaggggttgctgtaccaggatggggtccctccgctcccctggccaggggaagcatgacctgctgagctggagtctggccccgcaagctgttgtggcctgacctgtagttgcttgcccagtaccagcccctctctcccatcttctctccctgctcacctgaagggagaggccagtatcaggctttccaggctacgtgctggtgatcataccatggcaaagggcggtgtctcacacaggctgcgggagagctgccctgcttaagcagagaattctggagcagcagtgggtatcccaggcagccacagggattgccatggcaacacgcgaggcagcagcagcgggagacgggatggagcagggcgttttctaggctagagctgaattcctggggtggtcaggagagggctggagggcaagagatccataaacccacagctgccccgacagagggcagtctgcctttgctcctggccccttgtcagtggaaatccgacaccccctggtacatgtctgttaggtgtccagcctgggcagagggccttgttggtgtgtggagagggggaaggggaatcaaacttaactgcagagatttgttttctcagtacatctgaagaatagaaatgggttttaggctgcgccagcctgcgtttttctacctgggggactctgagccatcaaagttaacctagactagtaaccggggatcaattacagacctgtaatatgctttccaacttcccattgtaaaaagcagcctatttggaaaaaaaataaatgaaagtgcttttcttggtctctggtgccactgagccagtgtgattccctccctccccagttgcctttgcccacttctcactcattacctctactcaattagccaaggctgggttaacccctcaagagccaggatttgggtgagaggggattgctgtcaccttctacaggcacgccctcctcctagcacagttctcgggagcgcagagcgggccaagtccagagcctgccaacctcctccccaccctctctctcgctgccaaatctgactttgattagcgtgtggagggggaagaaagcaggaaaatagaatattaaaatcttaattcaatttaaaacactgtcattcacaggcatgccaaacagtgagatgactaattattatgcaaatgaggcagatagaaaagtgattaataattgcacaaattaaaaattattgtaaacatcctgtgacgagtgataagtccgatggagaggcgagagcggtgggccggggaggccatggcggagctggctcctgcatccttatttcctcattagggggatttaattagcgcctgatgatggggctccgtgctgccaggggtggaggagttggtggccagggcctgggcggtctgtgctggatcctcggtttttctccagttcctccttattgctctgtggctggggctggagctggctgcacaggaggaggggtgggttggggttggggaaaggctggacaccatagaggagcaggtgctgtgcaaagcttctctgccgccttccactggccttttgcccgctgctacctctgtcatttaagtcctcaggtgtgggtcaggggtgttggtgaggaggcccctgggtgaggaactgtgggcacatcctgattcagccaggcatattcctcagggtttagtgaagcgacacacagggcacgtccccaatgctgaaaggtcccagggaggatagacaggtatatgatgctgcctggggcctcagggttgccttgggtccaagtttccctgactctgctgggcacatagaaactaagtcagtgtcctgagccaagtgacttgcccttagccatgttcaacctgcatccaagcgccccgcagcagggaatgcttaggagtcactttggcttgggtgggcatggctgctgcaaggcaggggacagaggaggccgagttctttgggcccagcagcaccaggtgctttccagggcatgcagccctcctggattcctcctgcccaaatagggaagcagctgatatgttcttgacaaaacataccagccagccacagtacagcttccccctgccagtccccgagccctgaactctgagcaggaggggatgggactgggtctaggctcaaggaaaggtggctgatgatgggaaatccatctccctgctccacccccagtcaccatggaccttagggagatggtcttccttgtccagagaagaccacagaataagctgggaggtgaagagcctggtgaagagcttttgctcttcagaggaacagtggatgctggaaattctggcagttgggaaggcttctggacagggccaaggtgggaatctggcctttgaaagatgtagaggactcagaaaagagggttggaggtgagaaatagagagaatagcgttatccaagtctttaccttctctctaagagcccagattttcctaacggggctaagaagctgaggttttcacaggccaaaggcccagcctgtgtgtccctaaatttctcttcctgagactgtggtgtaaaattaatttttttttcctggtgagggtttggaggtgagaaaggtttatttctgtttcaggactcacacagttatcccagggttaataacctgcacacagccttgtcaccacagccactagtacccctcttcccaccccctctctgccagcctcatgtcttcactctggttcctgggtttctctagtccctttctgattcctggaggaggaaagttctttcctattgggactgcaagtccacccctctgacttctggctaaagcttcctggactctgaaggctctcccttcactgtgaactgtttagccatagctgccaaccttcaacaaactcccttctccatctcctctctttgcccactcctgactcctgccctatgctaccctgagcccctcctgggttcttattctgtttttcttttgttttggtttctctggggctctgctgaggcagcaaagggtgatggggtatggtactgattctgtgtttaccttggacaaactatatcacctctctgtttcctcattgttataactgggaaaatgctacaatctcatggaatctttcactttcccatcttcatcagttcatttattccatcaagacttcacctctacgcgtcaagtgctggggatgcaaagctgcagatggtctcaaggaacgcgagccctgttgggaggcagataaaggaacagcacgcgcagaggcccggaggagggaggagaatagaagtgtggagactggcaagggatttgggatggcaggaggacaggttgcactggggagtggtagagttggaggccttttgttccctggaatttcaatacgattttgtgggctctgagcaggtaagaaaggactttgttggggtggcaggcaggactaacactgtgttttagaaagaycgttttggcagcagtgtggagatgggcaaggctggaggcagggtccccacccagaggcagctgctgctgtctgtatgaaagacgatgaactgcagccatcaggtgtggctcaagagtcacgggaagaggagagagctttgtcatgagtcagaaaaggcaggatgcagcatctcagtgactgtgagggtgaccgagggggcgtcttgggtgacacctgggtttctggcatggagcaatgctcttctttgagaagagggatctagggaagaaggagagatgatgatggtgttgctttgggaatcctgggaatgaccaccctgggtttagttgcctgggacggagtgggaattgaatatctgctattgaatatttgatattgaacatgctgaatttggcgcttcatctgccttgcctgttttatttctcactctgaccatacctagatcttttgaaaggaggcttgaccctatcctgaagcttgaccctgaccttctacctttcaacattttggttagcgggcgcctgcttgcggactttctgagatgcacttctcagttattcagctgtgtctacattcattcccacaggctctgggcaggttggaagaacccacatttgaccctatggcattggacttgggttgttgctacctctattctgtgccatggggaagaaggctcttctctgctgagtcctaacaggcagatactggctgtaaatcctttctagacccctccccggtgcccacgctctgaggctactcggagggcggtgtgttgttgctgagacccctgaatcgcactacttcaggctgtcccctcacctcgggggtggagataaggcctggggtctgagtttgaggggactgacgccaggcagggctcctagcagtaaggtggaactgcttccctcctcagagcgactttctaaaccagcttcccattcctctgaggctgccgccacccctgggaagcacacatttgtggttgccgaacagttgaagggcagtggctctttcttccagggcagtgtgggcctgcctctggctcccccgggagggctgcacccccacccacctgtgcccctcattaagagtaagcagcccagagtgcagcacttggtgggacccatgaagaccccaccaggagtccctagtggtccccagtgtctccattatgccctctgcagttctcacagtgccctaggaaagtccatgtgcttttgctctccagagagctcctggtcactttctaatatgtctccagcaactcagcattatcagtattgccccaatatctctttgatggaatccctatcaggctactggcattcatagctacttcccacagaatcccaggaacactccagtaattcccagtgagctcccagtagtgccaggttgctcccatacctcacagtgggcttctagtgatatcacattgtgtacccaggagcacctggtgttcccccaagaacgttccagcatgttcccaagaatgctccaacacatggcatcaacattccactattcctgcaagatttccagtagatgccattacttctcaagttatctccaccaatgccactgatgtgcttagaggagaatctgctgtcctgcccaccaggaatgaagagaggttctttgcctactcctcctgcaagacacctgggctttcctgctttgggaggctggtagggaaggcaaaggagtgggaataacatttgttgagtgatagccgtggcttaggcatttatttgtgcaataaactgcaaggtgggtggtatccctattttatagactcagaaactgttacccagagagtggagtaatttttttccaaaggcagtaagtggcagggagtgatcagaagggcagctctgtcaggtctgacatccctgcttgtccggagcctgcagagacccagagaaggcaaatcccagggccagagatgcacagggtaggggcgggggagggtgggcagcgggctctaggctccttccttccctccctactttgttttctcctttggattgcaggcgtattcattcctgagaaagaaaggaaaggggttaggactggttgagctgtcctggtttctttggagagaagctgtggttcccggaacgtcttggctctctggggactgcaggggtcagatccatcccctccagtttcacatgcctactcagcttccacattccacatccagtggctcaccagacccacccaccggcccagatgacccagttccagggccggcctcagggttctgggctccctcctgggcctgccctcagccctgggcacaacctcaaactccaggtcctgggttgtacatccgcctttctgcccctttcccacactcgctgctgctctcccactggctgccttccctggcatgggtgaccttgctagtgccaggggactctgaacagcgctgactcagcaggtggggcgatggagccactctgcaggtggggaaacaggggagcaaggctggttctttcctttccctgacccggcggggtccctttgcccttgggagtgtggacggaagggcagggagctggacagggaggatgaggtccagcgccctgggtgagggccctggtcggggagacggtgcccggtggcttggcctccctagcaaggactctgccccttgttcctcagcctgtcagggagaagaggaagggctctctctggtgctgtgtcgagcagcagcctcccacacggagtggggaggggaaagttgaaacgcaccttgactcctgaccatctcctccccaccctcacccccacccccaccacggcagacattgttggaaggcatattaatggaggggttaggcagtttggagacagactgcctaggttcatgtccttctctatctcattcttggacatatcacttagggtctctgaaccctgatttctccatttataaaatgtggctaataatgttacttacctgtcacatggtaaatgctcaattgaaaaggctaacatgagaatgccattttctgttatgtacatgatgcttcatacaatcacactggcgcacatatgcgattatgtgtgggctttctgtgcgctctgcccttccccagctttgctgtctgtccttgttgcttccagaaggttgagagggaggtgaggggtctgctcctgacaatgctgactactgaggggctgagtgctgcccaggaaagtaggtagcagaggagagaagacttggcctaagcgagggagccagggccttacagaggaaaggaaaagggggcagggggaaggaggacagggagggtggctgggtatggcaggagtcagggcatctcagaggcatgaaggagctgggggtgctgccttcctactcgggctcacccgtccagcccccacactgccctccacaccagacacccagcagtgctgctaaggccactggccagggggtgtgggccacctgcacctactgcttgcctttgggggagattttttttggggggattcttggctctcttgggttatgctcctccttctggtgtaccctaggcagggaagagtttggggagaatgggaggctgctgtgagggttataggtgggttacttcacggactccgtgagtctggggctccttctgtgaagtctcagtgacaggacacgaacccaagaactgttggcatgtggcatccttgcctcaggagctcttcagcaagtgctggagttcacatggccagcctgagggaggggtcttctgtgttctctctgcaccccttcccctccctgcagcccagtgtcctcagggcaggggtgggtggcagtggggaggagggaggggagatggtctgtgatctctggttgcagtgaatttgggactaaacatcattaatgctgacagatgccagccatactaacttgtagaataacaggacaatctaggg

SEQ ID NO: 2 - DNA, 1879 bp, Homo sapiens
  5′UTR 1..29
  CDS 30..1121
  3′UTR 1122..1879
  allele 1153: 17-41-250       : polymorphic base C or T
agacgtgagcagagcaggtaatggcaagcatggctgccgtgctcacctgggctctggctcttctttcagcgttttcggccacccaggcacggaaaggcttctgggactacttcagccagaccagcggggacaaaggcagggtggagcagatccatcagcagaagatggctcgcgagcccgcgaccctgaaagacagccttgagcaagacctcaacaatatgaacaagttcctggaaaagctgaggcctctgagtgggagcgaggctcctcggctcccacaggacccggtgggcatgcggcggcagctgcaggaggagttggaggaggtgaaggctcgcctccagccctacatggcagaggcgcacgagctggtgggctggaatttggagggcttgcggcagcaactgaagccctacacgatggatctgatggagcaggtggccctgcgcgtgcaggagctgcaggagcagttgcgcgtggtgggggaagacaccaaggcccagttgctggggggcgtggacgaggcttgggctttgctgcagggactgcagagccgcgtggtgcaccacaccggccgcttcaaagagctcttccacccatacgccgagagcctggtgagcggcatcgggcgccacgtgcaggagctgcaccgcagtgtggctccgcacgcccccgccagccccgcgcgcctcagtcgctgcgtgcaggtgctctcccggaagctcacgctcaaggccaaggccctgcacgcacgcatccagcagaacctggaccagctgcgcgaagagctcagcagagcctttgcaggcactgggactgaggaaggggccggcccggacccccagatgctctccgaggaggtgcgccagcgacttcaggctttccgccaggacacctacctgcagatagctgccttcactcgcgccatcgaccaggagactgaggaggtccagcagcagctggcgccacctccaccaggccacagtgccttcgccccagagtttcaacaaacagacagtggcaaggttctgagcaagctgcaggcccgtctggatgacctgtgggaagacatcactcacagccttcatgaccagggccacagccatctgggggacccctgaggatctacctgcccaggcccattcccagctcyttgtctggggagccttggctctgagcctctagcatggttcagtccttgaaagtggcctgttgggtggagggtggaaggtcctgtgcaggacagggaggccaccaaaggggctgctgtctcctgcatatccagcctcctgcgactccccaatctggatgcattacattcaccaggctttgcaaacccagcctcccagtgctcatttgggaatgctcatgagttactccattcaagggtgagggagtagggagggagaggcaccatgcatgtgggtgattatctgcaagcctgtttgccgtgatgctggaagcctgtgccactacatcctggagtttggctctagtcacttctggctgcctggtggccactgctacagctggtccacagagaggagcacttgtctccccagggctgccatggcagctatcaggggaatagaagggagaaagagaatatcatggggagaacatgtgatggtgtgtgaatatccctgctggctctgatgctggtgggtacgaaaggtgtgggctgtgataggagagggcagagcccatgtttcctgacatagctctacacctaaataagggactgaaccctcccaactgtgggagctccttaaaccctctggggagcatactgtgtgctctccccatctccagcccctccctctgggttcccaagttgaagcctagacttctggctcaaatgaaatagatgtttatgata

SEQ ID NO: 3 - protein, 366 aa, Homo sapiens
MASMAAVLTWALALLSAFSATQARKGFWDYFSQTSGDKGRVEQIHQQKMAREPATLKDSLEQDLNNMNKFLEKLRPLSGSEAPRLPQDPVGMRRQLQEELEEVKARLQPYMAEAHELVGWNLEGLRQQLKPYTMDLMEQVALRVQELQEQLRVVGEDTKAQLLGGVDEAWALLQGLQSRVVHHTGRFKELFHPYAESLVSGIGRHVQELHRSVAPHAPASPARLSRCVQVLSRKLTLKAKALHARIQQNLDQLREELSRAFAGTGTEEGAGPDPQMLSEEVRQRLQAFRQDTYLQIAAFTRAIDQETEEVQQQLAPPPPGHSAFAPEFQQTDSGKVLSKLQARLDDLWEDITHSLHDQGHSHLGDP

SEQ ID NO: 4 - DNA, 5381 bp, Homo sapiens
  misc_feature 1..918: 5′regulatory region
  exon 919..930: exon 1
  exon 1442..1498: exon 2
  exon 1613..1724: exon 3
  exon 2243..3940: exon 4
  misc_feature 3941..5381: 3′regulatory region
  allele 319: 17-42-319 : polymorphic base C or T
  allele 3213: 17-41-250 : polymorphic base C or T
  conflict 1241: 17-39-343 : T in ref genbank AC007707
  conflict 1447: 17-40-202 : G in ref genbank AC007707
  primer_bind 1..11022: 17-42.pu
  primer_bind 553..11575: 17-42.rp complement
  primer_bind 899..11920: 17-39.pu
  primer_bind 1246..12267: 17-40.pu
  primer_bind 1441..12461: 17-39.rp complement
  primer_bind 1632..12651: 17-40.rp complement
  primer_bind 2964..13984: 17-41.pu
  primer_bind 3432..14454: 17-41.rp complement
  primer_bind 300..318: 17-42-319.mis
  primer_bind 320..338: 17-42-319.mis complement
  primer_bind 3194..3212: 17-41-250.mis
  primer_bind 3214..3232: 17-41-250.mis complement
  misc_binding 307..331: 17-42-319.probe
  misc_binding 3201..3225: 17-41-250.probe
cagcagatgagactggaaatgagtcaggatgagccacagtggaggatgaattaaatgggcaggagtgtggtagaaagacctgttggaggctatgaatgcaatcaaggtgacagacaactggtgcaatgatggtagtggaaatggaggagaggggattgattcaagatgcatttaggaccaagaatcgggagcttgtgaacgtgtgtatgagtactgtagacggagtgggtgtgtcatcagagaagatctgagcatttgggcttgctctcctcagaggccctgcgagtggagttcagcttttcctcatggggcaaatctyactttcgctccagttcctggggctcagagtccctggcccagatgcctcttgccatctcatcttcaccctgcctggcttcccttgcttgttccaggattgtttcataaagagggatgtggttggtctttaaccctatgaatgctggctgaggatgcctgcggaacctgtagtgaagctttcaggggctgctcgggttctggctggtaggtgaacactgtccatcttgccggctgggacacagtgactctgggtagttgtgtaagagaggggcccttggcagacaaacaggttcttctctgttggtgggccagccagcaggtcagtgggaaggttaaaggtcatggggtttgggagaaactgggtgaggagttcagccccatcccccgtaaagctcctgggaagcacttctctactggggcagcccctgataccagggcactcattaaccctctgggtgccagggaaagggcaggaggtgagtgctgggaggcagctgaggtcaacttcttttgaacttccacgtggtatttactcagagcaattggtgccagaggctcagggccctggagtataaagcagaatgtctgctctctgtgcccagacgtgagcaggtgagcagctggggcagagggatgggggtcacagtcctaagggagggcattgcaggtggcctcaggggagagcctggggtggcccctaagacgtcctcttggaacattttggcagagttgcctcttcgccctcattatggctcagtttttccaccatgaaatgggagggagggagacaggtgggcaggggagaggtggtagaagtggcctagagaactgttcctggggtctgggacctttgcgaaggggttagagcaccacgctccctgctatgtgactgaggtagcaagagcacgccctcttcccatgtctgaggaagacaccctagcctccttgactcacctaggtcagtcctcttgagccccaacagctctgtgctccccagcccaaggaaggggtaacaggatttcgggcagttgcccctgcagaggccccctgggcaagtcccctgcgccatgtcccttcgtctccttcttcccctaaccaggcctccctccacctgtcttctcagagcagataatggcaagcatggctgccgtgctcacctgggctctggctcttctttcaggtgggtctccgaccctgacttcaacgtgggggtgtgggtggaggctggccagagggccctgtccaccctgggggaggagagcccaggccctgattacctagtccctctccacagcgttttcggccacccaggcacggaaaggcttctgggactacttcagccagaccagcggggacaaaggcagggtggagcagatccatcagcagaagatggctcgcgagcccgcgtgagtgcccaggggaaggggtgtaggcgaagggaggagacagctgggccatgccatgatgacctgcctctgctgcctcaacctctgtggccgctgctgggacagaggaaaggagcggtgctagctctgtctgcagatcccggccatcctgggctctttagcgccctctgcctgcagcccccgccttgacaactccgtagctgttgcccccttgctcactgaggcgcgggacctgggatcaatcgggaggacgcccgctgcagtccccagaatcaaaggatgatgtggcgcatctatgtttctttggagagtgttgtaggtctggatttgtatgggcaatgtgtttgtgcttcgtgcgtgagttgttactggccagggctaggacaagagccctcgaccctggggccaacgccctgcgtccttggttcccccagaggatcagtgcgcgatgacttggggacaaaggagatgatgggggctagcagtctgacggcctggatatctgtccccttctccaggaccctgaaagacagccttgagcaagacctcaacaatatgaacaagttcctggaaaagctgaggcctctgagtgggagcgaggctcctcggctcccacaggacccggtgggcatgcggcggcagctgcaggaggagttggaggaggtgaaggctcgcctccagccctacatggcagaggcgcacgagctggtgggctggaatttggagggcttgcggcagcaactgaagccctacacgatggatctgatggagcaggtggccctgcgcgtgcaggagctgcaggagcagttgcgcgtggtgggggaagacaccaaggcccagttgctggggggcgtggacgaggcttgggctttgctgcagggactgcagagccgcgtggtgcaccacaccggccgcttcaaagagctcttccacccatacgccgagagcctggtgagcggcatcgggcgccacgtgcaggagctgcaccgcagtgtggctccgcacgcccccgccagccccgcgcgcctcagtcgctgcgtgcaggtgctctcccggaagctcacgctcaaggccaaggccctgcacgcacgcatccagcagaacctggaccagctgcgcgaagagctcagcagagcctttgcaggcactgggactgaggaaggggccggcccggacccccagatgctctccgaggaggtgcgccagcgacttcaggctttccgccaggacacctacctgcagatagctgccttcactcgcgccatcgaccaggagactgaggaggtccagcagcagctggcgccacctccaccaggccacagtgccttcgccccagagtttcaacaaacagacagtggcaaggttctgagcaagctgcaggcccgtctggatgacctgtgggaagacatcactcacagccttcatgaccagggccacagccatctgggggacccctgaggatctacctgcccaggcccattcccagctycttgtctggggagccttggctctgagcctctagcatggttcagtccttgaaagtggcctgttgggtggagggtggaaggtcctgtgcaggacagggaggccaccaaaggggctgctgtctcctgcatatccagcctcctgcgactccccaatctggatgcattacattcaccaggctttgcaaacccagcctcccagtgctcatttgggaatgctcatgagttactccattcaagggtgagggagtagggagggagaggcaccatgcatgtgggtgattatctgcaagcctgtttgccgtgatgctggaagcctgtgccactacatcctggagtttggctctagtcacttctggctgcctggtggccactgctacagctggtccacagagaggagcacttgtctccccagggctgccatggcagctatcaggggaatagaagggagaaagagaatatcatggggagaacatgtgatggtgtgtgaatatccctgctggctctgatgctggtgggtacgaaaggtgtgggctgtgataggagagggcagagcccatgtttcctgacatagctctacacctaaataagggactgaaccctcccaactgtgggagctccttaaaccctctggggagcatactgtgtgctctccccatctccagcccctccctctgggttcccaagttgaagcctagacttctggctcaaatgaaatagatgtttatgatagaagtttgcctggcgtgactctcatttggaccatgtctgaaagcagtggcctcaccactatccccaaagcacacccatcacccactccattcccttgctgctctttctcatccacccactcccagtccaggtctgtcaaagggggtctggctgggctctgcttcagggatcctggctagacaacggctgtctgtcacacctggcaggagggcctgggttacgggcccttcctctgcacctgcactgttcactagcctgctcccccacaggacactgtgcatggaatgcaggctgtgtctggaagagctgtggccctggtggacctaagattcctgaggtgggctgcctcctttgttcctgctgttctagagtttgaatggcctctttttatgccggactctcttctggggactcccctcactcaggggcaccaatgctccctatagatcccctgggaactgaaactggggtgtggtggaggacgtggaaagggtaaacacagctccttgtctttggacttccctgtccggccccctttcctcccagctcagcctactgtccccgggttctcagcacctgcctgctccccaaccccatagcacagaccccacacatatgtaggctcatcatgcctgcaggctggtcttccctgacaccgtggattttgacaatgttggcaacagaactgggttgtggacccagcacctggagagaggaagtgctagaaaggtagaaataataaaaggtgtttttgttgttgttaggaaactggaaaagcataggtcaagggctatgatggggatgaggaggtaggagtgaaaatgagggctgtgtacttgaggctgggattggggaaggtagtgatgaggacagaatagggagtgggaagaacagaaagggacagagggattcagggattgtgagagaggggaagaggctgagccacccggaggggcgacctagcacgcaagcagtatgtggcccaacactggaaccaagcagcccggctccgggcgcaccttctcagggattcctcagggacaagtccagccccttgtcgtcaaggctcttgtagaccgacgtagggaccaatagaaccccgtgcggtggagctattgtgaaggagcaaaaaagtgccctggttctaagaggacgtcttaggggaagtgacggctgagttgaggtggatccggctggcgatgtaaggttcgagccatataaacccgggaaccgggagcccttgacgacattgttccccgagtgcccggagtctgcggctttttttggggtggtggcagctggcggaagtgacgggagaggggtggggccgcgagagcggcggaagtaggaagccgaggtctgaattgcgcgtggtggccatggcggccagcggggctgtggaaccagggcccccgggggctgccgtcgccccgtcgcccgc

SEQ ID NO: 5 - DNA, 18 bp, Artificial Sequence
  sequencing oligonucleotide PrimerPU
tgtaaaacgacggccagt

SEQ ID NO: 6 - DNA, 18 bp, Artificial Sequence
  sequencing oligonucleotide PrimerRP
caggaaacagctatgacc

(19) United States
(12) Patent Application Publication
(10) Pub. No.: US 2003/0032783 A1
(43) Pub. Date: Feb. 13, 2003

(54) Apolipoprotein A-IV-related protein: polypeptide, polynucleotide sequences and biallelic markers thereof

(76) Inventors: Frances Yen-Potin, San Diego, CA (US); Blake Denison, San Diego, CA (US); Jean Baptiste Dumas Milne Edwards, Paris (FR); Bernard Bihain, Carlsbad, CA (US); Barbara Bour, San Diego, CA (US); Aymeric Duclert, Saint-Maur (FR); Lydie Bougueleret, Petit Lancy (CH)

Correspondence Address: KNOBBE MARTENS OLSON & BEAR LLP, 2040 MAIN STREET, FOURTEENTH FLOOR, IRVINE, CA, 92614, US

(21) Appl. No.: 09/842,364

(22) Filed: Apr. 25, 2001

Related U.S. Application Data
(63) Continuation of application No. 09/599,362, filed on Jun. 21, 2000, now abandoned.
(63) Continuation-in-part of application No. 09/469,099, filed on Dec. 21, 1999, now abandoned.
(60) Provisional application No. 60/113,686, filed on Dec. 22, 1998.
(60) Provisional application No. 60/141,032, filed on Jun. 25, 1999.

(30) Foreign Application Priority Data
Dec. 20, 1999 (WO) PCT/IB99/02058

(51) Int. Cl. C07H 21/02; C07H 21/04

(52) U.S. Cl. 536/23.1

(57) ABSTRACT

The invention provides the genomic sequence of AA4RP, AA4RP cDNAs and AA4RP polypeptides. Further the invention provides polynucleotides including biallelic markers derived from the AA4RP gene and from genomic regions flanking the gene. This invention also provides polynucleotides and methods suitable for genotyping a nucleic acid containing sample for one or more biallelic markers of the invention. Further, the invention provides methods to detect a statistical correlation between a biallelic marker allele and a phenotype and/or between a biallelic marker haplotype and a phenotype. The invention also relates to diagnostic methods for determining whether an individual is at risk of developing a lipid metabolism related disorder and/or a liver related disorder, or whether said individual suffers from a lipid metabolism related disorder and/or a liver related disorder as a result of a polymorphism in the AA4RP gene.

### RELATED APPLICATIONS

The present application is a continuation of U.S. patent application Ser. No. 09/599,362, filed Jun. 21, 2000, which claims priority to PCT Patent Application No. PCT/IB99/02058 filed Dec. 20, 1999 and is a continuation-in-part of U.S. patent application Ser. No. 09/469,099 filed Dec. 21, 1999, both of which claim priority to U.S. Provisional Patent Application Serial No. 60/113,686, filed Dec. 22, 1998, and U.S. Provisional Patent Application Serial No. 60/141,032, filed Jun. 25, 1999, all of which are hereby incorporated by reference herein in their entirety, including any figures, tables, or drawings.

### FIELD OF THE INVENTION

The present invention is directed to polynucleotides encoding apolipoprotein A-IV-related protein (AA4RP) as well as the regulatory regions located at the 5′- and 3′-end of the coding region. The invention also concerns polypeptides encoded by the AA4RP gene. The invention also deals with antibodies directed specifically against such polypeptides that are useful as diagnostic reagents. The invention further encompasses biallelic markers of the AA4RP gene useful in genetic analysis.

### BACKGROUND OF THE INVENTION

Obesity is a public health problem that is both serious and widespread. In industrialized countries a third of the population is at least 20% overweight. In the United States, the percentage of obese people has increased from 25% at the end of the 70 s, to 33% at the beginning of the 90 s.

Obesity considerably increases the risk of developing cardiovascular or metabolic diseases, including hypertension, hyperlipidemia, diabetes, cerebral apoplexy, arteriosclerosis, myocardial infarction, etc. Coronary insufficiency, atheromatous disease, and cardiac insufficiency are at the forefront of the cardiovascular complications induced by obesity. It is estimated that if the entire population had an ideal weight, the risk of coronary insufficiency would decrease by 25%, and the risk of cardiac insufficiency and of cerebral vascular accidents by 35%. The incidence of coronary diseases is doubled in subjects under 50 years who are 30% overweight. Studies carried out for other diseases are equally eloquent: the risk of high blood pressure is doubled in subjects 20% overweight; the risk of developing a non-insulin-dependent diabetes is tripled in subjects 30% overweight; and the risk of hyperlipidemias is multiplied by 6. The list of diseases whose onset is promoted by obesity includes: hyperuricemia (11.4% in obese subjects, against 3.4% in the general population), digestive pathologies, abnormalities in hepatic functions, and even certain cancers.

Whether the physiological changes in obesity are characterized by an increase in the number of adipose cells, or by an increase in the quantity of triglycerides stored in each adipose cell, or by both, this excess weight results mainly from an imbalance between the quantities of calories consumed and of calories used by the body. Studies on the causes of this imbalance have focused on the mechanism of absorption of foods, and therefore the molecules which control food intake and the feeling of satiety.

One such class of molecules is lipoproteins, high molecular weight particles that are primarily responsible for lipid transport (triglycerides and cholesterol in the form of cholesteryl esters) through the plasma. Lipoproteins include chylomicrons and chylomicron remnant particles, very low density lipoprotein (VLDL), intermediate density lipoprotein (IDL), low density lipoprotein (LDL), and high density lipoprotein (HDL), each differing in density, size, lipid composition, apolipoprotein composition and elctrophoretic mobility. Elevated levels of lipoproteins have been positively correlated with atherosclerosis, which accounts for approximately half of all deaths in the United States. In addition, strong clinical evidence correlates a reduction in plasma lipoprotein concentration with a reduced risk of atherosclerosis (Noma, A., et al. (1987)).

Lipoproteins are composed of a non-polar core region, a surrounding phospholipid surface coating containing small amounts of cholesterol, and apolipoproteins. Apolipoproteins are the protein component of lipoproteins and are responsible for binding to receptors on cell membranes and directing the lipoproteins to their intended site of metabolism. In addition, individual apolipoproteins have unique functions such as the formation of specific associations with lipoprotein particles of distinct density classes. Some apolipoproteins act as ligands controlling the interaction of lipoproteins with cell surface receptors, while others function as cofactors for essential enzymes in lipid metabolism.

At least ten different apolipoprotein molecules have been identified, and each class of lipoprotein particle contains a specific apolipoprotein or combination of apolipoproteins embedded in its surface. These apolipoproteins are encoded by genes localized to sites on chromosomes 1, 2, 6, 11 and 19, and mutations thereof are thought to play a role in a wide range of lipid metabolism related disorders such as atherogenesis and obesity.

One particular apolipoprotein believed to play a major role in lipid metabolism and its related disorders is apolipoprotein A-IV (apo A-IV). Apo A-IV is a 46,000-Da polypeptide expressed primarily by the small intestine in humans, but also expressed at low levels in the liver (Swaney et al. (1988), Ochoa A. et al. (1993)). The apo A-IV structure consists of thirteen 22-amino acid tandem repeats (each 22-mer is actually a tandem array of two, a and b, related 11-mers), nine of which are predicted to be highly alpha-helical. Many of these helices are amphipathic; and are therefore believed to serve as lipid-binding domains with lecithin.

During secretion from the small intestine epithelial cells, the twenty amino acid pre-apo A-IV signal peptide is cleaved (Gordon et al. (1982)). The remaining apo A-IV molecule is secreted into the lymph as a major constituent of newly synthesized triglyceride-rich lipoproteins as well as the HDL fraction of blood.

Apo A-IV circulates in the blood, and is therefore easily amenable to therapeutic intervention, by direct administration into the blood of synthetic peptide analogs that mimic its activity or function as competitive antagonists (dominant negatives). Since this protein is involved in lipid metabolism and mediates the changes in blood cholesterol in response to dietary changes, interventions targeted at this protein will be useful for cholesterol lowering and anti-atherosclerosis therapeutics, and in the control of diabetes and obesity (WO 99/50286). For example, peptides derived from apo A-IV possess lipid oxidation suppressant properties as well as hypolipidaemic properties. They show the capability to prevent and/or delay the oxidative modification of LDLs; thus representing a viable means for treating atherosclerosis and other oxidative disorders (PCT/US99/06580). In addition, apo A-IV may serve as a therapeutic agent in a pharmaceutical composition in the treatment of septic shock or disease conditions associated with elevated serum levels of Lipoprotein(a) (U.S. Pat. Nos. 5,932,536 and 5,948,756).

### SUMMARY OF THE INVENTION

The present invention stems from research focusing on lipid metabolism and its role in the pathophysiology of various disorders and diseases, including but not limited to obesity, diabetes and coronary heart disease. In particular, the inventors discovered and characterized a gene and its associated protein, apolipoprotein A-IV-related protein (AA4RP). Experiments have shown that it is differentially expressed in obese mice; being over-expressed in mice on a high-fat diet compared to mice on a normal diet. The protein is a homolog of the regeneration associated protein 3 (RAP3), a secreted protein whose plasma level increases after liver damage.

Apolipoproteins are the protein components of lipoproteins found in the plasma. A TBLASTN database revealed apolipoprotein A-IV-related protein (AA4RP) is a member of the apolipoprotein family, having 52% similarity and 29% identity to apolipoprotein A-IV. See FIGS. 7 and 8. Apo A-IV is found associated with the chylomicron and HDL fraction of blood. It is expressed in the liver and intestine and is up-regulated by high fat meals and down regulated by leptin (Ochoa A. et al. (1993), Elshourbagy N. A. et al. (1987), Simonet W. S. et al. (1993)). Levels of apo A-IV are correlated with glycemic control in young type I diabetes (IDDM) patients. Over-expression of the protein is protective against atherosclerosis in mice with ApoE knockouts. Lack of ApoE, a well established anti-atherogenic protein, results in a greater risk of developing coronary heart disease due to a more severe atherosclerotic lipid profile (Duverger, N. et al. (1996)). Finally, apo A-IV is responsible for part of the inter-individual variability in blood cholesterol response to changes in dietary fat/cholesterol intake.

Expression of apolipoproteins is known to be under the control of developmental, hormonal, dietary and tissue specific regulation. The inventors found AA4RP is differentially expressed in obese mouse models: up regulated in mice fed a high fat diet (cafeteria diet) and in naturally obese mice (NZO), while it was not differentially expressed in either mice lacking the gene for leptin (ob/ob) or in mice lacking the gene for the leptin receptor (db/db), suggesting AA4RP is regulated by diet. Thus inhibitors of gene expression and antagonists protein activity that decrease the concentration of AA4RP should serve as important therapeutic compounds in the treatment of lipid metabolism related disorders, while up-regulators of the gene and protein agonists could serve as a means of weight gain for patients.

Since the rat homolog of AA4RP (RAP3) is associated with liver regeneration and specifically with increased serum concentration following liver damage, antagonists and agonists of AA4RP may be useful in treatment involving liver regeneration. See FIGS. 9 and 10. Antibodies can be used in the diagnosis of liver disease and damage, by detecting, for example, the presence of AA4RP secreted into the bloodstream (Wu, Chuan-Ging et al. (1997)).

A first embodiment of the invention is a recombinant, purified or isolated polynucleotide comprising, or consisting of a mammalian genomic sequence, gene, or fragments thereof. In one aspect the sequence is derived from a human, mouse or other mammal. In a preferred aspect, the genomic sequence includes isolated, purified, or recombinant polynucleotides comprising a contiguous span of at least 12, 15, 18, 20, 22, 25, 30, 35, 40, 50, 60, 70, 80, 90, 100, 150, 200, 500, 1000, 2000, 5000, 10000 or 50000 nucleotides of SEQ ID No 1, or the complements thereof, wherein said contiguous span comprises at least 1, 2, 3, 5, or 10 of the following nucleotide positions of SEQ ID No 1: 739-1739; 10946-12958; 13470-13526; 13641-13752; 14271-17969; 41718-42718; 44942-45942; and 76558-77558. Further preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 1, or the complements thereof, wherein said contiguous contains one or more of the nucleotides at positions 1239, 12347, 15241, 42218, 45442, or 77058. Optionally, the polynucleotide consists of, consists essentially of, or comprises a contiguous span of nucleotides of a human genomic sequence, preferably a sequence selected from SEQ ID No 1, wherein said contiguous span is at least 6, 8, 10, 12, 15, 20, 25, 30, 50, 100, 200, 500 or 1000 nucleotides in length and contains one or more of the nucleotides at positions 13269 or 13475.

Another embodiment of the invention is a recombinant, purified or isolated polynucleotide comprising, or consisting of a mammalian genomic sequence, gene, or fragments thereof. In one aspect the sequence is derived from a human, mouse or other mammal. In a preferred aspect, the genomic sequence is selected from the human genomic sequence of SEQ ID No 4. Optionally, the polynucleotide consists of, consists essentially of, or comprises a contiguous span of nucleotides of a human genomic sequence, preferably a sequence selected from SEQ ID No 4, wherein said contiguous span is at least 6, 8, 10, 12, 15, 20, 25, 30, 50, 100, 200, 500 or 1000 nucleotides in length and contains one or more of the nucleotides at positions 1241 or 1447. Optionally, the polynucleotide consists of, consists essentially of, or comprises a contiguous span of nucleotides of a human genomic sequence, preferably SEQ ID No 4, wherein said contiguous span comprises at least 6, 8, 10, 12, 15, 20, 25, 30, 50, 100, 200, 500 or 1000 nucleotides of the following nucleotide positions of SEQ ID No 4: 1-1498, 1613-1724, 2243-3940, and 3941-5381.

A second embodiment of the present invention is a recombinant, purified or isolated polynucleotide comprising, or consisting of a mammalian cDNA sequence, or fragments thereof. In one aspect the sequence is derived from a human, mouse or other mammal. In a preferred aspect, the cDNA sequence is selected from the human cDNA sequence of SEQ ID No 2 or the complement thereto. Optionally, said polynucleotide consists of, consists essentially of, or comprises a contiguous span of nucleotides of a mammalian cDNA sequence, preferably SEQ ID No 2. Preferred fragments of said cDNA include the fragments delineated by the exons of SEQ ID NO:4 (1-1498, 1613-1724, 2243-3940 and 3941-5381).

A third embodiment of the present invention is a recombinant, purified or isolated polynucleotide, or the complement thereof, encoding a mammalian AA4RP protein, or a fragment thereof. In one aspect the AA4RP protein sequence is from a human, mouse or other mammal. In a preferred aspect, the AA4RP protein sequence is selected from the human AA4RP protein sequence of SEQ ID No 3. Optionally, said fragment of AA4RP polynucleotide consists of, consists essentially of, or comprises a contiguous stretch of at least 8, 10, 12, 15, 20, 25, 30, 50, 100, 200 or 500 nucleotides from SEQ ID No 2, as well as any other human, mouse or mammalian AA4RP polynucleotides.

A fourth embodiment of the invention are the polynucleotide primers and probes disclosed herein.

A fifth embodiment of the present invention is a recombinant, purified or isolated polypeptide comprising or consisting of a mammalian AA4RP protein, or a fragment thereof. In one aspect the AA4RP protein sequence is from a human, mouse or other mammal. In a preferred aspect, the AA4RP protein sequence is selected from the human AA4RP protein sequence of SEQ ID No 3. Optionally, said fragment of AA4RP polypeptide consists of, consists essentially of, or comprises a contiguous stretch of at least 8, 10, 12, 15, 20, 25, 30, 50, 100 or 200 amino acids from SEQ ID No 3, as well as any other human, mouse or mammalian AA4RP polypeptide.

A sixth embodiment of the present invention is an antibody composition capable of specifically binding to a polypeptide of the invention. Optionally, said antibody is polyclonal or monoclonal. Optionally, said polypeptide is an epitope-containing fragment of at least 8, 10, 12, 15, 20, 25, or 30 amino acids of a human, mouse, or mammalian AA4RP protein, preferably a sequence selected from SEQ ID No 3.

A seventh embodiment of the present invention is a vector comprising any polynucleotide of the invention. Optionally, said vector is an expression vector, gene therapy vector, amplification vector, gene targeting vector, or knock-out vector.

An eighth embodiment of the present invention is a host cell comprising any vector of the invention.

A ninth embodiment of the present invention is a mammalian host cell comprising a AA4RP gene disrupted by homologous recombination with a knock out vector.

A tenth embodiment of the present invention is a nonhuman host mammal or animal comprising a vector of the invention.

A further embodiment of the present invention is a nonhuman host mammal comprising a AA4RP gene disrupted by homologous recombination with a knock out vector.

Another embodiment of the present invention is a method of determining whether an individual is at risk of developing a disease involving lipid metabolism and/or a liver related disorder at a later date or whether the individual suffers from a lipid metabolism related disorder and/or a liver related disorder as a result of a mutation in the AA4RP gene comprising obtaining a nucleic acid sample from the individual; and determining whether the nucleotides present at one or more of the AA4RP-related biallelic markers of the invention are indicative of a risk of developing a lipid metabolism related disorder and/or a liver related disorder at a later date or indicative of a lipid metabolism related disorder and/or a liver related disorder resulting from a mutation in the AA4RP gene. Optionally, said AA4RP-related biallelic is a AA4RP-related biallelic marker positioned in SEQ ID Nos 1, 2 or 4; one or more AA4RP-related biallelic marker selected from the group consisting of 20-828-311, 17-42-319, 17-41-250, 20-841-149, 20-842-115, and 20-853-415;

or more preferably a AA4RP-related biallelic marker selected from the group consisting of 17-42-319 and 17-41-250.

Another embodiment of the present invention is a method of determining whether an individual is at risk of developing a lipid metabolism related disorder and/or a liver related disorder at a later date or whether the individual suffers from a lipid metabolism related disorder and/or a liver related disorder as a result of a mutation in the AA4RP gene comprising obtaining a nucleic acid sample from the individual and determining whether the nucleotides present at one or more of the polymorphic bases in a AA4RP-related biallelic marker. Optionally, said AA4RP-related biallelic is a AA4RP-related biallelic marker positioned in SEQ ID Nos 1, 2 or 4; one or more of the AA4RP-related biallelic marker selected from the group consisting of 20-828-311, 17-42-319, 17-41-250, 20-841-149, 20-842-115, and 20-853-415; or more preferably a AA4RP-related biallelic marker selected from the group consisting of 17-42-319 and 17-41-250.

Another embodiment of the present invention is a method of obtaining an allele of the AA4RP gene which is associated with a detectable phenotype comprising obtaining a nucleic acid sample from an individual expressing the detectable phenotype, contacting the nucleic acid sample with an agent capable of specifically detecting a nucleic acid encoding the AA4RP protein, and isolating the nucleic acid encoding the AA4RP protein. In one aspect of this method, the contacting step comprises contacting the nucleic acid sample with at least one nucleic acid probe capable of specifically hybridizing to said nucleic acid encoding the AA4RP protein. In another aspect of this embodiment, the contacting step comprises contacting the nucleic acid sample with an antibody capable of specifically binding to the AA4RP protein. In another aspect of this embodiment, the step of obtaining a nucleic acid sample from an individual expressing a detectable phenotype comprises obtaining a nucleic acid sample from an individual suffering from a lipid metabolism related disorder and/or a liver related disorder.

Another embodiment of the present invention is a method of obtaining an allele of the AA4RP gene which is associated with a detectable phenotype comprising obtaining a nucleic acid sample from an individual expressing the detectable phenotype, contacting the nucleic acid sample with an agent capable of specifically detecting a sequence within the 11 q23 region of the human genome, identifying a nucleic acid encoding the AA4RP protein in the nucleic acid sample, and isolating the nucleic acid encoding the AA4RP protein. In one aspect of this embodiment, the nucleic acid sample is obtained from an individual suffering from a lipid metabolism related disorder and/or a liver related disorder.

Another embodiment of the present invention is a method of categorizing the risk of a lipid metabolism related disorder and/or a liver related disorder in an individual comprising the step of assaying a sample taken from the individual to determine whether the individual carries an allelic variant of AA4RP associated with an increased risk of a lipid metabolism related disorder and/or a liver related disorder. In one aspect of this embodiment, the sample is a nucleic acid sample. In another aspect a nucleic acid sample is assayed by determining the frequency of the AA4RP transcripts present. In another aspect of this embodiment, the sample is a protein sample. In another aspect of this embodiment, the method further comprises determining whether the AA4RP protein in the sample binds an antibody specific for a AA4RP isoform associated with a lipid metabolism related disorder and/or a liver related disorder.

Another embodiment of the present invention is a method of categorizing the risk of a lipid metabolism related disorder and/or a liver related disorder in an individual comprising the step of determining whether the identities of the polymorphic bases of one or more biallelic markers which are in linkage disequilibrium with the AA4RP gene are indicative of an increased risk of a lipid metabolism related disorder and/or a liver related disorder.

Amother embodiment of the present invention features a method of treating or preventing a lipid metabolism related disorder and/or a liver-related disorder in an individual comprising administering to an individual in need of such treatment an AA4RP polypeptide of the invention in a pharmaceutically acceptable composition. Alternatively, antagonists or agonists of AA4RP activity can be provided, or compounds that enhance or inhibit the expression of AA4RP.

Another embodiment of the present invention comprises a method of identifying molecules which specifically bind to a AA4RP protein, preferably the protein of SEQ ID No 3 or a portion thereof: comprising the steps of introducing a nucleic a nucleic acid encoding the protein of SEQ ID No 3 or a portion thereof into a cell such that the protein of SEQ ID No 3 or a portion thereof contacts proteins expressed in the cell and identifying those proteins expressed in the cell which specifically interact with the protein of SEQ ID No 3 or a portion thereof.

Another embodiment of the present invention is a method of identifying molecules which specifically bind to the protein of SEQ ID No 3 or a portion thereof. One step of the method comprises linking a first nucleic acid encoding the protein of SEQ ID No 3 or a portion thereof to a first indicator nucleic acid encoding a first indicator polypeptide to generate a first chimeric nucleic acid encoding a first fusion protein. The first fusion protein comprises the protein of SEQ ID No 3 or a portion thereof and the first indicator polypeptide. Another step of the method comprises linking a second nucleic acid nucleic acid encoding a test polypeptide to a second indicator nucleic acid encoding a second indicator polypeptide to generate a second chimeric nucleic acid encoding a second fusion protein. The second fusion protein comprises the test polypeptide and the second indicator polypeptide. Association between the first indicator protein and the second indicator protein produces a detectable result. Another step of the method comprises introducing the first chimeric nucleic acid and the second chimeric nucleic acid into a cell. Another step comprises detecting the detectable result.

A further embodiment of the invention is a purified or isolated mammalian AA4RP gene or cDNA sequence.

Further embodiments of the present invention include the nucleic acid and amino acid sequences of mutant or low frequency AA4RP alleles derived from lipid metabolism related disorder and/or liver related disorder patients, tissues or cell lines. The present invention also encompasses methods which utilize detection of these mutant AA4RP sequences in an individual or tissue sample to diagnosis a lipid metabolism related disorder and/or a liver related disorder, assess the risk of developing a lipid metabolism related disorder and/or a liver related disorder or assess the likely severity of said disorder.

Another embodiment of the invention encompasses any polynucleotide of the invention attached to a solid support. In addition, the polynucleotides of the invention which are attached to a solid support encompass polynucleotides with any further limitation described in this disclosure, or those following: Optionally, said polynucleotides is specified as attached individually or in groups of at least 2, 5, 8, 10, 12, 15, 20, or 25 distinct polynucleotides of the inventions to a single solid support. Optionally, polynucleotides other than those of the invention may be attached to the same solid support as polynucleotides of the invention. Optionally, when multiple polynucleotides are attached to a solid support they are attached at random locations, or in an ordered array. Optionally, said ordered array is addressable.

An additional embodiment of the invention encompasses the use of any polynucleotide for, or any polynucleotide for use in, determining the identity of an allele at a AA4RP-related biallelic marker. In addition, the polynucleotides of the invention for use in determining the identity of an allele at a AA4RP-related biallelic marker encompass polynucleotides with any further limitation described in this disclosure, or those following: Optionally, said AA4RP-related biallelic marker is a AA4RP-related biallelic marker positioned in SEQ ID Nos 1, 2 or 4; one or more AA4RP-related biallelic marker selected from the group consisting of 20-828-311, 17-42-319, 17-41-250, 20-841-149, 20-842-115, and 20-853-415; or more preferably a AA4RP-related biallelic marker selected from the group consisting of 17-42-319 and 17-41-250. Optionally, said polynucleotide may comprise a sequence disclosed in the present specification. Optionally, said polynucleotide may consist of, or consist essentially of any polynucleotide described in the present specification. Optionally, said determining is performed in a hybridization assay, sequencing assay, microsequencing assay, or allele-specific amplification assay. Optionally, said polynucleotide is attached to a solid support, array, or addressable array. Optionally, said polynucleotide is labeled.

Another embodiment of the invention encompasses the use of any polynucleotide for, or any polynucleotide for use in, amplifying a segment of nucleotides comprising an AA4RP-related biallelic marker. In addition, the polynucleotides of the invention for use in amplifying a segment of nucleotides comprising a AA4RP-related biallelic marker encompass polynucleotides with any further limitation described in this disclosure, or those following: Optionally, said AA4RP-related biallelic marker is a AA4RP-related biallelic marker positioned in SEQ ID Nos 1, 2 or 4; one or more AA4RP-related biallelic marker selected from the group consisting of 20-828-311, 17-42-319, 17-41-250, 20-841-149, 20-842-115, and 20-853-415; or more preferably a AA4RP-related biallelic marker selected from the group consisting of 17-42-319 and 17-41-250. Optionally, said polynucleotide may comprise a sequence disclosed in the present specification. Optionally, said polynucleotide may consist of, or consist essentially of any polynucleotide described in the present specification. Optionally, said amplifying is performed by a PCR or LCR. Optionally, said polynucleotide is attached to a solid support, array, or addressable array. Optionally, said polynucleotide is labeled.

A further embodiment of the invention encompasses methods of genotyping a biological sample comprising determining the identity of an allele at an AA4RP-related biallelic marker. In addition, the genotyping methods of the invention encompass methods with any further limitation described in this disclosure, or those following: Optionally, said AA4RP-related biallelic marker is a AA4RP-related biallelic marker positioned in SEQ ID Nos 1, 2 or 4; one or more AA4RP-related biallelic marker selected from the group consisting of 20-828-311, 17-42-319, 17-41-250, 20-841-149, 20-842-115, and 20-853-415; or more preferably a AA4RP-related biallelic marker selected from the group consisting of 17-42-319 and 17-41-250. Optionally, said method further comprises determining the identity of a second allele at said biallelic marker, wherein said first allele and second allele are not base paired (by Watson & Crick base pairing) to one another. Optionally, said biological sample is derived from a single individual or subject. Optionally, said method is performed in vitro. Optionally, said biallelic marker is determined for both copies of said biallelic marker present in said individual's genome. Optionally, said biological sample is derived from multiple subjects or individuals. Optionally, said method further comprises amplifying a portion of said sequence comprising the biallelic marker prior to said determining step. Optionally, wherein said amplifying is performed by PCR, LCR, or replication of a recombinant vector comprising an origin of replication and said portion in a host cell. Optionally, wherein said determining is performed by a hybridization assay, sequencing assay, microsequencing assay, or allele-specific amplification assay.

An additional embodiment of the invention comprises methods of estimating the frequency of an allele in a population comprising determining the proportional representation of an allele at a AA4RP-related biallelic marker in said population. In addition, the methods of estimating the frequency of an allele in a population of the invention encompass methods with any further limitation described in this disclosure, or those following: Optionally, said AA4RP-related biallelic marker is a AA4RP-related biallelic marker positioned in SEQ ID Nos 1, 2 or 4; one or more AA4RP-related biallelic marker selected from the group consisting of 20-828-311, 17-42-319, 17-41-250, 20-841-149, 20-842-115, and 20-853-415; or more preferably a AA4RP-related biallelic marker selected from the group consisting of 17-42-319 and 17-41-250. Optionally, determining the proportional representation of an allele at a AA4RP-related biallelic marker is accomplished by determining the identity of the alleles for both copies of said biallelic marker present in the genome of each individual in said population and calculating the proportional representation of said allele at said AA4RP-related biallelic marker for the population. Optionally, determining the proportional representation is accomplished by performing a genotyping method of the invention on a pooled biological sample derived from a representative number of individuals, or each individual, in said population, and calculating the proportional amount of said nucleotide compared with the total.

A further embodiment of the invention comprises methods of detecting an association between a genotype and a phenotype, comprising the steps of a) genotyping at least one AA4RP-related biallelic marker in a trait positive population according to a genotyping method of the invention; b) genotyping said AA4RP-related biallelic marker in a control population according to a genotyping method of the invention; and c) determining whether a statistically significant association exists between said genotype and said phenotype. In addition, the methods of detecting an association between a genotype and a phenotype of the invention encompass methods with any further limitation described in this disclosure, or those following: SEQ ID Nos 1, 2 or 4; one or more AA4RP-related biallelic marker selected from the group consisting of 20-828-311, 17-42-319, 17-41-250, 20-841-149, 20-842-115, and 20-853-415; or more preferably a AA4RP-related biallelic marker selected from the group consisting of 17-42-319 and 17-41-250. Optionally, said control population is a trait negative population, or a random population. Optionally, each of said genotyping steps a) and b) is performed on a single pooled biological sample derived from each of said populations. Optionally, each of said genotyping of steps a) and b) is performed separately on biological samples derived from each individual in said population or a subsample thereof. Optionally, said phenotype is a lipid metabolism related disorder and/or a liver related disorder; a response to an agent acting on lipid metabolism and/or liver related disorders; or a side effect to an agent acting on lipid metabolism. Optionally, said method comprises the additional steps of determining the phenotype in said trait positive and said control populations prior to step c).

An additional embodiment of the present invention encompasses methods of estimating the frequency of a haplotype for a set of biallelic markers in a population, comprising the steps of: a) genotyping at least one AA4RP-related biallelic marker for both copies of said set of biallelic marker present in the genome of each individual in said population or a subsample thereof, according to a genotyping method of the invention; b) genotyping a second biallelic marker by determining the identity of the allele at said second biallelic marker for both copies of said second biallelic marker present in the genome of each individual in said population or said subsample, according to a genotyping method of the invention; and c) applying a haplotype determination method to the identities of the nucleotides determined in steps a) and b) to obtain an estimate of said frequency. In addition, the methods of estimating the frequency of a haplotype of the invention encompass methods with any further limitation described in this disclosure, or those following: Optionally, said AA4RP-related biallelic marker is a AA4RP-related biallelic marker positioned in SEQ ID Nos 1, 2 or 4; one or more AA4RP-related biallelic marker selected from the group consisting of 20-828-311, 17-42-319, 17-41-250, 20-841-149, 20-842-115, and 20-853-415; or more preferably a AA4RP-related biallelic marker selected from the group consisting of 17-42-319 and 17-41-250. Optionally, said haplotype determination method is an expectation-maximization algorithm.

An additional embodiment of the present invention encompasses methods of detecting an association between a haplotype and a phenotype, comprising the steps of: a) estimating the frequency of at least one haplotype in a trait positive population, according to a method of the invention for estimating the frequency of a haplotype; b) estimating the frequency of said haplotype in a control population, according to a method of the invention for estimating the frequency of a haplotype; and c) determining whether a statistically significant association exists between said haplotype and said phenotype. In addition, the methods of detecting an association between a haplotype and a phenotype of the invention encompass methods with any further limitation described in this disclosure, or those following: Optionally, said AA4RP-related biallelic is a AA4RP-related biallelic marker positioned in SEQ ID Nos 1, 2 or 4; one or more AA4RP-related biallelic marker selected from the group consisting of 20-828-311, 17-42-319, 17-41-250, 20-841-149, 20-842-115, and 20-853-415; or more preferably a AA4RP-related biallelic marker selected from the group consisting of 17-42-319 and 17-41-250. Optionally, said haplotype exhibits a p-value of <1×10⁻³ in an association with a trait positive population with a disorder, preferably a lipid metabolism related disorder and/or a liver related disorder. Optionally, said control population is a trait negative population, or a random population. Optionally, said phenotype is a lipid metabolism related disorder and/or a liver related disorder; a response to an agent acting on lipid metabolism and/or liver related disorders; or a side effect to an agent acting on lipid metabolism. Optionally, said method comprises the additional steps of determining the phenotype in said trait positive and said control populations prior to step c).

Another embodiment of the present invention is a method of administering a drug or a treatment comprising the steps of: a) obtaining a nucleic acid sample from an individual; b) determining the identity of the polymorphic base of at least one AA4RP-related biallelic marker which is associated with a positive response to the treatment or the drug; or at least one biallelic AA4RP-related biallelic marker which is associated with a negative response to the treatment or the drug; and c) administering the treatment or the drug to the individual if the nucleic acid sample contains said biallelic marker associated with a positive response to the treatment or the drug or if the nucleic acid sample lacks said biallelic marker associated with a negative response to the treatment or the drug. In addition, the methods of the present invention for administering a drug or a treatment encompass methods with any further limitation described in this disclosure, or those following, specified alone or in any combination: optionally, said AA4RP-related biallelic marker may be in a sequence selected individually or in any combination from the group consisting of SEQ ID Nos. 1, 2 and 4; and the complements thereof; or optionally, the administering step comprises administering the drug or the treatment to the individual if the nucleic acid sample contains said biallelic marker associated with a positive response to the treatment or the drug and the nucleic acid sample lacks said biallelic marker associated with a negative response to the treatment or the drug.

Another embodiment of the present invention is a method of selecting an individual for inclusion in a clinical trial of a treatment or drug comprising the steps of: a) obtaining a nucleic acid sample from an individual; b) determining the identity of the polymorphic base of at least one AA4RP-related biallelic marker which is associated with a positive response to the treatment or the drug, or at least one AA4RP-related biallelic marker which is associated with a negative response to the treatment or the drug in the nucleic acid sample, and c) including the individual in the clinical trial if the nucleic acid sample contains said AA4RP-related biallelic marker associated with a positive response to the treatment or the drug or if the nucleic acid sample lacks said biallelic marker associated with a negative response to the treatment or the drug. In addition, the methods of the present invention for selecting an individual for inclusion in a clinical trial of a treatment or drug encompass methods with any further limitation described in this disclosure, or those following, specified alone or in any combination: Optionally, said AA4RP-related biallelic marker may be in a sequence selected individually or in any combination from the group consisting of SEQ ID Nos. 1, 2 and 4; and the complements thereof; optionally, the including step comprises administering the drug or the treatment to the individual if the nucleic acid sample contains said biallelic marker associated with a positive response to the treatment or the drug and the nucleic acid sample lacks said biallelic marker associated with a negative response to the treatment or the drug.

Additional embodiments and aspects of the present invention are set forth in the Detailed Description of the Invention and the Examples.

### BRIEF DESCRIPTION OF THE DRAWINGS

FIG. 1 is a chart containing a list of the AA4RP-related biallelic markers. Each marker is described by indicating its SEQ ID NO., the biallelic marker ID, and the “ORIGINAL” allele and the “ALTERNATIVE” allele.

FIG. 2 is a chart containing a list of biallelic markers surrounded by preferred sequences. In the column labeled, “POSITION RANGE OF PREFERRED SEQUENCE” of FIG. 2, regions of particularly preferred sequences are listed for each SEQ ID which contain a AA4RP-related biallelic marker, as well as particularly preferred regions of sequences that may not contain a AA4RP-related biallelic marker but, which are in sufficiently close proximity to a AA4RP-related biallelic marker to be useful as amplification or sequencing primers.

FIGS. 3A and 3B are charts containing two nucleotide changes that conflict with existing genomic sequence. The SEQ ID NO., the position of conflict in SEQ ID No 1 and the corresponding position of conflict in SEQ ID No 4 as well as the “original” nucleotide present at the position of conflict in SEQ ID No 1 and the “alternative” nucleotide present at the position of conflict in SEQ ID No 4 are provided.

FIG. 4 is a chart listing microsequencing primers which may be used to genotype AA4RP-related biallelic markers and other preferred microsequencing primers for use in genotyping AA4RP-related biallelic markers. Each of the primers which falls within the strand of nucleotides included in the Sequence Listing are described by indicating their Sequence ID number and the positions of the first and last nucleotides (position range) of the primers in the Sequence ID. Since the sequences in the Sequence Listing are single stranded and half the possible microsequencing primers are composed of nucleotide sequences from the complementary strand, the primers that are composed of nucleotides in the complementary strand are described by indicating their SEQ ID numbers and the positions of the first and last nucleotides to which they are complementary (complementary position range) in the Sequence ID.

FIG. 5 is a chart listing amplification primers which may be used to amplify polynucleotides containing one or more AA4RP-related biallelic markers. Each of the primers which falls within the strand of nucleotides included in the Sequence Listing are described by indicating their Sequence ID number and the positions of the first and last nucleotides (position range) of the primers in the Sequence ID. Since the sequences in the Sequence Listing are single stranded and half the possible amplification primers are composed of nucleotide sequences from the complementary strand, the primers that are composed of nucleotides in the complementary strand are defined by the SEQ ID numbers and the positions of the first and last nucleotides to which they are complementary (complementary position range) in the Sequence ID.

FIG. 6 is a chart listing preferred probes useful in genotyping AA4RP-related biallelic markers by hybridization assays. The probes are generally 25-mers with a AA4RP-related biallelic marker in the center position, and described by indicating their Sequence ID number and the positions of the first and last nucleotides (position range) of the probes in the Sequence ID. The probes complementary to the sequences in each position range in each Sequence ID are also understood to be a part of this preferred list even though they are not specified separately.

FIGS. 7A and 7B contain a chart showing the cDNA alignment of apo A-IV-related protein with human apo A-IV and swine apo A-IV.

FIG. 8 is a chart showing the protein alignment of apo A-IV-related protein with human apo A-IV and swine apo A-IV.

FIGS. 9A and 9B contain a chart showing the cDNA alignment of apo A-IV-related protein with rat RAP3 cDNA's (rn_RAP3_a.seq and m_RAP3_b.seq).

FIG. 10 is a chart showing the protein alignment of apo A-IV-related protein with rat RAP3 proteins (RAP3 a and RAP3 b).

FIG. 11 is a block diagram of an exemplary computer system.

FIG. 12 is a flow diagram illustrating one embodiment of a process 200 for comparing a new nucleotide or protein sequence with a database of sequences in order to determine the homology levels between the new sequence and the sequences in the database.

FIG. 13 is a flow diagram illustrating one embodiment of a process 250 in a computer for determining whether two sequences are homologous.

FIG. 14 is a flow diagram illustrating one embodiment of an identifier process 300 for detecting the presence of a feature in a sequence.

### BRIEF DESCRIPTION OF THE SEQUENCES PROVIDED IN THE SEQUENCE LISTING

SEQ ID No 1, Genbank Accession No. 007707, contains a partial genomic sequence from chromosome 11. The sequence comprises the 5′ regulatory region (upstream untranscribed region), the exons and introns, and the 3′ regulatory region (downstream untranscribed region) of AA4RP.

SEQ ID No 2 contains a cDNA sequence of AA4RP.

SEQ ID No 3 contains the amino acid sequence encoded by the cDNA of SEQ ID No 2.

SEQ ID No 4 contains an alternative genomic sequence of AA4RP comprising the 5′ regulatory region (upstream untranscribed region), the exons and introns, and the 3′ regulatory region (downstream untranscribed region).

SEQ ID No 5 contains a primer containing the additional PU 5′ sequence described further in Example 1.

SEQ ID No 6 contains a primer containing the additional RP 5′ sequence described further in Example 1.

In accordance with the regulations relating to Sequence Listings, the following codes have been used in the Sequence Listing to indicate the locations of biallelic markers within the sequences and to identify each of the alleles present at the polymorphic base. The code “r” in the sequences indicates that one allele of the polymorphic base is a guanine, while the other allele is an adenine. The code “y” in the sequences indicates that one allele of the polymorphic base is a thymine, while the other allele is a cytosine. The code “m” in the sequences indicates that one allele of the polymorphic base is an adenine, while the other allele is an cytosine. The code “k” in the sequences indicates that one allele of the polymorphic base is a guanine, while the other allele is a thymine. The code “s” in the sequences indicates that one allele of the polymorphic base is a guanine, while the other allele is a cytosine. The code “w” in the sequences indicates that one allele of the polymorphic base is an adenine, while the other allele is an thymine. The nucleotide code of the original allele for each biallelic marker is the following:

| Biallelic marker | Original allele |
|---|---|
| 5-124-273 | A (for example) |

In some instances, the polymorphic bases of the biallelic markers alter the identity of an amino acids in the encoded polypeptide. This is indicated in the accompanying Sequence Listing by use of the feature VARIANT, placement of an Xaa at the position of the polymorphic amino acid, and definition of Xaa as the two alternative amino acids. For example if one allele of a biallelic marker is the codon CAC, which encodes histidine, while the other allele of the biallelic marker is CAA, which encodes glutamine, the Sequence Listing for the encoded polypeptide will contain an Xaa at the location of the polymorphic amino acid. In this instance, Xaa would be defined as being histidine or glutamine.

In other instances, Xaa may indicate an amino acid whose identity is unknown because of nucleotide sequence ambiguity. In this instance, the feature UNSURE is used, placement of an Xaa at the position of the unknown amino acid and definition of Xaa as being any of the 20 amino acids or a limited number of amino acids suggested by the genetic code.

### DETAILED DESCRIPTION OF THE INVENTION

The AA4RP gene and associated protein share homology with both apolipoprotein A-IV and regeneration associated protein and are expected to have similar functions. In addition, experiments have shown that AA4RP is differentially expressed in obese mice models, further indicating its role in lipid metabolism related disorders and/or liver related disorders. In particular, the invention is drawn to AA4RP polypeptides, polynucleotides encoding AA4RP polypeptides, vectors comprising AA4RP polynucleotides, and cells comprising AA4RP polynucleotides, as well as to pharmaceutical compositions comprising AA4RP polypeptides and methods of administering AA4RP pharmaceutical compositions in order to reduce body weight or to treat lipid metabolism related disorders and/or liver related disorders.

The human AA4RP cDNA was cloned and given the internal designation 117-005-2-0-E10-FLC. Clone 117-005-2-0-E10-FLC was deposited as part of a pool of clones with the ECACC and given the accession No. 99061735. SEQ ID No 2 represents the nucleotide sequence of the AA4RP cDNA. SEQ ID No 3 represents the protein encoded by SEQ ID No 2.

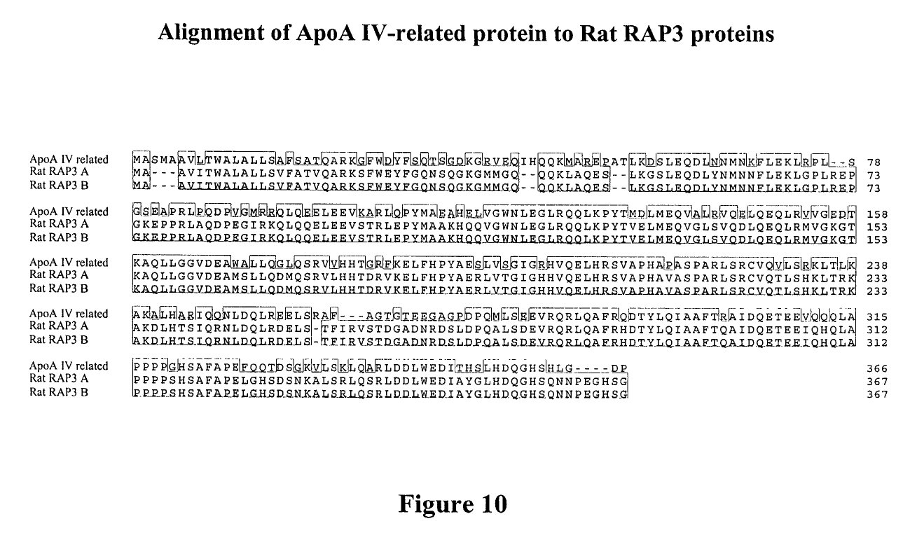

The protein of SEQ ID No 3 encoded by the cDNA of SEQ ID No 2 exhibits significant homology with rat regeneration associated protein (RAP3). See FIG. 10. It appears to be the human homolog of rat RAP3 and is likely to have a similar function. RAP3 is believed to be involved in liver regeneration and its concentration in serum increases following liver damage.

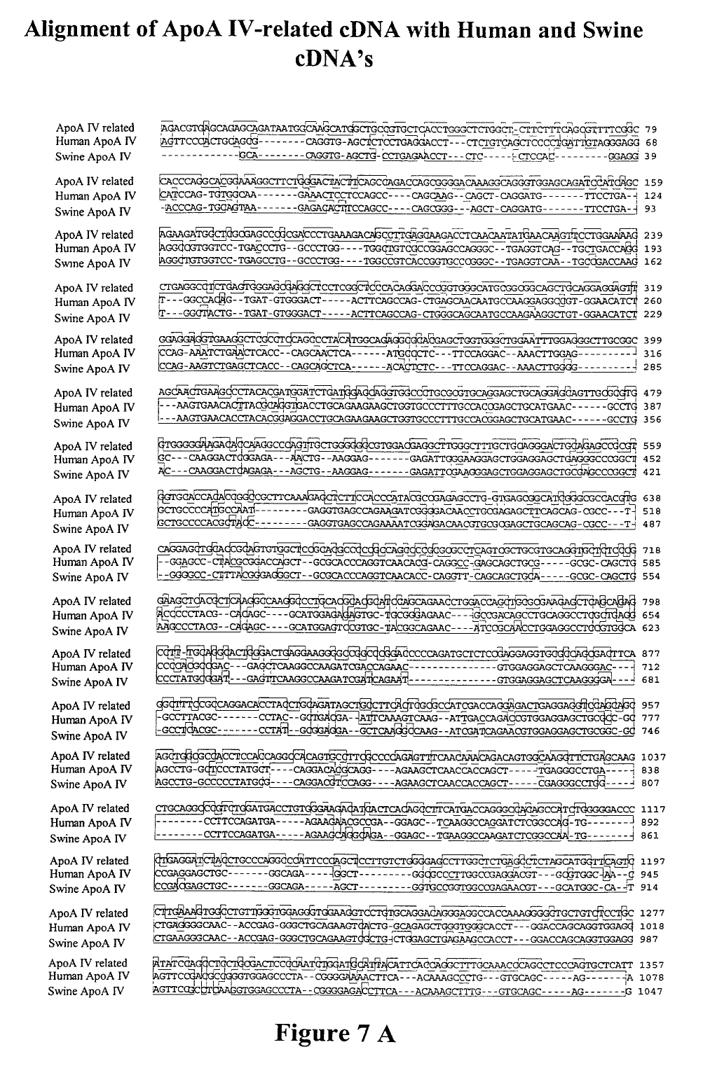

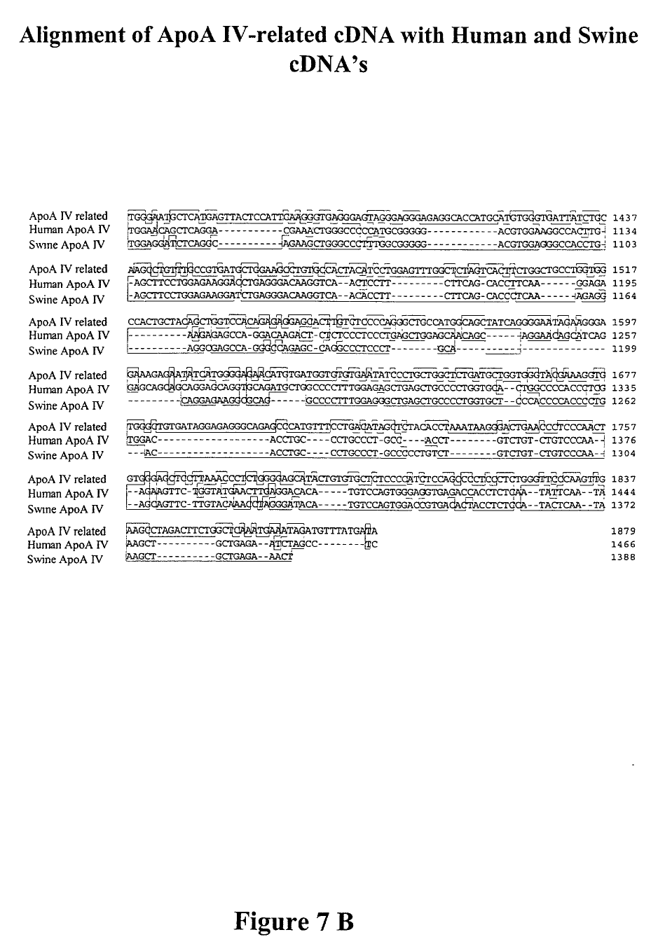

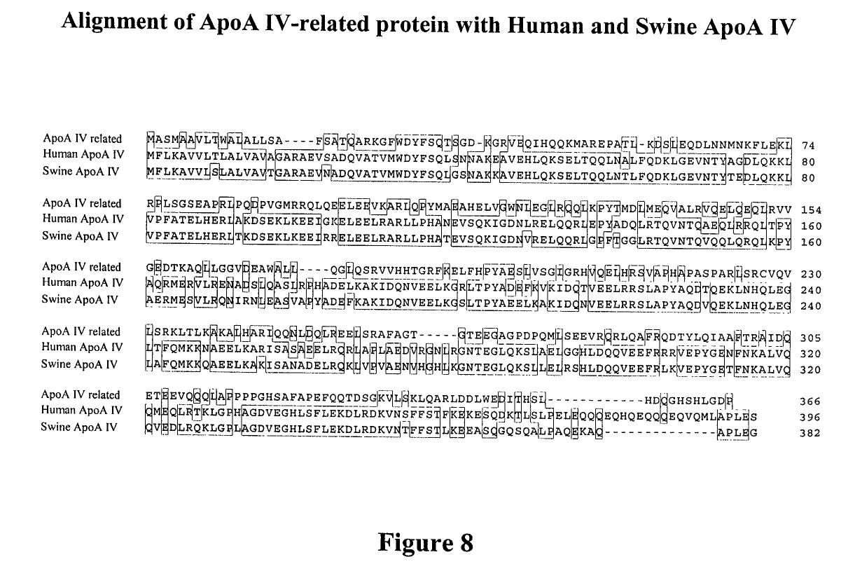

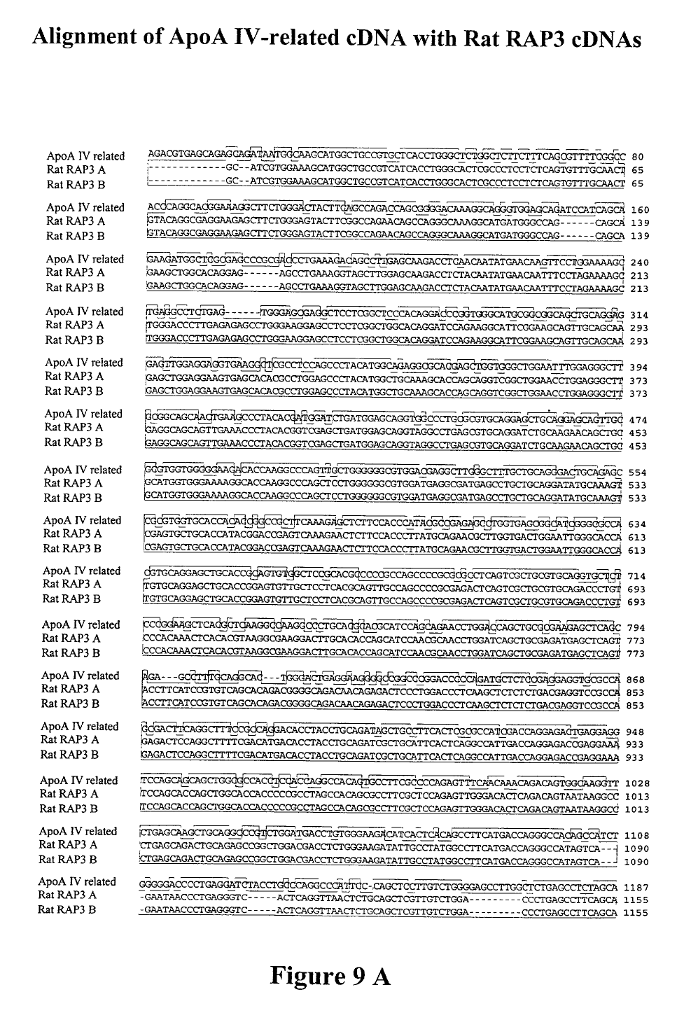

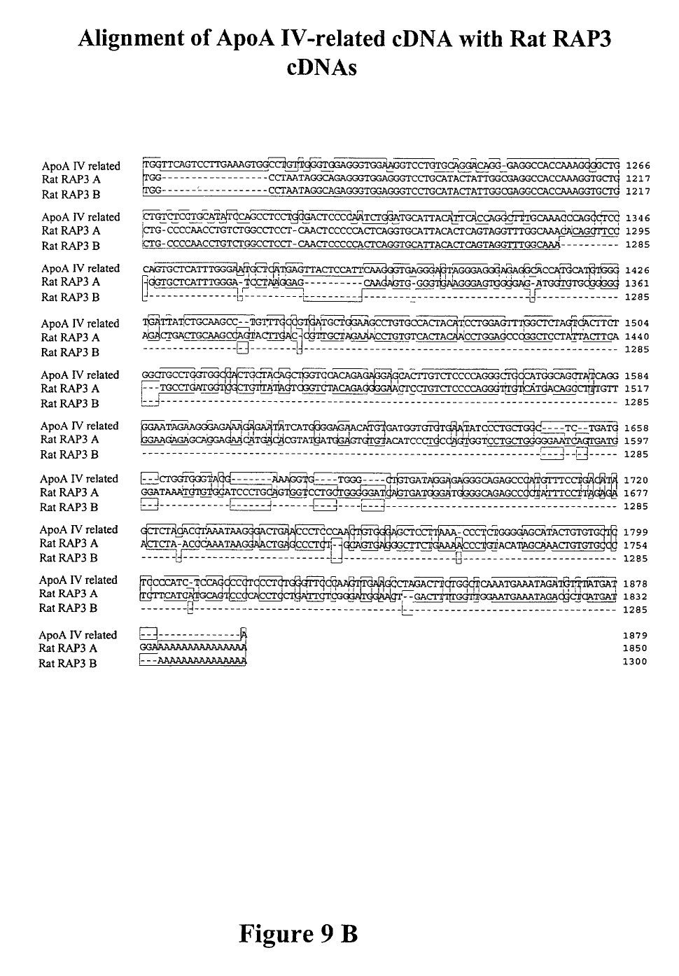

The protein of SEQ ID No. 3 encoded by the cDNA of SEQ ID No. 2 also exhibits homology to apolipoprotein A-IV-related protein. Lipoproteins such as HDL and LDL contain characteristic apolipoproteins that are responsible for targeting them to certain tissues and for activating enzymes required for the trafficking of the lipid fraction of the lipoprotein, including cholesterol. Apolipoprotein A-IV-related protein (AA4RP) is a member of the apolipoprotein family; it is 52% similar (29% identical) to apolipoprotein A-IV (apo A-IV) and therefore is likely to have a similar function. See FIGS. 7 and 8.

Expression of apolipoproteins is known to be under the control of developmental, hormonal, dietary and tissue specific regulation. In particular, the inventors found AA4RP is differentially expressed in obese mouse models: up regulated in mice fed a high fat diet (cafeteria diet) and in naturally obese mice (NZO), while it was not differentially expressed in transgenic mice lacking the gene for leptin (ob/ob) or in mice lacking the gene for the leptin receptor (db/db); thus suggesting AA4RP is regulated by diet (See Examples 4 and 6). In addition, potential inhibitors and antagonists of the gene that decrease the concentration of AA4RP will serve as important therapeutic compounds in the treatment lipid metabolism related disorders.

Although apo A-IV was discovered more than twenty years ago, its physiological function is not completely understood (Swaney et al. (1977)). Apo A-IV is associated with the chylomicron and HDL fraction of blood, and recently it has been demonstrated that apo A-IV synthesis by the small intestine increases markedly after the ingestion of lipid with the resultant effect being a marked increase in apo A-IV output in mesenteric lymph (Hayashi et al. (1990)). Because intestinal synthesis and secretion of apo A-IV increases after triacylglycerol feeding, it is thought that apo A-IV may be involved in the biogenesis and/or metabolism of intestinal triglyceride-rich lipoproteins (Gordon et al. (1984)). It has also been demonstrated that this increase in biosynthesis and secretion of apo A-IV by the small intestine after fat feeding is triggered by the formation and secretion of intestinal chylomicrons (Hayashi et al. (1990)). Further, it has been shown that the apo A-IV appearing in mesenteric lymph after a lipid meal suppresses food intake, thus suggesting that apo A-IV may also act as a satiety factor that circulates in the blood after fat feeding (Fujimoto et al., (1992)).

Apo A-IV is also considered to play a role in triglyceride-rich lipoprotein metabolism, in reverse cholesterol transport, and in facilitation of CETP (Cholesterol Ester Transfer Protein) activity (Verges (1995)). As a result, apo A-IV is responsible for part of the inter-individual variability in blood cholesterol response to changes in dietary fat/cholesterol intake. Moreover, apo A-IV has similar efficiency as the HDL'S, i.e. a strong ability to activate LCAT, and may be effectively used instead of natural HDL to prevent the development of atherosclerosis (Wang Z. et al. (1995)). Over-expression of the protein is protective against atherosclerosis in mice with ApoE knockouts (ApoE is a well established anti-atherogenic protein).

In addition to its role in atherosclerosis, apo A-IV is known to play a significant role in the pathophysiology of diabetes. Levels of apo A-IV are correlated with glycemic control in young type I diabetes (IDDM) patients and non-insulin-dependent diabetes mellitus (NIDDM) patients. In addition, NIDDM patients have a high myocardial infarction risk apo A-IV phenotype that is particularly deleterious in obese patients (Rewers M. et al. (1994)).

I. Definitions

Before describing the invention in greater detail, the following definitions are set forth to illustrate and define the meaning and scope of the terms used to describe the invention herein.

The terms “AA4RP gene,” when used herein, encompasses genomic, mRNA and cDNA sequences encoding the apolipoprotein A-IV-related protein (AA4RP) protein, including the untranslated regulatory regions of the genomic DNA.

The term “heterologous protein,” when used herein, is intended to designate any protein or polypeptide other than the AA4RP protein. More particularly, the heterologous protein is a compound which can be used as a marker in further experiments with a AA4RP regulatory region.

The term “isolated” requires that the material be removed from its original environment (e.g., the natural environment if it is naturally occurring). For example, a naturally-occurring polynucleotide or polypeptide present in a living animal is not isolated, but the same polynucleotide or DNA or polypeptide, separated from some or all of the coexisting materials in the natural system, is isolated. Such polynucleotide could be part of a vector and/or such polynucleotide or polypeptide could be part of a composition, and still be isolated in that the vector or composition is not part of its natural environment.

The term “isolated” further requires that the material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated. Specifically excluded from the definition of “isolated” are: naturally-occurring chromosomes (such as chromosome spreads), artificial chromosome libraries, genomic libraries, and cDNA libraries that exist either as an in vitro nucleic acid preparation or as a transfected/transformed host cell preparation, wherein the host cells are either an in vitro heterogeneous preparation or plated as a heterogeneous population of single colonies. Also specifically excluded are the above libraries wherein a specified polynucleotide of the present invention makes up less than 5% of the number of nucleic acid inserts in the vector molecules. Further specifically excluded are whole cell genomic DNA or whole cell RNA preparations (including said whole cell preparations which are mechanically sheared or enzymaticly digested). Further specifically excluded are the above whole cell preparations as either an in vitro preparation or as a heterogeneous mixture separated by electrophoresis (including blot transfers of the same) wherein the polynucleotide of the invention has not further been separated from the heterologous polynucleotides in the electrophoresis medium (e.g., further separating by excising a single band from a heterogeneous band population in an agarose gel or nylon blot).

The term “purified” does not require absolute purity; rather, it is intended as a relative definition. Purification of starting material or natural material to at least one order of magnitude, preferably two or three orders, and more preferably four or five orders of magnitude is expressly contemplated. As an example, purification from 0.1% concentration to 10% concentration is two orders of magnitude. The term “purified polynucleotide” is used herein to describe a polynucleotide or polynucleotide vector of the invention which has been separated from other compounds including, but not limited to other nucleic acids, carbohydrates, lipids and proteins (such as the enzymes used in the synthesis of the polynucleotide), or the separation of covalently closed polynucleotides from linear polynucleotides. A polynucleotide is substantially pure when at least about 50%, preferably 60 to 75% of a sample exhibits a single polynucleotide sequence and conformation (linear versus covalently close). A substantially pure polynucleotide typically comprises about 50%, preferably 60 to 90% weight/weight of a nucleic acid sample, more usually about 95%, and preferably is over about 99% pure. Polynucleotide purity or homogeneity is indicated by a number of means well known in the art, such as agarose or polyacrylamide gel electrophoresis of a sample, followed by visualizing a single polynucleotide band upon staining the gel. For certain purposes higher resolution can be provided by using HPLC or other means well known in the art.

The term “polypeptide” refers to a polymer of amino acids without regard to the length of the polymer; thus, peptides, oligopeptides, and proteins are included within the definition of polypeptide. This term also does not specify or exclude post-expression modifications of polypeptides, for example, polypeptides which include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

The term “recombinant polypeptide” is used herein to refer to polypeptides that have been artificially designed and which comprise at least two polypeptide sequences that are not found as contiguous polypeptide sequences in their initial natural environment, or to refer to polypeptides which have been expressed from a recombinant polynucleotide.

The term “purified polypeptide” is used herein to describe a polypeptide of the invention which has been separated from other compounds including, but not limited to nucleic acids, lipids, carbohydrates and other proteins. A polypeptide is substantially pure when at least about 50%, preferably 60 to 75% of a sample exhibits a single polypeptide sequence. A substantially pure polypeptide typically comprises about 50%, preferably 60 to 90% weight/weight of a protein sample, more usually about 95%, and preferably is over about 99% pure. Polypeptide purity or homogeneity is indicated by a number of means well known in the art, such as polyacrylamide gel electrophoresis of a sample, followed by visualizing a single polypeptide band upon staining the gel. For certain purposes higher resolution can be provided by using HPLC or other means well known in the art.

As used herein, the term “non-human animal” refers to any non-human vertebrate, birds and more usually mammals, preferably primates, farm animals such as swine, goats, sheep, donkeys, and horses, rabbits or rodents, more preferably rats or mice. As used herein, the term “animal” is used to refer to any vertebrate, preferable a mammal. Both the terms “animal” and “mammal” expressly embrace human subjects unless preceded with the term “non-human”.

As used herein, the term “antibody” refers to a polypeptide or group of polypeptides which are comprised of at least one binding domain, where an antibody binding domain is formed from the folding of variable domains of an antibody molecule to form three-dimensional binding spaces with an internal surface shape and charge distribution complementary to the features of an antigenic determinant of an antigen, which allows an immunological reaction with the antigen. Antibodies include recombinant proteins comprising the binding domains, as wells as fragments, including Fab, Fab′, F(ab)₂, and F(ab′)₂ fragments.

As used herein, an “antigenic determinant” is the portion of an antigen molecule, in this case a AA4RP polypeptide, that determines the specificity of the antigen-antibody reaction. An “epitope” refers to an antigenic determinant of a polypeptide. An epitope can comprise as few as 3 amino acids in a spatial conformation which is unique to the epitope. Generally an epitope comprises at least 6 such amino acids, and more usually at least 8-10 such amino acids. Methods for determining the amino acids which make up an epitope include x-ray crystallography, 2-dimensional nuclear magnetic resonance, and epitope mapping e.g. the Pepscan method described by Geysen et al. 1984; PCT Publication No. WO 84/03564; and PCT Publication No. WO 84/03506.

Throughout the present specification, the expression “nucleotide sequence” may be employed to designate indifferently a polynucleotide or a nucleic acid. More precisely, the expression “nucleotide sequence” encompasses the nucleic material itself and is thus not restricted to the sequence information (i.e. the succession of letters chosen among the four base letters) that biochemically characterizes a specific DNA or RNA molecule.

As used interchangeably herein, the terms “nucleic acids”, “oligonucleotides”, and “polynucleotides” include RNA, DNA, or RNA/DNA hybrid sequences of more than one nucleotide in either single chain or duplex form. The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. Although the term “nucleotide” is also used herein to encompass “modified nucleotides” which comprise at least one modifications (a) an alternative linking group, (b) an analogous form of purine, (c) an analogous form of pyrimidine, or (d) an analogous sugar, for examples of analogous linking groups, purine, pyrimidines, and sugars see for example PCT publication No. WO 95/04064. The polynucleotide sequences of the invention may be prepared by any known method, including synthetic, recombinant, ex vivo generation, or a combination thereof, as well as utilizing any purification methods known in the art.

A “promoter” refers to a DNA sequence recognized by the synthetic machinery of the cell required to initiate the specific transcription of a gene.

A sequence which is “operably linked” to a regulatory sequence such as a promoter means that said regulatory element is in the correct location and orientation in relation to the nucleic acid to control RNA polymerase initiation and expression of the nucleic acid of interest.

As used herein, the term “operably linked” refers to a linkage of polynucleotide elements in a functional relationship. For instance, a promoter or enhancer is operably linked to a coding sequence if it affects the transcription of the coding sequence. More precisely, two DNA molecules (such as a polynucleotide containing a promoter region and a polynucleotide encoding a desired polypeptide or polynucleotide) are said to be “operably linked” if the nature of the linkage between the two polynucleotides does not (1) result in the introduction of a frame-shift mutation or (2) interfere with the ability of the polynucleotide containing the promoter to direct the transcription of the coding polynucleotide.

The term “primer” denotes a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase.

The term “probe” denotes a defined nucleic acid segment (or nucleotide analog segment, e.g., polynucleotide as defined herein) which can be used to identify a specific polynucleotide sequence present in samples, said nucleic acid segment comprising a nucleotide sequence complementary of the specific polynucleotide sequence to be identified.

The terms “trait” and “phenotype” are used interchangeably herein and refer to any visible, detectable or otherwise measurable property of an organism such as symptoms of, or susceptibility to a disease for example. Typically the terms “trait” or “phenotype” are used herein to refer to symptoms of, or susceptibility to a disease, a beneficial response to or side effects related to a treatment. Preferably, said trait can be, but not limited to, lipid metabolism related disorders and/or liver related disorders.

The term “allele” is used herein to refer to variants of a nucleotide sequence. A biallelic polymorphism has two forms. Diploid organisms may be homozygous or heterozygous for an allelic form.

The term “heterozygosity rate” is used herein to refer to the incidence of individuals in a population which are heterozygous at a particular allele. In a biallelic system, the heterozygosity rate is on average equal to 2Pₐ(1−Pₐ), where Pₐ is the frequency of the least common allele. In order to be useful in genetic studies, a genetic marker should have an adequate level of heterozygosity to allow a reasonable probability that a randomly selected person will be heterozygous.

The term “genotype” as used herein refers the identity of the alleles present in an individual or a sample. In the context of the present invention, a genotype preferably refers to the description of the biallelic marker alleles present in an individual or a sample. The term “genotyping” a sample or an individual for a biallelic marker involves determining the specific allele or the specific nucleotide carried by an individual at a biallelic marker.

The term “mutation” as used herein refers to a difference in DNA sequence between or among different genomes or individuals which has a frequency below 1%. The term “haplotype” refers to a combination of alleles present in an individual or a sample. In the context of the present invention, a haplotype preferably refers to a combination of biallelic marker alleles found in a given individual and which may be associated with a phenotype.

The term “polymorphism” as used herein refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A single nucleotide polymorphism is the replacement of one nucleotide by another nucleotide at the polymorphic site. Deletion of a single nucleotide or insertion of a single nucleotide also gives rise to single nucleotide polymorphisms. In the context of the present invention, “single nucleotide polymorphism” preferably refers to a single nucleotide substitution. Typically, between different individuals, the polymorphic site may be occupied by two different nucleotides.

The term “biallelic polymorphism” and “biallelic marker” are used interchangeably herein to refer to a single nucleotide polymorphism having two alleles at a fairly high frequency in the population. A “biallelic marker allele” refers to the nucleotide variants present at a biallelic marker site. Typically, the frequency of the less common allele of the biallelic markers of the present invention has been validated to be greater than 1%, preferably the frequency is greater than 10%, more preferably the frequency is at least 20% (i.e. heterozygosity rate of at least 0.32), even more preferably the frequency is at least 30% (i.e. heterozygosity rate of at least 0.42). A biallelic marker wherein the frequency of the less common allele is 30% or more is termed a “high quality biallelic marker”.

The invention also concerns apolipoprotein A-IV-related protein (AA4RP)-related biallelic markers. The term “AA4RP-related biallelic marker” is used interchangeably herein to relate to all biallelic markers in linkage disequilibrium with the biallelic markers of the AA4RP gene. The term AA4RP-related biallelic marker includes both the genic and non-genic biallelic markers described in Table 1.

The term “non-genic” is used herein to describe AA4RP-related biallelic markers, as well as polynucleotides and primers which occur outside the nucleotide positions shown in the human AA4RP genomic sequence of SEQ ID No 1. The term “genic” is used herein to describe AA4RP-related biallelic markers as well as polynucleotides and primers which do occur in the nucleotide positions shown in the human AA4RP genomic sequence of SEQ ID Nos 1 and 4.

The location of nucleotides in a polynucleotide with respect to the center of the polynucleotide are described herein in the following manner. When a polynucleotide has an odd number of nucleotides, the nucleotide at an equal distance from the 3′ and 5′ ends of the polynucleotide is considered to be “at the center” of the polynucleotide, and any nucleotide immediately adjacent to the nucleotide at the center, or the nucleotide at the center itself is considered to be “within 1 nucleotide of the center.” With an odd number of nucleotides in a polynucleotide any of the five nucleotides positions in the middle of the polynucleotide would be considered to be within 2 nucleotides of the center, and so on. When a polynucleotide has an even number of nucleotides, there would be a bond and not a nucleotide at the center of the polynucleotide. Thus, either of the two central nucleotides would be considered to be “within 1 nucleotide of the center” and any of the four nucleotides in the middle of the polynucleotide would be considered to be “within 2 nucleotides of the center”, and so on. For polymorphisms which involve the substitution, insertion or deletion of 1 or more nucleotides, the polymorphism, allele or biallelic marker is “at the center” of a polynucleotide if the difference between the distance from the substituted, inserted, or deleted polynucleotides of the polymorphism and the 3′ end of the polynucleotide, and the distance from the substituted, inserted, or deleted polynucleotides of the polymorphism and the 5′ end of the polynucleotide is zero or one nucleotide. If this difference is 0 to 3, then the polymorphism is considered to be “within 1 nucleotide of the center.” If the difference is 0 to 5, the polymorphism is considered to be “within 2 nucleotides of the center.” If the difference is 0 to 7, the polymorphism is considered to be “within 3 nucleotides of the center,” and so on.

The term “upstream” is used herein to refer to a location which is toward the 5′ end of the polynucleotide from a specific reference point.

The terms “base paired” and “Watson & Crick base paired” are used interchangeably herein to refer to nucleotides which can be hydrogen bonded to one another be virtue of their sequence identities in a manner like that found in double-helical DNA with thymine or uracil residues linked to adenine residues by two hydrogen bonds and cytosine and guanine residues linked by three hydrogen bonds (See Stryer, L., Biochemistry, 4th edition, 1995).

The terms “complementary” or “complement thereof” are used herein to refer to the sequences of polynucleotides which is capable of forming Watson & Crick base pairing with another specified polynucleotide throughout the entirety of the complementary region. For the purpose of the present invention, a first polynucleotide is deemed to be complementary to a second polynucleotide when each base in the first polynucleotide is paired with its complementary base. Complementary bases are, generally, A and T (or A and U), or C and G. “Complement” is used herein as a synonym from “complementary polynucleotide”, “complementary nucleic acid” and “complementary nucleotide sequence”. These terms are applied to pairs of polynucleotides based solely upon their sequences and not any particular set of conditions under which the two polynucleotides would actually bind.

The term “original nucleotide” refers to the nucleotides present at the conflict positions 1241 and 1447 of SEQ ID No 4 as previously identified in Genbank. They were previously identified as a T at position 13269 of SEQ ID No 1 and a G at position 13475 of SEQ ID No 1.

The term “alternative nucleotide” refers to the nucleotides present at the conflict positions 1241 and 1447 of SEQ ID No 4 as determined by the inventors. They are a C at position 1241 and an A at position 1447.

The term “disease involving lipid metabolism” refers to a condition linked to disturbances in expression, production or cellular response to lipoproteins such as VLDL, LDL, HDL, chylomicrons and their components which include triglycerides, cholesterol, cholesterol ester, phospholipids, and apolipoproteins such as apo A-IV. “Diseases involving lipid metabolism” include obesity and obesity-related disorders such as obesity-related atherosclerosis, obesity-related insulin resistance, obesity-related hypertension, microangiopathic lesions resulting from obesity-related Type II diabetes, ocular lesions caused by microangiopathy in obese individuals with Type II diabetes, and renal lesions caused by microangiopathy in obese individuals with Type II diabetes. “Diseases involving lipid metabolism” also include atherosclerosis, cardiovascular disorders such as coronary heart disease, neurodegenerative disorders such as Alzheimer's disease or dementia, coronary artery disease, mitochondriocytopathies, hyperlipidemia, familial combined hyperlipidemia (FCHL) and hypercholesterolemia.

The terms “agent acting on lipid metabolism and/or lipid metabolism” refers to a drug or a compound modulating the activity or concentration of lipoproteins such as VLDL, LDL, HDL, chylomicrons and their components which include triglycerides, cholesterol, cholesterol ester, phospholipids, and apolipoproteins such as apo A-IV.

The terms “response to an agent acting on lipid metabolism and/or liver related disorders” refer to drug efficacy, including but not limited to ability to metabolize a compound, to the ability to convert a pro-drug to an active drug, and to the pharmacokinetics (absorption, distribution, elimination) and the pharmacodynamics (receptor-related) of a drug in an individual.

The terms “side effects to an agent acting on lipid metabolism and/or a liver related disorder” refer to adverse effects of therapy resulting from extensions of the principal pharmacological action of the drug or to idiosyncratic adverse reactions resulting from an interaction of the drug with unique host factors. “Side effects to an agent acting on lipid metabolism and/or a liver related disorder” include, but are not limited to, adverse reactions such as dermatological, hematological or hepatologic toxicities and further includes gastric and intestinal ulceration, disturbance in platelet function, renal injury, nephritis, vasomotor rhinitis with profuse watery secretions, angioneurotic edema, generalized urticaria, and bronchial asthma to laryngeal edema and bronchoconstriction, hypotension, and shock.

The term “liver related disorders” refers to a condition linked to disturbances in expression, production or cellular response to regeneration associated protein (RAP3). Such disorders include, but are not limited to hepatitis, cirrhosis, hepatoma, and FHP.

The term “patient” as used herein refers to a mammal, including animals, preferably mice, rats, dogs, cattle, sheep, or primates, most preferably humans that are in need of treatment. The term “in need of such treatment” as used herein refers to a judgment made by a care giver such as a physician, nurse, or nurse practitioner in the case of humans that a patient requires or would benefit from treatment. This judgement is made based on a variety of factors that are in the realm of a care giver's expertise, but that include the knowledge that the patient is ill, or will be ill, as the result of a condition that is treatable by the compounds of the invention.

II. Variants and Fragments

A. Polynucleotides

The invention also relates to variants and fragments of the polynucleotides described herein, particularly of a AA4RP gene containing one or more biallelic markers according to the invention.

Variants of polynucleotides, as the term is used herein, are polynucleotides that differ from a reference polynucleotide. A variant of a polynucleotide may be a naturally occurring variant such as a naturally occurring allelic variant, or it may be a variant that is not known to occur naturally. Such non-naturally occurring variants of the polynucleotide may be made by mutagenesis techniques, including those applied to polynucleotides, cells or organisms. Generally, differences are limited so that the nucleotide sequences of the reference and the variant are closely similar overall and, in many regions, identical.

Variants of polynucleotides according to the invention include, without being limited to, nucleotide sequences which are at least 95% identical to a polynucleotide selected from the group consisting of the nucleotide sequences of SEQ ID Nos 1, 2 and 4, or to any polynucleotide fragment of at least 12 consecutive nucleotides of a polynucleotide selected from the group consisting of the nucleotide sequences of SEQ ID Nos 1, 2 and 4, and preferably at least 99% identical, more particularly at least 99.5% identical, and most preferably at least 99.8% identical to a polynucleotide selected from the group consisting of the nucleotide sequences of SEQ ID Nos 1,2 and 4 or to any polynucleotide fragment of at least 12 consecutive nucleotides of a polynucleotide selected from the group consisting of the nucleotide sequences of SEQ ID Nos 1, 2 and 4.

Nucleotide changes present in a variant polynucleotide may be silent, which means that they do not alter the amino acids encoded by the polynucleotide. However, nucleotide changes may also result in amino acid substitutions, additions, deletions, fusions and truncations in the polypeptide encoded by the reference sequence. The substitutions, deletions or additions may involve one or more nucleotides. The variants may be altered in coding or non-coding regions or both. Alterations in the coding regions may produce conservative or non-conservative amino acid substitutions, deletions or additions.

In the context of the present invention, particularly preferred embodiments are those in which the polynucleotides encode polypeptides which retain substantially the same biological function or activity as the mature AA4RP protein, or those in which the polynucleotides encode polypeptides which maintain or increase a particular biological activity, while reducing a second biological activity.

A polynucleotide fragment is a polynucleotide having a sequence that is entirely the same as part but not all of a given nucleotide sequence, preferably the nucleotide sequence of a AA4RP gene, and variants thereof. The fragment can be a portion of an intron or an exon of a AA4RP gene. It can also be a portion of the regulatory regions of AA4RP. Preferably, such fragments comprise at least one of the biallelic markers 20-828-311, 17-42-319, 17-41-250, 20-841-149, 20-842-115, and 20-853-415, or the complements thereto, or a biallelic marker in linkage disequilibrium with one or more of the biallelic markers 20-828-311, 17-42-319, 17-41-250, 20-841-149, 20-842-115, and 20-853-415.

Such fragments may be “free-standing”, i.e. not part of or fused to other polynucleotides, or they may be comprised within a single larger polynucleotide of which they form a part or region. Indeed, several of these fragments may be present within a single larger polynucleotide.

Optionally, such fragments may consist of, or consist essentially of a contiguous span of at least 8, 10, 12, 15, 18, 20, 25, 35, 40, 50, 70, 80, 100, 250, 500 or 1000 nucleotides in length. A set of preferred fragments contain at least one of the biallelic markers 20-828-311, 17-42-319, 17-41-250, 20-841-149, 20-842-115, and 20-853-415 of the AA4RP gene which are described herein or the complements thereto.

B. Polypeptides

The invention also relates to variants, fragments, analogs and derivatives of the polypeptides described herein, including mutated AA4RP proteins.

The variant may be 1) one in which one or more of the amino acid residues are substituted with a conserved or non-conserved amino acid residue and such substituted amino acid residue may or may not be one encoded by the genetic code, or 2) one in which one or more of the amino acid residues includes a substituent group, or 3) one in which the mutated AA4RP is fused with another compound, such as a compound to increase the half-life of the polypeptide (for example, polyethylene glycol), or 4) one in which the additional amino acids are fused to the mutated AA4RP, such as a leader or secretory sequence or a sequence which is employed for purification of the mutated AA4RP or a preprotein sequence. Such variants are deemed to be within the scope of those skilled in the art.

A polypeptide fragment is a polypeptide having a sequence that entirely is the same as part but not all of a given polypeptide sequence, preferably a polypeptide encoded by a AA4RP gene and variants thereof.

In the case of an amino acid substitution in the amino acid sequence of a polypeptide according to the invention, one or several amino acids can be replaced by “equivalent” amino acids. The expression “equivalent” amino acid is used herein to designate any amino acid that may be substituted for one of the amino acids having similar properties, such that one skilled in the art of peptide chemistry would expect the secondary structure and hydropathic nature of the polypeptide to be substantially unchanged. Generally, the following groups of amino acids represent equivalent changes: (1) Ala, Pro, Gly, Glu, Asp, Gln, Asn, Ser, Thr; (2) Cys, Ser, Tyr, Thr; (3) Val, Ile, Leu, Met, Ala, Phe; (4) Lys, Arg, His; (5) Phe, Tyr, Trp, His.

In addition to the above preferred nucleic acid sizes, further preferred sub-genuses of nucleic acids comprise at least 8 nucleotides, wherein “at least 8” is defined as any integer between 8 and the integer representing the 3′ most nucleotide position as set forth in the sequence listing or elsewhere herein. Further included as preferred polynucleotides of the present invention are nucleic acid fragments at least 8 nucleotides in length, as described above, that are further specified in terms of their 5′ and 3′ position. The 5′ and 3′ positions are represented by the position numbers set forth in the sequence listing below. For allelic and degenerate variants, position 1 is defined as the 5′ most nucleotide of the ORF, i.e., the nucleotide “A” of the start codon with the remaining nucleotides numbered consecutively. Therefore, every combination of a 5′ and 3′ nucleotide position that a polynucleotide fragment of the present invention, at least 8 contiguous nucleotides in length, could occupy is included in the invention as an individual species. The polynucleotide fragments specified by 5′ and 3′ positions can be immediately envisaged and are therefore not individually listed solely for the purpose of not unnecessarily lengthening the specifications.

It is noted that the above species of polynucleotide fragments of the present invention may alternatively be described by the formula “a to b”; where “x” equals the 5″ most nucleotide position and “y” equals the 3 ″ most nucleotide position of the polynucleotide; and further where “x” equals an integer between 1 and the number of nucleotides of the polynucleotide sequence of the present invention minus 8, and where “y” equals an integer between 9 and the number of nucleotides of the polynucleotide sequence of the present invention; and where “x” is an integer smaller then “y” by at least 8.

The present invention also provides for the exclusion of any species of polynucleotide fragments of the present invention specified by 5′ and 3′ positions or sub-genuses of polynucleotides specified by size in nucleotides as described above. Any number of fragments specified by 5′ and 3′ positions or by size in nucleotides, as described above, may be excluded.

In addition to the above polypeptide fragments, further preferred sub-genuses of polypeptides comprise at least 8 amino acids, wherein “at least 8” is defined as any integer between 8 and the integer representing the C-terminal amino acid of the polypeptide of the present invention including the polypeptide sequences of the sequence listing below. Further included are species of polypeptide fragments at least 8 amino acids in length, as described above, that are further specified in terms of their N-terminal and C-terminal positions. Preferred species of polypeptide fragments specified by their N-terminal and C-terminal positions include the signal peptides delineated in the sequence listing below. However, included in the present invention as individual species are all polypeptide fragments, at least 8 amino acids in length, as described above, and may be particularly specified by a N-terminal and C-terminal position. That is, every combination of a N-terminal and C-terminal position that a fragment at least 8 contiguous amino acid residues in length could occupy, on any given amino acid sequence of the sequence listing or of the present invention is included in the present invention

The present invention also provides for the exclusion of any fragment species specified by N-terminal and C-terminal positions or of any fragment sub-genus specified by size in amino acid residues as described above. Any number of fragments specified by N-terminal and C-terminal positions or by size in amino acid residues as described above may be excluded as individual species.

The above polypeptide fragments of the present invention can be immediately envisaged using the above description and are therefore not individually listed solely for the purpose of not unnecessarily lengthening the specification. Moreover, the above fragments need not be active since they would be useful, for example, in immunoassays, in epitope mapping, epitope tagging, as vaccines, and as molecular weight markers. The above fragments may also be used to generate antibodies to a particular portion of the polypeptide. These antibodies can then be used in immunoassays well known in the art to distinguish between human and non-human cells and tissues or to determine whether cells or tissues in a biological sample are or are not of the same type which express the polypeptide of the present invention. Preferred polypeptide fragments of the present invention comprising a signal peptide may be used to facilitate secretion of either the polypeptide of the same gene or a heterologous polypeptide using methods well known in the art. Another embodiment of the present invention is an isolated or purified polypeptide comprising a signal peptide of one of the polypeptides of SEQ ID No 3.

A specific embodiment of a modified AA4RP peptide molecule of interest according to the present invention, includes, but is not limited to, a peptide molecule which is resistant to proteolysis, is a peptide in which the —CONH— peptide bond is modified and replaced by a (CH2NH) reduced bond, a (NHCO) retro inverso bond, a (CH2—O) methylene-oxy bond, a (CH2—S) thiomethylene bond, a (CH2CH2) carba bond, a (CO—CH2) cetomethylene bond, a (CHOH—CH2) hydroxyethylene bond), a (N—N) bound, a E-alcene bond or also a —CH═CH— bond. The invention also encompasses a human AA4RP polypeptide or a fragment or a variant thereof in which at least one peptide bond has been modified as described above.

Such fragments may be “free-standing”, i.e. not part of or fused to other polypeptides, or they may be comprised within a single larger polypeptide of which they form a part or region. However, several fragments may be comprised within a single larger polypeptide.

As representative examples of polypeptide fragments of the invention, there may be mentioned those which have from about 5, 6, 7, 8, 9 or 10 to 15, 10 to 20, 15 to 40, or 30 to 55 amino acids long. Preferred are those fragments containing at least one amino acid mutation in the AA4RP protein.

III. Identity Between Nucleic Acids or Polypeptides

The terms “percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Homology is evaluated using any of the variety of sequence comparison algorithms and programs known in the art. Such algorithms and programs include, but are by no means limited to, TBLASTN, BLASTP, FASTA, TFASTA, and CLUSTALW (Pearson and Lipman, 1988; Altschul et al., 1990; Thompson et al., 1994; Higgins et al., 1996; Altschul et al., 1990; Altschul et al., 1993). In a particularly preferred embodiment, protein and nucleic acid sequence homologies are evaluated using the Basic Local Alignment Search Tool (“BLAST”) which is well known in the art (see, e.g., Karlin and Altschul, 1990; Altschul et al., 1990, 1993, 1997). In particular, five specific BLAST programs are used to perform the following task:

(1) BLASTP and BLAST3 compare an amino acid query sequence against a protein sequence database;

(2) BLASTN compares a nucleotide query sequence against a nucleotide sequence database;

(3) BLASTX compares the six-frame conceptual translation products of a query nucleotide sequence (both strands) against a protein sequence database;

(4) TBLASTN compares a query protein sequence against a nucleotide sequence database translated in all six reading frames (both strands); and

(5) TBLASTX compares the six-frame translations of a nucleotide query sequence against the six-frame translations of a nucleotide sequence database.

The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., 1992; Henikoff and Henikoff, 1993). Less preferably, the PAM or PAM250 matrices may also be used (see, e.g., Schwartz and Dayhoff, eds., 1978). The BLAST programs evaluate the statistical significance of all high-scoring segment pairs identified, and preferably selects those segments which satisfy a user-specified threshold of significance, such as a user-specified percent homology. Preferably, the statistical significance of a high-scoring segment pair is evaluated using the statistical significance formula of Karlin (see, e.g., Karlin and Altschul (1990)).

The BLAST programs may be used with the default parameters or with modified parameters provided by the user.

IV. Stringent Hybridization Conditions

By way of example and not limitation, procedures using conditions of high stringency are as follows: Prehybridization of filters containing DNA is carried out for 8 hours to overnight at 65° C. in buffer composed of 6× SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C., the preferred hybridization temperature, in prehybridization mixture containing 100 μg/ml denatured salmon sperm DNA and 5-20×10⁶ cpm of ³²P-labeled probe. Alternatively, the hybridization step can be performed at 65° C. in the presence of SSC buffer, 1 × SSC corresponding to 0.15 M NaCl and 0.05 M Na citrate. Subsequently, filter washes can be done at 37° C. for 1 h in a solution containing 2 × SSC, 0.01% PVP, 0.01% Ficoll, and 0.01% BSA, followed by a wash in 0.1 × SSC at 50C for 45 min. Alternatively, filter washes can be performed in a solution containing 2× SSC and 0.1% SDS, or 0.5× SSC and 0.1% SDS, or 0.1× SSC and 0.1% SDS at 68° C. for 15 minute intervals. Following the wash steps, the hybridized probes are detectable by autoradiography. Other conditions of high stringency which may be used are well known in the art and as cited in Sambrook et al., 1989; and Ausubel et al., 1989, are incorporated herein in their entirety. These hybridization conditions are suitable for a nucleic acid molecule of about 20 nucleotides in length. There is no need to say that the hybridization conditions described above are to be adapted according to the length of the desired nucleic acid, following techniques well known to the one skilled in the art. The suitable hybridization conditions may for example be adapted according to the teachings disclosed in the book of Hames and Higgins (1985) or in Sambrook et al.(1989).

### PREFERRED EMBODIMENTS OF THE INVENTION

I. Polynucleotides of the Present Invention

A. Genomic Sequences of the AA4RP Gene

The present invention concerns the genomic sequence of AA4RP. The present invention encompasses the AA4RP gene, or AA4RP genomic sequences consisting of, consisting essentially of, or comprising the sequence of SEQ ID Nos 1 and 4, a sequence complementary thereto, as well as fragments and variants thereof. These polynucleotides may be purified, isolated, or recombinant.

The invention also encompasses a purified, isolated, or recombinant polynucleotide comprising a nucleotide sequence having at least 70, 75, 80, 85, 90, 95, 99, 99.8% nucleotide identity with a nucleotide sequence of SEQ ID Nos 1 and 4 or a complementary sequence thereto or a fragment thereof. The nucleotide differences in regards to the nucleotide sequence of SEQ ID Nos 1 and 4 may be randomly distributed throughout the entire nucleic acid. Nevertheless, preferred nucleic acids are those wherein the nucleotide differences as regards to the nucleotide sequence of SEQ ID Nos 1 and 4 are predominantly located outside the coding sequences contained in the exons. These nucleic acids, as well as their fragments and variants, may be used as oligonucleotide primers or probes in order to detect the presence of a copy of the AA4RP gene in a test sample, or alternatively in order to amplify a target nucleotide sequence within the AA4RP sequences.

Another object of the invention consists of a purified, isolated, or recombinant nucleic acid that hybridizes with the nucleotide sequence of SEQ ID Nos 1 and 4 or a complementary sequence thereto or a variant thereof, under the stringent hybridization conditions as defined above.

Particularly preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 1, or the complements thereof, wherein said contiguous span comprises at least 1, 2, 3, 5, or 10 of the following nucleotide positions of SEQ ID No 1: 739-1739; 10946-12958; 13470-13526; 13641-13752; 14271-17969; 41718-42718; 44942-45942; and 76558-77558. Further preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 1, or the complements thereof, wherein said contiguous span comprises a T at position 1239, a T at position 12347, a T at position 15241, a G at position 42218, an A at 45442, or a T at 77058. See Table 1 below. It should be noted that nucleic acid fragments of any size and sequence may also be comprised by the polynucleotides described in this section.

Particularly preferred nucleic acids of the invention also include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 4, or the complements thereof, wherein said contiguous span comprises at least 1, 2, 3, 5, or 10 of the following nucleotide positions of SEQ ID No 4: 1-1498; 1613-1724; 2243-3940; and 3941-5381. Additional preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 4, or the complements thereof, wherein said contiguous span comprises one or more of the nucleotides at positions 1241 and 1447. Further preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 4, or the complements thereof, wherein said contiguous span comprises a T at position 319 or a T at position 3213. See Table 1 below. It should be noted that nucleic acid fragments of any size and sequence may also be comprised by the polynucleotides described in this section.

TABLE 1

| | | |
|---|---|---|
| | | POSITION OF BIALLELIC |
| BIALLELIC MARKER ID | ALLELES | MARKER IN SEQ ID |

Genic Biallelic Markers (SEQ ID No 1)

| | | |
|---|---|---|
| 17-42-319 | C/T | SEQ ID No 1, position 12347 |
| 17-41-250 | C/T | SEQ ID No 1, position 15241 |

Non-Genic Biallelic Markers (SEQ ID No 1)

| | | |
|---|---|---|
| 20-828-311 | C/T | SEQ ID No 1, position 1239 |
| 20-841-149 | A/G | SEQ ID No 1, position 42218 |
| 20-842-115 | A/G | SEQ ID No 1, position 45442 |
| 20-853-415 | C/T | SEQ ID No 1, position 77058 |

Genic Biallelic markers (SEQ ID No 2)

| | | |
|---|---|---|
| 17-41-250 | C/T | SEQ ID No 2, position 1153 |

Genic Biallelic markers (SEQ ID No 4)

| | | |
|---|---|---|
| 17-42-319 | C/T | SEQ ID No 4, position 319 |
| 17-41-250 | C/T | SEQ ID No 4, position 3213 |

The AA4RP genomic nucleic acid comprises 4 exons. The exon positions in SEQ ID Nos 1 and 4 are detailed below in Table 2.

TABLE 2

| | |
|---|---|
| Position in | Position in |
| SEQ ID No 1 | SEQ ID No 1 |

| Exon | Beginning | End | Intron | Beginning | End |
|---|---|---|---|---|---|
| 1 | 12947 | 12958 | 1 | 12959 | 13469 |
| 2 | 13470 | 13526 | 2 | 13527 | 13640 |
| 3 | 13641 | 13752 | 3 | 13753 | 14270 |
| 4 | 14271 | 15968 | | | |

| | |
|---|---|
| Position in | Position in |
| SEQ ID No 4 | SEQ ID No 4 |

| Exon | Beginning | End | Intron | Beginning | End |
|---|---|---|---|---|---|
| 1 | 919 | 930 | 1 | 931 | 1441 |
| 2 | 1442 | 1498 | 2 | 1499 | 1612 |
| 3 | 1613 | 1724 | 3 | 1725 | 2242 |
| 4 | 2243 | 3940 | | | |

Thus, the invention embodies purified, isolated, or recombinant polynucleotides comprising a nucleotide sequence selected from the group consisting of the 4 exons of the AA4RP gene, or a sequence complementary thereto. The invention also deals with purified, isolated, or recombinant nucleic acids comprising a combination of at least two exons of the AA4RP gene, wherein the polynucleotides are arranged within the nucleic acid, from the 5′-end to the 3′-end of said nucleic acid, in the same order as in SEQ ID Nos 1 and 4.

Intron 1 refers to the nucleotide sequence located between Exon 1 and Exon 2, and so on. The position of the introns is detailed in Table 2. Thus, the invention embodies purified, isolated, or recombinant polynucleotides comprising a nucleotide sequence selected from the group consisting of the 3 introns of the AA4RP gene, or a sequence complementary thereto.

While this section is entitled “Genomic Sequences of AA4RP,” it should be noted that nucleic acid fragments of any size and sequence may also be comprised by the polynucleotides described in this section, flanking the genomic sequences of AA4RP on either side or between two or more such genomic sequences.

B. cDNA Sequences

The expression of the AA4RP gene has been shown to lead to the production of at least one mRNA species, the nucleic acid sequence of which is set forth in SEQ ID No 2.

Another object of the invention is a purified, isolated, or recombinant nucleic acid comprising the nucleotide sequence of SEQ ID No 2, complementary sequences thereto, as well as allelic variants, and fragments thereof. Moreover, preferred polynucleotides of the invention include purified, isolated, or recombinant AA4RP cDNAs consisting of, consisting essentially of, or comprising the sequence of SEQ ID No 2. Particularly preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 2, or the complements thereof, wherein said contiguous span comprises at least 1, 2, 3, 5, or 10 of the following nucleotide positions of SEQ ID No 2: 1-1879. Further preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 2, or the complements thereof, wherein said contiguous span comprises a T at position 1153. See Table 1 above.

The invention also pertains to a purified or isolated nucleic acid comprising a polynucleotide having at least 95% nucleotide identity with a polynucleotide of SEQ ID No 2, advantageously 99% nucleotide identity, preferably 99.5% nucleotide identity and most preferably 99.8% nucleotide identity with a polynucleotide of SEQ ID No 2, or a sequence complementary thereto or a biologically active fragment thereof.

Another object of the invention relates to purified, isolated or recombinant nucleic acids comprising a polynucleotide that hybridizes, under the stringent hybridization conditions defined herein, with a polynucleotide of SEQ ID No 2, or a sequence complementary thereto or a variant thereof or a biologically active fragment thereof.

TABLE 3

| | Position range | | | Position range |
|---|---|---|---|---|
| | of 5′UTR | Position range of ORF | | of 3′UTR |
| SEQ ID No 2 | 1-20 | 21 | 1121 | 1122-1879 |

The cDNA of SEQ ID No 2 includes a 5′-UTR region starting from the nucleotide at position 1 and ending at the nucleotide in position 20 of SEQ ID No 2. The cDNA of SEQ ID No 2 includes a 3′-UTR region starting from the nucleotide at position 1122 and ending at the nucleotide at position 1879 of SEQ ID No 2.

Consequently, the invention concerns a purified, isolated, and recombinant nucleic acid comprising a nucleotide sequence of the 5 ′ UTR of the AA4RP cDNA, a sequence complementary thereto, or an allelic variant thereof. The invention also concerns a purified, isolated, and recombinant nucleic acid comprising a nucleotide sequence of the 3 ′ UTR of the AA4RP cDNA, a sequence complementary thereto, or an allelic variant thereof.

While this section is entitled “AA4RP cDNA Sequences,” it should be noted that nucleic acid fragments of any size and sequence may also be comprised by the polynucleotides described in this section, flanking the genomic sequences of AA4RP on either side or between two or more such genomic sequences.

Coding Regions

The AA4RP open reading frame is contained in the corresponding mRNA of SEQ ID No 2. More precisely, the effective AA4RP coding sequence (CDS) includes the region between nucleotide position 21 (first nucleotide of the ATG codon) and nucleotide position 1121 (end nucleotide of the TGA codon) of SEQ ID No 2.

The above disclosed polynucleotide that contains the coding sequence of the AA4RP gene may be expressed in a desired host cell or a desired host organism, when this polynucleotide is placed under the control of suitable expression signals. The expression signals may be either the expression signals contained in the regulatory regions in the AA4RP gene of the invention or in contrast the signals may be exogenous regulatory nucleic sequences. Such a polynucleotide, when placed under the suitable expression signals, may also be inserted in a vector for its expression and/or amplification.

C. Regulatory Sequences of AA4RP

As mentioned, the genomic sequence of the AA4RP gene contains regulatory sequences both in the non-coding 5′-flanking region and in the non-coding 3′-flanking region that border the AA4RP coding region containing the three exons of this gene.

The 5′-regulatory sequence of the AA4RP gene is localized between the nucleotide in position 10946 and the nucleotide in position 12946 of the nucleotide sequence of SEQ ID No 1. The 3′-regulatory sequence of the AA4RP gene is localized between nucleotide position 15969 and nucleotide position 17969 of SEQ ID No 1.

The 5′-regulatory sequence of the AA4RP gene is localized between the nucleotide in position 1 and the nucleotide in position 918 of the nucleotide sequence of SEQ ID No 4. The 3′-regulatory sequence of the AA4RP gene is localized between nucleotide position 3941 and nucleotide position 5381 of SEQ ID No 4.

Polynucleotides derived from the 5′ and 3′ regulatory regions are useful in order to detect the presence of at least a copy of a nucleotide sequence of SEQ ID Nos 1 and 4 or a fragment thereof in a test sample.

The promoter activity of the 5′ regulatory regions contained in AA4RP can be assessed as described below.

In order to identify the relevant biologically active polynucleotide fragments or variants of SEQ ID Nos 1 and 4, one of skill in the art will refer to the book of Sambrook et al.(Sambrook, 1989) which describes the use of a recombinant vector carrying a marker gene (i.e. beta galactosidase, chloramphenicol acetyl transferase, etc.) the expression of which will be detected when placed under the control of a biologically active polynucleotide fragments or variants of SEQ ID Nos 1 and 4. Genomic sequences located upstream of the first exon of the AA4RP gene are cloned into a suitable promoter reporter vector, such as the pSEAP-Basic, pSEAP-Enhancer, pβgal-Basic, pβgal-Enhancer, or pEGFP-1 Promoter Reporter vectors available from Clontech, or pGL2-basic or pGL3-basic promoterless luciferase reporter gene vector from Promega. Briefly, each of these promoter reporter vectors include multiple cloning sites positioned upstream of a reporter gene encoding a readily assayable protein such as secreted alkaline phosphatase, luciferase, β galactosidase, or green fluorescent protein. The sequences upstream the AA4RP coding region are inserted into the cloning sites upstream of the reporter gene in both orientations and introduced into an appropriate host cell. The level of reporter protein is assayed and compared to the level obtained from a vector which lacks an insert in the cloning site. The presence of an elevated expression level in the vector containing the insert with respect to the control vector indicates the presence of a promoter in the insert. If necessary, the upstream sequences can be cloned into vectors which contain an enhancer for increasing transcription levels from weak promoter sequences. A significant level of expression above that observed with the vector lacking an insert indicates that a promoter sequence is present in the inserted upstream sequence.

Promoter sequence within the upstream genomic DNA may be further defined by constructing nested 5′ and/or 3′ deletions in the upstream DNA using conventional techniques such as Exonuclease III or appropriate restriction endonuclease digestion. The resulting deletion fragments can be inserted into the promoter reporter vector to determine whether the deletion has reduced or obliterated promoter activity, such as described, for example, by Coles et al.(1998), the disclosure of which is incorporated herein by reference in its entirety. In this way, the boundaries of the promoters may be defined. If desired, potential individual regulatory sites within the promoter may be identified using site directed mutagenesis or linker scanning to obliterate potential transcription factor binding sites within the promoter individually or in combination. The effects of these mutations on transcription levels may be determined by inserting the mutations into cloning sites in promoter reporter vectors. This type of assay is well-known to those skilled in the art and is described in WO 97/17359, U.S. Pat. No. 5,374,544; EP 582 796; U.S. Pat. Nos. 5,698,389; 5,643,746; 5,502,176; and 5,266,488; the disclosures of which are incorporated by reference herein in their entirety.

The strength and the specificity of the promoter of the AA4RP gene can be assessed through the expression levels of a detectable polynucleotide operably linked to the AA4RP promoter in different types of cells and tissues. The detectable polynucleotide may be either a polynucleotide that specifically hybridizes with a predefined oligonucleotide probe, or a polynucleotide encoding a detectable protein, including a AA4RP polypeptide or a fragment or a variant thereof. This type of assay is well-known to those skilled in the art and is described in U.S. Pat. Nos. 5,502,176; and 5,266,488; the disclosures of which are incorporated by reference herein in their entirety. Some of the methods are discussed in more detail below.

Polynucleotides carrying the regulatory elements located at the 5′ end and at the 3′ end of the AA4RP coding region may be advantageously used to control the transcriptional and translational activity of an heterologous polynucleotide of interest.

Thus, the present invention also concerns a purified or isolated nucleic acid comprising a polynucleotide which is selected from the group consisting of the 5′ and 3′ regulatory regions, or a sequence complementary thereto or a biologically active fragment or variant thereof. “5′regulatory region” refers to the nucleotide sequence located between positions 10946 and 12946 of SEQ ID No 1. “3′regulatory region” refers to the nucleotide sequence located between positions 15969 and 17969 of SEQ ID No 1.

Thus, the present invention further concerns a purified or isolated nucleic acid comprising a polynucleotide which is selected from the group consisting of the 5′ and 3′ regulatory regions, or a sequence complementary thereto or a biologically active fragment or variant thereof. “5′regulatory region” refers to the nucleotide sequence located between positions 1 and 918 of SEQ ID No 4. “3′regulatory region” refers to the nucleotide sequence located between positions 3941 and 5381 of SEQ ID No 4.

The invention also pertains to a purified or isolated nucleic acid comprising a polynucleotide having at least 95% nucleotide identity with a polynucleotide selected from the group consisting of the 5′ and 3′ regulatory regions, advantageously 99% nucleotide identity, preferably 99.5% nucleotide identity and most preferably 99.8% nucleotide identity with a polynucleotide selected from the group consisting of the 5′ and 3′ regulatory regions, or a sequence complementary thereto or a variant thereof or a biologically active fragment thereof.

Another object of the invention consists of purified, isolated or recombinant nucleic acids comprising a polynucleotide that hybridizes, under the stringent hybridization conditions defined herein, with a polynucleotide selected from the group consisting of the nucleotide sequences of the 5′- and 3′ regulatory regions, or a sequence complementary thereto or a variant thereof or a biologically active fragment thereof.

Preferred fragments of the 5′ regulatory region have a length of about 1500 or 1000 nucleotides, preferably of about 500 nucleotides, more preferably about 400 nucleotides, even more preferably 300 nucleotides and most preferably about 200 nucleotides.

Preferred fragments of the 3′ regulatory region are at least 50, 100, 150, 200, 300 or 400 bases in length.

“Biologically active” polynucleotide derivatives of SEQ ID Nos 1 and 4 are polynucleotides comprising or alternatively consisting in a fragment of said polynucleotide which is functional as a regulatory region for expressing a recombinant polypeptide or a recombinant polynucleotide in a recombinant cell host. It could act either as an enhancer or as a repressor.

For the purpose of the invention, a nucleic acid or polynucleotide is “functional” as a regulatory region for expressing a recombinant polypeptide or a recombinant polynucleotide if said regulatory polynucleotide contains nucleotide sequences which contain transcriptional and translational regulatory information, and such sequences are “operably linked” to nucleotide sequences which encode the desired polypeptide or the desired polynucleotide.

The regulatory polynucleotides of the invention may be prepared from the nucleotide sequence of SEQ ID Nos 1 and 4 by cleavage using suitable restriction enzymes, as described for example in the book of Sambrook et al.(1989). The regulatory polynucleotides may also be prepared by digestion of SEQ ID Nos 1 and 4 by an exonuclease enzyme, such as Bal31 (Wabiko et al., 1986). These regulatory polynucleotides can also be prepared by nucleic acid chemical synthesis, as described elsewhere in the specification.

The regulatory polynucleotides according to the invention may be part of a recombinant expression vector that may be used to express a coding sequence in a desired host cell or host organism. The recombinant expression vectors according to the invention are described elsewhere in the specification.

A preferred 5′-regulatory polynucleotide of the invention includes the 5′-untranslated region (5′-UTR) of the AA4RP cDNA, or a biologically active fragment or variant thereof.

A preferred 3′-regulatory polynucleotide of the invention includes the 3′-untranslated region (3′-UTR) of the AA4RP cDNA, or a biologically active fragment or variant thereof.

A further object of the invention consists of a purified or isolated nucleic acid comprising:

a) a nucleic acid comprising a regulatory nucleotide sequence selected from the group consisting of:

(i) a nucleotide sequence comprising a polynucleotide of the 5′ regulatory region or a complementary sequence thereto;

(ii) a nucleotide sequence comprising a polynucleotide having at least 95% of nucleotide identity with the nucleotide sequence of the 5′ regulatory region or a complementary sequence thereto;

(iii) a nucleotide sequence comprising a polynucleotide that hybridizes under stringent hybridization conditions with the nucleotide sequence of the 5′ regulatory region or a complementary sequence thereto; and

(iv) a biologically active fragment or variant of the polynucleotides in (i), (ii) and (iii);

b) a polynucleotide encoding a desired polypeptide or a nucleic acid of interest, operably linked to the nucleic acid defined in (a) above;

c) Optionally, a nucleic acid comprising a 3′-regulatory polynucleotide, preferably a 3′-regulatory polynucleotide of the AA4RP gene.

In a specific embodiment of the nucleic acid defined above, said nucleic acid includes the 5′-untranslated region (5′-UTR) of the AA4RP cDNA, or a biologically active fragment or variant thereof.

In a second specific embodiment of the nucleic acid defined above, said nucleic acid includes the 3′-untranslated region (3′-UTR) of the AA4RP cDNA, or a biologically active fragment or variant thereof.

The regulatory polynucleotide of the 5′ regulatory region, or its biologically active fragments or variants, is operably linked at the 5′-end of the polynucleotide encoding the desired polypeptide or polynucleotide.

The regulatory polynucleotide of the 3′ regulatory region, or its biologically active fragments or variants, is advantageously operably linked at the 3′-end of the polynucleotide encoding the desired polypeptide or polynucleotide.

The desired polypeptide encoded by the above-described nucleic acid may be of various nature or origin, encompassing proteins of prokaryotic or eukaryotic origin. Among the polypeptides expressed under the control of a AA4RP regulatory region include bacterial, fungal or viral antigens. Also encompassed are eukaryotic proteins such as intracellular proteins, like “house keeping” proteins, membrane-bound proteins, like receptors, and secreted proteins like endogenous mediators such as cytokines. The desired polypeptide may be the AA4RP protein, especially the protein of the amino acid sequence of SEQ ID No 3, or a fragment or a variant thereof.

The desired nucleic acids encoded by the above-described polynucleotide, usually an RNA molecule, may be complementary to a desired coding polynucleotide, for example to the AA4RP coding sequence, and thus useful as an antisense polynucleotide.

Such a polynucleotide may be included in a recombinant expression vector in order to express the desired polypeptide or the desired nucleic acid in host cell or in a host organism. Suitable recombinant vectors that contain a polynucleotide such as described herein are disclosed elsewhere in the specification.

D. Polynucleotide Constructs

The terms “polynucleotide construct” and “recombinant polynucleotide” are used interchangeably herein to refer to linear or circular, purified or isolated polynucleotides that have been artificially designed and which comprise at least two nucleotide sequences that are not found as contiguous nucleotide sequences in their initial natural environment.

i. DNA Construct That Enables Directing Temporal and Spatial AA4RP Gene Expression in Recombinant Cell Hosts and in Transgenic Animals

In order to study the physiological and phenotypic consequences of a lack of synthesis of the AA4RP protein, both at the cell level and at the multi cellular organism level, the invention also encompasses DNA constructs and recombinant vectors enabling a conditional expression of a specific allele of the AA4RP genomic sequence or cDNA and also of a copy of this genomic sequence or cDNA harboring substitutions, deletions, or additions of one or more bases as regards to the AA4RP nucleotide sequence of SEQ ID Nos 1, 2 or 4, or a fragment thereof, these base substitutions, deletions or additions being located either in an exon, an intron or a regulatory sequence, but preferably in the 5′-regulatory sequence or in an exon of the AA4RP genomic sequence or within the AA4RP cDNA of SEQ ID No 2. In a preferred embodiment, the AA4RP sequence comprises a biallelic marker of the present invention. In a preferred embodiment, the AA4RP sequence comprises a biallelic marker of the present invention, preferably one of the biallelic markers 20-828-311, 17-42-319, 17-41-250, 20-841-149, 20-842-115, and 20-853-415. In a more preferred embodiment, the AA4RP sequence comprises a biallelic marker of the present invention, preferably one of the biallelic markers 17-42-319 or 17-41-250.

The present invention embodies recombinant vectors comprising any one of the polynucleotides described in the present invention. More particularly, the polynucleotide constructs according to the present invention can comprise any of the polynucleotides described in the “Genomic Sequences of the AA4RP Gene” section, the “AA4RP cDNA Sequences” section, the “Coding Regions” section, and the “Oligonucleotide Probes and Primers” section.

A first preferred DNA construct is based on the tetracycline resistance operon tet from E. coli transposon Tn10 for controlling the AA4RP gene expression, such as described by Gossen et al.(1992, 1995) and Furth et al.(1994). Such a DNA construct contains seven tet operator sequences from Tn10 (tetop) that are fused to either a minimal promoter or a 5′-regulatory sequence of the AA4RP gene, said minimal promoter or said AA4RP regulatory sequence being operably linked to a polynucleotide of interest that codes either for a sense or an antisense oligonucleotide or for a polypeptide, including a AA4RP polypeptide or a peptide fragment thereof. This DNA construct is functional as a conditional expression system for the nucleotide sequence of interest when the same cell also comprises a nucleotide sequence coding for either the wild type (tTA) or the mutant (rTA) repressor fused to the activating domain of viral protein VP 16 of herpes simplex virus, placed under the control of a promoter, such as the HCMVIE1 enhancer/promoter or the MMTV-LTR. Indeed, a preferred DNA construct of the invention comprise both the polynucleotide containing the tet operator sequences and the polynucleotide containing a sequence coding for the tTA or the rTA repressor.

In a specific embodiment, the conditional expression DNA construct contains the sequence encoding the mutant tetracycline repressor rTA, the expression of the polynucleotide of interest is silent in the absence of tetracycline and induced in its presence.

ii. DNA Constructs Allowing Homologous Recombination: Replacement Vectors

A second preferred DNA construct will comprise, from 5′-end to 3′-end: (a) a first nucleotide sequence that is comprised in the AA4RP genomic sequence; (b) a nucleotide sequence comprising a positive selection marker, such as the marker for neomycine resistance (neo); and (c) a second nucleotide sequence that is comprised in the AA4RP genomic sequence, and is located on the genome downstream the first AA4RP nucleotide sequence (a).

In a preferred embodiment, this DNA construct also comprises a negative selection marker located upstream the nucleotide sequence (a) or downstream the nucleotide sequence (c). Preferably, the negative selection marker comprises the thymidine kinase (tk) gene (Thomas et al., 1986), the hygromycine beta gene (Te Riele et al., 1990), the hprt gene (Van der Lugt et al., 1991; Reid et al., 1990) or the Diphteria toxin A fragment (Dt-A) gene (Nada et al., 1993; Yagi et al.1990). Preferably, the positive selection marker is located within a AA4RP exon sequence so as to interrupt the sequence encoding a AA4RP protein. These replacement vectors are described, for example, by Thomas et al.(1986; 1987), Mansour et al.(1988) and Koller et al.(1992).

The first and second nucleotide sequences (a) and (c) may be indifferently located within a AA4RP regulatory sequence, an intronic sequence, an exon sequence or a sequence containing both regulatory and/or intronic and/or exon sequences. The size of the nucleotide sequences (a) and (c) ranges from 1 to 50 kb, preferably from 1 to 10 kb, more preferably from 2 to 6 kb and most preferably from 2 to 4 kb.

iii. DNA Constructs Allowing Homologous Recombination: Cre-LoxP System

These new DNA constructs make use of the site specific recombination system of the P1 phage. The P1 phage possesses a recombinase called Cre which interacts specifically with a 34 base pairs loxP site. The loxP site is composed of two palindromic sequences of 13 bp separated by a 8 bp conserved sequence (Hoess et al., 1986). The recombination by the Cre enzyme between two loxP sites having an identical orientation leads to the deletion of the DNA fragment.

The Cre-loxP system used in combination with a homologous recombination technique has been first described by Gu et al.(1993, 1994). Briefly, a nucleotide sequence of interest to be inserted in a targeted location of the genome harbors at least two loxP sites in the same orientation and located at the respective ends of a nucleotide sequence to be excised from the recombinant genome. The excision event requires the presence of the recombinase (Cre) enzyme within the nucleus of the recombinant cell host. The recombinase enzyme may be brought at the desired time either by (a) incubating the recombinant cell hosts in a culture medium containing this enzyme, by injecting the Cre enzyme directly into the desired cell, such as described by Araki et al.(1995), or by lipofection of the enzyme into the cells, such as described by Baubonis et al.(1993); (b) transfecting the cell host with a vector comprising the Cre coding sequence operably linked to a promoter functional in the recombinant cell host, which promoter being optionally inducible, said vector being introduced in the recombinant cell host, such as described by Gu et al.(1993) and Sauer et al.(1988); (c) introducing in the genome of the cell host a polynucleotide comprising the Cre coding sequence operably linked to a promoter functional in the recombinant cell host, which promoter is optionally inducible, and said polynucleotide being inserted in the genome of the cell host either by a random insertion event or an homologous recombination event, such as described by Gu et al.(1994).

In a specific embodiment, the vector containing the sequence to be inserted in the AA4RP gene by homologous recombination is constructed in such a way that selectable markers are flanked by loxP sites of the same orientation, it is possible, by treatment by the Cre enzyme, to eliminate the selectable markers while leaving the AA4RP sequences of interest that have been inserted by an homologous recombination event. Again, two selectable markers are needed: a positive selection marker to select for the recombination event and a negative selection marker to select for the homologous recombination event. Vectors and methods using the Cre-loxP system are described by Zou et al.(1994).

Thus, a third preferred DNA construct of the invention comprises, from 5′-end to 3′-end: (a) a first nucleotide sequence that is comprised in the AA4RP genomic sequence; (b) a nucleotide sequence comprising a polynucleotide encoding a positive selection marker, said nucleotide sequence comprising additionally two sequences defining a site recognized by a recombinase, such as a loxP site, the two sites being placed in the same orientation; and (c) a second nucleotide sequence that is comprised in the AA4RP genomic sequence, and is located on the genome downstream of the first AA4RP nucleotide sequence (a).

The sequences defining a site recognized by a recombinase, such as a loxP site, are preferably located within the nucleotide sequence (b) at suitable locations bordering the nucleotide sequence for which the conditional excision is sought. In one specific embodiment, two loxP sites are located at each side of the positive selection marker sequence, in order to allow its excision at a desired time after the occurrence of the homologous recombination event.

In a preferred embodiment of a method using the third DNA construct described above, the excision of the polynucleotide fragment bordered by the two sites recognized by a recombinase, preferably two loxP sites, is performed at a desired time, due to the presence within the genome of the recombinant host cell of a sequence encoding the Cre enzyme operably linked to a promoter sequence, preferably an inducible promoter, more preferably a tissue-specific promoter sequence and most preferably a promoter sequence which is both inducible and tissue-specific, such as described by Gu et al.(1994).

The presence of the Cre enzyme within the genome of the recombinant cell host may result from the breeding of two transgenic animals, the first transgenic animal bearing the AA4RP-derived sequence of interest containing the loxP sites as described above and the second transgenic animal bearing the Cre coding sequence operably linked to a suitable promoter sequence, such as described by Gu et al.(1994).

Spatio-temporal control of the Cre enzyme expression may also be achieved with an adenovirus based vector that contains the Cre gene thus allowing infection of cells, or in vivo infection of organs, for delivery of the Cre enzyme, such as described by Anton and Graham (1995) and Kanegae et al.(1995).

The DNA constructs described above may be used to introduce a desired nucleotide sequence of the invention, preferably a AA4RP genomic sequence or a AA4RP cDNA sequence, and most preferably an altered copy of a AA4RP genomic or cDNA sequence, within a predetermined location of the targeted genome, leading either to the generation of an altered copy of a targeted gene (knock-out homologous recombination) or to the replacement of a copy of the targeted gene by another copy sufficiently homologous to allow an homologous recombination event to occur (knock-in homologous recombination). In a specific embodiment, the DNA constructs described above may be used to introduce a AA4RP genomic sequence or a AA4RP cDNA sequence comprising at least one biallelic marker of the present invention, preferably at least one biallelic marker selected from the group consisting of 20-828-311, 17-42-319, 17-41-250, 20-841-149, 20-842-115, and 20-853-415.

iv. Nuclear Antisense DNA Constructs

Other compositions containing a vector of the invention comprising an oligonucleotide fragment of the nucleic sequence SEQ ID No 2, preferably a fragment including the start codon of the AA4RP gene, as an antisense tool that inhibits the expression of the corresponding AA4RP gene. Preferred methods using antisense polynucleotide according to the present invention are the procedures described by Sczakiel et al. (1995) or those described in PCT Application No WO 95/24223, the disclosures of which are incorporated by reference herein in their entirety.

Preferably, the antisense tools are chosen among the polynucleotides (15-200 bp long) that are complementary to the 5 ′ end of the AA4RP mRNA. In one embodiment, a combination of different antisense polynucleotides complementary to different parts of the desired targeted gene are used.

Preferred antisense polynucleotides according to the present invention are complementary to a sequence of the mRNAs of AA4RP that contains either the translation initiation codon ATG or a splicing site. Further preferred antisense polynucleotides according to the invention are complementary of the splicing site of the AA4RP mRNA.

Preferably, the antisense polynucleotides of the invention have a 3′ polyadenylation signal that has been replaced with a self-cleaving ribozyme sequence, such that RNA polymerase II transcripts are produced without poly(A) at their 3′ ends, these antisense polynucleotides being incapable of export from the nucleus, such as described by Liu et al.(1994). In a preferred embodiment, these AA4RP antisense polynucleotides also comprise, within the ribozyme cassette, a histone stem-loop structure to stabilize cleaved transcripts against 3′-5′ exonucleolytic degradation, such as the structure described by Eckner et al. (1991).

E. Oligonucleotide Primers and Probes

Polynucleotides derived from the AA4RP gene are useful in order to detect the presence of at least a copy of a nucleotide sequence of SEQ ID Nos 1 and 4, or a fragment, complement, or variant thereof in a test sample.

Particularly preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 1, or the complements thereof, wherein said contiguous span comprises at least 1, 2, 3, 5, or 10 of the following nucleotide positions of SEQ ID No 1: 739-1739; 10946-12958; 13470-13526; 13641-13752; 14271-17969; 41718-42718; 44942-45942; and 76558-77558. Additional preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 1, or the complements thereof, wherein said contiguous span comprises a T at position 1239, a T at position 12347, a T at position 15241, a G at position 42218, an A at 45442, or a T at 77058.

Particularly preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 4, or the complements thereof, wherein said contiguous span comprises at least 1, 2, 3, 5, or 10 of the following nucleotide positions of SEQ ID No 4: 1-1498; 1613-1724; 2243-3940; and 3941-5381. Additional preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 4, or the complements thereof, wherein said contiguous span comprises one or more of the nucleotides at positions 1241 or 1447. Further preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 4, or the complements thereof, wherein said contiguous span comprises a T at position 319 or a T at position 3213.

Another object of the invention is a purified, isolated, or recombinant nucleic acid comprising the nucleotide sequence of SEQ ID No 2, complementary sequences thereto, as well as allelic variants, and fragments thereof. Moreover, preferred probes and primers of the invention include purified, isolated, or recombinant AA4RP cDNAs consisting of, consisting essentially of, or comprising the sequence of SEQ ID No 2. Particularly preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 2, or the complements thereof, wherein said contiguous span comprises at least 1, 2, 3, 5, or 10 of the following nucleotide positions of SEQ ID No 2: 1-1879. Additional preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 2, or the complements thereof, wherein said contiguous span comprises a T at position 1153.

Thus, the invention also relates to nucleic acid probes characterized in that they hybridize specifically, under the stringent hybridization conditions defined above, with a nucleic acid selected from the group consisting of the nucleotide sequences 739-1739; 10946-12958; 13470-13526; 13641-13752; 14271-17969; 41718-42718; 44942-45942; and 76558-77558 of SEQ ID No 1 or a variant thereof or a sequence complementary thereto.

Thus, the invention also relates to nucleic acid probes characterized in that they hybridize specifically, under the stringent hybridization conditions defined above, with a nucleic acid selected from the group consisting of the nucleotide sequences 1-1498; 1613-1724; 2243-3940; and 3941-5381 of SEQ ID No 4 or a variant thereof or a sequence complementary thereto.

In one embodiment the invention encompasses isolated, purified, and recombinant polynucleotides consisting of, or consisting essentially of a contiguous span of 8 to 50 nucleotides of any one of SEQ ID Nos 1, 2 or 4 and the complement thereof, wherein said span includes a AA4RP-related biallelic marker in said sequence; optionally, wherein said AA4RP-related biallelic marker is selected from the group consisting of 20-828-311, 17-42-319, 17-41-250, 20-841-149, 20-842-115, and 20-853-415, and the complements thereof, or optionally the biallelic markers in linkage disequilibrium therewith; more preferably said AA4RP-related biallelic marker is selected from the group consisting of 17-42-319 and 17-41-250, and the complements thereof; optionally, wherein said contiguous span is 18 to 35 nucleotides in length and said biallelic marker is within 4 nucleotides of the center of said polynucleotide; optionally, wherein said polynucleotide consists of said contiguous span and said contiguous span is 25 nucleotides in length and said biallelic marker is at the center of said polynucleotide; optionally, wherein the 3′ end of said contiguous span is present at the 3′ end of said polynucleotide; and optionally, wherein the 3′ end of said contiguous span is located at the 3′ end of said polynucleotide and said biallelic marker is present at the 3′ end of said polynucleotide. In a preferred embodiment, said probes comprises, consists of, or consists essentially of a sequence selected from the following sequences of SEQ ID No 1: 1227-1251, 12335-12359, 15229-15253, 42206-42230, 45430-45454 and 77046-77070 and the complementary sequences thereto; and from the following sequences of SEQ ID No 4: 307-331 and 3201-3225 and the complementary sequences thereto.

In another embodiment the invention encompasses isolated, purified and recombinant polynucleotides comprising, consisting of, or consisting essentially of a contiguous span of 8 to 50 nucleotides of SEQ ID Nos 1, 2 or 4, or the complements thereof, wherein the 3′ end of said contiguous span is located at the 3′ end of said polynucleotide, and wherein the 3′ end of said polynucleotide is located within 20 nucleotides upstream of a AA4RP-related biallelic marker in said sequence; optionally, wherein said AA4RP-related biallelic marker is selected from the group consisting of 20-828-311, 17-42-319, 17-41-250, 20-841-149, 20-842-115, and 20-853-415, and the complements thereof, or optionally the biallelic markers in linkage disequilibrium therewith; optionally, wherein said AA4RP-related biallelic marker is selected from the group consisting of 17-42-319 and 17-41-250, and the complements thereof, or optionally the biallelic markers in linkage disequilibrium therewith; optionally, wherein the 3′ end of said polynucleotide is located 1 nucleotide upstream of said AA4RP-related biallelic marker in said sequence; and optionally, wherein said polynucleotide consists essentially of a sequence selected from the following sequences of SEQ ID No 1: 1220-1238, 12328-12346, 15222-15240, 42199-42217, 45423-45441, 77039-77057, 1240-1258, 12348-12366, 15242-15260, 42219-42237, 45443-45461 and 77059-77077; and from the following sequences of SEQ ID No 4: 300-318, 3194-3212, 320-338 and 3214-3232.

In a further embodiment, the invention encompasses isolated, purified, or recombinant polynucleotides comprising, consisting of, or consisting essentially of a sequence selected from the following sequences of SEQ ID No 1: 929-949, 12029-12050, 14992-15012, 42070-42090, 45328-45347, 76644-76664, 1357-1377, 12581-12603, 15460-15482, 42572-42591, 45863-45883, and 77166-77185; and from the following sequences of SEQ ID No 4: 1-11022, 899-11920, 1246-12267, 2964-13984, 553-11575, 1441-12461, 1632-12651, and 3432-14454.

In an additional embodiment, the invention encompasses polynucleotides for use in hybridization assays, sequencing assays, and enzyme-based mismatch detection assays for determining the identity of the nucleotide at a AA4RP-related biallelic marker in SEQ ID Nos 1, 2 or 4, or the complements thereof, as well as polynucleotides for use in amplifying segments of nucleotides comprising a AA4RP-related biallelic marker in SEQ ID Nos 1, 2 or 4, or the complements thereof; optionally, wherein said AA4RP-related biallelic marker is selected from the group consisting of 20-828-311, 17-42-319, 17-41-250, 20-841-149, 20-842-115, and 20-853-415, and the complements thereof, or more preferably the biallelic markers in linkage disequilibrium therewith; optionally, wherein said AA4RP-related biallelic marker is selected from the group consisting of 17-42-319 and 17-41-250, and the complements thereof.

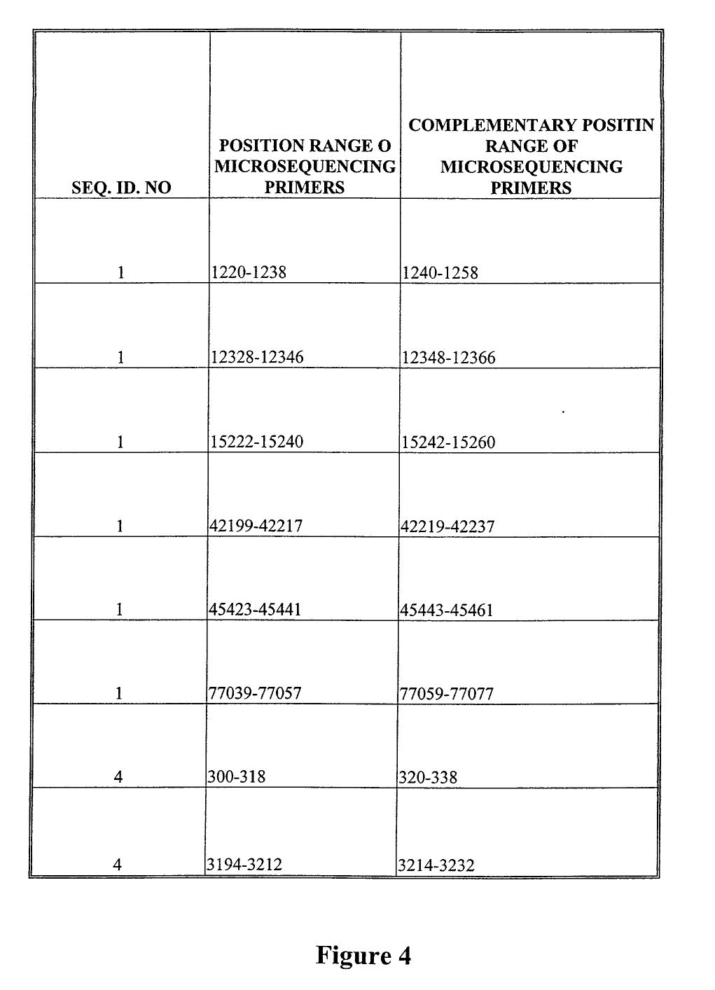

A probe or a primer according to the invention has between 8 and 1000 nucleotides in length, or is specified to be at least 12, 15, 18, 20, 25, 35, 40, 50, 60, 70, 80, 100, 250, 500 or 1000 nucleotides in length. More particularly, the length of these probes and primers can range from 8, 10, 15, 20, or 30 to 100 nucleotides, preferably from 10 to 50, more preferably from 15 to 30 nucleotides. Shorter probes and primers tend to lack specificity for a target nucleic acid sequence and generally require cooler temperatures to form sufficiently stable hybrid complexes with the template. Longer probes and primers are expensive to produce and can sometimes self-hybridize to form hairpin structures. The appropriate length for primers and probes under a particular set of assay conditions may be empirically determined by one of skill in the art. A preferred probe or primer consists of a nucleic acid comprising a polynucleotide selected from the group of the nucleotide sequences of 1227-1251, 12335-12359, 15229-15253, 42206-42230, 45430-45454, 77046-77070, 929-949, 12029-12050, 14992-15012, 42070-42090, 45328-45347, 76644-76664, 1357-1377, 12581-12603, 15460-15482, 42572-42591, 45863-45883, 77166-77185, 1220-1238, 12328-12346, 15222-15240, 42199-42217, 45423-45441, 77039-77057, 1240-1258, 12348-12366, 15242-15260, 42219-42237,45443-45461 and 77059-77077 of SEQ ID No 1 and the complementary sequence thereto; and 307-331, 3201-3225, 1-11022, 899-11920, 1246-12267, 2964-13984, 553-11575, 1441-12461, 1632-12651, 3432-14454, 300-318, 3194-3212, 320-338 and 3214-3232 of SEQ ID No 4 and the complementary sequence thereto; for which the respective locations in the sequence listing are provided in FIGS. 4, 5 and 6.

The formation of stable hybrids depends on the melting temperature (Tm) of the DNA. The Tm depends on the length of the primer or probe, the ionic strength of the solution and the G+C content. The higher the G+C content of the primer or probe, the higher is the melting temperature because G:C pairs are held by three H bonds whereas A:T pairs have only two. The GC content in the probes of the invention usually ranges between 10 and 75%, preferably between 35 and 60%, and more preferably between 40 and 55%.

The primers and probes can be prepared by any suitable method, including, for example, cloning and restriction of appropriate sequences and direct chemical synthesis by a method such as the phosphodiester method of Narang et al.(1979), the phosphodiester method of Brown et al.(1979), the diethylphosphoramidite method of Beaucage et al.(1981) and the solid support method described in EP 0 707 592.

Detection probes are generally nucleic acid sequences or uncharged nucleic acid analogs such as, for example peptide nucleic acids which are disclosed in International Patent Application WO 92/20702, morpholino analogs which are described in U.S. Pat. Nos. 5,185,444; 5,034,506 and 5,142,047. The probe may have to be rendered “non-extendable” in that additional dNTPs cannot be added to the probe. In and of themselves analogs usually are non-extendable and nucleic acid probes can be rendered non-extendable by modifying the 3′ end of the probe such that the hydroxyl group is no longer capable of participating in elongation. For example, the 3′ end of the probe can be functionalized with the capture or detection label to thereby consume or otherwise block the hydroxyl group. Alternatively, the 3′ hydroxyl group simply can be cleaved, replaced or modified, U.S. patent application Ser. No. 07/049,061 filed Apr. 19, 1993 describes modifications, which can be used to render a probe non-extendable.

Any of the polynucleotides of the present invention can be labeled, if desired, by incorporating any label known in the art to be detectable by spectroscopic, photochemical, biochemical, immunochemical, or chemical means. For example, useful labels include radioactive substances (including, ³²P, ³⁵S, ³H, ¹²⁵I), fluorescent dyes (including, 5-bromodesoxyuridin, fluorescein, acetylaminofluorene, digoxigenin) or biotin. Preferably, polynucleotides are labeled at their 3′ and 5′ ends. Examples of non-radioactive labeling of nucleic acid fragments are described in the French patent No. FR-7810975 or by Urdea et al (1988) or Sanchez-Pescador et al (1988). In addition, the probes according to the present invention may have structural characteristics such that they allow the signal amplification, such structural characteristics being, for example, branched DNA probes as those described by Urdea et al. in 1991 or in the European patent No. EP 0 225 807 (Chiron).

A label can also be used to capture the primer, so as to facilitate the immobilization of either the primer or a primer extension product, such as amplified DNA, on a solid support. A capture label is attached to the primers or probes and can be a specific binding member which forms a binding pair with the solid's phase reagent's specific binding member (e.g. biotin and streptavidin). Therefore depending upon the type of label carried by a polynucleotide or a probe, it may be employed to capture or to detect the target DNA. Further, it will be understood that the polynucleotides, primers or probes provided herein, may, themselves, serve as the capture label. For example, in the case where a solid phase reagent's binding member is a nucleic acid sequence, it may be selected such that it binds a complementary portion of a primer or probe to thereby immobilize the primer or probe to the solid phase. In cases where a polynucleotide probe itself serves as the binding member, those skilled in the art will recognize that the probe will contain a sequence or “tail” that is not complementary to the target. In the case where a polynucleotide primer itself serves as the capture label, at least a portion of the primer will be free to hybridize with a nucleic acid on a solid phase. DNA Labeling techniques are well known to the skilled technician.

The probes of the present invention are useful for a number of purposes. They can be notably used in Southern hybridization to genomic DNA. The probes can also be used to detect PCR amplification products. They may also be used to detect mismatches in the AA4RP gene or mRNA using other techniques.

Any of the polynucleotides, primers and probes of the present invention can be conveniently immobilized on a solid support. Solid supports are known to those skilled in the art and include the walls of wells of a reaction tray, test tubes, polystyrene beads, magnetic beads, nitrocellulose strips, membranes, microparticles such as latex particles, sheep (or other animal) red blood cells, duracytes and others. The solid support is not critical and can be selected by one skilled in the art. Thus, latex particles, microparticles, magnetic or non-magnetic beads, membranes, plastic tubes, walls of microtiter wells, glass or silicon chips, sheep (or other suitable animal's) red blood cells and duracytes are all suitable examples. Suitable methods for immobilizing nucleic acids on solid phases include ionic, hydrophobic, covalent interactions and the like.

A solid support, as used herein, refers to any material which is insoluble, or can be made insoluble by a subsequent reaction. The solid support can be chosen for its intrinsic ability to attract and immobilize the capture reagent. Alternatively, the solid phase can retain an additional receptor which has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, duracytes® and other configurations known to those of ordinary skill in the art. The polynucleotides of the invention can be attached to or immobilized on a solid support individually or in groups of at least 2, 5, 8, 10, 12, 15, 20, or 25 distinct polynucleotides of the invention to a single solid support. In addition, polynucleotides other than those of the invention may be attached to the same solid support as one or more polynucleotides of the invention.

Consequently, the invention also comprises a method for detecting the presence of a nucleic acid comprising a nucleotide sequence selected from a group consisting of SEQ ID Nos 1, 2 or 4, a fragment or a variant thereof and a complementary sequence thereto in a sample, said method comprising the following steps of:

a) bringing into contact a nucleic acid probe or a plurality of nucleic acid probes which can hybridize with a nucleotide sequence included in a nucleic acid selected form the group consisting of the nucleotide sequences of SEQ ID Nos 1, 2 or 4, a fragment or a variant thereof and a complementary sequence thereto and the sample to be assayed; and

b) detecting the hybrid complex formed between the probe and a nucleic acid in the sample.

The invention further concerns a kit for detecting the presence of a nucleic acid comprising a nucleotide sequence selected from a group consisting of SEQ ID Nos 1, 2 or 4, a fragment or a variant thereof and a complementary sequence thereto in a sample, said kit comprising:

a) a nucleic acid probe or a plurality of nucleic acid probes which can hybridize with a nucleotide sequence included in a nucleic acid selected form the group consisting of the nucleotide sequences of SEQ ID Nos 1, 2 or 4, a fragment or a variant thereof and a complementary sequence thereto; and

b) optionally, the reagents necessary for performing the hybridization reaction.

In a first preferred embodiment of this detection method and kit, said nucleic acid probe or the plurality of nucleic acid probes are labeled with a detectable molecule. In a second preferred embodiment of said method and kit, said nucleic acid probe or the plurality of nucleic acid probes has been immobilized on a substrate. In a third preferred embodiment, the nucleic acid probe or the plurality of nucleic acid probes comprise either a sequence which is selected from the group consisting of the nucleotide sequences of 1227-1251, 12335-12359, 15229-15253, 42206-42230, 45430-45454, 77046-77070, 929-949, 12029-12050, 14992-15012, 42070-42090, 45328-45347, 76644-76664, 1357-1377, 12581-12603, 15460-15482, 42572-42591, 45863-45883, 77166-77185, 1220-1238, 12328-12346, 15222-15240, 42199-42217, 45423-45441, 77039-77057, 1240-1258, 12348-12366, 15242-15260, 42219-42237, 45443-45461 and 77059-77077 of SEQ ID No 1 or the complementary sequence thereto; and 307-331, 3201-3225, 1-11022, 899-11920, 1246-12267, 2964-13984, 553-11575, 1441-12461, 1632-12651, 3432-14454, 300-318, 3194-3212, 320-338 and 3214-3232 of SEQ ID No 4 or the complementary sequence thereto.

F. Oligonucleotide Arrays

A substrate comprising a plurality of oligonucleotide primers or probes of the invention may be used either for detecting or amplifying targeted sequences in the AA4RP gene and may also be used for detecting mutations in the coding or in the non-coding sequences of the AA4RP gene.

Any polynucleotide provided herein may be attached in overlapping areas or at random locations on the solid support. Alternatively the polynucleotides of the invention may be attached in an ordered array wherein each polynucleotide is attached to a distinct region of the solid support which does not overlap with the attachment site of any other polynucleotide. Preferably, such an ordered array of polynucleotides is designed to be “addressable” where the distinct locations are recorded and can be accessed as part of an assay procedure. Addressable polynucleotide arrays typically comprise a plurality of different oligonucleotide probes that are coupled to a surface of a substrate in different known locations. The knowledge of the precise location of each polynucleotides location makes these “addressable” arrays particularly useful in hybridization assays. Any addressable array technology known in the art can be employed with the polynucleotides of the invention. One particular embodiment of these polynucleotide arrays is known as the Genechips™, and has been generally described in U.S. Pat. No. 5,143,854; PCT publications WO 90/15070 and 92/10092. These arrays may generally be produced using mechanical synthesis methods or light directed synthesis methods which incorporate a combination of photolithographic methods and solid phase oligonucleotide synthesis (Fodor et al., 1991). The immobilization of arrays of oligonucleotides on solid supports has been rendered possible by the development of a technology generally identified as “Very Large Scale Immobilized Polymer Synthesis” (VLSIPS™) in which, typically, probes are immobilized in a high density array on a solid surface of a chip. Examples of VLSIPS™ technologies are provided in U.S. Pat. Nos. 5,143,854; and 5,412,087 and in PCT Publications WO 90/15070, WO 92/10092 and WO 95/11995, which describe methods for forming oligonucleotide arrays through techniques such as light-directed synthesis techniques. In designing strategies aimed at providing arrays of nucleotides immobilized on solid supports, further presentation strategies were developed to order and display the oligonucleotide arrays on the chips in an attempt to maximize hybridization patterns and sequence information. Examples of such presentation strategies are disclosed in PCT Publications WO 94/12305, WO 94/11530, WO 97/29212 and WO 97/31256, the disclosures of which are incorporated herein by reference in their entireties.

In another embodiment of the oligonucleotide arrays of the invention, an oligonucleotide probe matrix may advantageously be used to detect mutations occurring in the AA4RP gene and preferably in its regulatory region. For this particular purpose, probes are specifically designed to have a nucleotide sequence allowing their hybridization to the genes that carry known mutations (either by deletion, insertion or substitution of one or several nucleotides). By known mutations, it is meant, mutations on the AA4RP gene that have been identified according, for example to the technique used by Huang et al.(1996) or Samson et al.(1996).

Another technique that is used to detect mutations in the AA4RP gene is the use of a high-density DNA array. Each oligonucleotide probe constituting a unit element of the high density DNA array is designed to match a specific subsequence of the AA4RP genomic DNA or cDNA. Thus, an array consisting of oligonucleotides complementary to subsequences of the target gene sequence is used to determine the identity of the target sequence with the wild gene sequence, measure its amount, and detect differences between the target sequence and the reference wild gene sequence of the AA4RP gene. In one such design, termed 4L tiled array, is implemented a set of four probes (A, C, G, T), preferably 15-nucleotide oligomers. In each set of four probes, the perfect complement will hybridize more strongly than mismatched probes. Consequently, a nucleic acid target of length L is scanned for mutations with a tiled array containing 4L probes, the whole probe set containing all the possible mutations in the known wild reference sequence. The hybridization signals of the 15-mer probe set tiled array are perturbed by a single base change in the target sequence. As a consequence, there is a characteristic loss of signal or a “footprint” for the probes flanking a mutation position. This technique was described by Chee et al. in 1996.

Consequently, the invention concerns an array of nucleic acid molecules comprising at least one polynucleotide described above as probes and primers. Preferably, the invention concerns an array of nucleic acid comprising at least two polynucleotides described above as probes and primers.

A further object of the invention consists of an array of nucleic acid sequences comprising either at least one of the sequences selected from the group consisting of 1227-1251, 12335-12359, 15229-15253, 42206-42230, 45430-45454, 77046-77070, 929-949, 12029-12050, 14992-15012, 42070-42090, 45328-45347, 76644-76664, 1357-1377, 12581-12603, 15460-15482, 42572-42591, 45863-45883, 77166-77185, 1220-1238, 12328-12346, 15222-15240, 42199-42217, 45423-45441, 77039-77057, 1240-1258, 12348-12366, 15242-15260, 42219-42237,45443-45461 and 77059-77077 of SEQ ID No 1, and the complementary sequence thereto; and 307-331, 3201-3225, 1-11022, 899-11920, 1246-12267, 2964-13984, 553-11575, 1441-12461, 1632-12651, 3432-14454, 300-318, 3194-3212, 320-338 and 3214-3232 of SEQ ID No 4, and the complementary sequence thereto; a fragment thereof of at least 8, 10, 12, 15, 18, 20, 25, 30, or 40 consecutive nucleotides thereof, and at least one sequence comprising a biallelic marker selected from the group consisting of 20-828-311, 17-42-319, 17-41-250, 20-841-149, 20-842-115, and 20-853-415, and the complements thereto.

The invention also pertains to an array of nucleic acid sequences comprising either at least two of the sequences selected from the group consisting of 1227-1251, 12335-12359, 15229-15253, 42206-42230, 45430-45454, 77046-77070, 929-949, 12029-12050, 14992-15012, 42070-42090, 45328-45347, 76644-76664, 1357-1377, 12581-12603, 15460-15482, 42572-42591, 45863-45883, 77166-77185, 1220-1238, 12328-12346, 15222-15240, 42199-42217, 45423-45441, 77039-77057, 1240-1258, 12348-12366, 15242-15260, 42219-42237, 45443-45461 and 77059-77077 of SEQ ID No 1, and the complementary sequence thereto; and 307-331, 3201-3225, 1-11022, 899-11920, 1246-12267, 2964-13984, 553-11575, 1441-12461, 1632-12651, 3432-14454, 300-318, 3194-3212, 320-338 and 3214-3232 of SEQ ID No 4, and the complementary sequence thereto, a fragment thereof of at least 8 consecutive nucleotides thereof, and at least two sequences comprising a biallelic marker selected from the group consisting of 20-828-311, 17-42-319, 17-41-250, 20-841-149, 20-842-115, and 20-853-415, and the complements thereof.

II. AA4RP Proteins and Polypeptide Fragments

The term “AA4RP polypeptides” is used herein to embrace all of the proteins and polypeptides of the present invention. Also forming part of the invention are polypeptides encoded by the polynucleotides of the invention, as well as fusion polypeptides comprising such polypeptides. The invention embodies AA4RP proteins from humans, including isolated or purified AA4RP proteins consisting of, consisting essentially of, or comprising the sequence of SEQ ID No 3.

The present invention embodies isolated, purified, and recombinant polypeptides comprising a contiguous span of at least 6 amino acids, preferably at least 8 to 10 amino acids, more preferably at least 12, 15, 20, 25,30, 40, 50, 100, 200 or 300 amino acids of SEQ ID No 3. The present invention also embodies isolated, purified, and recombinant polypeptides comprising a contiguous span of at least 6 amino acids, preferably at least 8 to 10 amino acids, more preferably at least 12, 15, 20, 25, 30, 40, 50, 100, 200 or 300 amino acids of SEQ ID No 3. In other preferred embodiments the contiguous stretch of amino acids comprises the site of a mutation or functional mutation, including a deletion, addition, swap or truncation of the amino acids in the AA4RP protein sequence.

The invention also encompasses a purified, isolated, or recombinant polypeptides comprising an amino acid sequence having at least 70, 75, 80, 85, 90, 95, 98 or 99% amino acid identity with the amino acid sequence of SEQ ID No 3 or a fragment thereof.

AA4RP proteins are preferably isolated from human or mammalian tissue samples or expressed from human or mammalian genes. The AA4RP polypeptides of the invention can be made using routine expression methods known in the art or as described herein in Example 4. The polynucleotide encoding the desired polypeptide, is ligated into an expression vector suitable for any convenient host. Both eukaryotic and prokaryotic host systems are used in forming recombinant polypeptides, and a summary of some of the more common systems are provided herein. The polypeptide is then isolated from lysed cells or from the culture medium and purified to the extent needed for its intended use. Purification is by any technique known in the art, for example, differential extraction, salt fractionation, chromatography, centrifugation, and the like.

In addition, shorter protein fragments is produced by chemical synthesis. Alternatively the proteins of the invention is extracted from cells or tissues of humans or non-human animals. Methods for purifying proteins are known in the art, and include the use of detergents or chaotropic agents to disrupt particles followed by differential extraction and separation of the polypeptides by ion exchange chromatography, affinity chromatography, sedimentation according to density, and gel electrophoresis.

Any AA4RP cDNA, including SEQ ID No 2, is used to express AA4RP proteins and polypeptides. The nucleic acid encoding the AA4RP protein or polypeptide to be expressed is operably linked to a promoter in an expression vector using conventional cloning technology. The AA4RP insert in the expression vector may comprise the full coding sequence for the AA4RP protein or a portion thereof. For example, the AA4RP derived insert may encode a polypeptide comprising at least 10 consecutive amino acids of the AA4RP protein of SEQ ID No 3.

The expression vector is any of the mammalian, yeast, insect or bacterial expression systems known in the art. Commercially available vectors and expression systems are available from a variety of suppliers including Genetics Institute (Cambridge, Mass.), Stratagene (La Jolla, Calif.), Promega (Madison, Wis.), and Invitrogen (San Diego, Calif.). If desired, to enhance expression and facilitate proper protein folding, the codon context and codon pairing of the sequence is optimized for the particular expression organism in which the expression vector is introduced, as explained by Hatfield, et al., U.S. Pat. No. 5,082,767, the disclosures of which are incorporated by reference herein in their entirety.

In one embodiment, the entire coding sequence of the AA4RP cDNA through the poly A signal of the cDNA are operably linked to a promoter in the expression vector. Alternatively, if the nucleic acid encoding a portion of the AA4RP protein lacks a methionine to serve as the initiation site, an initiating methionine can be introduced next to the first codon of the nucleic acid using conventional techniques. Similarly, if the insert from the AA4RP cDNA lacks a poly A signal, this sequence can be added to the construct by, for example, splicing out the Poly A signal from pSG5 (Stratagene) using BglI and SalI restriction endonuclease enzymes and incorporating it into the mammalian expression vector pXT1 (Stratagene). pXT1 contains the LTRs and a portion of the gag gene from Moloney Murine Leukemia Virus. The position of the LTRs in the construct allow efficient stable transfection. The vector includes the Herpes Simplex Thymidine Kinase promoter and the selectable neomycin gene. The nucleic acid encoding the AA4RP protein or a portion thereof is obtained by PCR from a bacterial vector containing the AA4RP cDNA of SEQ ID No 2 using oligonucleotide primers complementary to the AA4RP cDNA or portion thereof and containing restriction endonuclease sequences for Pst I incorporated into the 5′primer and BglII at the 5′ end of the corresponding cDNA 3′ primer, taking care to ensure that the sequence encoding the AA4RP protein or a portion thereof is positioned properly with respect to the poly A signal. The purified fragment obtained from the resulting PCR reaction is digested with PstI, blunt ended with an exonuclease, digested with Bgl II, purified and ligated to pXT1, now containing a poly A signal and digested with BglII.

The ligated product is transfected into mouse NIH 3T3 cells using Lipofectin (Life Technologies, Inc., Grand Island, N.Y.) under conditions outlined in the product specification. Positive transfectants are selected after growing the transfected cells in 600 ug/ml G418 (Sigma, St. Louis, Mo.).

The above procedures may also be used to express a mutant AA4RP protein responsible for a detectable phenotype or a portion thereof.

The expressed protein is purified using conventional purification techniques such as ammonium sulfate precipitation or chromatographic separation based on size or charge. The protein encoded by the nucleic acid insert may also be purified using standard immunochromatography techniques. In such procedures, a solution containing the expressed AA4RP protein or portion thereof, such as a cell extract, is applied to a column having antibodies against the AA4RP protein or portion thereof is attached to the chromatography matrix. The expressed protein is allowed to bind the immunochromatography column. Thereafter, the column is washed to remove non-specifically bound proteins. The specifically bound expressed protein is then released from the column and recovered using standard techniques.

To confirm expression of the AA4RP protein or a portion thereof, the proteins expressed from host cells containing an expression vector containing an insert encoding the AA4RP protein or a portion thereof can be compared to the proteins expressed in host cells containing the expression vector without an insert. The presence of a band in samples from cells containing the expression vector with an insert which is absent in samples from cells containing the expression vector without an insert indicates that the AA4RP protein or a portion thereof is being expressed. Generally, the band will have the mobility expected for the AA4RP protein or portion thereof. However, the band may have a mobility different than that expected as a result of modifications such as glycosylation, ubiquitination, or enzymatic cleavage.

Antibodies capable of specifically recognizing the expressed AA4RP protein or a portion thereof are described below.

If antibody production is not possible, the nucleic acids encoding the AA4RP protein or a portion thereof is incorporated into expression vectors designed for use in purification schemes employing chimeric polypeptides. In such strategies the nucleic acid encoding the AA4RP protein or a portion thereof is inserted in frame with the gene encoding the other half of the chimera. The other half of the chimera is β-globin or a nickel binding polypeptide encoding sequence. A chromatography matrix having antibody to β-globin or nickel attached thereto is then used to purify the chimeric protein. Protease cleavage sites is engineered between the β-globin gene or the nickel binding polypeptide and the AA4RP protein or portion thereof. Thus, the two polypeptides of the chimera is separated from one another by protease digestion.

One useful expression vector for generating β-globin chimeric proteins is pSG5 (Stratagene), which encodes rabbit β-globin. Intron II of the rabbit β-globin gene facilitates splicing of the expressed transcript, and the polyadenylation signal incorporated into the construct increases the level of expression. These techniques are well known to those skilled in the art of molecular biology. Standard methods are published in methods texts such as Davis et al., (1986) and many of the methods are available from Stratagene, Life Technologies, Inc., or Promega. Polypeptide may additionally be produced from the construct using in vitro translation systems such as the In vitro Express™ Translation Kit (Stratagene).

A. Antibodies That Bind AA4RP Polypeptides of the Invention

Any AA4RP polypeptide or whole protein may be used to generate antibodies capable of specifically binding to an expressed AA4RP protein or fragments thereof as described.

One antibody composition of the invention is capable of specifically binding or specifically bind to the AA4RP protein of SEQ ID No 3. For an antibody composition to specifically bind to a first variant of AA4RP, it must demonstrate at least a 5%, 10%, 15%, 20%, 25%, 50%, or 100% greater binding affinity for a full length first variant of the AA4RP protein than for a full length second variant of the AA4RP protein in an ELISA, RIA, or other antibody-based binding assay.

In a preferred embodiment, the invention concerns antibody compositions, either polyclonal or monoclonal, capable of selectively binding, or selectively bind to an epitope-containing a polypeptide comprising a contiguous span of at least 6 amino acids, preferably at least 8 to 10 amino acids, more preferably at least 12, 15, 20, 25, 30, 40, 50, or 100 amino acids of SEQ ID No 3.

The invention also concerns a purified or isolated antibody capable of specifically binding to a mutated AA4RP protein or to a fragment or variant thereof comprising an epitope of the mutated AA4RP protein. In another preferred embodiment, the present invention concerns an antibody capable of binding to a polypeptide comprising at least 10 consecutive amino acids of a AA4RP protein and including at least one of the amino acids which can be encoded by the trait causing mutations.

In a preferred embodiment, the invention concerns the use in the manufacture of antibodies of a polypeptide comprising a contiguous span of at least 6 amino acids, preferably at least 8 to 10 amino acids, more preferably at least 12, 15, 20, 25, 30, 40, 50, or 100 amino acids of SEQ ID No 3.

Non-human animals or mammals, whether wild-type or transgenic, which express a different species of AA4RP than the one to which antibody binding is desired, and animals which do not express AA4RP (i.e. a AA4RP knock out animal as described herein) are particularly useful for preparing antibodies. AA4RP knock out animals will recognize all or most of the exposed regions of a AA4RP protein as foreign antigens, and therefore produce antibodies with a wider array of AA4RP epitopes. Moreover, smaller polypeptides with only 10 to 30 amino acids may be useful in obtaining specific binding to AA4RP proteins. In addition, the humoral immune system of animals which produce a species of AA4RP that resembles the antigenic sequence will preferentially recognize the differences between the animal's native AA4RP species and the antigen sequence, and produce antibodies to these unique sites in the antigen sequence. Such a technique will be particularly useful in obtaining antibodies that specifically bind to the AA4RP protein.

Antibody preparations prepared according to either protocol are useful in quantitative immunoassays which determine concentrations of antigen-bearing substances in biological samples; they are also used semi-quantitatively or qualitatively to identify the presence of antigen in a biological sample. The antibodies may also be used in therapeutic compositions for killing cells expressing the protein or reducing the levels of the protein in the body.

The antibodies of the invention may be labeled by any one of the radioactive, fluorescent or enzymatic labels known in the art.

Consequently, the invention is also directed to a method for detecting specifically the presence of a AA4RP polypeptide according to the invention in a biological sample, said method comprising the following steps:

a) bringing into contact the biological sample with a polyclonal or monoclonal antibody that specifically binds a AA4RP polypeptide comprising an amino acid sequence of SEQ ID No 3, or to a peptide fragment or variant thereof; and

b) detecting the antigen-antibody complex formed.

The invention also concerns a diagnostic kit for detecting in vitro the presence of a AA4RP polypeptide according to the present invention in a biological sample, wherein said kit comprises:

a) a polyclonal or monoclonal antibody that specifically binds a AA4RP polypeptide comprising an amino acid sequence of SEQ ID No 3, or to a peptide fragment or variant thereof, optionally labeled;

b) a reagent allowing the detection of the antigen-antibody complexes formed, said reagent carrying optionally a label, or being able to be recognized itself by a labeled reagent, more particularly in the case when the above-mentioned monoclonal or polyclonal antibody is not labeled by itself.

The present invention further relates to antibodies and T-cell antigen receptors (TCR) which specifically bind the polypeptides of the present invention. The antibodies of the present invention include IgG (including IgG1, IgG2, IgG3, and IgG4), IgA (including IgA1 and IgA2), IgD, IgE, or IgM, and IgY. As used herein, the term “antibody” (Ab) is meant to include whole antibodies, including single-chain whole antibodies, and antigen-binding fragments thereof. In a preferred embodiment the antibodies are human antigen binding antibody fragments of the present invention include, but are not limited to, Fab, Fab° F.(ab)2 and F(ab′)2, Fd, single-chain Fvs (scFv), single-chain antibodies, disulfide-linked Fvs (sdFv) and fragments comprising either a VL or VH domain. The antibodies may be from any animal origin including birds and mammals. Preferably, the antibodies are human, murine, rabbit, goat, guinea pig, camel, horse, or chicken.

Antigen-binding antibody fragments, including single-chain antibodies, may comprise the variable region(s) alone or in combination with the entire or partial of the following: hinge region, CH1, CH2, and CH3 domains. Also included in the invention are any combinations of variable region(s) and hinge region, CH1, CH2, and CH3 domains. The present invention further includes chimeric, humanized, and human monoclonal and polyclonal antibodies which specifically bind the polypeptides of the present invention. The present invention further includes antibodies which are anti-idiotypic to the antibodies of the present invention.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for heterologous compositions, such as a heterologous polypeptide or solid support material. See, e.g., WO 93/17715; WO 92/08802; WO 91/00360; WO 92/05793; Tuft, A. et al. (1991); U.S. Pat. Nos. 5,573,920, 4,474,893, 5,601,819, 4,714,681, 4,925,648; Kostelny, S. A. et al. (1992).

In some embodiments, the antibodies may be capable of specifically binding to a protein or polypeptide encoded by AA4RP-related nucleic acids, fragments of AA4RP-related nucleic acids, positional segments of AA4RP-related nucleic acids or fragments of positional segments of AA4RP-related nucleic acids. In some embodiments, the antibody may be capable of binding an antigenic determinant or an epitope in a protein or polypeptide encoded by AA4RP-related nucleic acids, fragments of AA4RP-related nucleic acids, positional segments of AA4RP-related nucleic acids or fragments of positional segments of AA4RP-related nucleic acids.

In other embodiments, the antibodies may be capable of specifically binding to an AA4RP-related polypeptide, fragment of an AA4RP-related polypeptide, positional segment of an AA4RP-related polypeptide or fragment of a positional segment of an AA4RP-related polypeptide. In some embodiments, the antibody may be capable of binding an antigenic determinant or an epitope in an AA4RP-related polypeptide, fragment of an AA4RP-related polypeptide, positional segment of an AA4RP-related polypeptide or fragment of a positional segment of an AA4RP-related polypeptide.

Antibodies of the present invention may be described or specified in terms of the epitope(s) or portion(s) of a polypeptide of the present invention which are recognized or specifically bound by the antibody. In the case of secreted proteins, the antibodies may specifically bind a full-length protein encoded by a nucleic acid of the present invention, a mature protein (i.e. the protein generated by cleavage of the signal peptide) encoded by a nucleic acid of the present invention, or a signal peptide encoded by a nucleic acid of the present invention. Moreover, the epitope(s) or polypeptide portion(s) may be specified as described herein, e.g., by N-terminal and C-terminal positions, by size in contiguous amino acid residues, or listed in the figures and sequence listing. Antibodies which specifically bind any epitope or polypeptide of the present invention may also be excluded. Therefore, the present invention includes antibodies that specifically bind polypeptides of the present invention, and allows for the exclusion of the same.

Antibodies of the present invention may also be described or specified in terms of their cross-reactivity. Antibodies that do not bind any other analog, ortholog, or homolog of the polypeptides of the present invention are included. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. Further included in the present invention are antibodies which only bind polypeptides encoded by polynucleotides which hybridize to a polynucleotide of the present invention under stringent hybridization conditions (as described herein). Antibodies of the present invention may also be described or specified in terms of their binding affinity. Preferred binding affinities include those with a dissociation constant or Kd less than 5×10⁻⁶M, 10⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10¹³M, 10³M, 5×10⁻¹⁴M, 10⁻¹⁴M, 5×10⁻¹⁵M, and 10⁻¹⁵M.

Antibodies of the present invention have uses that include, but are not limited to, methods known in the art to purify, detect, and target the polypeptides of the present invention including both in vitro and in vivo diagnostic and therapeutic methods. For example, the antibodies have use in immunoassays for qualitatively and quantitatively measuring levels of the polypeptides of the present invention in biological samples. See, e.g., Harlow et al., 1988 (incorporated by reference in the entirety).

The antibodies of the present invention may be used either alone or in combination with other compositions. The antibodies may further be recombinantly fused to a heterologous polypeptide at the N- or C-terminus or chemically conjugated (including covalent and non-covalent conjugations) to polypeptides or other compositions. For example, antibodies of the present invention may be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP0396 387.

The antibodies of the present invention may be prepared by any suitable method known in the art. For example, a polypeptide of the present invention or an antigenic fragment thereof can be administered to an animal in order to induce the production of sera containing polyclonal antibodies. The term “monoclonal antibody” is not limited to antibodies produced through hybridoma technology. The term “antibody” refers to a polypeptide or group of polypeptides which are comprised of at least one binding domain, where a binding domain is formed from the folding of variable domains of an antibody molecule to form three-dimensional binding spaces with an internal surface shape and charge distribution complementary to the features of an antigenic determinant of an antigen., which allows an immunological reaction with the antigen. The term “monoclonal antibody” refers to an antibody that is derived from a single clone, including eukaryotic, prokaryotic, or phage clone, and not the method by which it is produced. Monoclonal antibodies can be prepared using a wide variety of techniques known in the art including the use of hybridoma, recombinant, and phage display technology.

Hybridoma techniques include those known in the art (See, e.g., Harlow et al., 1988; Hammerling, et al., 1981; (said references incorporated by reference in their entireties). Fab and F(ab′)2 fragments may be produced, for example, from hybridoma-produced antibodies by proteolytic cleavage, using enzymes such as papain (to produce Fab fragments) or pepsin (to produce F(ab′)2 fragments).

Alternatively, antibodies of the present invention can be produced through the application of recombinant DNA technology or through synthetic chemistry using methods known in the art. For example, the antibodies of the present invention can be prepared using various phage display methods known in the art. In phage display methods, functional antibody domains are displayed on the surface of a phage particle which carries polynucleotide sequences encoding them. Phage with a desired binding property are selected from a repertoire or combinatorial antibody library (e.g. human or murine) by selecting directly with antigen, typically antigen bound or captured to a solid surface or bead. Phage used in these methods are typically filamentous phage including fd and M13 with Fab, Fv or disulfide stabilized Fv antibody domains recombinantly fused to either the phage gene III or gene VIII protein. Examples of phage display methods that can be used to make the antibodies of the present invention include those disclosed in Brinkman U. et al. (1995); Ames, R. S. et al. (1995); Kettleborough, C. A. et al. (1994); Persic, L. et al. (1997); Burton, D. R. et al. (1994); PCT/GB91/01134; WO 90/02809; WO 91/10737; WO 92/01047; WO 92/18619; WO 93/11236; WO 95/15982; WO 95/20401; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743 (said references incorporated by reference in their entireties).

As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ F(ab)2 and F(ab′)2 fragments can also be employed using methods known in the art such as those disclosed in WO 92/22324; Mullinax, R. L. et al. (1992); and Sawai, H. et al. (1995); and Better, M. et al. (1988) (said references incorporated by reference in their entireties).

Examples of techniques which can be used to produce single-chain Fvs and antibodies include those described in U.S. Pat. Nos. 4,946,778 and 5,258,498; Huston et al. (1991); Shu, L. et al. (1993); and Skerra, A. et al. (1988). For some uses, including in vivo use of antibodies in humans and in vitro detection assays, it may be preferable to use chimeric, humanized, or human antibodies. Methods for producing chimeric antibodies are known in the art. See e.g., Morrison, (1985); Oi et al., (1986); Gillies, S. D. et al. (1989); and U.S. Pat. No. 5,807,715. Antibodies can be humanized using a variety of techniques including CDR-grafting (EP 0 239 400; WO 91/09967; U.S. Pat. No. 5,530,101; and 5,585,089), veneering or resurfacing (EP 0 592 106; EP 0 519 596; Padlan E. A., (1991); Studnicka G. M. et al. (1994); Roguska M. A. et al. (1994), and chain shuffling (U.S. Pat. No. 5,565,332). Human antibodies can be made by a variety of methods known in the art including phage display methods described above. See also, U.S. Pat. Nos. 4,444,887, 4,716,111, 5,545,806, and 5,814,318; WO 98/46645; WO 98/50433; WO 98/24893; WO 96/34096; WO 96/33735; and WO 91/10741 (said references incorporated by reference in their entireties).

Further included in the present invention are antibodies recombinantly fused or chemically conjugated (including both covalently and non-covalently conjugations) to a polypeptide of the present invention. The antibodies may be specific for antigens other than polypeptides of the present invention. For example, antibodies may be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in in vitro immunoassays and purification methods using methods known in the art. See e.g., Harbor et al. supra and WO 93/21232; EP 0 439 095; Naramura, M. et al. (1994); U.S. Pat. No. 5,474,981; Gillies, S. O. et al. (1992); Fell, H. P. et al. (1991) (said references incorporated by reference in their entireties).

The present invention further includes compositions comprising the polypeptides of the present invention fused or conjugated to antibody domains other than the variable regions. For example, the polypeptides of the present invention may be fused or conjugated to an antibody Fc region, or portion thereof. The antibody portion fused to a polypeptide of the present invention may comprise the hinge region, CH1 domain, CH2 domain, and CH3 domain or any combination of whole domains or portions thereof. The polypeptides of the present invention may be fused or conjugated to the above antibody portions to increase the in vivo half life of the polypeptides or for use in immunoassays using methods known in the art. The polypeptides may also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See e.g., U.S. Pat. Nos. 5,336,603, 5,622,929, 5,359,046, 5,349,053, 5,447,851, 5,112,946; EP 0 307 434, EP 0 367 166; WO 96/04388, WO 91/06570; Ashkenazi, A. et al. (1991); Zheng, X. X. et al. (1995); and Vil, H. et al. (1992) (said references incorporated by reference in their entireties).

The invention further relates to antibodies which act as agonists or antagonists of the polypeptides of the present invention. For example, the present invention includes antibodies which disrupt the receptor/ligand interactions with the polypeptides of the invention either partially or fully. Included are both receptor-specific antibodies and ligand-specific antibodies. Included are receptor-specific antibodies which do not prevent ligand binding but prevent receptor activation. Receptor activation (i.e., signaling) may be determined by techniques described herein or otherwise known in the art. Also include are receptor-specific antibodies which both prevent ligand binding and receptor activation. Likewise, included are neutralizing antibodies which bind the ligand and prevent binding of the ligand to the receptor, as well as antibodies which bind the ligand, thereby preventing receptor activation, but do not prevent the ligand from binding the receptor. Further included are antibodies which activate the receptor. These antibodies may act as agonists for either all or less than all of the biological activities affected by ligand-mediated receptor activation. The antibodies may be specified as agonists or antagonists for biological activities comprising specific activities disclosed herein. The above antibody agonists can be made using methods known in the art. See e.g., WO 96/40281; U.S. Pat. No. 5,811,097; Deng, B. et al. (1998); Chen, Z. et al. (1998); Harrop, J. A. et al. (1998); Zhu, Z. et al. (1998); Yoon, D. Y. et al. (1998); Prat, M. et al. (1998); Pitard, V. et al. (1997); Liautard, J. et al. (1997); Carlson, N. G. et al. (1997); Taryman, R. E. et al. (1995); Muller, Y. A. et al. (1998); Bartunek, P. et al. (1996) (said references incorporated by reference in their entireties).

As discussed above, antibodies of the polypeptides of the invention can, in turn, be utilized to generate anti-idiotypic antibodies that “mimic” polypeptides of the invention using techniques well known to those skilled in the art. See, e.g. Greenspan and Bona, (1989); Nissinoff, (1991). For example, antibodies which bind to and competitively inhibit polypeptide multimerization or binding of a polypeptide of the invention to ligand can be used to generate anti-idiotypes that “mimic” the polypeptide multimerization or binding domain and, as a consequence, bind to and neutralize polypeptide or its ligand. Such neutralization anti-idiotypic antibodies can be used to bind a polypeptide of the invention or to bind its ligands/receptors, and thereby block its biological activity.

B. Epitopes and Antibody Fusions

A preferred embodiment of the present inventions directed to epitope-bearing polypeptides and epitope-bearing polypeptide fragments. These epitopes may be “antigenic epitopes” or both an “antigenic epitope” and an “immunogenic epitope.” An “immunogenic epitope” is defined as a part of a protein that elicits an antibody response in vivo when the polypeptide is the immunogen. On the other hand, a region of polypeptide to which an antibody binds is defined as an “antigenic determinant” or “antigenic epitope.” The number of immunogenic epitopes of a protein generally is less than the number of antigenic epitopes (See, e.g., Geysen, et al., 1983). It is particularly noted that although a particular epitope may not be immunogenic, it is nonetheless useful since antibodies can be made to both immunogenic and antigenic epitopes.

An epitope can comprise as few as 3 amino acids in a spatial conformation, which is unique to the epitope. Generally an epitope consists of at least 6 such amino acids, and more often at least 8-10 such amino acids. In preferred embodiment, antigenic epitopes comprise a number of amino acids that is any integer between 3 and 50. Fragments which function as epitopes may be produced by any conventional means (See, e.g., Houghten, R. A., 1985), also, further described in U.S. Pat. No. 4,631,211. Methods for determining the amino acids which make up an epitope include x-ray crystallography, 2-dimensional nuclear magnetic resonance, and epitope mapping, e.g., the Pepscan method described by Mario H. Geysen et al. (1984); PCT Publication No. WO 84/03564; and PCT Publication No. WO 84/03506. Another example is the algorithm of Jameson and Wolf, (1988) (said references incorporated by reference in their entireties). The Jameson-Wolf antigenic analysis, for example, may be performed using the computer program PROTEAN, using default parameters (Version 4.0 Windows, DNASTAR, Inc., 1228 South Park Street Madison, Wis.

Predicted antigenic epitopes are shown below. It is pointed out that the immunogenic epitope list describe only amino acid residues comprising epitopes predicted to have the highest degree of immunogenicity by a particular algorithm. Polypeptides of the present invention that are not specifically described as immunogenic are not considered non-antigenic. This is because they may still be antigenic in vivo but merely not recognized as such by the particular algorithm used. Alternatively, the polypeptides are probably antigenic in vitro using methods such a phage display. Thus, listed below are the amino acid residues comprising only preferred epitopes, not a complete list. In fact, all fragments of the polypeptides of the present invention, at least 6 amino acids residues in length, are included in the present invention as being useful as antigenic epitope. Moreover, listed below are only the critical residues of the epitopes determined by the Jameson-Wolf analysis. Thus, additional flanking residues on either the N-terminal, C-terminal, or both N- and C-terminal ends may be added to the sequences listed to generate an epitope-bearing portion at least 6 residues in length. Amino acid residues comprising other immunogenic epitopes may be determined by algorithms similar to the Jameson-Wolf analysis or by in vivo testing for an antigenic response using the methods described herein or those known in the art.

The epitope-bearing fragments of the present invention preferably comprises 6 to 50 amino acids (i.e. any integer between 6 and 50, inclusive) of a polypeptide of the present invention. Also, included in the present invention are antigenic fragments between the integers of 6 and the full length AA4RP sequence of the sequence listing. All combinations of sequences between the integers of 6 and the full-length sequence of a AA4RP polypeptide are included. The epitope-bearing fragments may be specified by either the number of contiguous amino acid residues (as a sub-genus) or by specific N-terminal and C-terminal positions (as species) as described above for the polypeptide fragments of the present invention. Any number of epitope-bearing fragments of the present invention may also be excluded in the same manner.

Antigenic epitopes are useful, for example, to raise antibodies, including monoclonal antibodies that specifically bind the epitope (See, Wilson et al., 1984; and Sutcliffe, J. G. et al., 1983). The antibodies are then used in various techniques such as diagnostic and tissue/cell identification techniques, as described herein, and in purification methods.

Similarly, immunogenic epitopes can be used to induce antibodies according to methods well known in the art (See, Sutcliffe et al., supra; Wilson et al., supra; Chow, M. et al.;(1985) and Bittle, F. J. et al., (1985). A preferred immunogenic epitope includes the nature AA4RP protein. The immunogenic epitopes may be presented together with a carrier protein, such as an albumin, to an animal system (such as rabbit or mouse) or, if it is long enough (at least about 25 amino acids), without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting.).

Epitope-bearing polypeptides of the present invention are used to induce antibodies according to methods well known in the art including, but not limited to, in vivo immunization, in vitro immunization, and phage display methods (See, e.g., Sutcliffe, et al., supra; Wilson, et al., supra, and Bittle, et al., 1985). If in vivo immunization is used, animals may be immunized with free peptide; however, anti-peptide antibody titer may be boosted by coupling of the peptide to a macromolecular carrier, such as keyhole limpet hemacyanin (KLH) or tetanus toxoid. For instance, peptides containing cysteine residues may be coupled to a carrier using a linker such as -maleimidobenzoyl-N-hydroxysuccinimide ester (NBS), while other peptides may be coupled to carriers using a more general linking agent such as glutaraldehyde. Animals such as rabbits, rats and mice are immunized with either free or carrier-coupled peptides, for instance, by intraperitoneal and/or intradermal injection of emulsions containing about 100 μgs of peptide or carrier protein and Freund's adjuvant. Several booster injections may be needed, for instance, at intervals of about two weeks, to provide a useful titer of anti-peptide antibody, which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

As one of skill in the art will appreciate, and discussed above, the polypeptides of the present invention comprising an immunogenic or antigenic epitope can be fused to heterologous polypeptide sequences. For example, the polypeptides of the present invention may be fused with the constant domain of immunoglobulins (IgA, IgE, IgG, IgM), or portions thereof (CH1, CH2, CH3, any combination thereof including both entire domains and portions thereof) resulting in chimeric polypeptides. These fusion proteins facilitate purification, and show an increased half-life in vivo. This has been shown, e.g., for chimeric proteins consisting of the first two domains of the human CD4-polypeptide and various domains of the constant regions of the heavy or light chains of mammalian immunoglobulins (See, e.g., EPA 0,394,827; and Traunecker et al., 1988). Fusion proteins that have a disulfide-linked dimeric structure due to the IgG portion can also be more efficient in binding and neutralizing other molecules than monomeric polypeptides or fragments thereof alone (See, e.g., Fountoulakis et al., 1995). Nucleic acids encoding the above epitopes can also be recombined with a gene of interest as an epitope tag to aid in detection and purification of the expressed polypeptide.

Additional fusion proteins of the invention may be generated through the techniques of gene-shuffling, motif-shuffling, exon-shuffling, or codon-shuffling (collectively referred to as “DNA shuffling”). DNA shuffling may be employed to modulate the activities of polypeptides of the present invention thereby effectively generating agonists and antagonists of the polypeptides. See, for example, U.S. Pat. Nos. 5,605,793; 5,811,238; 5,834,252; 5,837,458; and Patten, P. A., et al., (1997); Harayama, S., (1998); Hansson, L. O., et al (1999); and Lorenzo, M. M. and Blasco, R., (1998). (Each of these documents are hereby incorporated by reference). In one embodiment, one or more components, motifs, sections, parts, domains, fragments, etc., of coding polynucleotides of the invention, or the polypeptides encoded thereby may be recombined with one or more components, motifs, sections, parts, domains, fragments, etc. of one or more heterologous molecules.

Preferred AA4RP immunogenic epitopes:

| Gln22 to Phe27 Gln33 to Arg40 | |
|---|---|
| Ser78 to Met92 Gln128 to Thr133 | |
| Gly265 to Pro274 | Phe288 to Thr292 |
| Leu355 to His360 | |

| | | Antigenic Index |
|---|---|---|
| Residue | Position | (Jameson-Wolf) |
| Met | 1 | −0.60 |
| Ala | 2 | −0.60 |
| Ser | 3 | −0.60 |
| Met | 4 | −0.60 |
| Ala | 5 | −0.60 |
| Ala | 6 | −0.60 |
| Val | 7 | −0.60 |
| Leu | 8 | −0.60 |
| Thr | 9 | −0.60 |
| Trp | 10 | −0.60 |
| Ala | 11 | −0.60 |
| Leu | 12 | −0.60 |
| Ala | 13 | −0.60 |
| Leu | 14 | −0.60 |
| Leu | 15 | −0.60 |
| Ser | 16 | −0.60 |
| Ala | 17 | −0.60 |
| Phe | 18 | −0.60 |
| Ser | 19 | −0.60 |
| Ala | 20 | −0.60 |
| Thr | 21 | 0.00 |
| Gln | 22 | 1.18 |
| Ala | 23 | 1.46 |
| Arg | 24 | 2.24 |
| Lys | 25 | 1.77 |
| Gly | 26 | 2.80 |
| Phe | 27 | 2.37 |
| Trp | 28 | 0.84 |
| Asp | 29 | 0.36 |
| Tyr | 30 | 0.43 |
| Phe | 31 | 0.15 |
| Ser | 32 | 0.94 |
| Gln | 33 | 1.03 |
| Thr | 34 | 2.42 |
| Ser | 35 | 2.86 |
| Gly | 36 | 3.40 |
| Asp | 37 | 3.06 |
| Lys | 38 | 2.52 |
| Gly | 39 | 2.18 |
| Arg | 40 | 1.64 |
| Val | 41 | 0.75 |
| Glu | 42 | 0.45 |
| Gln | 43 | 0.30 |
| Ile | 44 | −0.15 |
| His | 45 | 0.90 |
| Gln | 46 | 0.60 |
| Gln | 47 | 0.00 |
| Lys | 48 | 0.90 |
| Met | 49 | 0.90 |
| Ala | 50 | 0.90 |
| Arg | 51 | 0.90 |
| Glu | 52 | 0.60 |
| Pro | 53 | 0.60 |
| Ala | 54 | 0.90 |
| Thr | 55 | 0.90 |
| Leu | 56 | 1.30 |
| Lys | 57 | 1.00 |
| Asp | 58 | 1.30 |
| Ser | 59 | 1.30 |
| Leu | 60 | 0.90 |
| Glu | 61 | 0.90 |
| Gln | 62 | 0.60 |
| Asp | 63 | 0.60 |
| Leu | 64 | 0.60 |
| Asn | 65 | 0.60 |
| Asn | 66 | 0.85 |
| Met | 67 | 0.25 |
| Asn | 68 | 0.10 |
| Lys | 69 | 0.70 |
| Phe | 70 | 0.75 |
| Leu | 71 | 0.30 |
| Glu | 72 | 0.75 |
| Lys | 73 | 0.60 |
| Leu | 74 | 0.90 |
| Arg | 75 | 0.90 |
| Pro | 76 | 0.65 |
| Leu | 77 | 0.60 |
| Ser | 78 | 1.35 |
| Gly | 79 | 1.35 |
| Ser | 80 | 1.80 |
| Glu | 81 | 2.20 |
| Ala | 82 | 2.50 |
| Pro | 83 | 3.00 |
| Arg | 84 | 2.70 |
| Leu | 85 | 2.20 |
| Pro | 86 | 2.35 |
| Gln | 87 | 1.85 |
| Asp | 88 | 1.35 |
| Pro | 89 | 2.05 |
| Val | 90 | 2.50 |
| Gly | 91 | 2.20 |
| Met | 92 | 1.20 |
| Arg | 93 | 0.95 |
| Arg | 94 | 0.85 |
| Gln | 95 | 0.90 |
| Leu | 96 | 0.90 |
| Gln | 97 | 0.90 |
| Glu | 98 | 0.90 |
| Glu | 99 | 0.90 |
| Leu | 100 | 0.90 |
| Glu | 101 | 0.90 |
| Glu | 102 | 0.75 |
| Val | 103 | 0.90 |
| Lys | 104 | 0.75 |
| Ala | 105 | 0.60 |
| Arg | 106 | 0.45 |
| Leu | 107 | 0.45 |
| Gln | 108 | 0.25 |
| Pro | 109 | 0.10 |
| Tyr | 110 | 0.25 |
| Met | 111 | 0.10 |
| Ala | 112 | −0.30 |
| Glu | 113 | 0.30 |
| Ala | 114 | 0.30 |
| His | 115 | 0.30 |
| Glu | 116 | 0.30 |
| Leu | 117 | −0.60 |
| Val | 118 | −0.60 |
| Gly | 119 | −0.60 |
| Trp | 120 | −0.60 |
| Asn | 121 | −0.60 |
| Leu | 122 | −0.60 |
| Glu | 123 | −0.30 |
| Gly | 124 | 0.45 |
| Leu | 125 | 0.60 |
| Arg | 126 | 0.45 |
| Gln | 127 | 0.60 |
| Gln | 128 | 1.25 |
| Leu | 129 | 1.30 |
| Lys | 130 | 1.35 |
| Pro | 131 | 1.60 |
| Tyr | 132 | 2.50 |
| Thr | 133 | 1.85 |
| Met | 134 | 0.15 |
| Asp | 135 | 0.20 |
| Leu | 136 | 0.55 |
| Met | 137 | 0.30 |
| Glu | 138 | 0.30 |
| Gln | 139 | −0.60 |
| Val | 140 | 0.30 |
| Ala | 141 | 0.30 |
| Leu | 142 | −0.30 |
| Arg | 143 | 0.30 |
| Val | 144 | 0.30 |
| Gln | 145 | 0.45 |
| Glu | 146 | 0.90 |
| Leu | 147 | 0.60 |
| Gln | 148 | 0.60 |
| Glu | 149 | 0.90 |
| Gln | 150 | 0.60 |
| Leu | 151 | 0.30 |
| Arg | 152 | 0.30 |
| Val | 153 | 0.30 |
| Val | 154 | 0.60 |
| Gly | 155 | 1.15 |
| Glu | 156 | 1.30 |
| Asp | 157 | 1.30 |
| Thr | 158 | 1.30 |
| Lys | 159 | 0.90 |
| Ala | 160 | 0.45 |
| Gln | 161 | −0.30 |
| Leu | 162 | −0.30 |
| Leu | 163 | −0.60 |
| Gly | 164 | 0.05 |
| Gly | 165 | 0.65 |
| Val | 166 | 0.45 |
| Asp | 167 | 0.45 |
| Glu | 168 | 0.30 |
| Ala | 169 | −0.30 |
| Trp | 170 | −0.30 |
| Ala | 171 | −0.60 |
| Leu | 172 | −0.60 |
| Leu | 173 | −0.60 |
| Gln | 174 | −0.60 |
| Gly | 175 | −0.45 |
| Leu | 176 | 0.60 |
| Gln | 177 | 0.45 |
| Ser | 178 | −0.15 |
| Arg | 179 | −0.15 |
| Val | 180 | −0.30 |
| Val | 181 | −0.30 |
| His | 182 | −0.10 |
| His | 183 | 0.30 |
| Thr | 184 | 0.60 |
| Gly | 185 | 1.20 |
| Arg | 186 | 1.30 |
| Phe | 187 | 0.90 |
| Lys | 188 | 0.45 |
| Glu | 189 | 0.30 |
| Leu | 190 | −0.15 |
| Phe | 191 | −0.30 |
| His | 192 | −0.20 |
| Pro | 193 | −0.05 |
| Tyr | 194 | 0.25 |
| Ala | 195 | 0.25 |
| Glu | 196 | −0.40 |
| Ser | 197 | −0.10 |
| Leu | 198 | −0.10 |
| Val | 199 | −0.10 |
| Ser | 200 | −0.25 |
| Gly | 201 | 0.45 |
| Ile | 202 | 0.05 |
| Gly | 203 | 0.25 |
| Arg | 204 | 0.25 |
| His | 205 | 0.65 |
| Val | 206 | 0.65 |
| Gln | 207 | 0.50 |
| Glu | 208 | 0.65 |
| Leu | 209 | 0.65 |
| His | 210 | 0.50 |
| Arg | 211 | 0.90 |
| Ser | 212 | 0.30 |
| Val | 213 | 0.50 |
| Ala | 214 | 0.70 |
| Pro | 215 | 0.10 |
| His | 216 | −0.20 |
| Ala | 217 | 0.25 |
| Pro | 218 | 0.25 |
| Ala | 219 | 0.25 |
| Ser | 220 | 1.00 |
| Pro | 221 | 0.85 |
| Ala | 222 | 1.00 |
| Arg | 223 | 1.15 |
| Leu | 224 | 0.70 |
| Ser | 225 | 0.70 |
| Arg | 226 | 0.70 |
| Cys | 227 | 0.10 |
| Val | 228 | −0.30 |
| Gln | 229 | −0.30 |
| Val | 230 | −0.30 |
| Leu | 231 | 0.45 |
| Ser | 232 | 0.45 |
| Arg | 233 | 0.60 |
| Lys | 234 | 0.60 |
| Leu | 235 | 0.90 |
| Thr | 236 | 0.60 |
| Leu | 237 | 0.60 |
| Lys | 238 | 0.60 |
| Ala | 239 | 0.45 |
| Lys | 240 | 0.60 |
| Ala | 241 | 0.30 |
| Leu | 242 | 0.45 |
| His | 243 | 0.30 |
| Ala | 244 | −0.30 |
| Arg | 245 | −0.15 |
| Ile | 246 | 0.45 |
| Gln | 247 | 0.00 |
| Gln | 248 | 0.60 |
| Asn | 249 | 0.80 |
| Leu | 250 | 0.80 |
| Asp | 251 | 0.80 |
| Gln | 252 | 1.10 |
| Leu | 253 | 0.90 |
| Arg | 254 | 0.90 |
| Glu | 255 | 0.90 |
| Glu | 256 | 0.90 |
| Leu | 257 | 0.90 |
| Ser | 258 | 0.75 |
| Arg | 259 | 0.30 |
| Ala | 260 | −0.30 |
| Phe | 261 | −0.30 |
| Ala | 262 | 0.00 |
| Gly | 263 | 0.75 |
| Thr | 264 | 1.35 |
| Gly | 265 | 2.70 |
| Thr | 266 | 3.00 |
| Glu | 267 | 2.35 |
| Glu | 268 | 2.49 |
| Gly | 269 | 2.58 |
| Ala | 270 | 2.32 |
| Gly | 271 | 2.31 |
| Pro | 272 | 2.40 |
| Asp | 273 | 2.16 |
| Pro | 274 | 1.72 |
| Gln | 275 | 0.93 |
| Met | 276 | 0.69 |
| Leu | 277 | 0.45 |
| Ser | 278 | 0.45 |
| Glu | 279 | 0.90 |
| Glu | 280 | 0.90 |
| Val | 281 | 0.90 |
| Arg | 282 | 0.90 |
| Gln | 283 | 0.90 |
| Arg | 284 | 0.60 |
| Leu | 285 | 0.30 |
| Gln | 286 | 0.73 |
| Ala | 287 | 0.86 |
| Phe | 288 | 1.69 |
| Arg | 289 | 2.52 |
| Gln | 290 | 2.80 |
| Asp | 291 | 1.92 |
| Thr | 292 | 2.24 |
| Tyr | 293 | −0.04 |
| Leu | 294 | −0.32 |
| Gln | 295 | −0.60 |
| Ile | 296 | −0.60 |
| Ala | 297 | −0.60 |
| Ala | 298 | −0.30 |
| Phe | 299 | −0.60 |
| Thr | 300 | −0.60 |
| Arg | 301 | 0.30 |
| Ala | 302 | 0.45 |
| Ile | 303 | 0.90 |
| Asp | 304 | 0.90 |
| Gln | 305 | 0.90 |
| Glu | 306 | 0.90 |
| Thr | 307 | 0.90 |
| Glu | 308 | 0.90 |
| Glu | 309 | 0.90 |
| Val | 310 | 0.90 |
| Gln | 311 | 0.60 |
| Gln | 312 | −0.15 |
| Gln | 313 | 0.10 |
| Leu | 314 | 0.20 |
| Ala | 315 | 0.20 |
| Pro | 316 | 0.20 |
| Pro | 317 | 0.40 |
| Pro | 318 | 0.60 |
| Pro | 319 | 0.80 |
| Gly | 320 | 0.65 |
| His | 321 | 0.00 |
| Ser | 322 | −0.40 |
| Ala | 323 | −0.40 |
| Phe | 324 | −0.10 |
| Ala | 325 | −0.30 |
| Pro | 326 | −0.30 |
| Glu | 327 | 0.00 |
| Phe | 328 | 0.60 |
| Gln | 329 | 0.90 |
| Gln | 330 | 0.90 |
| Thr | 331 | 0.60 |
| Asp | 332 | 1.70 |
| Ser | 333 | 1.70 |
| Gly | 334 | 1.25 |
| Lys | 335 | 0.85 |
| Val | 336 | 0.45 |
| Leu | 337 | 0.45 |
| Ser | 338 | 0.45 |
| Lys | 339 | −0.15 |
| Leu | 340 | 0.45 |
| Gln | 341 | 0.45 |
| Ala | 342 | 0.60 |
| Arg | 343 | 0.75 |
| Leu | 344 | 0.60 |
| Asp | 345 | 0.45 |
| Sp | 346 | 0.75 |
| Leu | 347 | 0.90 |
| Trp | 348 | 0.75 |
| Glu | 349 | 0.45 |
| Asp | 350 | −0.15 |
| Ile | 351 | 0.45 |
| Thr | 352 | 0.30 |
| His | 353 | −0.30 |
| Ser | 354 | 0.13 |
| Leu | 355 | 1.21 |
| His | 356 | 1.49 |
| Asp | 357 | 2.52 |
| Gln | 358 | 2.80 |
| Gly | 359 | 2.52 |
| His | 360 | 2.09 |
| Ser | 361 | 0.66 |
| His | 362 | 0.98 |
| Leu | 363 | 0.70 |
| Gly | 364 | 0.70 |
| Asp | 365 | 0.70 |
| Pro | 366 | 0.85 |

III. AA4RP-related Biallelic Markers

A. Advantages of the Biallelic Markers of the Present Invention

The AA4RP-related biallelic markers of the present invention offer a number of important advantages over other genetic markers such as RFLP (Restriction fragment length polymorphism) and VNTR (Variable Number of Tandem Repeats) markers.

The first generation of markers, were RFLPs, which are variations that modify the length of a restriction fragment. But methods used to identify and to type RFLPs are relatively wasteful of materials, effort, and time. The second generation of genetic markers were VNTRs, which can be categorized as either minisatellites or microsatellites. Minisatellites are tandemly repeated DNA sequences present in units of 5-50 repeats which are distributed along regions of the human chromosomes ranging from 0.1 to 20 kilobases in length. Since they present many possible alleles, their informative content is very high. Minisatellites are scored by performing Southern blots to identify the number of tandem repeats present in a nucleic acid sample from the individual being tested. However, there are only 10⁴ potential VNTRs that can be typed by Southern blotting. Moreover, both RFLP and VNTR markers are costly and time-consuming to develop and assay in large numbers.

Single nucleotide polymorphism or biallelic markers can be used in the same manner as RFLPs and VNTRs but offer several advantages. SNP are densely spaced in the human genome and represent the most frequent type of variation. An estimated number of more than 10⁷ sites are scattered along the 3×10⁹ base pairs of the human genome. Therefore, SNP occur at a greater frequency and with greater uniformity than RFLP or VNTR markers which means that there is a greater probability that such a marker will be found in close proximity to a genetic locus of interest. SNP are less variable than VNTR markers but are mutationally more stable.

Also, the different forms of a characterized single nucleotide polymorphism, such as the biallelic markers of the present invention, are often easier to distinguish and can therefore be typed easily on a routine basis. Biallelic markers have single nucleotide based alleles and they have only two common alleles, which allows highly parallel detection and automated scoring. The biallelic markers of the present invention offer the possibility of rapid, high throughput genotyping of a large number of individuals.

Biallelic markers are densely spaced in the genome, sufficiently informative and can be assayed in large numbers. The combined effects of these advantages make biallelic markers extremely valuable in genetic studies. Biallelic markers can be used in linkage studies in families, in allele sharing methods, in linkage disequilibrium studies in populations, in association studies of case-control populations or of trait positive and trait negative populations. An important aspect of the present invention is that biallelic markers allow association studies to be performed to identify genes involved in complex traits. Association studies examine the frequency of marker alleles in unrelated case- and control-populations and are generally employed in the detection of polygenic or sporadic traits. Association studies may be conducted within the general population and are not limited to studies performed on related individuals in affected families (linkage studies). Biallelic markers in different genes can be screened in parallel for direct association with disease or response to a treatment. This multiple gene approach is a powerful tool for a variety of human genetic studies as it provides the necessary statistical power to examine the synergistic effect of multiple genetic factors on a particular phenotype, drug response, sporadic trait, or disease state with a complex genetic etiology.

B. Candidate Gene of the Present Invention

Different approaches can be employed to perform association studies: genome-wide association studies, candidate region association studies and candidate gene association studies. Genome-wide association studies rely on the screening of genetic markers evenly spaced and covering the entire genome. The candidate gene approach is based on the study of genetic markers specifically located in genes potentially involved in a biological pathway related to the trait of interest. In the present invention, AA4RP is the candidate gene. The candidate gene analysis clearly provides a short-cut approach to the identification of genes and gene polymorphisms related to a particular trait when some information concerning the biology of the trait is available. However, it should be noted that all of the biallelic markers disclosed in the instant application can be employed as part of genome-wide association studies or as part of candidate region association studies and such uses are specifically contemplated in the present invention and claims.

C. AA4RP-Related Biallelic Markers and Polynucleotides Related Thereto

The invention also concerns AA4RP-related biallelic markers. As used herein the term “AA4RP-related biallelic marker” relates to a set of biallelic markers in linkage disequilibrium with the AA4RP gene. The term AA4RP-related biallelic marker includes the biallelic markers designated 20-828-311, 17-42-319, 17-41-250, 20-841-149, 20-842-115, and 20-853-415.

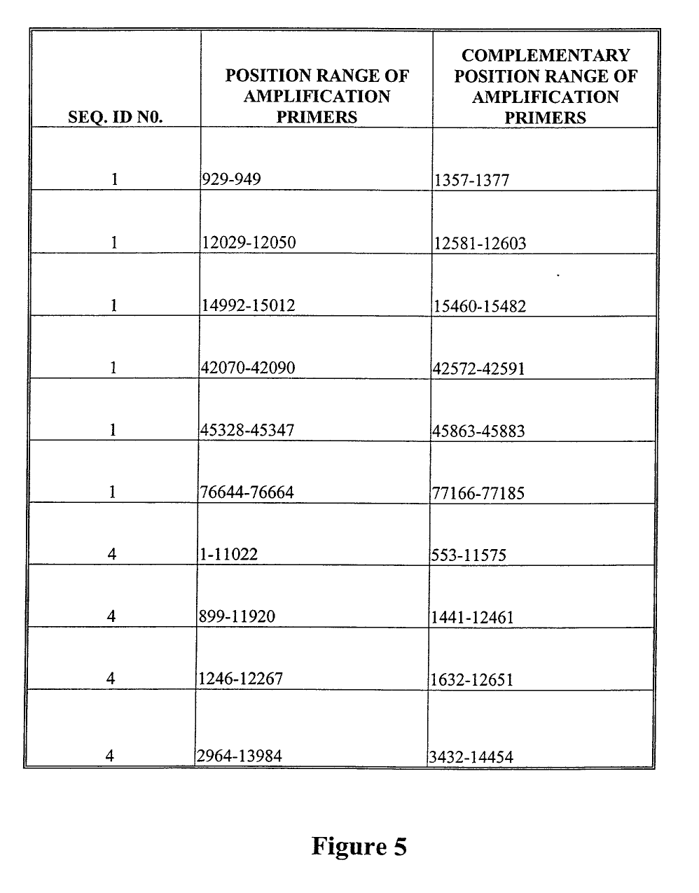

The biallelic markers of the present invention are disclosed in Table 1. Their location on the AA4RP gene is indicated in Table 1 and also as a single base polymorphism in the features of SEQ ID Nos 1, 2 and 4. The pairs of primers allowing the amplification of a nucleic acid containing the polymorphic base of one AA4RP biallelic marker are listed in FIG. 5.

Two AA4RP-related biallelic markers, 17-42-319 and 17-41-250, are located in the genomic sequence of AA4RP. Both markers are located in SEQ ID Nos 1 and 4. Biallelic marker 17-42-319 is located in the 5′ Regulatory region (position 12347 of SEQ ID No 1 and position 319 of SEQ ID No 4), and therefore may alter enhancer regions or regulatory regions. 17-41-250 is located in exon 4 (position 15241 of SEQ ID No 1 and 3213 of SEQ ID No 4), and therefore may alter transcription in the gene.

The invention also relates to a purified and/or isolated nucleotide sequence comprising a polymorphic base of a AA4RP-related biallelic marker, preferably of a biallelic marker selected from the group consisting of 20-828-311, 17-42-319, 17-41-250, 20-841-149, 20-842-115, and 20-853-415, and the complements thereof. The sequence has between 8 and 1000 nucleotides in length, and preferably comprises at least 8, 10, 12, 15, 18, 20, 25, 35, 40, 50, 60, 70, 80, 100, 250, 500 or 1000 contiguous nucleotides of a nucleotide sequence selected from the group consisting of SEQ ID Nos 1, 2 and 4 or a variant thereof or a complementary sequence thereto. These nucleotide sequences comprise the polymorphic base of either allele 1 or allele 2 of the considered biallelic marker. Optionally, said biallelic marker may be within 6, 5, 4, 3, 2, or 1 nucleotides of the center of said polynucleotide or at the center of said polynucleotide. Optionally, the 3′ end of said contiguous span may be present at the 3′ end of said polynucleotide. Optionally, biallelic marker may be present at the 3′ end of said polynucleotide. Optionally, said polynucleotide may further comprise a label. Optionally, said polynucleotide can be attached to solid support. In a further embodiment, the polynucleotides defined above can be used alone or in any combination.

The invention also relates to a purified and/or isolated nucleotide sequence comprising a between 8 and 1000 nucleotides in length, and preferably at least 8, 10, 12, 15, 18, 20, 25, 35, 40, 50, 60, 70, 80, 100, 250, 500 or 1000 contiguous nucleotides of a nucleotide sequence selected from the group consisting of SEQ ID Nos 1, 2 and 4 or a variant thereof or a complementary sequence thereto. Optionally, the 3′ end of said polynucleotide may be located within or at least 2, 4, 6, 8, 10, 12, 15, 18, 20, 25, 50, 100, 250, 500, or 1000 nucleotides upstream of a AA4RP-related biallelic marker in said sequence. Optionally, said AA4RP-related biallelic marker is selected from the group consisting of 20-828-311, 17-42-319, 17-41-250,20-841-149, 20-842-115, and 20-853-415; Optionally, the 3′ end of said polynucleotide may be located within or at least 2, 4, 6, 8, 10, 12, 15, 18, 20, 25, 50, 100, 250, 500, or 1000 nucleotides upstream of a AA4RP-related biallelic marker in said sequence. Optionally, the 3′ end of said polynucleotide may be located 1 nucleotide upstream of a AA4RP-related biallelic marker in said sequence. Optionally, said polynucleotide may further comprise a label. Optionally, said polynucleotide can be attached to solid support. In a further embodiment, the polynucleotides defined above can be used alone or in any combination.

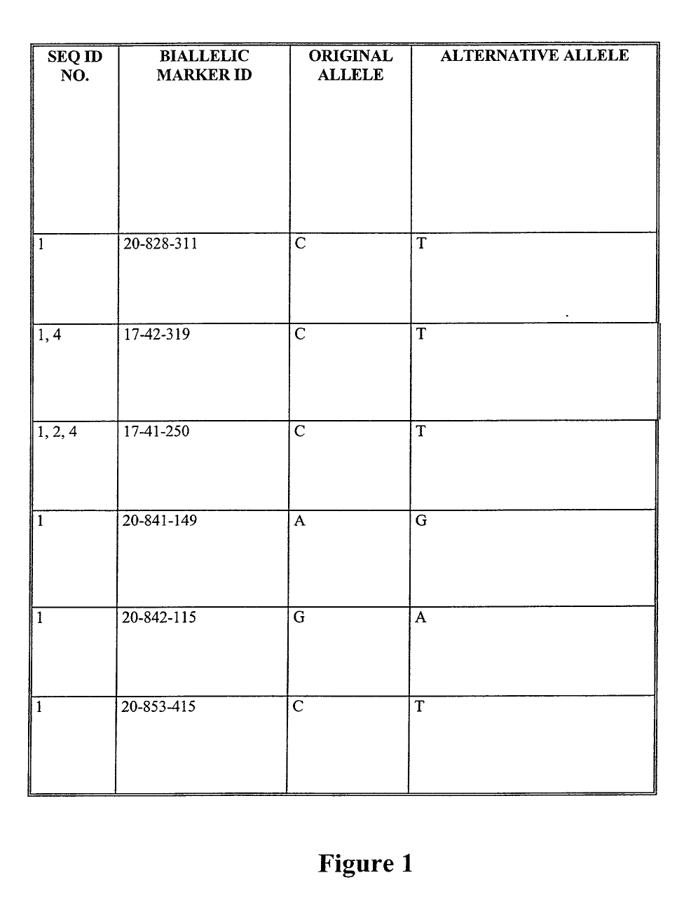

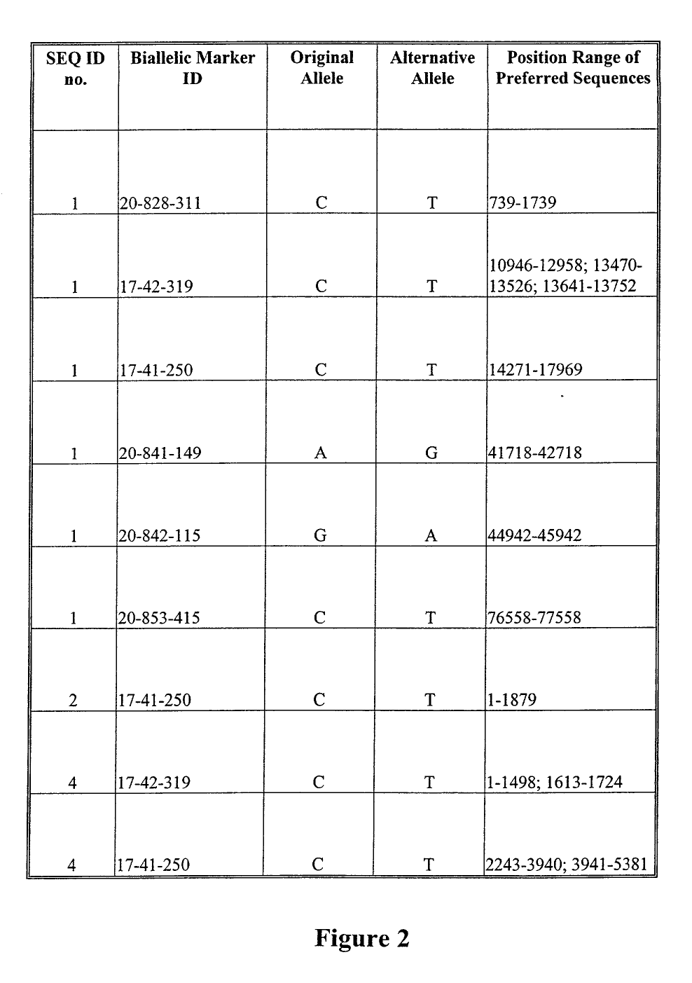

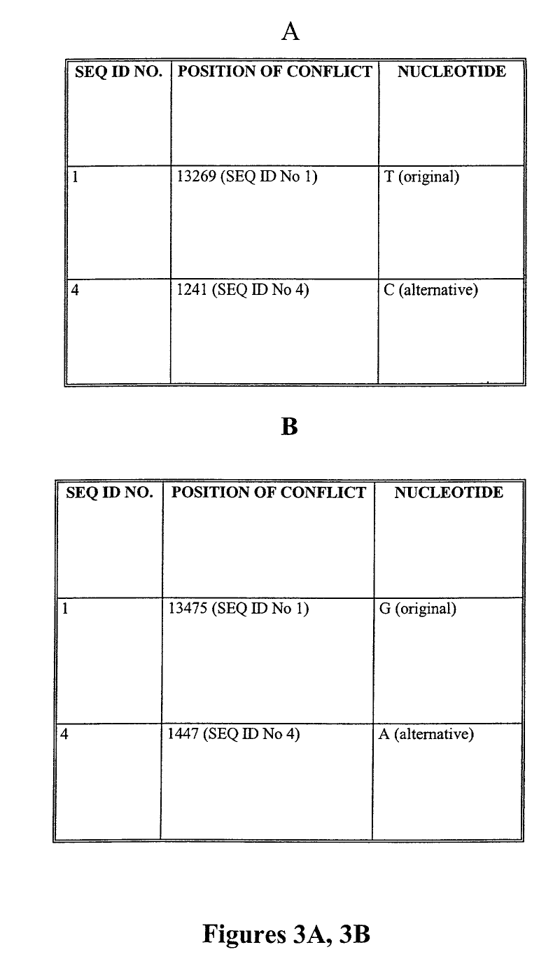

In a preferred embodiment, the sequences comprising a polymorphic base of one of the biallelic markers listed in FIG. 1 are selected from the group consisting of the nucleotide sequences that have a contiguous span of, that consist of, that are comprised in, or that comprises a polynucleotide selected from the group consisting of the nucleic acids of the sequences set forth as the amplicons listed in FIG. 5 or a variant thereof or a complementary sequence thereto.

The invention further concerns a nucleic acid encoding the AA4RP protein, wherein said nucleic acid comprises a polymorphic base of a biallelic marker selected from the group consisting of 20-828-311, 17-42-319, 17-41-250, 20-841-149, 20-842-115, and 20-853-415, and the complements thereof.

The invention also encompasses the use of any polynucleotide for, or any polynucleotide for use in, determining the identity of one or more nucleotides at a AA4RP-related biallelic marker. In addition, the polynucleotides of the invention for use in determining the identity of one or more nucleotides at a AA4RP-related biallelic marker encompass polynucleotides with any further limitation described in this disclosure, or those following, specified alone or in any combination. Optionally, said AA4RP-related biallelic marker is selected from the group consisting of 20-828-311, 17-42-319, 17-41-250, 20-841-149, 20-842-115, and 20-853-415, and the complements thereof, or optionally the biallelic markers in linkage disequilibrium therewith; optionally, said AA4RP-related biallelic marker is selected from the group consisting of 17-42-319 and 17-41-250, and the complements thereof, or optionally the biallelic markers in linkage disequilibrium therewith; Optionally, said polynucleotide may comprise a sequence disclosed in the present specification; Optionally, said polynucleotide may consist of, or consist essentially of any polynucleotide described in the present specification; Optionally, said determining may be performed in a hybridization assay, sequencing assay, microsequencing assay, or an enzyme-based mismatch detection assay; Optionally, said polynucleotide may be attached to a solid support, array, or addressable array; Optionally, said polynucleotide may be labeled. A preferred polynucleotide may be used in a hybridization assay for determining the identity of the nucleotide at a AA4RP-related biallelic marker. Another preferred polynucleotide may be used in a sequencing or microsequencing assay for determining the identity of the nucleotide at a AA4RP-related biallelic marker. A third preferred polynucleotide may be used in an enzyme-based mismatch detection assay for determining the identity of the nucleotide at a AA4RP-related biallelic marker. A fourth preferred polynucleotide may be used in amplifying a segment of polynucleotides comprising a AA4RP-related biallelic marker. Optionally, any of the polynucleotides described above may be attached to a solid support, array, or addressable array; Optionally, said polynucleotide may be labeled.

Additionally, the invention encompasses the use of any polynucleotide for, or any polynucleotide for use in, amplifying a segment of nucleotides comprising a AA4RP-related biallelic marker. In addition, the polynucleotides of the invention for use in amplifying a segment of nucleotides comprising a AA4RP-related biallelic marker encompass polynucleotides with any further limitation described in this disclosure, or those following, specified alone or in any combination: Optionally, said AA4RP-related biallelic marker is selected from the group consisting of 20-828-311, 17-42-319, 17-41-250, 20-841-149, 20-842-115, and 20-853-415, and the complements thereof, or optionally the biallelic markers in linkage disequilibrium therewith; optionally, said AA4RP-related biallelic marker is selected from the group consisting of 17-42-319 and 17-41-250, and the complements thereof. Optionally, said polynucleotide may comprise a sequence disclosed in the present specification; Optionally, said polynucleotide may consist of, or consist essentially of any polynucleotide described in the present specification; Optionally, said amplifying may be performed by a PCR or LCR. Optionally, said polynucleotide may be attached to a solid support, array, or addressable array. Optionally, said polynucleotide may be labeled.

The primers for amplification or sequencing reaction of a polynucleotide comprising a biallelic marker of the invention may be designed from the disclosed sequences for any method known in the art. A preferred set of primers are fashioned such that the 3′ end of the contiguous span of identity with a sequence selected from the group consisting of SEQ ID Nos 1, 2 and 4 or a sequence complementary thereto or a variant thereof is present at the 3′ end of the primer. Such a configuration allows the 3′ end of the primer to hybridize to a selected nucleic acid sequence and dramatically increases the efficiency of the primer for amplification or sequencing reactions. Allele specific primers may be designed such that a polymorphic base of a biallelic marker is at the 3′ end of the contiguous span and the contiguous span is present at the 3′ end of the primer. Such allele specific primers tend to selectively prime an amplification or sequencing reaction so long as they are used with a nucleic acid sample that contains one of the two alleles present at a biallelic marker. The 3′ end of the primer of the invention may be located within or at least 2, 4, 6, 8, 10, 12, 15, 18, 20, 25, 50, 100, 250, 500, or 1000 nucleotides upstream of a AA4RP-related biallelic marker in said sequence or at any other location which is appropriate for their intended use in sequencing, amplification or the location of novel sequences or markers. Thus, another set of preferred amplification primers comprise an isolated polynucleotide consisting essentially of a contiguous span of 8 to 50 nucleotides in a sequence selected from the group consisting of SEQ ID Nos 1, 2 and 4 or a sequence complementary thereto or a variant thereof, wherein the 3′ end of said contiguous span is located at the 3 ′end of said polynucleotide, and wherein the 3 ′end of said polynucleotide is located upstream of a AA4RP-related biallelic marker in said sequence. Preferably, those amplification primers comprise a sequence selected from the group consisting of the sequences 929-949, 12029-12050, 14992-15012, 42070-42090, 45328-45347, 76644-76664, 1357-1377, 12581-12603, 15460-15482, 42572-42591, 45863-45883, and 77166-77185 of SEQ ID No 1; and 1-11022, 899-11920, 1246-12267, 2964-13984, 553-11575, 1441-12461, 1632-12651, and 3432-14454 of SEQ ID No 4. Primers with their 3′ ends located 1 nucleotide upstream of a biallelic marker of AA4RP have a special utility as microsequencing assays. Preferred microsequencing primers are described in FIG. 4. Optionally, said AA4RP-related biallelic marker is selected from the group consisting of 20-828-311, 17-42-319, 17-41-250, 20-841-149, 20-842-115, and 20-853-415, and the complements thereof, or optionally the biallelic markers in linkage disequilibrium therewith; optionally, said AA4RP-related biallelic marker is selected from the group consisting of 17-42-319 and 17-41-250, and the complements thereof, or optionally the biallelic markers in linkage disequilibrium therewith.

The probes of the present invention may be designed from the disclosed sequences for any method known in the art, particularly methods which allow for testing if a marker disclosed herein is present. A preferred set of probes may be designed for use in the hybridization assays of the invention in any manner known in the art such that they selectively bind to one allele of a biallelic marker, but not the other under any particular set of assay conditions. Preferred hybridization probes comprise the polymorphic base of either allele 1 or allele 2 of the considered biallelic marker. Optionally, said biallelic marker may be within 6, 5, 4, 3, 2, or 1 nucleotides of the center of the hybridization probe or at the center of said probe. In a preferred embodiment, the probes are selected in the group consisting of the sequences 1227-1251, 12335-12359, 15229-15253, 42206-42230, 45430-45454, and 77046-77070 of SEQ ID No 1, and the complementary sequence thereto; and 307-331 and 3201-3225 of SEQ ID No 4, and the complementary sequence thereto.

It should be noted that the polynucleotides of the present invention are not limited to having the exact flanking sequences surrounding the polymorphic bases which are enumerated in Sequence Listing. Rather, it will be appreciated that the flanking sequences surrounding the biallelic markers may be lengthened or shortened to any extent compatible with their intended use and the present invention specifically contemplates such sequences. The flanking regions outside of the contiguous span need not be homologous to native flanking sequences which actually occur in human subjects. The addition of any nucleotide sequence which is compatible with the nucleotides intended use is specifically contemplated.

Primers and probes may be labeled or immobilized on a solid support as described in “Oligonucleotide Probes and Primers”.

The polynucleotides of the invention which are attached to a solid support encompass polynucleotides with any further limitation described in this disclosure, or those following, specified alone or in any combination: Optionally, said polynucleotides may be specified as attached individually or in groups of at least 2, 5, 8, 10, 12, 15, 20, or 25 distinct polynucleotides of the invention to a single solid support. Optionally, polynucleotides other than those of the invention may attached to the same solid support as polynucleotides of the invention. Optionally, when multiple polynucleotides are attached to a solid support they may be attached at random locations, or in an ordered array. Optionally, said ordered array may be addressable.

The present invention also encompasses diagnostic kits comprising one or more polynucleotides of the invention with a portion or all of the necessary reagents and instructions for genotyping a test subject by determining the identity of a nucleotide at a AA4RP-related biallelic marker. The polynucleotides of a kit may optionally be attached to a solid support, or be part of an array or addressable array of polynucleotides. The kit may provide for the determination of the identity of the nucleotide at a marker position by any method known in the art including, but not limited to, a sequencing assay method, a microsequencing assay method, a hybridization assay method, or an enzyme-based mismatch detection assay method.

IV. Methods for De Novo Identification of Biallelic Markers

Any of a variety of methods can be used to screen a genomic fragment for single nucleotide polymorphisms such as differential hybridization with oligonucleotide probes, detection of changes in the mobility measured by gel electrophoresis or direct sequencing of the amplified nucleic acid. A preferred method for identifying biallelic markers involves comparative sequencing of genomic DNA fragments from an appropriate number of unrelated individuals.

In a first embodiment, DNA samples from unrelated individuals are pooled together, following which the genomic DNA of interest is amplified and sequenced. The nucleotide sequences thus obtained are then analyzed to identify significant polymorphisms. One of the major advantages of this method resides in the fact that the pooling of the DNA samples substantially reduces the number of DNA amplification reactions and sequencing reactions, which must be carried out. Moreover, this method is sufficiently sensitive so that a biallelic marker obtained thereby usually demonstrates a sufficient frequency of its less common allele to be useful in conducting association studies.

In a second embodiment, the DNA samples are not pooled and are therefore amplified and sequenced individually. This method is usually preferred when biallelic markers need to be identified in order to perform association studies within candidate genes. Preferably, highly relevant gene regions such as promoter regions or exon regions may be screened for biallelic markers. A biallelic marker obtained using this method may show a lower degree of informativeness for conducting association studies, e.g. if the frequency of its less frequent allele may be less than about 10%. Such a biallelic marker will, however, be sufficiently informative to conduct association studies and it will further be appreciated that including less informative biallelic markers in the genetic analysis studies of the present invention, may allow in some cases the direct identification of causal mutations, which may, depending on their penetrance, be rare mutations.

The following is a description of the various parameters of a preferred method used by the inventors for the identification of the biallelic markers of the present invention.

A. Genomic DNA Samples

The genomic DNA samples from which the biallelic markers of the present invention are generated are preferably obtained from unrelated individuals corresponding to a heterogeneous population of known ethnic background. The number of individuals from whom DNA samples are obtained can vary substantially, preferably from about 10 to about 1000, preferably from about 50 to about 200 individuals. It is usually preferred to collect DNA samples from at least about 100 individuals in order to have sufficient polymorphic diversity in a given population to identify as many markers as possible and to generate statistically significant results.

As for the source of the genomic DNA to be subjected to analysis, any test sample can be foreseen without any particular limitation. These test samples include biological samples, which can be tested by the methods of the present invention described herein, and include human and animal body fluids such as whole blood, serum, plasma, cerebrospinal fluid, urine, lymph fluids, and various external secretions of the respiratory, intestinal and genitourinary tracts, tears, saliva, milk, white blood cells, myelomas and the like; biological fluids such as cell culture supernatants; fixed tissue specimens including tumor and non-tumor tissue and lymph node tissues; bone marrow aspirates and fixed cell specimens. The preferred source of genomic DNA used in the present invention is from peripheral venous blood of each donor. Techniques to prepare genomic DNA from biological samples are well known to the skilled technician. Details of a preferred embodiment are provided in Example 1. The person skilled in the art can choose to amplify pooled or unpooled DNA samples.

B. DNA Amplification

The identification of biallelic markers in a sample of genomic DNA may be facilitated through the use of DNA amplification methods. DNA samples can be pooled or unpooled for the amplification step. DNA amplification techniques are well known to those skilled in the art.

Amplification techniques that can be used in the context of the present invention include, but are not limited to, the ligase chain reaction (LCR) described in EP-A-320 308, WO 9320227 and EP-A-439 182, the polymerase chain reaction (PCR, RT-PCR) and techniques such as the nucleic acid sequence based amplification (NASBA) described in Guatelli J. C., et al.(1990) and in Compton J.(1991), Q-beta amplification as described in European Patent Application No 4544610, strand displacement amplification as described in Walker et al.(1996) and EP A 684 315 and, target mediated amplification as described in PCT Publication WO 9322461.

LCR and Gap LCR are exponential amplification techniques, both depend on DNA ligase to join adjacent primers annealed to a DNA molecule. In Ligase Chain Reaction (LCR), probe pairs are used which include two primary (first and second) and two secondary (third and fourth) probes, all of which are employed in molar excess to target. The first probe hybridizes to a first segment of the target strand and the second probe hybridizes to a second segment of the target strand, the first and second segments being contiguous so that the primary probes abut one another in 5′ phosphate-3 ′ hydroxyl relationship, and so that a ligase can covalently fuse or ligate the two probes into a fused product. In addition, a third (secondary) probe can hybridize to a portion of the first probe and a fourth (secondary) probe can hybridize to a portion of the second probe in a similar abutting fashion. Of course, if the target is initially double stranded, the secondary probes also will hybridize to the target complement in the first instance. Once the ligated strand of primary probes is separated from the target strand, it will hybridize with the third and fourth probes, which can be ligated to form a complementary, secondary ligated product. It is important to realize that the ligated products are functionally equivalent to either the target or its complement. By repeated cycles of hybridization and ligation, amplification of the target sequence is achieved. A method for multiplex LCR has also been described (WO 9320227). Gap LCR (GLCR) is a version of LCR where the probes are not adjacent but are separated by 2 to 3 bases.

For amplification of mRNAs, it is within the scope of the present invention to reverse transcribe mRNA into cDNA followed by polymerase chain reaction (RT-PCR); or, to use a single enzyme for both steps as described in U.S. Pat. No. 5,322,770 or, to use Asymmetric Gap LCR (RT-AGLCR) as described by Marshall et al.(1 994). AGLCR is a modification of GLCR that allows the amplification of RNA.

The PCR technology is the preferred amplification technique used in the present invention. A variety of PCR techniques are familiar to those skilled in the art. For a review of PCR technology, see White (1997) and the publication entitled “PCR Methods and Applications” (1991, Cold Spring Harbor Laboratory Press). In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195; 4,683,202; and 4,965,188, the disclosures of which are incorporated herein by reference in their entireties.

The PCR technology is the preferred amplification technique used to identify new biallelic markers. A typical example of a PCR reaction suitable for the purposes of the present invention is provided in Example 2.

One of the aspects of the present invention is a method for the amplification of the human AA4RP gene, particularly of a fragment of the genomic sequence of SEQ ID No 1 or 4 or of the cDNA sequence of SEQ ID No 2, or a fragment or a variant thereof in a test sample, preferably using the PCR technology. This method comprises the steps of:

a) contacting a test sample with amplification reaction reagents comprising a pair of amplification primers as described above and located on either side of the polynucleotide region to be amplified, and

b) optionally, detecting the amplification products.

The invention also concerns a kit for the amplification of a AA4RP gene sequence, particularly of a portion of the genomic sequence of SEQ ID No 1 or 4 or of the cDNA sequence of SEQ ID No 2, or a variant thereof in a test sample, wherein said kit comprises:

a) a pair of oligonucleotide primers located on either side of the AA4RP region to be amplified;

b) optionally, the reagents necessary for performing the amplification reaction.

In one embodiment of the above amplification method and kit, the amplification product is detected by hybridization with a labeled probe having a sequence which is complementary to the amplified region. In another embodiment of the above amplification method and kit, primers comprise a sequence which is selected from the group consisting of the nucleotide sequences of 929-949, 12029-12050, 14992-15012, 42070-42090, 45328-45347, 76644-76664, 1357-1377, 12581-12603, 15460-15482, 42572-42591, 45863-45883, 77166-77185, 1220-1238, 12328-12346, 15222-15240, 42199-42217, 45423-45441, 77039-77057, 1240-1258, 12348-12366, 15242-15260, 42219-42237, 45443-45461 and 77059-77077 of SEQ ID No 1; and 1-11022, 899-11920, 1246-12267,2964-13984, 553-11575, 1441-12461, 1632-12651, 3432-14454, 300-318, 3194-3212, 320-338 and 3214-3232 of SEQ ID No 4.

In a first embodiment of the present invention, biallelic markers are identified using genomic sequence information generated by the inventors. Sequenced genomic DNA fragments are used to design primers for the amplification of 500 bp fragments. These 500 bp fragments are amplified from genomic DNA and are scanned for biallelic markers. Primers may be designed using the OSP software (Hillier L. and Green P., 1991). All primers may contain, upstream of the specific target bases, a common oligonucleotide tail that serves as a sequencing primer. Those skilled in the art are familiar with primer extensions, which can be used for these purposes.

Preferred primers, useful for the amplification of genomic sequences encoding the candidate genes, focus on promoters, exons and splice sites of the genes. A biallelic marker presents a higher probability to be an eventual causal mutation if it is located in these functional regions of the gene. Preferred amplification primers of the invention include the nucleotide sequences 929-949, 12029-12050, 14992-15012, 42070-42090, 45328-45347, 76644-76664, 1357-1377, 12581-12603, 15460-15482, 42572-42591, 45863-45883, and 77166-77185 of SEQ ID No 1; and 1-11022, 899-11920, 1246-12267, 2964-13984, 553-11575, 1441-12461, 1632-12651, and 3432-14454 of SEQ ID No 4; detailed further in Example 2.

C. Sequencing of Amplified Genomic DNA and Identification of Single Nucleotide Polymorphisms

The amplification products generated as described above, are then sequenced using any method known and available to the skilled technician. Methods for sequencing DNA using either the dideoxy-mediated method (Sanger method) or the Maxam-Gilbert method are widely known to those of ordinary skill in the art. Such methods are for example disclosed in Sambrook et al.(1989). Alternative approaches include hybridization to high-density DNA probe arrays as described in Chee et al.(1996).

Preferably, the amplified DNA is subjected to automated dideoxy terminator sequencing reactions using a dye-primer cycle sequencing protocol. The products of the sequencing reactions are run on sequencing gels and the sequences are determined using gel image analysis. The polymorphism search is based on the presence of superimposed peaks in the electrophoresis pattern resulting from different bases occurring at the same position. Because each dideoxy terminator is labeled with a different fluorescent molecule, the two peaks corresponding to a biallelic site present distinct colors corresponding to two different nucleotides at the same position on the sequence. However, the presence of two peaks can be an artifact due to background noise. To exclude such an artifact, the two DNA strands are sequenced and a comparison between the peaks is carried out. In order to be registered as a polymorphic sequence, the polymorphism has to be detected on both strands.

The above procedure permits those amplification products, which contain biallelic markers to be identified. The detection limit for the frequency of biallelic polymorphisms detected by sequencing pools of 100 individuals is approximately 0.1 for the minor allele, as verified by sequencing pools of known allelic frequencies. However, more than 90% of the biallelic polymorphisms detected by the pooling method have a frequency for the minor allele higher than 0.25. Therefore, the biallelic markers selected by this method have a frequency of at least 0.1 for the minor allele and less than 0.9 for the major allele. Preferably at least 0.2 for the minor allele and less than 0.8 for the major allele, more preferably at least 0.3 for the minor allele and less than 0.7 for the major allele, thus a heterozygosity rate higher than 0.18, preferably higher than 0.32, more preferably higher than 0.42.

In another embodiment, biallelic markers are detected by sequencing individual DNA samples, the frequency of the minor allele of such a biallelic marker may be less than 0.1.

D. Validation of the Biallelic Markers of the Present Invention

The polymorphisms are evaluated for their usefulness as genetic markers by validating that both alleles are present in a population. Validation of the biallelic markers is accomplished by genotyping a group of individuals by a method of the invention and demonstrating that both alleles are present. Microsequencing is a preferred method of genotyping alleles. The validation by genotyping step may be performed on individual samples derived from each individual in the group or by genotyping a pooled sample derived from more than one individual. The group can be as small as one individual if that individual is heterozygous for the allele in question. Preferably the group contains at least three individuals, more preferably the group contains five or six individuals, so that a single validation test will be more likely to result in the validation of more of the biallelic markers that are being tested. It should be noted, however, that when the validation test is performed on a small group it may result in a false negative result if as a result of sampling error none of the individuals tested carries one of the two alleles. Thus, the validation process is less useful in demonstrating that a particular initial result is an artifact, than it is at demonstrating that there is a bonafide biallelic marker at a particular position in a sequence. All of the genotyping, haplotyping, association, and interaction study methods of the invention may optionally be performed solely with validated biallelic markers.

E. Evaluation of the Frequency of the Biallelic Markers of the Present Invention

The validated biallelic markers are further evaluated for their usefulness as genetic markers by determining the frequency of the least common allele at the biallelic marker site. The higher the frequency of the less common allele the greater the usefulness of the biallelic marker is association and interaction studies. The determination of the least common allele is accomplished by genotyping a group of individuals by a method of the invention and demonstrating that both alleles are present. This determination of frequency by genotyping step may be performed on individual samples derived from each individual in the group or by genotyping a pooled sample derived from more than one individual. The group must be large enough to be representative of the population as a whole. Preferably the group contains at least 20 individuals, more preferably the group contains at least 50 individuals, most preferably the group contains at least 100 individuals. Of course the larger the group the greater the accuracy of the frequency determination because of reduced sampling error. A biallelic marker wherein the frequency of the less common allele is 30% or more is termed a “high quality biallelic marker.” All of the genotyping, haplotyping, association, and interaction study methods of the invention may optionally be performed solely with high quality biallelic markers.

V. Methods for Genotyping an Individual for Biallelic Markers

Methods are provided to genotype a biological sample for one or more biallelic markers of the present invention, all of which may be performed in vitro. Such methods of genotyping comprise determining the identity of a nucleotide at a AA4RP biallelic marker site by any method known in the art.

These methods find use in genotyping case-control populations in association studies as well as individuals in the context of detection of alleles of biallelic markers which are known to be associated with a given trait, in which case both copies of the biallelic marker present in individual's genome are determined so that an individual may be classified as homozygous or heterozygous for a particular allele.

These genotyping methods can be performed on nucleic acid samples derived from a single individual or pooled DNA samples.

Genotyping can be performed using similar methods as those described above for the identification of the biallelic markers, or using other genotyping methods such as those further described below. In preferred embodiments, the comparison of sequences of amplified genomic fragments from different individuals is used to identify new biallelic markers whereas microsequencing is used for genotyping known biallelic markers in diagnostic and association study applications.

In one embodiment the invention encompasses methods of genotyping comprising determining the identity of a nucleotide at a AA4RP-related biallelic marker or the complement thereof in a biological sample; optionally, wherein said AA4RP-related biallelic marker is selected from the group consisting of 20-828-311, 17-42-319, 17-41-250, 20-841-149, 20-842-115, and 20-853-415, and the complements thereof, or optionally the biallelic markers in linkage disequilibrium therewith; optionally, wherein said AA4RP-related biallelic marker is selected from the group consisting of 17-42-319 and 17-41-250, and the complements thereof, or optionally the biallelic markers in linkage disequilibrium therewith; optionally, wherein said biological sample is derived from a single subject; optionally, wherein the identity of the nucleotides at said biallelic marker is determined for both copies of said biallelic marker present in said individual's genome; optionally, wherein said biological sample is derived from multiple subjects; Optionally, the genotyping methods of the invention encompass methods with any further limitation described in this disclosure, or those following, specified alone or in any combination; Optionally, said method is performed in vitro; optionally, further comprising amplifying a portion of said sequence comprising the biallelic marker prior to said determining step; Optionally, wherein said amplifying is performed by PCR, LCR, or replication of a recombinant vector comprising an origin of replication and said fragment in a host cell; optionally, wherein said determining is performed by a hybridization assay, a sequencing assay, a microsequencing assay, or an enzyme-based mismatch detection assay.

A. Source of Nucleic Acids for Genotyping

Any source of nucleic acids, in purified or non-purified form, can be utilized as the starting nucleic acid, provided it contains or is suspected of containing the specific nucleic acid sequence desired. DNA or RNA may be extracted from cells, tissues, body fluids and the like as described above. While nucleic acids for use in the genotyping methods of the invention can be derived from any mammalian source, the test subjects and individuals from which nucleic acid samples are taken are generally understood to be human.

B. Amplification of DNA Fragments Comprising Biallelic Markers

Methods and polynucleotides are provided to amplify a segment of nucleotides comprising one or more biallelic marker of the present invention. It will be appreciated that amplification of DNA fragments comprising biallelic markers may be used in various methods and for various purposes and is not restricted to genotyping. Nevertheless, many genotyping methods, although not all, require the previous amplification of the DNA region carrying the biallelic marker of interest. Such methods specifically increase the concentration or total number of sequences that span the biallelic marker or include that site and sequences located either distal or proximal to it. Diagnostic assays may also rely on amplification of DNA segments carrying a biallelic marker of the present invention. Amplification of DNA may be achieved by any method known in the art. Amplification techniques are described above in the section entitled, “DNA Amplification.”

Some of these amplification methods are particularly suited for the detection of single nucleotide polymorphisms and allow the simultaneous amplification of a target sequence and the identification of the polymorphic nucleotide as it is further described below.

The identification of biallelic markers as described above allows the design of appropriate oligonucleotides, which can be used as primers to amplify DNA fragments comprising the biallelic markers of the present invention. Amplification can be performed using the primers initially used to discover new biallelic markers which are described herein or any set of primers allowing the amplification of a DNA fragment comprising a biallelic marker of the present invention.

In some embodiments the present invention provides primers for amplifying a DNA fragment containing one or more biallelic markers of the present invention. Preferred amplification primers are listed in FIG. 5. It will be appreciated that the primers listed are merely exemplary and that any other set of primers which produce amplification products containing one or more biallelic markers of the present invention are also of use.

The spacing of the primers determines the length of the segment to be amplified. In the context of the present invention, amplified segments carrying biallelic markers can range in size from at least about 25 bp to 35 kbp. Amplification fragments from 25-3000 bp are typical, fragments from 50-1000 bp are preferred and fragments from 100-600 bp are highly preferred. It will be appreciated that amplification primers for the biallelic markers may be any sequence which allow the specific amplification of any DNA fragment carrying the markers. Amplification primers may be labeled or immobilized on a solid support as described in “Oligonucleotide Probes and Primers.”

C. Methods of Genotyping DNA Samples for Biallelic Markers

Any method known in the art can be used to identify the nucleotide present at a biallelic marker site. Since the biallelic marker allele to be detected has been identified and specified in the present invention, detection will prove simple for one of ordinary skill in the art by employing any of a number of techniques. Many genotyping methods require the previous amplification of the DNA region carrying the biallelic marker of interest. While the amplification of target or signal is often preferred at present, ultrasensitive detection methods which do not require amplification are also encompassed by the present genotyping methods. Methods well-known to those skilled in the art that can be used to detect biallelic polymorphisms include methods such as, conventional dot blot analyzes, single strand conformational polymorphism analysis (SSCP) described by Orita et al.(1989), denaturing gradient gel electrophoresis (DGGE), heteroduplex analysis, mismatch cleavage detection, and other conventional techniques as described in Sheffield et al.(1991), White et al.(1992), Grompe et al.(1989 and 1993). Another method for determining the identity of the nucleotide present at a particular polymorphic site employs a specialized exonuclease-resistant nucleotide derivative as described in U.S. Pat. No. 4,656,127.

Preferred methods involve directly determining the identity of the nucleotide present at a biallelic marker site by sequencing assay, enzyme-based mismatch detection assay, or hybridization assay. The following is a description of some preferred methods. A highly preferred method is the microsequencing technique. The term “sequencing” is generally used herein to refer to polymerase extension of duplex primer/template complexes and includes both traditional sequencing and microsequencing.

i. Sequencing Assays

The nucleotide present at a polymorphic site can be determined by sequencing methods. In a preferred embodiment, DNA samples are subjected to PCR amplification before sequencing as described above. DNA sequencing methods are described in “Sequencing Of Amplified Genomic DNA And Identification Of Single Nucleotide Polymorphisms”.

Preferably, the amplified DNA is subjected to automated dideoxy terminator sequencing reactions using a dye-primer cycle sequencing protocol. Sequence analysis allows the identification of the base present at the biallelic marker site.

ii. Microsequencing Assays

In microsequencing methods, the nucleotide at a polymorphic site in a target DNA is detected by a single nucleotide primer extension reaction. This method involves appropriate microsequencing primers which, hybridize just upstream of the polymorphic base of interest in the target nucleic acid. A polymerase is used to specifically extend the 3′ end of the primer with one single ddNTP (chain terminator) complementary to the nucleotide at the polymorphic site. Next the identity of the incorporated nucleotide is determined in any suitable way.

Typically, microsequencing reactions are carried out using fluorescent ddNTPs and the extended microsequencing primers are analyzed by electrophoresis on ABI 377 sequencing machines to determine the identity of the incorporated nucleotide as described in EP 412 883, the disclosure of which is incorporated herein by reference in its entirety. Alternatively capillary electrophoresis can be used in order to process a higher number of assays simultaneously. An example of a typical microsequencing procedure that can be used in the context of the present invention is provided in Example 4.

Different approaches can be used for the labeling and detection of ddNTPs. A homogeneous phase detection method based on fluorescence resonance energy transfer has been described by Chen and Kwok (1997) and Chen et al.(1997). In this method, amplified genomic DNA fragments containing polymorphic sites are incubated with a 5′-fluorescein-labeled primer in the presence of allelic dye-labeled dideoxyribonucleoside triphosphates and a modified Taq polymerase. The dye-labeled primer is extended one base by the dye-terminator specific for the allele present on the template. At the end of the genotyping reaction, the fluorescence intensities of the two dyes in the reaction mixture are analyzed directly without separation or purification. All these steps can be performed in the same tube and the fluorescence changes can be monitored in real time. Alternatively, the extended primer may be analyzed by MALDI-TOF Mass Spectrometry. The base at the polymorphic site is identified by the mass added onto the microsequencing primer (see Haff and Smirnov, 1997).

Microsequencing may be achieved by the established microsequencing method or by developments or derivatives thereof. Alternative methods include several solid-phase microsequencing techniques. The basic microsequencing protocol is the same as described previously, except that the method is conducted as a heterogeneous phase assay, in which the primer or the target molecule is immobilized or captured onto a solid support. To simplify the primer separation and the terminal nucleotide addition analysis, oligonucleotides are attached to solid supports or are modified in such ways that permit affinity separation as well as polymerase extension. The 5′ ends and internal nucleotides of synthetic oligonucleotides can be modified in a number of different ways to permit different affinity separation approaches, e.g., biotinylation. If a single affinity group is used on the oligonucleotides, the oligonucleotides can be separated from the incorporated terminator regent. This eliminates the need of physical or size separation. More than one oligonucleotide can be separated from the terminator reagent and analyzed simultaneously if more than one affinity group is used. This permits the analysis of several nucleic acid species or more nucleic acid sequence information per extension reaction. The affinity group need not be on the priming oligonucleotide but could alternatively be present on the template. For example, immobilization can be carried out via an interaction between biotinylated DNA and streptavidin-coated microtitration wells or avidin-coated polystyrene particles. In the same manner, oligonucleotides or templates may be attached to a solid support in a high-density format. In such solid phase microsequencing reactions, incorporated ddNTPs can be radiolabeled (Syvänen, 1994) or linked to fluorescein (Livak and Hainer, 1994). The detection of radiolabeled ddNTPs can be achieved through scintillation-based techniques. The detection of fluorescein-linked ddNTPs can be based on the binding of antifluorescein antibody conjugated with alkaline phosphatase, followed by incubation with a chromogenic substrate (such as p-nitrophenyl phosphate). Other possible reporter-detection pairs include: ddNTP linked to dinitrophenyl (DNP) and anti-DNP alkaline phosphatase conjugate (Harju et al., 1993) or biotinylated ddNTP and horseradish peroxidase-conjugated streptavidin with o-phenylenediamine as a substrate (WO 92/15712, the disclosure of which is incorporated herein by reference in its entirety). As yet another alternative solid-phase microsequencing procedure, Nyren et al.(1993) described a method relying on the detection of DNA polymerase activity by an enzymatic luminometric inorganic pyrophosphate detection assay (ELIDA).

Pastinen et al.(l 997) describe a method for multiplex detection of single nucleotide polymorphism in which the solid phase minisequencing principle is applied to an oligonucleotide array format. High-density arrays of DNA probes attached to a solid support (DNA chips) are further described below.

In one aspect the present invention provides polynucleotides and methods to genotype one or more biallelic markers of the present invention by performing a microsequencing assay. Preferred microsequencing primers include the nucleotide sequences 1220-1238, 12328-12346, 15222-15240, 42199-42217, 45423-45441, 77039-77057, 1240-1258, 12348-12366, 15242-15260, 42219-42237, 45443-45461 and 77059-77077 of SEQ ID No 1; and 300-318,3194-3212, 320-338 and 3214-3232 of SEQ ID No 4. It will be appreciated that the microsequencing primers listed in FIG. 4 are merely exemplary and that, any primer having a 3′ end immediately adjacent to the polymorphic nucleotide may be used. Similarly, it will be appreciated that microsequencing analysis may be performed for any biallelic marker or any combination of biallelic markers of the present invention. One aspect of the present invention is a solid support which includes one or more microsequencing primers listed in FIG. 4, or fragments comprising at least 8, 12, 15, 20, 25, 30, 40, or 50 consecutive nucleotides thereof, to the extent that such lengths are consistent with the primer described, and having a 3′ terminus immediately upstream of the corresponding biallelic marker, for determining the identity of a nucleotide at a biallelic marker site.

iii. Mismatch Detection Assays Based on Polymerases and Ligases

In one aspect the present invention provides polynucleotides and methods to determine the allele of one or more biallelic markers of the present invention in a biological sample, by mismatch detection assays based on polymerases and/or ligases. These assays are based on the specificity of polymerases and ligases. Polymerization reactions places particularly stringent requirements on correct base pairing of the 3′ end of the amplification primer and the joining of two oligonucleotides hybridized to a target DNA sequence is quite sensitive to mismatches close to the ligation site, especially at the 3′ end. Methods, primers and various parameters to amplify DNA fragments comprising biallelic markers of the present invention are further described above in “Amplification Of DNA Fragments Comprising Biallelic Markers.”

Allele Specific Amplification Primers

Discrimination between the two alleles of a biallelic marker can also be achieved by allele specific amplification, a selective strategy, whereby one of the alleles is amplified without amplification of the other allele. For allele specific amplification, at least one member of the pair of primers is sufficiently complementary with a region of a AA4RP gene comprising the polymorphic base of a biallelic marker of the present invention to hybridize therewith and to initiate the amplification. Such primers are able to discriminate between the two alleles of a biallelic marker.

This is accomplished by placing the polymorphic base at the 3′ end of one of the amplification primers. Because the extension forms from the 3′ end of the primer, a mismatch at or near this position has an inhibitory effect on amplification. Therefore, under appropriate amplification conditions, these primers only direct amplification on their complementary allele. Determining the precise location of the mismatch and the corresponding assay conditions are well within the ordinary skill in the art.

Ligation/Amplification Based Methods

The “Oligonucleotide Ligation Assay” (OLA) uses two oligonucleotides which are designed to be capable of hybridizing to abutting sequences of a single strand of a target molecules. One of the oligonucleotides is biotinylated, and the other is detectably labeled. If the precise complementary sequence is found in a target molecule, the oligonucleotides will hybridize such that their termini abut, and create a ligation substrate that can be captured and detected. OLA is capable of detecting single nucleotide polymorphisms and may be advantageously combined with PCR as described by Nickerson et al.(1990). In this method, PCR is used to achieve the exponential amplification of target DNA, which is then detected using OLA.

Other amplification methods which are particularly suited for the detection of single nucleotide polymorphism include LCR (ligase chain reaction), Gap LCR (GLCR) which are described above in “DNA Amplification”. LCR uses two pairs of probes to exponentially amplify a specific target. The sequences of each pair of oligonucleotides, is selected to permit the pair to hybridize to abutting sequences of the same strand of the target. Such hybridization forms a substrate for a template-dependant ligase. In accordance with the present invention, LCR can be performed with oligonucleotides having the proximal and distal sequences of the same strand of a biallelic marker site. In one embodiment, either oligonucleotide will be designed to include the biallelic marker site. In such an embodiment, the reaction conditions are selected such that the oligonucleotides can be ligated together only if the target molecule either contains or lacks the specific nucleotide that is complementary to the biallelic marker on the oligonucleotide. In an alternative embodiment, the oligonucleotides will not include the biallelic marker, such that when they hybridize to the target molecule, a “gap” is created as described in WO 90/01069, the disclosure of which is incorporated herein by reference in its entirety. This gap is then “filled” with complementary dNTPs (as mediated by DNA polymerase), or by an additional pair of oligonucleotides. Thus at the end of each cycle, each single strand has a complement capable of serving as a target during the next cycle and exponential allele-specific amplification of the desired sequence is obtained.

Ligase/Polymerase-mediated Genetic Bit Analysis™ is another method for determining the identity of a nucleotide at a preselected site in a nucleic acid molecule (WO 95/21271). This method involves the incorporation of a nucleoside triphosphate that is complementary to the nucleotide present at the preselected site onto the terminus of a primer molecule, and their subsequent ligation to a second oligonucleotide. The reaction is monitored by detecting a specific label attached to the reaction's solid phase or by detection in solution.

iv. Hybridization Assay Methods

A preferred method of determining the identity of the nucleotide present at a biallelic marker site involves nucleic acid hybridization. The hybridization probes, which can be conveniently used in such reactions, preferably include the probes defined herein. Any hybridization assay may be used including Southern hybridization, Northern hybridization, dot blot hybridization and solid-phase hybridization (see Sambrook et al., 1989).

Hybridization refers to the formation of a duplex structure by two single stranded nucleic acids due to complementary base pairing. Hybridization can occur between exactly complementary nucleic acid strands or between nucleic acid strands that contain minor regions of mismatch. Specific probes can be designed that hybridize to one form of a biallelic marker and not to the other and therefore are able to discriminate between different allelic forms. Allele-specific probes are often used in pairs, one member of a pair showing perfect match to a target sequence containing the original allele and the other showing a perfect match to the target sequence containing the alternative allele. Hybridization conditions should be sufficiently stringent that there is a significant difference in hybridization intensity between alleles, and preferably an essentially binary response, whereby a probe hybridizes to only one of the alleles. Stringent, sequence specific hybridization conditions, under which a probe will hybridize only to the exactly complementary target sequence are well known in the art (Sambrook et al., 1989). Stringent conditions are sequence dependent and will be different in different circumstances. Generally, stringent conditions are selected to be about 5° C. lower than the thermal melting point (Tm) for the specific sequence at a defined ionic strength and pH. Although such hybridization can be performed in solution, it is preferred to employ a solid-phase hybridization assay. The target DNA comprising a biallelic marker of the present invention may be amplified prior to the hybridization reaction. The presence of a specific allele in the sample is determined by detecting the presence or the absence of stable hybrid duplexes formed between the probe and the target DNA. The detection of hybrid duplexes can be carried out by a number of methods. Various detection assay formats are well known which utilize detectable labels bound to either the target or the probe to enable detection of the hybrid duplexes. Typically, hybridization duplexes are separated from unhybridized nucleic acids and the labels bound to the duplexes are then detected. Those skilled in the art will recognize that wash steps may be employed to wash away excess target DNA or probe as well as unbound conjugate. Further, standard heterogeneous assay formats are suitable for detecting the hybrids using the labels present on the primers and probes.

Two recently developed assays allow hybridization-based allele discrimination with no need for separations or washes (see Landegren U. et al., 1998). The TaqMan assay takes advantage of the 5′ nuclease activity of Taq DNA polymerase to digest a DNA probe annealed specifically to the accumulating amplification product. TaqMan probes are labeled with a donor-acceptor dye pair that interacts via fluorescence energy transfer. Cleavage of the TaqMan probe by the advancing polymerase during amplification dissociates the donor dye from the quenching acceptor dye, greatly increasing the donor fluorescence. All reagents necessary to detect two allelic variants can be assembled at the beginning of the reaction and the results are monitored in real time (see Livak et al., 1995). In an alternative homogeneous hybridization based procedure, molecular beacons are used for allele discriminations. Molecular beacons are hairpin-shaped oligonucleotide probes that report the presence of specific nucleic acids in homogeneous solutions. When they bind to their targets they undergo a conformational reorganization that restores the fluorescence of an internally quenched fluorophore (Tyagi et al., 1998).

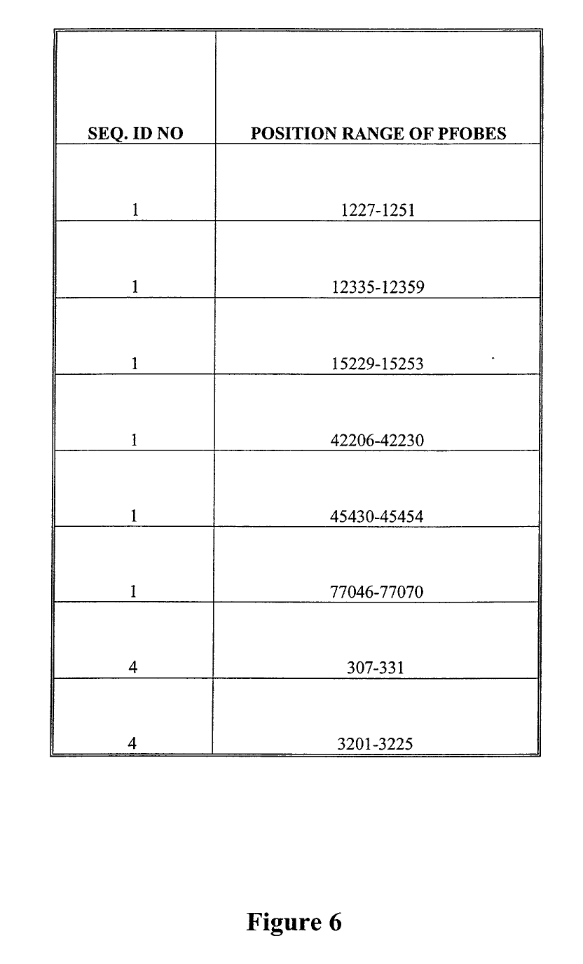

The polynucleotides provided herein can be used to produce probes which can be used in hybridization assays for the detection of biallelic marker alleles in biological samples. These probes are characterized in that they preferably comprise between 8 and 50 nucleotides, and in that they are sufficiently complementary to a sequence comprising a biallelic marker of the present invention to hybridize thereto and preferably sufficiently specific to be able to discriminate the targeted sequence for only one nucleotide variation. A particularly preferred probe is 25 nucleotides in length. Preferably the biallelic marker is within 4 nucleotides of the center of the polynucleotide probe. In particularly preferred probes, the biallelic marker is at the center of said polynucleotide. Preferred probes comprise a nucleotide sequence selected from the group consisting of amplicons listed in FIG. 6 and the sequences complementary thereto, or a fragment thereof, said fragment comprising at least about 8 consecutive nucleotides, preferably 10, 15, 20, more preferably 25, 30, 40, 47, or 50 consecutive nucleotides and containing a polymorphic base. Preferred probes comprise a nucleotide sequence selected from the group consisting of 1227-1251, 12335-12359, 15229-15253, 42206-42230, 45430-45454, and 77046-77070 of SEQ ID No 1; and 307-331 and 3201-3225 of SEQ ID No 4 and the sequences complementary thereto. In preferred embodiments the polymorphic base(s) are within 5, 4, 3, 2, 1, nucleotides of the center of the said polynucleotide, more preferably at the center of said polynucleotide.

Preferably the probes of the present invention are labeled or immobilized on a solid support. Labels and solid supports are further described in “Oligonucleotide Probes and Primers.” The probes can be non-extendable as described in “Oligonucleotide Probes and Primers.”

By assaying the hybridization to an allele specific probe, one can detect the presence or absence of a biallelic marker allele in a given sample. High-Throughput parallel hybridization in array format is specifically encompassed within “Hybridization Assays” and are described below.

v. Hybridization to Addressable Arrays of Oligonucleotides

Hybridization assays based on oligonucleotide arrays rely on the differences in hybridization stability of short oligonucleotides to perfectly matched and mismatched target sequence variants. Efficient access to polymorphism information is obtained through a basic structure comprising high-density arrays of oligonucleotide probes attached to a solid support (e.g., the chip) at selected positions. Each DNA chip can contain thousands to millions of individual synthetic DNA probes arranged in a grid-like pattern and miniaturized to the size of a dime.

The chip technology has already been applied with success in numerous cases. For example, the screening of mutations has been undertaken in the BRCA 1 gene, in S. cerevisiae mutant strains, and in the protease gene of HIV-1 virus (Hacia et al., 1996; Shoemaker et al., 1996; Kozal et al., 1996). Chips of various formats for use in detecting biallelic polymorphisms can be produced on a customized basis by Affymetrix (GeneChip™), Hyseq (HyChip and HyGnostics), and Protogene Laboratories.

In general, these methods employ arrays of oligonucleotide probes that are complementary to target nucleic acid sequence segments from an individual, which target sequences including a polymorphic marker. EP 785280, the disclosure of which is incorporated herein by reference in its entirety, describes a tiling strategy for the detection of single nucleotide polymorphisms. Briefly, arrays may generally be “tiled” for a large number of specific polymorphisms. By “tiling” is generally meant the synthesis of a defined set of oligonucleotide probes which is made up of a sequence complementary to the target sequence of interest, as well as preselected variations of that sequence, e.g., substitution of one or more given positions with one or more members of the basis set of nucleotides. Tiling strategies are further described in PCT application No.

WO 95/11995. In a particular aspect, arrays are tiled for a number of specific, identified biallelic marker sequences. In particular, the array is tiled to include a number of detection blocks, each detection block being specific for a specific biallelic marker or a set of biallelic markers. For example, a detection block may be tiled to include a number of probes, which span the sequence segment that includes a specific polymorphism. To ensure probes that are complementary to each allele, the probes are synthesized in pairs differing at the biallelic marker. In addition to the probes differing at the polymorphic base, monosubstituted probes are also generally tiled within the detection block. These monosubstituted probes have bases at and up to a certain number of bases in either direction from the polymorphism, substituted with the remaining nucleotides (selected from A, T, G, C and U). Typically the probes in a tiled detection block will include substitutions of the sequence positions up to and including those that are 5 bases away from the biallelic marker. The monosubstituted probes provide internal controls for the tiled array, to distinguish actual hybridization from artefactual cross-hybridization. Upon completion of hybridization with the target sequence and washing of the array, the array is scanned to determine the position on the array to which the target sequence hybridizes. The hybridization data from the scanned array is then analyzed to identify which allele or alleles of the biallelic marker are present in the sample. Hybridization and scanning may be carried out as described in PCT application No. WO 92/10092 and WO 95/11995 and U.S. Pat. No. 5,424,186.

Thus, in some embodiments, the chips may comprise an array of nucleic acid sequences of fragments of about 15 nucleotides in length. In further embodiments, the chip may comprise an array including at least one of the sequences selected from the group consisting of amplicons listed in FIG. 5 and the sequences complementary thereto, or a fragment thereof, said fragment comprising at least about 8 consecutive nucleotides, preferably 10, 15, 20, more preferably 25, 30, 40, 47, or 50 consecutive nucleotides and containing a polymorphic base. In preferred embodiments the polymorphic base is within 5, 4, 3, 2, 1, nucleotides of the center of the said polynucleotide, more preferably at the center of said polynucleotide. In some embodiments, the chip may comprise an array of at least 2, 3, 4, 5, 6, 7, 8 or more of these polynucleotides of the invention. Solid supports and polynucleotides of the present invention attached to solid supports are further described in “Oligonucleotide Probes and Primers.”

vi. Integrated Systems

Another technique, which may be used to analyze polymorphisms, includes multicomponent integrated systems, which miniaturize and compartmentalize processes such as PCR and capillary electrophoresis reactions in a single functional device. An example of such technique is disclosed in U.S. Pat. No. 5,589,136, the disclosure of which is incorporated herein by reference in its entirety, which describes the integration of PCR amplification and capillary electrophoresis in chips.

Integrated systems can be envisaged mainly when microfluidic systems are used. These systems comprise a pattern of microchannels designed onto a glass, silicon, quartz, or plastic wafer included on a microchip. The movements of the samples are controlled by electric, electroosmotic or hydrostatic forces applied across different areas of the microchip to create functional microscopic valves and pumps with no moving parts.

For genotyping biallelic markers, the microfluidic system may integrate nucleic acid amplification, microsequencing, capillary electrophoresis and a detection method such as laser-induced fluorescence detection.

VI. Methods of Genetic Analysis Using the Biallelic Markers of the Present Invention

Different methods are available for the genetic analysis of complex traits (see Lander and Schork, 1994). The search for disease-susceptibility genes is conducted using two main methods: the linkage approach in which evidence is sought for cosegregation between a locus and a putative trait locus using family studies, and the association approach in which evidence is sought for a statistically significant association between an allele and a trait or a trait causing allele (Khoury et al., 1993). In general, the biallelic markers of the present invention find use in any method known in the art to demonstrate a statistically significant correlation between a genotype and a phenotype. The biallelic markers may be used in parametric and non-parametric linkage analysis methods. Preferably, the biallelic markers of the present invention are used to identify genes associated with detectable traits using association studies, an approach which does not require the use of affected families and which permits the identification of genes associated with complex and sporadic traits.

The genetic analysis using the biallelic markers of the present invention may be conducted on any scale. The whole set of biallelic markers of the present invention or any subset of biallelic markers of the present invention corresponding to the candidate gene may be used. Further, any set of genetic markers including a biallelic marker of the present invention may be used. A set of biallelic polymorphisms that could be used as genetic markers in combination with the biallelic markers of the present invention has been described in WO 98/20165. As mentioned above, it should be noted that the biallelic markers of the present invention may be included in any complete or partial genetic map of the human genome. These different uses are specifically contemplated in the present invention and claims.

A. Linkage Analysis

Linkage analysis is based upon establishing a correlation between the transmission of genetic markers and that of a specific trait throughout generations within a family. Thus, the aim of linkage analysis is to detect marker loci that show cosegregation with a trait of interest in pedigrees.

i. Parametric Methods

When data are available from successive generations there is the opportunity to study the degree of linkage between pairs of loci. Estimates of the recombination fraction enable loci to be ordered and placed onto a genetic map. With loci that are genetic markers, a genetic map can be established, and then the strength of linkage between markers and traits can be calculated and used to indicate the relative positions of markers and genes affecting those traits (Weir, 1996). The classical method for linkage analysis is the logarithm of odds (lod) score method (see Morton, 1955; Ott, 1991). Calculation of lod scores requires specification of the mode of inheritance for the disease (parametric method). Generally, the length of the candidate region identified using linkage analysis is between 2 and 20 Mb. Once a candidate region is identified as described above, analysis of recombinant individuals using additional markers allows further delineation of the candidate region. Linkage analysis studies have generally relied on the use of a maximum of 5,000 microsatellite markers, thus limiting the maximum theoretical attainable resolution of linkage analysis to about 600 kb on average.

Linkage analysis has been successfully applied to map simple genetic traits that show clear Mendelian inheritance patterns and which have a high penetrance (i.e., the ratio between the number of trait positive carriers of allele a and the total number of a carriers in the population). However, parametric linkage analysis suffers from a variety of drawbacks. First, it is limited by its reliance on the choice of a genetic model suitable for each studied trait. Furthermore, as already mentioned, the resolution attainable using linkage analysis is limited, and complementary studies are required to refine the analysis of the typical 2 Mb to 20 Mb regions initially identified through linkage analysis. In addition, parametric linkage analysis approaches have proven difficult when applied to complex genetic traits, such as those due to the combined action of multiple genes and/or environmental factors. It is very difficult to model these factors adequately in a lod score analysis. In such cases, too large an effort and cost are needed to recruit the adequate number of affected families required for applying linkage analysis to these situations, as recently discussed by Risch, N. and Merikangas, K. (1996).

ii. Non-Parametric Methods

The advantage of the so-called non-parametric methods for linkage analysis is that they do not require specification of the mode of inheritance for the disease, they tend to be more useful for the analysis of complex traits. In non-parametric methods, one tries to prove that the inheritance pattern of a chromosomal region is not consistent with random Mendelian segregation by showing that affected relatives inherit identical copies of the region more often than expected by chance. Affected relatives should show excess “allele sharing” even in the presence of incomplete penetrance and polygenic inheritance. In non-parametric linkage analysis the degree of agreement at a marker locus in two individuals can be measured either by the number of alleles identical by state (IBS) or by the number of alleles identical by descent (IBD). Affected sib pair analysis is a well-known special case and is the simplest form of these methods.

The biallelic markers of the present invention may be used in both parametric and non-parametric linkage analysis. Preferably biallelic markers may be used in non-parametric methods which allow the mapping of genes involved in complex traits. The biallelic markers of the present invention may be used in both IBD- and IBS-methods to map genes affecting a complex trait. In such studies, taking advantage of the high density of biallelic markers, several adjacent biallelic marker loci may be pooled to achieve the efficiency attained by multi-allelic markers (Zhao et al., 1998).

B. Population Association Studies

The present invention comprises methods for identifying if the AA4RP gene is associated with a detectable trait using the biallelic markers of the present invention. In one embodiment the present invention comprises methods to detect an association between a biallelic marker allele or a biallelic marker haplotype and a trait. The trait may include, but is not limited to, the following: body mass; plasma levels of leptin, insulin, free fatty acids (FFA), triglycerides (TG), glucose and RAP3 expression. Further, the invention comprises methods to identify a trait causing allele in linkage disequilibrium with any biallelic marker allele of the present invention.

As described above, alternative approaches can be employed to perform association studies: genome-wide association studies, candidate region association studies and candidate gene association studies. In a preferred embodiment, the biallelic markers of the present invention are used to perform candidate gene association studies. The candidate gene analysis clearly provides a short-cut approach to the identification of genes and gene polymorphisms related to a particular trait when some information concerning the biology of the trait is available. Further, the biallelic markers of the present invention may be incorporated in any map of genetic markers of the human genome in order to perform genome-wide association studies. Methods to generate a high-density map of biallelic markers has been described in U.S. Provisional Patent application serial No. 60/082,614. The biallelic markers of the present invention may further be incorporated in any map of a specific candidate region of the genome (a specific chromosome or a specific chromosomal segment for example).

As mentioned above, association studies may be conducted within the general population and are not limited to studies performed on related individuals in affected families. Association studies are extremely valuable as they permit the analysis of sporadic or multifactor traits. Moreover, association studies represent a powerful method for fine-scale mapping enabling much finer mapping of trait causing alleles than linkage studies. Studies based on pedigrees often only narrow the location of the trait causing allele. Association studies using the biallelic markers of the present invention can therefore be used to refine the location of a trait causing allele in a candidate region identified by Linkage Analysis methods. Moreover, once a chromosome segment of interest has been identified, the presence of a candidate gene such as a candidate gene of the present invention, in the region of interest can provide a shortcut to the identification of the trait causing allele. Biallelic markers of the present invention can be used to demonstrate that a candidate gene is associated with a trait. Such uses are specifically contemplated in the present invention.

C. Determining the Frequency of a Biallelic Marker Allele or of a Biallelic Marker Haplotype in a Population

Association studies explore the relationships among frequencies for sets of alleles between loci.

i. Determining the Frequency of an Allele in a Population

Allelic frequencies of the biallelic markers in a populations can be determined using one of the methods described above under the heading “Methods for genotyping an individual for biallelic markers”, or any genotyping procedure suitable for this intended purpose. Genotyping pooled samples or individual samples can determine the frequency of a biallelic marker allele in a population. One way to reduce the number of genotypings required is to use pooled samples. A major obstacle in using pooled samples is in terms of accuracy and reproducibility for determining accurate DNA concentrations in setting up the pools. Genotyping individual samples provides higher sensitivity, reproducibility and accuracy and; is the preferred method used in the present invention. Preferably, each individual is genotyped separately and simple gene counting is applied to determine the frequency of an allele of a biallelic marker or of a genotype in a given population.

The invention also relates to methods of estimating the frequency of an allele in a population comprising: a) genotyping individuals from said population for said biallelic marker according to the method of the present invention; b) determining the proportional representation of said biallelic marker in said population. In addition, the methods of estimating the frequency of an allele in a population of the invention encompass methods with any further limitation described in this disclosure, or those following, specified alone or in any combination; optionally, wherein said AA4RP-related biallelic marker is selected from the group consisting of 20-828-311, 17-42-319, 17-41-250, 20-841-149, 20-842-115, and 20-853-415, and the complements thereof, or optionally the biallelic markers in linkage disequilibrium therewith; optionally, wherein said AA4RP-related biallelic marker is selected from the group consisting of 17-42-319 and 17-41-250, and the complements thereof. Optionally, determining the frequency of a biallelic marker allele in a population may be accomplished by determining the identity of the nucleotides for both copies of said biallelic marker present in the genome of each individual in said population and calculating the proportional representation of said nucleotide at said AA4RP-related biallelic marker for the population; Optionally, determining the proportional representation may be accomplished by performing a genotyping method of the invention on a pooled biological sample derived from a representative number of individuals, or each individual, in said population, and calculating the proportional amount of said nucleotide compared with the total.

ii. Determining the Frequency of a Haplotype in a Population

The gametic phase of haplotypes is unknown when diploid individuals are heterozygous at more than one locus. Using genealogical information in families gametic phase can sometimes be inferred (Perlin et al., 1994). When no genealogical information is available different strategies may be used. One possibility is that the multiple-site heterozygous diploids can be eliminated from the analysis, keeping only the homozygotes and the single-site heterozygote individuals, but this approach might lead to a possible bias in the sample composition and the underestimation of low-frequency haplotypes. Another possibility is that single chromosomes can be studied independently, for example, by asymmetric PCR amplification (see Newton et al, 1989; Wu et al., 1989) or by isolation of single chromosome by limit dilution followed by PCR amplification (see Ruano et al., 1990). Further, a sample may be haplotyped for sufficiently close biallelic markers by double PCR amplification of specific alleles (Sarkar, G. and Sommer S. S., 1991). These approaches are not entirely satisfying either because of their technical complexity, the additional cost they entail, their lack of generalization at a large scale, or the possible biases they introduce. To overcome these difficulties, an algorithm to infer the phase of PCR-amplified DNA genotypes introduced by Clark, A. G.(1990) may be used. Briefly, the principle is to start filling a preliminary list of haplotypes present in the sample by examining unambiguous individuals, that is, the complete homozygotes and the single-site heterozygotes. Then other individuals in the same sample are screened for the possible occurrence of previously recognized haplotypes. For each positive identification, the complementary haplotype is added to the list of recognized haplotypes, until the phase information for all individuals is either resolved or identified as unresolved. This method assigns a single haplotype to each multiheterozygous individual, whereas several haplotypes are possible when there are more than one heterozygous site. Alternatively, one can use methods estimating haplotype frequencies in a population without assigning haplotypes to each individual. Preferably, a method based on an expectation-maximization (EM) algorithm (Dempster et al., 1977) leading to maximum-likelihood estimates of haplotype frequencies under the assumption of Hardy-Weinberg proportions (random mating) is used (see Excoffier L. and Slatkin M., 1995). The EM algorithm is a generalized iterative maximum-likelihood approach to estimation that is useful when data are ambiguous and/or incomplete. The EM algorithm is used to resolve heterozygotes into haplotypes. Haplotype estimations are further described below under the heading “Statistical Methods.” Any other method known in the art to determine or to estimate the frequency of a haplotype in a population may be used.

The invention also encompasses methods of estimating the frequency of a haplotype for a set of biallelic markers in a population, comprising the steps of: a) genotyping at least one AA4RP-related biallelic marker according to a method of the invention for each individual in said population; b) genotyping a second biallelic marker by determining the identity of the nucleotides at said second biallelic marker for both copies of said second biallelic marker present in the genome of each individual in said population; and c) applying a haplotype determination method to the identities of the nucleotides determined in steps a) and b) to obtain an estimate of said frequency. In addition, the methods of estimating the frequency of a haplotype of the invention encompass methods with any further limitation described in this disclosure, or those following, specified alone or in any combination: optionally, wherein said AA4RP-related biallelic marker is selected from the group consisting of 20-828-311, 17-42-319, 17-41-250, 20-841-149, 20-842-115, and 20-853-415, and the complements thereof, or optionally the biallelic markers in linkage disequilibrium therewith; optionally, wherein said AA4RP-related biallelic marker is selected from the group consisting of 17-42-319 and 17-41-250, and the complements thereof, or optionally the biallelic markers in linkage disequilibrium therewith; Optionally, said haplotype determination method is performed by asymmetric PCR amplification, double PCR amplification of specific alleles, the Clark algorithm, or an expectation-maximization algorithm.

D. Linkage Disequilibrium Analysis

Linkage disequilibrium is the non-random association of alleles at two or more loci and represents a powerful tool for mapping genes involved in disease traits (see Ajioka R. S. et al., 1997). Biallelic markers, because they are densely spaced in the human genome and can be genotyped in greater numbers than other types of genetic markers (such as RFLP or VNTR markers), are particularly useful in genetic analysis based on linkage disequilibrium.

When a disease mutation is first introduced into a population (by a new mutation or the immigration of a mutation carrier), it necessarily resides on a single chromosome and thus on a single “background” or “ancestral” haplotype of linked markers. Consequently, there is complete disequilibrium between these markers and the disease mutation: one finds the disease mutation only in the presence of a specific set of marker alleles. Through subsequent generations recombination events occur between the disease mutation and these marker polymorphisms, and the disequilibrium gradually dissipates. The pace of this dissipation is a function of the recombination frequency, so the markers closest to the disease gene will manifest higher levels of disequilibrium than those that are further away. When not broken up by recombination, “ancestral” haplotypes and linkage disequilibrium between marker alleles at different loci can be tracked not only through pedigrees but also through populations. Linkage disequilibrium is usually seen as an association between one specific allele at one locus and another specific allele at a second locus.

The pattern or curve of disequilibrium between disease and marker loci is expected to exhibit a maximum that occurs at the disease locus. Consequently, the amount of linkage disequilibrium between a disease allele and closely linked genetic markers may yield valuable information regarding the location of the disease gene. For fine-scale mapping of a disease locus, it is useful to have some knowledge of the patterns of linkage disequilibrium that exist between markers in the studied region. As mentioned above the mapping resolution achieved through the analysis of linkage disequilibrium is much higher than that of linkage studies. The high density of biallelic markers combined with linkage disequilibrium analysis provides powerful tools for fine-scale mapping. Different methods to calculate linkage disequilibrium are described below under the heading “Statistical Methods.”

E. Population-Based Case-Control Studies of Trait-Marker Associations

As mentioned above, the occurrence of pairs of specific alleles at different loci on the same chromosome is not random and the deviation from random is called linkage disequilibrium. Association studies focus on population frequencies and rely on the phenomenon of linkage disequilibrium. If a specific allele in a given gene is directly involved in causing a particular trait, its frequency will be statistically increased in an affected (trait positive) population, when compared to the frequency in a trait negative population or in a random control population. As a consequence of the existence of linkage disequilibrium, the frequency of all other alleles present in the haplotype carrying the trait-causing allele will also be increased in trait positive individuals compared to trait negative individuals or random controls. Therefore, association between the trait and any allele (specifically a biallelic marker allele) in linkage disequilibrium with the trait-causing allele will suffice to suggest the presence of a trait-related gene in that particular region. Case-control populations can be genotyped for biallelic markers to identify associations that narrowly locate a trait causing allele. As any marker in linkage disequilibrium with one given marker associated with a trait will be associated with the trait. Linkage disequilibrium allows the relative frequencies in case-control populations of a limited number of genetic polymorphisms (specifically biallelic markers) to be analyzed as an alternative to screening all possible functional polymorphisms in order to find trait-causing alleles. Association studies compare the frequency of marker alleles in unrelated case-control populations, and represent powerful tools for the dissection of complex traits.

i. Case-Control Populations (Inclusion Criteria)

Population-based association studies do not concern familial inheritance but compare the prevalence of a particular genetic marker, or a set of markers, in case-control populations. They are case-control studies based on comparison of unrelated case (affected or trait positive) individuals and unrelated control (unaffected, trait negative or random) individuals. Preferably the control group is composed of unaffected or trait negative individuals. Further, the control group is ethnically matched to the case population. Moreover, the control group is preferably matched to the case-population for the main known confusion factor for the trait under study (for example age-matched for an age-dependent trait). Ideally, individuals in the two samples are paired in such a way that they are expected to differ only in their disease status. The terms “trait positive population”, “case population” and “affected population” are used interchangeably herein.

An important step in the dissection of complex traits using association studies is the choice of case-control populations (see Lander and Schork, 1994). A major step in the choice of case-control populations is the clinical definition of a given trait or phenotype. Any genetic trait may be analyzed by the association method proposed here by carefully selecting the individuals to be included in the trait positive and trait negative phenotypic groups. Four criteria are often useful: clinical phenotype, age at onset, family history and severity. The selection procedure for continuous or quantitative traits (such as blood pressure for example) involves selecting individuals at opposite ends of the phenotype distribution of the trait under study, so as to include in these trait positive and trait negative populations individuals with non-overlapping phenotypes. Preferably, case-control populations comprise phenotypically homogeneous populations. Trait positive and trait negative populations comprise phenotypically uniform populations of individuals representing each between 1 and 98%, preferably between 1 and 80%, more preferably between 1 and 50%, and more preferably between 1 and 30%, most preferably between 1 and 20% of the total population under study, and preferably selected among individuals exhibiting non-overlapping phenotypes. The clearer the difference between the two trait phenotypes, the greater the probability of detecting an association with biallelic markers. The selection of those drastically different but relatively uniform phenotypes enables efficient comparisons in association studies and the possible detection of marked differences at the genetic level, provided that the sample sizes of the populations under study are significant enough.

In preferred embodiments, a first group of between 50 and 300 trait positive individuals, preferably about 100 individuals, are recruited according to their phenotypes. A similar number of control individuals are included in such studies.

ii. Association Analysis

The invention also comprises methods of detecting an association between a genotype and a phenotype, comprising the steps of: a) determining the frequency of at least one AA4RP-related biallelic marker in a trait positive population according to a genotyping method of the invention; b) determining the frequency of said AA4RP-related biallelic marker in a control population according to a genotyping method of the invention; and c) determining whether a statistically significant association exists between said genotype and said phenotype. In addition, the methods of detecting an association between a genotype and a phenotype of the invention encompass methods with any further limitation described in this disclosure, or those following, specified alone or in any combination: optionally, wherein said AA4RP-related biallelic marker is selected from the group consisting of 20-828-311, 17-42-319, 17-41-250, 20-841-149, 20-842-115, and 20-853-415, and the complements thereof, or optionally the biallelic markers in linkage disequilibrium therewith; optionally, wherein said AA4RP-related biallelic marker is selected from the group consisting of 17-42-319 and 17-41-250, and the complements thereof, or optionally the biallelic markers in linkage disequilibrium therewith; Optionally, said control population may be a trait negative population, or a random population; Optionally, each of said genotyping steps a) and b) may be performed on a pooled biological sample derived from each of said populations; Optionally, each of said genotyping of steps a) and b) is performed separately on biological samples derived from each individual in said population or a subsample thereof.

The general strategy to perform association studies using biallelic markers derived from a region carrying a candidate gene is to scan two groups of individuals (case-control populations) in order to measure and statistically compare the allele frequencies of the biallelic markers of the present invention in both groups.

If a statistically significant association with a trait is identified for at least one or more of the analyzed biallelic markers, one can assume that: either the associated allele is directly responsible for causing the trait (i.e. the associated allele is the trait causing allele), or more likely the associated allele is in linkage disequilibrium with the trait causing allele. The specific characteristics of the associated allele with respect to the candidate gene function usually give further insight into the relationship between the associated allele and the trait (causal or in linkage disequilibrium). If the evidence indicates that the associated allele within the candidate gene is most probably not the trait causing allele but is in linkage disequilibrium with the real trait causing allele, then the trait causing allele can be found by sequencing the vicinity of the associated marker, and performing further association studies with the polymorphisms that are revealed in an iterative manner.

Association studies are usually run in two successive steps. In a first phase, the frequencies of a reduced number of biallelic markers from the candidate gene are determined in the trait positive and control populations. In a second phase of the analysis, the position of the genetic loci responsible for the given trait is further refined using a higher density of markers from the relevant region. However, if the candidate gene under study is relatively small in length, as is the case for AA4RP, a single phase may be sufficient to establish significant associations.

iii. Haplotype Analysis

As described above, when a chromosome carrying a disease allele first appears in a population as a result of either mutation or migration, the mutant allele necessarily resides on a chromosome having a set of linked markers: the ancestral haplotype. This haplotype can be tracked through populations and its statistical association with a given trait can be analyzed. Complementing single point (allelic) association studies with multi-point association studies also called haplotype studies increases the statistical power of association studies. Thus, a haplotype association study allows one to define the frequency and the type of the ancestral carrier haplotype. A haplotype analysis is important in that it increases the statistical power of an analysis involving individual markers.

In a first stage of a haplotype frequency analysis, the frequency of the possible haplotypes based on various combinations of the identified biallelic markers of the invention is determined. The haplotype frequency is then compared for distinct populations of trait positive and control individuals. The number of trait positive individuals, which should be, subjected to this analysis to obtain statistically significant results usually ranges between 30 and 300, with a preferred number of individuals ranging between 50 and 150. The same considerations apply to the number of unaffected individuals (or random control) used in the study. The results of this first analysis provide haplotype frequencies in case-control populations, for each evaluated haplotype frequency a p-value and an odd ratio are calculated. If a statistically significant association is found the relative risk for an individual carrying the given haplotype of being affected with the trait under study can be approximated.

An additional embodiment of the present invention encompasses methods of detecting an association between a haplotype and a phenotype, comprising the steps of: a) estimating the frequency of at least one haplotype in a trait positive population, according to a method of the invention for estimating the frequency of a haplotype; b) estimating the frequency of said haplotype in a control population, according to a method of the invention for estimating the frequency of a haplotype; and c) determining whether a statistically significant association exists between said haplotype and said phenotype. In addition, the methods of detecting an association between a haplotype and a phenotype of the invention encompass methods with any further limitation described in this disclosure, or those following: optionally, wherein said AA4RP-related biallelic marker is selected from the group consisting of 20-828-311, 17-42-319, 17-41-250, 20-841-149, 20-842-1 15, and 20-853-415, and the complements thereof, or optionally the biallelic markers in linkage disequilibrium therewith; optionally, wherein said AA4RP-related biallelic marker is selected from the group consisting of 17-42-319 and 17-41-250, and the complements thereof, or optionally the biallelic markers in linkage disequilibrium therewith; Optionally, said control population is a trait negative population, or a random population. Optionally, said method comprises the additional steps of determining the phenotype in said trait positive and said control populations prior to step c).

iv. Interaction Analysis

The biallelic markers of the present invention may also be used to identify patterns of biallelic markers associated with detectable traits resulting from polygenic interactions. The analysis of genetic interaction between alleles at unlinked loci requires individual genotyping using the techniques described herein. The analysis of allelic interaction among a selected set of biallelic markers with appropriate level of statistical significance can be considered as a haplotype analysis. Interaction analysis comprises stratifying the case-control populations with respect to a given haplotype for the first loci and performing a haplotype analysis with the second loci with each subpopulation.

Statistical methods used in association studies are further described below.

F. Testing for Linkage in the Presence of Association

The biallelic markers of the present invention may further be used in TDT (transmission/disequilibrium test). TDT tests for both linkage and association and is not affected by population stratification. TDT requires data for affected individuals and their parents or data from unaffected sibs instead of from parents (see Spielmann S. et al., 1993; Schaid D. J. et al., 1996, Spielmann S. and Ewens W. J., 1998). Such combined tests generally reduce the false-positive errors produced by separate analyses.

VII. Statistical Methods

In general, any method known in the art to test whether a trait and a genotype show a statistically significant correlation may be used.

A. Methods in Linkage Analysis

Statistical methods and computer programs useful for linkage analysis are well-known to those skilled in the art (see Terwilliger J. D. and Ott J., 1994; Ott J., 1991).

B. Methods to Estimate Haplotype Frequencies in a Population

As described above, when genotypes are scored, it is often not possible to distinguish heterozygotes so that haplotype frequencies cannot be easily inferred. When the gametic phase is not known, haplotype frequencies can be estimated from the multilocus genotypic data. Any method known to person skilled in the art can be used to estimate haplotype frequencies (see Lange K., 1997; Weir, B. S., 1996) Preferably, maximum-likelihood haplotype frequencies are computed using an Expectation-Maximization (EM) algorithm (see Dempster et al., 1977; Excoffier L. and Slatkin M., 1995). This procedure is an iterative process aiming at obtaining maximum-likelihood estimates of haplotype frequencies from multi-locus genotype data when the gametic phase is unknown. Haplotype estimations are usually performed by applying the EM algorithm using for example the EM-HAPLO program (Hawley M. E. et al., 1994) or the Arlequin program (Schneider et al., 1997). The EM algorithm is a generalized iterative maximum likelihood approach to estimation and is briefly described below.

Please note that in the present section, “Methods To Estimate Haplotype Frequencies In A Population,”of this text, phenotypes will refer to multi-locus genotypes with unknown phase. Genotypes will refer to known-phase multi-locus genotypes.

A sample of N unrelated individuals is typed for K markers. The data observed are the unknown-phase K-locus phenotypes that can categorized in F different phenotypes. Suppose that we have H underlying possible haplotypes (in case of K biallelic markers, H=2^{K}).

For phenotype j, suppose that cⱼ genotypes are possible. We thus have the following equation

$${P}_{j} = {\sum }_{i = 1}^{{c}_{j}} p r ( g e n o t y p {e}_{i} ) = {\sum }_{i = 1}^{{c}_{j}} p r ( {h}_{k} , {h}_{l} ) \qquad Equation 1$$

where Pj is the probability of the phenotypej, hₖ and hₗ are the two haplotypes constituent the genotype i. Under the Hardy-Weinberg equilibrium, pr(hₖ, hₗ) becomes:

pr(hₖ,hₗ)=pr(hₖ)² if hₖ=hₗ, pr(hₖ, hₗ)=2pr(hₖ).pr(hₗ) if hₖ≠hₗ. Equation 2

The successive steps of the E-M algorithm can be described as follows:

Starting with initial values of the of haplotypes frequencies, noted p₁⁽⁰⁾, p₂⁽⁰⁾, . . . p_{H}⁽⁰⁾, these initial values serve to estimate the genotype frequencies (Expectation step) and then estimate another set of haplotype frequencies (Maximization step), noted p₁⁽¹⁾, p₂⁽¹⁾, . . . p_{H}⁽¹⁾, these two steps are iterated until changes in the sets of haplotypes frequency are very small.

A stop criterion can be that the maximum difference between haplotype frequencies between two iterations is less than 10⁻⁷. These values can be adjusted according to the desired precision of estimations.

At a given iteration s, the Expectation step comprises calculating the genotypes frequencies by the following equation:

$$\begin{matrix}p {r ( g e n o t y p {e}_{i} )}^{( s )} = p r ( p h e n o t y p {e}_{j} ) \cdot \\ p {r ( g e n o t y p {e}_{i} \vert p h e n o t y p {e}_{j} )}^{( s )} \\ = \frac{{n}_{j}}{N} \cdot \frac{p {r ( {h}_{k} , {h}_{l} )}^{( s )}}{{P}_{j}^{( s )}}\end{matrix} \qquad Equation 3$$

where genotype i occurs in phenotypej, and where hₖ and hₗ constitute genotype i. Each probability is derived according to eq. 1, and eq. 2 described above.

Then the Maximization step simply estimates another set of haplotype frequencies given the genotypes frequencies. This approach is also known as the gene-counting method (Smith, 1957).

$${p}_{t}^{( s + 1 )} = \frac{1}{2} {\sum }_{j = 1}^{F} {\sum }_{i = 1}^{{c}_{j}} {δ}_{it} \cdot p {r ( g e n o t y p {e}_{i} )}^{( s )} \qquad Equation 4$$

Where δᵢₜ is an indicator variable which count the number of time haplotype t in genotype i. It takes the values of 0, 1 or 2.

To ensure that the estimation finally obtained is the maximum-likelihood estimation several values of departures are required. The estimations obtained are compared and if they are different the estimations leading to the best likelihood are kept.

Methods to Calculate Linkage Disequilibrium Between Markers

A number of methods can be used to calculate linkage disequilibrium between any two genetic positions, in practice linkage disequilibrium is measured by applying a statistical association test to haplotype data taken from a population.

Linkage disequilibrium between any pair of biallelic markers comprising at least one of the biallelic markers of the present invention (Mᵢ, Mⱼ) having alleles (aᵢ/bᵢ) at marker Mᵢ and alleles (aⱼ/bⱼ) at marker Mⱼ can be calculated for every allele combination (aᵢ,aⱼ; aᵢ,bⱼ, bᵢ,aⱼ and bᵢ,bⱼ), according to the Piazza formula:

Δₐᵢₐⱼ=√θ4−√(θ4+θ3) (θ4+θ2),

where:

θ4=−−=frequency of genotypes not having allele aᵢ at Mᵢ and not having allele aⱼ at Mⱼ

θ3=−+=frequency of genotypes not having allele aᵢ at Mᵢ and having allele aⱼ at Mⱼ

θ2=+−=frequency of genotypes having allele aᵢ at Mᵢ and not having allele aⱼ at Mⱼ

Linkage disequilibrium (LD) between pairs of biallelic markers (Mᵢ, Mⱼ) can also be calculated for every allele combination (aᵢ,aⱼ; aᵢ,bⱼ; bᵢ,aⱼ and bᵢ,bⱼ), according to the maximum-likelihood estimate (MLE) for delta (the composite genotypic disequilibrium coefficient), as described by Weir (Weir B. S., 1996). The MLE for the composite linkage disequilibrium is:

Dₐᵢₐⱼ=(2n₁+n₂+n₃+n₄/2)/N−2(pr(aᵢ). pr(aⱼ))

Where n₁=Σ phenotype (aᵢ/aᵢ, aⱼ/aⱼ), n₂=Σ phenotype (aᵢ/aᵢ;, aⱼ/bⱼ), n₃=Σ phenotype (aᵢ/bᵢ, aⱼ/aⱼ), n4=Σ phenotype (aᵢ/bᵢ, aⱼ/bⱼ) and N is the number of individuals in the sample.

This formula allows linkage disequilibrium between alleles to be estimated when only genotype, and not haplotype, data are available.

Another means of calculating the linkage disequilibrium between markers is as follows. For a couple of biallelic markers, Mᵢ(aᵢ/bᵢ) and Mⱼ(aⱼ/bⱼ), fitting the Hardy-Weinberg equilibrium, one can estimate the four possible haplotype frequencies in a given population according to the approach described above.

The estimation of gametic disequilibrium between ai and aj is simply:

Dₐᵢₐⱼ=pr(haplotype(aᵢ,aⱼ))−pr(aᵢ).pr(aⱼ).

Where pr(aᵢ) is the probability of allele aᵢ and pr(aⱼ) is the probability of allele aⱼ and where pr(haplotype (aᵢ, aⱼ)) is estimated as in Equation 3 above.

For a couple of biallelic marker only one measure of disequilibrium is necessary to describe the association between Mᵢ and Mⱼ.

Then a normalized value of the above is calculated as follows:

D′ₐᵢₐⱼ=Dₐᵢₐⱼ/max(−pr(aᵢ). pr(aⱼ), −pr(bᵢ). pr(bⱼ)) with Dₐᵢₐⱼ<0

D′ₐᵢₐⱼ=Dₐᵢₐⱼ/max(pr(bᵢ). pr(aⱼ), pr(aᵢ). pr(bⱼ)) with Dₐᵢₐⱼ>0

The skilled person will readily appreciate that other linkage disequilibrium calculation methods can be used.

Linkage disequilibrium among a set of biallelic markers having an adequate heterozygosity rate can be determined by genotyping between 50 and 1000 unrelated individuals, preferably between 75 and 200, more preferably around 100.

C. Testing for Association

Methods for determining the statistical significance of a correlation between a phenotype and a genotype, in this case an allele at a biallelic marker or a haplotype made up of such alleles, may be determined by any statistical test known in the art and with any accepted threshold of statistical significance being required. The application of particular methods and thresholds of significance are well with in the skill of the ordinary practitioner of the art.

Testing for association is performed by determining the frequency of a biallelic marker allele in case and control populations and comparing these frequencies with a statistical test to determine if their is a statistically significant difference in frequency which would indicate a correlation between the trait and the biallelic marker allele under study. Similarly, a haplotype analysis is performed by estimating the frequencies of all possible haplotypes for a given set of biallelic markers in case and control populations, and comparing these frequencies with a statistical test to determine if their is a statistically significant correlation between the haplotype and the phenotype (trait) under study. Any statistical tool useful to test for a statistically significant association between a genotype and a phenotype may be used. Preferably the statistical test employed is a chi-square test with one degree of freedom. A P-value is calculated (the P-value is the probability that a statistic as large or larger than the observed one would occur by chance).

i. Statistical Significance

In preferred embodiments, significance for diagnosis purposes, either as a positive basis for further diagnostic tests or as a preliminary starting point for early preventive therapy, the p value related to a biallelic marker association is preferably about 1×10⁻² or less, more preferably about 1×10⁻⁴ or less, for a single biallelic marker analysis and about 1×10⁻³ or less, still more preferably 1×10⁻⁶ or less and most preferably of about 1×10⁻⁸ or less, for a haplotype analysis involving two or more markers. These values are believed to be applicable to any association studies involving single or multiple marker combinations.

The skilled person can use the range of values set forth above as a starting point in order to carry out association studies with biallelic markers of the present invention. In doing so, significant associations between the biallelic markers of the present invention and a trait can be revealed and used for diagnosis and drug screening purposes.

ii. Phenotypic Permutation

In order to confirm the statistical significance of the first stage haplotype analysis described above, it might be suitable to perform further analyses in which genotyping data from case-control individuals are pooled and randomized with respect to the trait phenotype. Each individual genotyping data is randomly allocated to two groups, which contain the same number of individuals as the case-control populations used to compile the data obtained in the first stage. A second stage haplotype analysis is preferably run on these artificial groups, preferably for the markers included in the haplotype of the first stage analysis showing the highest relative risk coefficient. This experiment is reiterated preferably at least between 100 and 1000 times. The repeated iterations allow the determination of the probability to obtain the tested haplotype by chance.

iii. Assessment of Statistical Association

To address the problem of false positives similar analysis may be performed with the same case-control populations in random genomic regions. Results in random regions and the candidate region are compared as described in a co-pending US Provisional Patent Application entitled “Methods, Software And Apparati For Identifying Genomic Regions Harboring A Gene Associated With A Detectable Trait,” U.S. Serial No. 60/107,986, filed Nov. 10, 1998, the contents of which are incorporated herein by reference.

D. Evaluation of Risk Factors

The association between a risk factor (in genetic epidemiology the risk factor is the presence or the absence of a certain allele or haplotype at marker loci) and a disease is measured by the odds ratio (OR) and by the relative risk (RR). If P(R+) is the probability of developing the disease for individuals with R and P(R⁻) is the probability for individuals without the risk factor, then the relative risk is simply the ratio of the two probabilities, that is:

RR=P(R⁺)/P(R⁻)

$$O R = [ \frac{{F}^{+}}{1 - {F}^{+}} ] / [ \frac{{F}^{-}}{( 1 + {F}^{-} )} ]$$

In case-control studies, direct measures of the relative risk cannot be obtained because of the sampling design. However, the odds ratio allows a good approximation of the relative risk for low-incidence diseases and can be calculated:

OR=(F⁺/(1−F⁺))/(F⁻/(1−F⁻))

F⁺ is the frequency of the exposure to the risk factor in cases and F⁻ is the frequency of the exposure to the risk factor in controls. F⁺ and F⁻ are calculated using the allelic or haplotype frequencies of the study and further depend on the underlying genetic model (dominant, recessive, additive . . . ).

One can further estimate the attributable risk (AR) which describes the proportion of individuals in a population exhibiting a trait due to a given risk factor. This measure is important in quantifying the role of a specific factor in disease etiology and in terms of the public health impact of a risk factor. The public health relevance of this measure lies in estimating the proportion of cases of disease in the population that could be prevented if the exposure of interest were absent. AR is determined as follows:

AR=P_{E}(RR−1)/(P_{E}(RR−1)+1)

AR is the risk attributable to a biallelic marker allele or a biallelic marker haplotype. P_{E} is the frequency of exposure to an allele or a haplotype within the population at large; and RR is the relative risk which, is approximated with the odds ratio when the trait under study has a relatively low incidence in the general population.

VIII. Identification of Biallelic Markers in Linkage Disequilibrium with the Biallelic Markers of the Invention

Once a first biallelic marker has been identified in a genomic region of interest, the practitioner of ordinary skill in the art, using the teachings of the present invention, can easily identify additional biallelic markers in linkage disequilibrium with this first marker. As mentioned before any marker in linkage disequilibrium with a first marker associated with a trait will be associated with the trait. Therefore, once an association has been demonstrated between a given biallelic marker and a trait, the discovery of additional biallelic markers associated with this trait is of great interest in order to increase the density of biallelic markers in this particular region. The causal gene or mutation will be found in the vicinity of the marker or set of markers showing the highest correlation with the trait.

Identification of additional markers in linkage disequilibrium with a given marker involves: (a) amplifying a genomic fragment comprising a first biallelic marker from a plurality of individuals; (b) identifying of second biallelic markers in the genomic region harboring said first biallelic marker; (c) conducting a linkage disequilibrium analysis between said first biallelic marker and second biallelic markers; and (d) selecting said second biallelic markers as being in linkage disequilibrium with said first marker. Subcombinations comprising steps (b) and (c) are also contemplated.

Methods to identify biallelic markers and to conduct linkage disequilibrium analysis are described herein and can be carried out by the skilled person without undue experimentation. The present invention then also concerns biallelic markers which are in linkage disequilibrium with the biallelic markers 20-828-311, 17-42-319, 17-41-250, 20-841-149, 20-842-115, and 20-853-415 and which are expected to present similar characteristics in terms of their respective association with a given trait.

IX. Identification of Functional Mutations

Mutations in the AA4RP gene which are responsible for a detectable phenotype or trait may be identified by comparing the sequences of the AA4RP gene from trait positive and control individuals. Once a positive association is confirmed with a biallelic marker of the present invention, the identified locus can be scanned for mutations. In a preferred embodiment, functional regions such as exons and splice sites, promoters and other regulatory regions of the AA4RP gene are scanned for mutations. In a preferred embodiment the sequence of the AA4RP gene is compared in trait positive and control individuals. Preferably, trait positive individuals carry the haplotype shown to be associated with the trait and trait negative individuals do not carry the haplotype or allele associated with the trait. The detectable trait or phenotype may comprise a variety of manifestations of altered AA4RP function.

The mutation detection procedure is essentially similar to that used for biallelic marker identification. The method used to detect such mutations generally comprises the following steps:

amplification of a region of the AA4RP gene comprising a biallelic marker or a group of biallelic markers associated with the trait from DNA samples of trait positive patients and trait-negative controls,

sequencing of the amplified region;

comparison of DNA sequences from trait positive and control individuals;

determination of mutations specific to trait-positive patients.

In one embodiment, said biallelic marker is selected from the group consisting of 20-828-311, 17-42-319, 17-41-250, 20-841-149, 20-842-115, and 20-853-415, and the complements thereof. It is preferred that candidate polymorphisms be then verified by screening a larger population of cases and controls by means of any genotyping procedure such as those described herein, preferably using a microsequencing technique in an individual test format. Polymorphisms are considered as candidate mutations when present in cases and controls at frequencies compatible with the expected association results. Polymorphisms are considered as candidate “trait-causing” mutations when they exhibit a statistically significant correlation with the detectable phenotype.

X. Biallelic Markers of the Invention in Methods of Genetic Diagnostics

The biallelic markers of the present invention can also be used to develop diagnostics tests capable of identifying individuals who express a detectable trait as the result of a specific genotype or individuals whose genotype places them at risk of developing a detectable trait at a subsequent time. The trait analyzed using the present diagnostics may be any detectable trait, including body mass index (BMI), food intake, RAP3 expression, RAP3 concentration, liver regeneratoin, plasma levels of leptin, insulin, free fatty acids (FFA), triglycerides (TG) and glucose. Such a diagnosis can be useful in the staging, monitoring, prognosis and/or prophylactic or curative therapy of diseases involving lipid metabolism and/or liver related disorders.

The diagnostic techniques of the present invention may employ a variety of methodologies to determine whether a test subject has a biallelic marker pattern associated with an increased risk of developing a detectable trait or whether the individual suffers from a detectable trait as a result of a particular mutation, including methods which enable the analysis of individual chromosomes for haplotyping, such as family studies, single sperm DNA analysis or somatic hybrids.

The present invention provides diagnostic methods to determine whether an individual is at risk of developing a disease or suffers from a disease resulting from a mutation or a polymorphism in the AA4RP gene. The present invention also provides methods to determine whether an individual has a susceptibility to diseases involving lipid metabolism and/or liver related disorders.

These methods involve obtaining a nucleic acid sample from the individual and, determining, whether the nucleic acid sample contains at least one allele or at least one biallelic marker haplotype, indicative of a risk of developing the trait or indicative that the individual expresses the trait as a result of possessing a particular AA4RP polymorphism or mutation (trait-causing allele).

Preferably, in such diagnostic methods, a nucleic acid sample is obtained from the individual and this sample is genotyped using methods described above in “Methods of Genotyping DNA Samples for Biallelic Markers.” The diagnostics may be based on a single biallelic marker or a on group of biallelic markers.

In each of these methods, a nucleic acid sample is obtained from the test subject and the biallelic marker pattern of one or more of the biallelic markers 20-828-311, 17-42-319, 17-41-250, 20-841-149, 20-842-115, and 20-853-415 is determined.

In one embodiment, a PCR amplification is conducted on the nucleic acid sample to amplify regions in which polymorphisms associated with a detectable phenotype have been identified. The amplification products are sequenced to determine whether the individual possesses one or more AA4RP polymorphisms associated with a detectable phenotype. The primers used to generate amplification products may comprise the primers listed in FIG. 5. Alternatively, the nucleic acid sample is subjected to microsequencing reactions as described above to determine whether the individual possesses one or more AA4RP polymorphisms associated with a detectable phenotype resulting from a mutation or a polymorphism in the AA4RP gene. The primers used in the microsequencing reactions may include the primers listed in FIG. 4. In another embodiment, the nucleic acid sample is contacted with one or more allele specific oligonucleotide probes which, specifically hybridize to one or more AA4RP alleles associated with a detectable phenotype. The probes used in the hybridization assay may include the probes listed in FIG. 6. In another embodiment, the nucleic acid sample is contacted with a second AA4RP oligonucleotide capable of producing an amplification product when used with the allele specific oligonucleotide in an amplification reaction. The presence of an amplification product in the amplification reaction indicates that the individual possesses one or more AA4RP alleles associated with a detectable phenotype.

In a preferred embodiment the identity of the nucleotide present at, at least one, biallelic marker selected from the group consisting of 20-828-311, 17-42-319, 17-41-250, 20-841-149, 20-842-115, and 20-853-415, and the complements thereof, is determined and the detectable trait is a disease involving lipid metabolism and/or liver related disorders. Diagnostic kits comprise any of the polynucleotides of the present invention.

These diagnostic methods are extremely valuable as they can, in certain circumstances, be used to initiate preventive treatments or to allow an individual carrying a significant haplotype to foresee warning signs such as minor symptoms.

Diagnostics, which analyze and predict response to a drug or side effects to a drug, may be used to determine whether an individual should be treated with a particular drug. For example, if the diagnostic indicates a likelihood that an individual will respond positively to treatment with a particular drug, the drug may be administered to the individual. Conversely, if the diagnostic indicates that an individual is likely to respond negatively to treatment with a particular drug, an alternative course of treatment may be prescribed. A negative response may be defined as either the absence of an efficacious response or the presence of toxic side effects.

Clinical drug trials represent another application for the markers of the present invention. One or more markers indicative of response to an agent acting on lipid metabolism and/or liver related disorders or to side effects to an agent acting on lipid metabolism and/or a liver related disorder may be identified using the methods described above. Thereafter, potential participants in clinical trials of such an agent may be screened to identify those individuals most likely to respond favorably to the drug and exclude those likely to experience side effects. In that way, the effectiveness of drug treatment may be measured in individuals who respond positively to the drug, without lowering the measurement as a result of the inclusion of individuals who are unlikely to respond positively in the study and without risking undesirable safety problems.

XI. The Rat Homolog of AA4RP (RAP3) in the Diagnosis and Treatment of Liver Related Disorders

A. Methods for Diagnosing Liver Related Disorders

The antibodies of AA4RP can be used in the diagnosis of liver related disorders. Such disorders include, but are not limited to hepatitis, cirrhosis, hepatoma, and FHP. In such disorders, damage to the liver may result in the up-regulation of the expression of the AA4RP gene, or increased secretion/release from stores. In rats, liver damage results in increased levels of RAP3 in the serum, thus increases in the amount of AA4RP in the serum may also be expected as a result of or correlating with liver damage. In addition, up-regulation of the AA4RP gene may also give rise to the liver disorder. To detect such disorders, an appropriate biological sample (serum, for example) can be tested with antibody against AA4RP to determine the level of AA4RP being produced. A liver disorder will be indicated by an excess amount of AA4RP detected in comparison to that detected in the sample from a normal subject (U.S. Pat. No. 6,027,935).

B. Treatment of Intracorporeal Liver Tissue

AA4RP gene products or antagonists and agonists of AA4RP may be used to enhance the growth or regeneration of liver tissue in a variety of situations. In some cases, a patient's liver may be damaged but not beyond repair. For example, and not by way of limitation, excessive consumption of alcohol often leads to cirrhosis of the liver. Hepatocyte destruction can be arrested by discontinuation of alcohol consumption, but recovery will be facilitated and may require subsequent regeneration of the liver. In such cases, the natural regeneration process may be impaired due to extensive liver damage. In any event, treatment of the patient with pharmaceutical compositions, as described in section XIX. Pharmaceutical Compositions of the Invention, comprising AA4RP gene products or antagonists and agonists of AA4RP will enhance regeneration and thereby speed recovery.

In some situations, treatment may require transplanting all or a section of the liver of a donor.

Regeneration of both a living donor's and a recipient's liver during such transplantation treatments will be aided by administering pharmaceutical compositions, as described section XIX. Pharmaceutical Compositions of the Invention, comprising a AA4RP gene products or antagonists and agonists of AA4RP.

In other situations, an artificial liver may be implanted into a patient suffering from liver disease. It may be sufficient and desirable to implant such an artificial liver at a stage where it has not yet attained the biological capacity of a normal liver. To increase the capacity of such an implant, the growth rate can be enhanced by administering pharmaceutical compositions, as described in section XIX. Pharmaceutical Compositions of the Invention, comprising a AA4RP gene product or antagonists and agonists of AA4RP.

In cases where a patient's natural liver is damaged or diseased, it may be left intact or only partially removed, but still require support from implanted artificial liver tissue or liver tissue transplanted from a donor. Pharmaceutical compositions comprising a AA4RP gene product such as antagonists and agonists of AA4RP can be used also in such cases to enhance the growth of the patient's natural liver tissue, as well as the implanted or transplanted liver tissue.

The use of AA4RP gene product such as antagonists and agonists of AA4RP in enhancing cell growth may be applied to other tissues, as well, including, but not limited to, hematopoietic cells.

C. In Vitro Liver Tissue Cultures

In vitro liver tissue cultures have a variety of uses. In treating patients suffering from liver damage or disease, for example, the liver tissue cultures can be used to support or replace the natural liver, by direct implantation or as part of an extracorporeal liver device. In addition, such liver tissue cultures can serve as models for testing the toxicity of drugs and other compounds.

D. Methods for Treatment of Liver Disease by Affecting AA4RP Gene Expression

Described below are methods whereby liver related disorders may be treated with the nucleic acid sequences described in the “Polynucleotides” section, above. In certain cases, including but not limited to cirrhosis, an increase in AA4RP gene product activity would facilitate regeneration or amelioration of liver damage. Furthermore, certain liver diseases may be brought about, at least in part, by the absence or reduction of the level of AA4RP gene expression. As such, an increase in the level of gene expression would bring about the amelioration of liver disease symptoms.

In some cases, including but not limited to hepatoma, liver diseases may be brought about, at least in part, by an excessive level of AA4RP gene product, or by the presence of a AA4RP gene product exhibiting an abnormal or excessive activity. As such, the reduction in the level and/or activity of such gene products would bring about the amelioration of liver disease symptoms.

XII. Recombinant Vectors

The term “vector” is used herein to designate either a circular or a linear DNA or RNA molecule, which is either double-stranded or single-stranded, and which comprise at least one polynucleotide of interest that is sought to be transferred in a cell host or in a unicellular or multicellular host organism.

The present invention encompasses a family of recombinant vectors that comprise a regulatory polynucleotide derived from the AA4RP genomic sequence, and/or a coding polynucleotide from either the AA4RP genomic sequence or the cDNA sequence.

Generally, a recombinant vector of the invention may comprise any of the polynucleotides described herein, including regulatory sequences, coding sequences and polynucleotide constructs, as well as any AA4RP primer or probe as defined above. More particularly, the recombinant vectors of the present invention can comprise any of the polynucleotides described in the “Genomic Sequences Of tThe AA4RP Gene” section, the “AA4RP cDNA Sequences” section, the “Coding Regions” section, the “Polynucleotide constructs” section, and the “Oligonucleotide Probes And Primers” section.

In a first preferred embodiment, a recombinant vector of the invention is used to amplify the inserted polynucleotide derived from a AA4RP genomic sequence of SEQ ID No 1 and 4 or a AA4RP cDNA, for example the cDNA of SEQ ID No 2 in a suitable cell host, this polynucleotide being amplified at every time that the recombinant vector replicates.

A second preferred embodiment of the recombinant vectors according to the invention comprises expression vectors comprising either a regulatory polynucleotide or a coding nucleic acid of the invention, or both. Within certain embodiments, expression vectors are employed to express the AA4RP polypeptide which can be then purified and, for example be used in ligand screening assays or as an immunogen in order to raise specific antibodies directed against the AA4RP protein. In other embodiments, the expression vectors are used for constructing transgenic animals and also for gene therapy. Expression requires that appropriate signals are provided in the vectors, said signals including various regulatory elements, such as enhancers/promoters from both viral and mammalian sources that drive expression of the genes of interest in host cells. Dominant drug selection markers for establishing permanent, stable cell clones expressing the products are generally included in the expression vectors of the invention, as they are elements that link expression of the drug selection markers to expression of the polypeptide.

More particularly, the present invention relates to expression vectors which include nucleic acids encoding a AA4RP protein, preferably the AA4RP protein of the amino acid sequence of SEQ ID No 3 or variants or fragments thereof.

The invention also pertains to a recombinant expression vector useful for the expression of the AA4RP coding sequence, wherein said vector comprises a nucleic acid of SEQ ID No 2.

Recombinant vectors comprising a nucleic acid containing a AA4RP-related biallelic marker is also part of the invention. In a preferred embodiment, said biallelic marker is selected from the group consisting of 20-828-311, 17-42-319, 17-41-250, 20-841-149, 20-842-115, and 20-853-415, and the complements thereof.

Some of the elements which can be found in the vectors of the present invention are described in further detail in the following sections.

A. General Features of the Expression Vectors of the Invention

A recombinant vector according to the invention comprises, but is not limited to, a YAC (Yeast Artificial Chromosome), a BAC (Bacterial Artificial Chromosome), a phage, a phagemid, a cosmid, a plasmid or even a linear DNA molecule which may comprise a chromosomal, non-chromosomal, semi-synthetic and synthetic DNA. Such a recombinant vector can comprise a transcriptional unit comprising an assembly of:

(1) a genetic element or elements having a regulatory role in gene expression, for example promoters or enhancers. Enhancers are cis-acting elements of DNA, usually from about 10 to 300 bp in length that act on the promoter to increase the transcription.

(2) a structural or coding sequence which is transcribed into mRNA and eventually translated into a polypeptide, said structural or coding sequence being operably linked to the regulatory elements described in (1); and

(3) appropriate transcription initiation and termination sequences. Structural units intended for use in yeast or eukaryotic expression systems preferably include a leader sequence enabling extracellular secretion of translated protein by a host cell. Alternatively, when a recombinant protein is expressed without a leader or transport sequence, it may include a N-terminal residue. This residue may or may not be subsequently cleaved from the expressed recombinant protein to provide a final product.

Generally, recombinant expression vectors will include origins of replication, selectable markers permitting transformation of the host cell, and a promoter derived from a highly expressed gene to direct transcription of a downstream structural sequence. The heterologous structural sequence is assembled in appropriate phase with translation initiation and termination sequences, and preferably a leader sequence capable of directing secretion of the translated protein into the periplasmic space or the extracellular medium. In a specific embodiment wherein the vector is adapted for transfecting and expressing desired sequences in mammalian host cells, preferred vectors will comprise an origin of replication in the desired host, a suitable promoter and enhancer, and also any necessary ribosome binding sites, polyadenylation signal, splice donor and acceptor sites, transcriptional termination sequences, and 5′-flanking non-transcribed sequences. DNA sequences derived from the SV40 viral genome, for example SV40 origin, early promoter, enhancer, splice and polyadenylation signals may be used to provide the required non-transcribed genetic elements.

The in vivo expression of a AA4RP polypeptide of SEQ ID No 3 or fragments or variants thereof may be useful in order to correct a genetic defect related to the expression of the native gene in a host organism or to the production of a biologically inactive AA4RP protein.

Consequently, the present invention also comprises recombinant expression vectors mainly designed for the in vivo production of the AA4RP polypeptide of SEQ ID No 3 or fragments or variants thereof by the introduction of the appropriate genetic material in the organism of the patient to be treated. This genetic material may be introduced in vitro in a cell that has been previously extracted from the organism, the modified cell being subsequently reintroduced in the said organism, directly in vivo into the appropriate tissue.

B. Regulatory Elements

i. Promoters

The suitable promoter regions used in the expression vectors according to the present invention are chosen taking into account the cell host in which the heterologous gene has to be expressed. The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell, such as, for example, a human or a viral promoter.

A suitable promoter may be heterologous with respect to the nucleic acid for which it controls the expression or alternatively can be endogenous to the native polynucleotide containing the coding sequence to be expressed. Additionally, the promoter is generally heterologous with respect to the recombinant vector sequences within which the construct promoter/coding sequence has been inserted.

Promoter regions can be selected from any desired gene using, for example, CAT (chloramphenicol transferase) vectors and more preferably pKK232-8 and pCM7 vectors. Preferred bacterial promoters are the LacI, LacZ, the T3 or T7 bacteriophage RNA polymerase promoters, the gpt, lambda PR, PL and trp promoters (EP 0036776), the polyhedrin promoter, or the p10 protein promoter from baculovirus (Kit Novagen) (Smith et al., 1983; O'Reilly et al., 1992), the lambda PR promoter or also the trc promoter.

Eukaryotic promoters include CMV immediate early, HSV thymidine kinase, early and late SV40, LTRs from retrovirus, and mouse metallothionein-L. Selection of a convenient vector and promoter is well within the level of ordinary skill in the art.

The choice of a promoter is well within the ability of a person skilled in the field of genetic engineering. For example, one may refer to the book of Sambrook et al.(1989) or also to the procedures described by Fuller et al.(1996).

ii. Other Regulatory Elements

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

C. Selectable Markers

Such markers would confer an identifiable change to the cell permitting easy identification of cells containing the expression construct. The selectable marker genes for selection of transformed host cells are preferably dihydrofolate reductase or neomycin resistance for eukaryotic cell culture, TRP1 for S. cerevisiae or tetracycline, rifampicin or ampicillin resistance in E. coli, or levan saccharase for mycobacteria, this latter marker being a negative selection marker.

D. Preferred Vectors

i. Bacterial Vectors

As a representative but non-limiting example, useful expression vectors for bacterial use can comprise a selectable marker and a bacterial origin of replication derived from commercially available plasmids comprising genetic elements of pBR322 (ATCC 37017). Such commercial vectors include, for example, pKK223-3 (Pharmacia, Uppsala, Sweden), and GEM1 (Promega Biotec, Madison, Wis., USA). Large numbers of other suitable vectors are known to those of skill in the art, and commercially available, such as the following bacterial vectors: pQE70, pQE60, pQE-9 (Qiagen), pbs, pD 10, phagescript, psiX174, pbluescript SK, pbsks, pNH8A, pNH16A, pNH18A, pNH46A (Stratagene); ptrc99a, pKK223-3, pKK233-3, pDR540, pRIT5 (Pharmacia); pWLNEO, pSV2CAT, pOG44, pXT1, pSG (Stratagene); pSVK3, pBPV, pMSG, pSVL (Pharmacia); pQE-30 (QIAexpress).

ii. Bacteriophage Vectors

The P1 bacteriophage vector may contain large inserts ranging from about 80 to about 100 kb.

The construction of PI bacteriophage vectors such as p158 or p158/neo8 are notably described by Stemberg (1992, 1994). Recombinant P1 clones comprising AA4RP nucleotide sequences may be designed for inserting large polynucleotides of more than 40 kb (Linton et al., 1993). To generate P1 DNA for transgenic experiments, a preferred protocol is the protocol described by McCormick et al.(11994). Briefly, E. coli (preferably strain NS3529) harboring the P1 plasmid are grown overnight in a suitable broth medium containing 25 μg/ml of kanamycin. The P1 DNA is prepared from the E. coli by alkaline lysis using the Qiagen Plasmid Maxi kit (Qiagen, Chatsworth, Calif., USA), according to the manufacturer's instructions. The P1 DNA is purified from the bacterial lysate on two Qiagen-tip 500 columns, using the washing and elution buffers contained in the kit. A phenol/chloroform extraction is then performed before precipitating the DNA with 70% ethanol. After solubilizing the DNA in TE (10 mM Tris-HCl, pH 7.4, 1 mM EDTA), the concentration of the DNA is assessed by spectrophotometry.

When the goal is to express a P1 clone comprising AA4RP nucleotide sequences in a transgenic animal, typically in transgenic mice, it is desirable to remove vector sequences from the PI DNA fragment, for example by cleaving the P 1 DNA at rare-cutting sites within the P1 polylinker (SfiI, NotI or SalI). The P1 insert is then purified from vector sequences on a pulsed-field agarose gel, using methods similar using methods similar to those originally reported for the isolation of DNA from YACs (Schedl et al., 1993a; Peterson et al., 1993). At this stage, the resulting purified insert DNA can be concentrated, if necessary, on a Millipore Ultrafree-MC Filter Unit (Millipore, Bedford, Mass., USA—30,000 molecular weight limit) and then dialyzed against microinjection buffer (10 mM Tris-HCl, pH 7.4; 250 μM EDTA) containing 100 mM NaCl, 30 μM spermine, 70 μM spermidine on a microdyalisis membrane (type VS, 0.025 μM from Millipore). The intactness of the purified P1 DNA insert is assessed by electrophoresis on 1% agarose (Sea Kem GTG; FMC Bio-products) pulse-field gel and staining with ethidium bromide.

iii. Baculovirus Vectors

A suitable vector for the expression of the AA4RP polypeptide of SEQ ID No 3 or fragments or variants thereof is a baculovirus vector that can be propagated in insect cells and in insect cell lines. A specific suitable host vector system is the pVL1392/1393 baculovirus transfer vector (Pharmingen) that is used to transfect the SF9 cell line (ATCC N°CRL 1711) which is derived from Spodoptera frugiperda. See Example 4 for further details.

Other suitable vectors for the expression of the AA4RP polypeptide of SEQ ID No 3 or fragments or variants thereof in a baculovirus expression system include those described by Chai et al.(1993), Vlasak et al.(1983) and Lenhard et al.(1996).

iv. Viral Vectors

In one specific embodiment, the vector is derived from an adenovirus. Preferred adenovirus vectors according to the invention are those described by Feldman and Steg (1996) or Ohno et al.(1994). Another preferred recombinant adenovirus according to this specific embodiment of the present invention is the human adenovirus type 2 or 5 (Ad 2 or Ad 5) or an adenovirus of animal origin (French patent application N° FR-93.05954).

Retrovirus vectors and adeno-associated virus vectors are generally understood to be the recombinant gene delivery systems of choice for the transfer of exogenous polynucleotides in vivo, particularly to mammals, including humans. These vectors provide efficient delivery of genes into cells, and the transferred nucleic acids are stably integrated into the chromosomal DNA of the host.

Particularly preferred retroviruses for the preparation or construction of retroviral in vitro or in vitro gene delivery vehicles of the present invention include retroviruses selected from the group consisting of Mink-Cell Focus Inducing Virus, Murine Sarcoma Virus, Reticuloendotheliosis virus and Rous Sarcoma virus. Particularly preferred Murine Leukemia Viruses include the 4070A and the 1504A viruses, Abelson (ATCC No VR-999), Friend (ATCC No VR-245), Gross (ATCC No VR-590), Rauscher (ATCC No VR-998) and Moloney Murine Leukemia Virus (ATCC No VR-190; PCT Application No WO 94/24298). Particularly preferred Rous Sarcoma Viruses include Bryan high titer (ATCC Nos VR-334, VR-657, VR-726, VR-659 and VR-728). Other preferred retroviral vectors are those described in Roth et al.(1996), PCT Application No WO 93/25234, PCT Application No WO 94/06920, Roux et al., 1989, Julan et al., 1992 and Neda et al., 1991.

Yet another viral vector system that is contemplated by the invention comprises the adeno-associated virus (AAV). The adeno-associated virus is a naturally occurring defective virus that requires another virus, such as an adenovirus or a herpes virus, as a helper virus for efficient replication and a productive life cycle (Muzyczka et al., 1992). It is also one of the few viruses that may integrate its DNA into non-dividing cells, and exhibits a high frequency of stable integration (Flotte et al., 1992; Samulski et al., 1989; McLaughlin et al., 1989). One advantageous feature of AAV derives from its reduced efficacy for transducing primary cells relative to transformed cells.

v. BAC Vectors

The bacterial artificial chromosome (BAC) cloning system (Shizuya et al., 1992) has been developed to stably maintain large fragments of genomic DNA (100-300 kb) in E. coli. A preferred BAC vector comprises a pBeloBAC 11 vector that has been described by Kim et al.(1996). BAC libraries are prepared with this vector using size-selected genomic DNA that has been partially digested using enzymes that permit ligation into either the Bam HI or HindIII sites in the vector. Flanking these cloning sites are T7 and SP6 RNA polymerase transcription initiation sites that can be used to generate end probes by either RNA transcription or PCR methods. After the construction of a BAC library in E. coli, BAC DNA is purified from the host cell as a supercoiled circle. Converting these circular molecules into a linear form precedes both size determination and introduction of the BACs into recipient cells. The cloning site is flanked by two Not I sites, permitting cloned segments to be excised from the vector by Not I digestion. Alternatively, the DNA insert contained in the pBeloBAC11 vector may be linearized by treatment of the BAC vector with the commercially available enzyme lambda terminase that leads to the cleavage at the unique cosN site, but this cleavage method results in a full length BAC clone containing both the insert DNA and the BAC sequences.

E. Delivery of the Recombinant Vectors

In order to effect expression of the polynucleotides and polynucleotide constructs of the invention, these constructs must be delivered into a cell. This delivery may be accomplished in vitro, as in laboratory procedures for transforming cell lines, or in vivo or ex vivo, as in the treatment of certain diseases states.

One mechanism is viral infection where the expression construct is encapsulated in an infectious viral particle.

Several non-viral methods for the transfer of polynucleotides into cultured mammalian cells are also contemplated by the present invention, and include, without being limited to, calcium phosphate precipitation (Graham et al., 1973; Chen et al., 1987;), DEAE-dextran (Gopal, 1985), electroporation (Tur-Kaspa et al., 1986; Potter et al., 1984), direct microinjection (Harland et al., 1985), DNA-loaded liposomes (Nicolau et al., 1982; Fraley et al., 1979), and receptor-mediated transfection (Wu and Wu, 1987; 1988). Some of these techniques may be successfully adapted for in vivo or ex vivo use.

Once the expression polynucleotide has been delivered into the cell, it may be stably integrated into the genome of the recipient cell. This integration may be in the cognate location and orientation via homologous recombination (gene replacement) or it may be integrated in a random, non specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle.

One specific embodiment for a method for delivering a protein or peptide to the interior of a cell of a vertebrate in vivo comprises the step of introducing a preparation comprising a physiologically acceptable carrier and a naked polynucleotide operatively coding for the polypeptide of interest into the interstitial space of a tissue comprising the cell, whereby the naked polynucleotide is taken up into the interior of the cell and has a physiological effect. This is particularly applicable for transfer in vitro but it may be applied to in vivo as well.

Compositions for use in vitro and in vivo comprising a “naked” polynucleotide are described in PCT application N° WO 90/11092 (Vical Inc.) and also in PCT application No. WO 95/11307 (Institut Pasteur, INSERM, Universite d'Ottawa) as well as in the articles of Tacson et al.(l 996) and of Huygen et al.(1996).

In still another embodiment of the invention, the transfer of a naked polynucleotide of the invention, including a polynucleotide construct of the invention, into cells may be proceeded with a particle bombarAA4RPnt (biolistic), said particles being DNA-coated microprojectiles accelerated to a high velocity allowing them to pierce cell membranes and enter cells without killing them, such as described by Klein et al.(1987).

In a further embodiment, the polynucleotide of the invention may be entrapped in a liposome (Ghosh and Bacchawat, 1991; Wong et al., 1980; Nicolau et al., 1987) In a specific embodiment, the invention provides a composition for the in vivo production of the AA4RP protein or polypeptide described herein. It comprises a naked polynucleotide operatively coding for this polypeptide, in solution in a physiologically acceptable carrier, and suitable for introduction into a tissue to cause cells of the tissue to express the said protein or polypeptide.

The amount of vector to be injected to the desired host organism varies according to the site of injection. As an indicative dose, it will be injected between 0,1 and 100 μg of the vector in an animal body, preferably a mammal body, for example a mouse body.

In another embodiment of the vector according to the invention, it may be introduced in vitro in a host cell, preferably in a host cell previously harvested from the animal to be treated and more preferably a somatic cell such as a muscle cell. In a subsequent step, the cell that has been transformed with the vector coding for the desired AA4RP polypeptide or the desired fragment thereof is reintroduced into the animal body in order to deliver the recombinant protein within the body either locally or systemically.

XIII. Cell Hosts

Another object of the invention comprises a host cell that has been transformed or transfected with one of the polynucleotides described herein, and in particular a polynucleotide either comprising a AA4RP regulatory polynucleotide or the coding sequence of the AA4RP polypeptide selected from the group consisting of SEQ ID Nos 1, 2 and 4 or a fragment or a variant thereof. Also included are host cells that are transformed (prokaryotic cells) or that are transfected (eukaryotic cells) with a recombinant vector such as one of those described above. More particularly, the cell hosts of the present invention can comprise any of the polynucleotides described in the “Genomic Sequences of The AA4RP Gene” section, the “AA4RP cDNA Sequences” section, the “Coding Regions” section, the “Polynucleotide Constructs” section, and the “Oligonucleotide Probes and Primers” section.

A further recombinant cell host according to the invention comprises a polynucleotide containing a biallelic marker selected from the group consisting of 20-828-311, 17-42-319, 17-41-250, 20-841-149, 20-842-115, and 20-853-415, and the complements thereof.

An additional recombinant cell host according to the invention comprises any of the vectors described herein, more particularly any of the vectors described in the “Recombinant Vectors” section.

Preferred host cells used as recipients for the expression vectors of the invention are the following:

a) Prokaryotic host cells: Escherichia coli strains (I.E.DH5-α strain), Bacillus subtilis, Salmonella typhimurium, and strains from species like Pseudomonas, Streptomyces and Staphylococcus.

b) Eukaryotic host cells: HeLa cells (ATCC N°CCL2; N°CCL2.1; N°CCL2.2), Cv 1 cells (ATCC N°CCL70), COS cells (ATCC N°CRL1650; N°CRL1651), Sf-9 cells (ATCC N°CRL171 1), C127 cells (ATCC N°CRL-1804), 3T3 (ATCC N°CRL-6361), CHO (ATCC N°CCL-61), human kidney 293. (ATCC N° 45504; N° CRL-1573) and BHK (ECACC N° 84100501; N° 84111301).

c) Other Mammalian Host Cells.

The AA4RP gene expression in mammalian, and typically human, cells may be rendered defective, or alternatively it may be proceeded with the insertion of a AA4RP genomic or cDNA sequence with the replacement of the AA4RP gene counterpart in the genome of an animal cell by a AA4RP polynucleotide according to the invention. These genetic alterations may be generated by homologous recombination events using specific DNA constructs that have been previously described.

One kind of cell hosts that may be used are mammal zygotes, such as murine zygotes. For example, murine zygotes may undergo microinjection with a purified DNA molecule of interest, for example a purified DNA molecule that has previously been adjusted to a concentration range from 1 ng/ml—for BAC inserts—3 ng/μl—for P1 bacteriophage inserts—in 10 mM Tris-HCl, pH 7.4, 250 μM EDTA containing 100 mM NaCl, 30 μM spermine, and 70 μM spermidine. When the DNA to be microinjected has a large size, polyamines and high salt concentrations can be used in order to avoid mechanical breakage of this DNA, as described by Schedl et al (1993b).

Anyone of the polynucleotides of the invention, including the DNA constructs described herein, may be introduced in an embryonic stem (ES) cell line, preferably a mouse ES cell line. ES cell lines are derived from pluripotent, uncommitted cells of the inner cell mass of pre-implantation blastocysts. Preferred ES cell lines are the following: ES-E14TG2a (ATCC n° CRL-1821), ES-D3 (ATCC n° CRL1934 and n° CRL-11632), YS001 (ATCC n° CRL-1 1776), 36.5 (ATCC n° CRL-11116). To maintain ES cells in an uncommitted state, they are cultured in the presence of growth inhibited feeder cells which provide the appropriate signals to preserve this embryonic phenotype and serve as a matrix for ES cell adherence. Preferred feeder cells are primary embryonic fibroblasts that are established from tissue of day 13- day 14 embryos of virtually any mouse strain, that are maintained in culture, such as described by Abbondanzo et al.(1993) and are inhibited in growth by irradiation, such as described by Robertson (1987), or by the presence of an inhibitory concentration of LIF, such as described by Pease and Williams (1990).

The constructs in the host cells can be used in a conventional manner to produce the gene product encoded by the recombinant sequence.

Following transformation of a suitable host and growth of the host to an appropriate cell density, the selected promoter is induced by appropriate means, such as temperature shift or chemical induction, and cells are cultivated for an additional period.

Cells are typically harvested by centrifugation, disrupted by physical or chemical means, and the resulting crude extract retained for further purification.

Microbial cells employed in the expression of proteins can be disrupted by any convenient method, including freeze-thaw cycling, sonication, mechanical disruption, or use of cell lysing agents. Such methods are well known by the skill artisan.

The present invention also encompasses primary, secondary, and immortalized homologously recombinant host cells of vertebrate origin, preferably mammalian origin and particularly human origin, that have been engineered to: a) insert exogenous (heterologous) polynucleotides into the endogenous chromosomal DNA of a targeted gene, b) delete endogenous chromosomal DNA, and/or c) replace endogenous chromosomal DNA with exogenous polynucleotides. Insertions, deletions, and/or replacements of polynucleotide sequences may be to the coding sequences of the targeted gene and/or to regulatory regions, such as promoter and enhancer sequences, operably associated with the targeted gene.

The present invention further relates to a method of making a homologously recombinant host cell in vitro or in vivo, wherein the expression of a targeted gene not normally expressed in the cell is altered. Preferably the alteration causes expression of the targeted gene under normal growth conditions or under conditions suitable for producing the polypeptide encoded by the targeted gene. The method comprises the steps of: (a) transfecting the cell in vitro or in vivo with a polynucleotide construct, the a polynucleotide construct comprising; (i) a targeting sequence; (ii) a regulatory sequence and/or a coding sequence; and (iii) an unpaired splice donor site, if necessary, thereby producing a transfected cell; and (b) maintaining the transfected cell in vitro or in vivo under conditions appropriate for homologous recombination.

The present invention further relates to a method of altering the expression of a targeted gene in a cell in vitro or in vivo wherein the gene is not normally expressed in the cell, comprising the steps of: (a) transfecting the cell in vitro or in vivo with a polynucleotide construct, the a polynucleotide construct comprising: (i) a targeting sequence; (ii) a regulatory sequence and/or a coding sequence; and (iii) an unpaired splice donor site, if necessary, thereby producing a transfected cell; and (b) maintaining the transfected cell in vitro or in vivo under conditions appropriate for homologous recombination, thereby producing a homologously recombinant cell; and (c) maintaining the homologously recombinant cell in vitro or in vivo under conditions appropriate for expression of the gene.

The present invention further relates to a method of making a polypeptide of the present invention by altering the expression of a targeted endogenous gene in a cell in vitro or in vivo wherein the gene is not normally expressed in the cell, comprising the steps of: a) transfecting the cell in vitro with a polynucleotide construct, the a polynucleotide construct comprising: (i) a targeting sequence; (ii) a regulatory sequence and/or a coding sequence; and (iii) an unpaired splice donor site, if necessary, thereby producing a transfected cell; (b) maintaining the transfected cell in vitro or in vivo under conditions appropriate for homologous recombination, thereby producing a homologously recombinant cell; and c) maintaining the homologously recombinant cell in vitro or in vivo under conditions appropriate for expression of the gene thereby making the polypeptide.

The present invention further relates to a polynucleotide construct which alters the expression of a targeted gene in a cell type in which the gene is not normally expressed. This occurs when the a polynucleotide construct is inserted into the chromosomal DNA of the target cell, wherein the a polynucleotide construct comprises: a) a targeting sequence; b) a regulatory sequence and/or coding sequence; and c) an unpaired splice-donor site, if necessary. Further included are a polynucleotide constructs, as described above, wherein the construct further comprises a polynucleotide which encodes a polypeptide and is in-frame with the targeted endogenous gene after homologous recombination with chromosomal DNA.

The compositions may be produced, and methods performed, by techniques known in the art, such as those described in U.S. Patent Nos 6,054,288; 6,048,729; 6,048,724; 6,048,524; 5,994,127; 5,968,502; 5,965,125; 5,869,239; 5,817,789; 5,783,385; 5,733,761; 5,641,670; 5,580,734; International Publication Nos:WO96/29411, WO 94/12650; and scientific articles including 1994; Koller et al. (1989) (the disclosures of each of which are incorporated by reference in their entireties).

XIV. Transgenic Animals

The terms “transgenic animals” or “host animals” are used herein designate animals that have their genome genetically and artificially manipulated so as to include one of the nucleic acids according to the invention. Preferred animals are non-human mammals and include those belonging to a genus selected from Mus (e.g. mice), Rattus (e.g. rats) and Oryctogalus (e.g. rabbits) which have their genome artificially and genetically altered by the insertion of a nucleic acid according to the invention. In one embodiment, the invention encompasses non-human host mammals and animals comprising a recombinant vector of the invention or a AA4RP gene disrupted by homologous recombination with a knock out vector.

The transgenic animals of the invention all include within a plurality of their cells a cloned recombinant or synthetic DNA sequence, more specifically one of the purified or isolated nucleic acids comprising a AA4RP coding sequence, a AA4RP regulatory polynucleotide, a polynucleotide construct, or a DNA sequence encoding an antisense polynucleotide such as described in the present specification.

Generally, a transgenic animal according the present invention comprises any one of the polynucleotides, the recombinant vectors and the cell hosts described in the present invention. More particularly, the transgenic animals of the present invention can comprise any of the polynucleotides described in the “Genomic Sequences of the AA4RP Gene” section, the “AA4RP cDNA Sequences” section, the “Coding Regions” section, the “Polynucleotide constructs” section, the “Oligonucleotide Probes and Primers” section, the “Recombinant Vectors” section and the “Cell Hosts” section.

A further transgenic animals according to the invention contains in their somatic cells and/or in their germ line cells a polynucleotide comprising a biallelic marker selected from the group consisting of 20-828-311, 17-42-319, 17-41-250, 20-841-149, 20-842-115, and 20-853-415, and the complements thereof.

In a first preferred embodiment, these transgenic animals may be good experimental models in order to study the diverse pathologies related to cell differentiation, in particular concerning the transgenic animals within the genome of which has been inserted one or several copies of a polynucleotide encoding a native AA4RP protein, or alternatively a mutant AA4RP protein.

In a second preferred embodiment, these transgenic animals may express a desired polypeptide of interest under the control of the regulatory polynucleotides of the AA4RP gene, leading to good yields in the synthesis of this protein of interest, and eventually a tissue specific expression of this protein of interest.

The design of the transgenic animals of the invention may be made according to the conventional techniques well known from the one skilled in the art. For more details regarding the production of transgenic animals, and specifically transgenic mice, it may be referred to U.S. Pat. No. 4,873,191, issued Oct. 10, 1989; U.S. Pat. No. 5,464,764 issued Nov 7, 1995; and U.S. Pat. No. 5,789,215, issued Aug 4, 1998; these documents being herein incorporated by reference to disclose methods producing transgenic mice.

Transgenic animals of the present invention are produced by the application of procedures which result in an animal with a genome that has incorporated exogenous genetic material. The procedure involves obtaining the genetic material, or a portion thereof, which encodes either a AA4RP coding sequence, a AA4RP regulatory polynucleotide or a DNA sequence encoding a AA4RP antisense polynucleotide such as described in the present specification.

A recombinant polynucleotide of the invention is inserted into an embryonic or ES stem cell line. The insertion is preferably made using electroporation, such as described by Thomas et al.(1987). The cells subjected to electroporation are screened (e.g. by selection via selectable markers, by PCR or by Southern blot analysis) to find positive cells which have integrated the exogenous recombinant polynucleotide into their genome, preferably via an homologous recombination event. An illustrative positive-negative selection procedure that may be used according to the invention is described by Mansour et al.(11988).

Then, the positive cells are isolated, cloned and injected into 3.5 days old blastocysts from mice, such as described by Bradley (1987). The blastocysts are then inserted into a female host animal and allowed to grow to term.

Alternatively, the positive ES cells are brought into contact with embryos at the 2.5 days old 8-16 cell stage (morulae) such as described by Wood et al.(1993) or by Nagy et al.(1993), the ES cells being internalized to colonize extensively the blastocyst including the cells which will give rise to the germ line.

The offspring of the female host are tested to determine which animals are transgenic e.g. include the inserted exogenous DNA sequence and which are wild-type.

Thus, the present invention also concerns a transgenic animal containing a nucleic acid, a recombinant expression vector or a recombinant host cell according to the invention.

A. Recombinant Cell Lines Derived from the Transgenic Animals of the Invention

A further object of the invention comprises recombinant host cells obtained from a transgenic animal described herein. In one embodiment the invention encompasses cells derived from non-human host mammals and animals comprising a recombinant vector of the invention or a AA4RP gene disrupted by homologous recombination with a knock out vector.

Recombinant cell lines may be established in vitro from cells obtained from any tissue of a transgenic animal according to the invention, for example by transfection of primary cell cultures with vectors expressing onc-genes such as SV40 large T antigen, as described by Chou (1989) and Shay et al.(1991).

XV. Methods for Screening Substances Interacting with a AA4RP Polypeptide

For the purpose of the present invention, a ligand means a molecule, such as a protein, a peptide, an antibody or any synthetic chemical compound capable of binding to the AA4RP protein or one of its fragments or variants or to modulate the expression of the polynucleotide coding for AA4RP or a fragment or variant thereof.

In the ligand screening method according to the present invention, a biological sample or a defined molecule to be tested as a putative ligand of the AA4RP protein is brought into contact with the corresponding purified AA4RP protein, for example the corresponding purified recombinant AA4RP protein produced by a recombinant cell host as described hereinbefore, in order to form a complex between this protein and the putative ligand molecule to be tested.

As an illustrative example, to study the interaction of the AA4RP protein, or a fragment comprising a contiguous span of at least 6 amino acids, preferably at least 8 to 10 amino acids, more preferably at least 12, 15, 20, 25, 30, 40, 50, or 100 amino acids of SEQ ID No 3, with drugs or small molecules, such as molecules generated through combinatorial chemistry approaches, the microdialysis coupled to HPLC method described by Wang et al. (1997) or the affinity capillary electrophoresis method described by Bush et al. (1997), the disclosures of which are incorporated by reference, can be used.

In further methods, peptides, drugs, fatty acids, lipoproteins, or small molecules which interact with the AA4RP protein, or a fragment comprising a contiguous span of at least 6 amino acids, preferably at least 8 to 10 amino acids, more preferably at least 12, 15, 20, 25, 30, 40, 50, or 100 amino acids of SEQ ID No 3, may be identified using assays such as the following. The molecule to be tested for binding is labeled with a detectable label, such as a fluorescent, radioactive, or enzymatic tag and placed in contact with immobilized AA4RP protein, or a fragment thereof under conditions which permit specific binding to occur. After removal of non-specifically bound molecules, bound molecules are detected using appropriate means.

Another object of the present invention comprises methods and kits for the screening of candidate substances that interact with AA4RP polypeptide.

The present invention pertains to methods for screening substances of interest that interact with a AA4RP protein or one fragment or variant thereof. By their capacity to bind covalently or non-covalently to a AA4RP protein or to a fragment or variant thereof, these substances or molecules may be advantageously used both in vitro and in vivo.

In vitro, said interacting molecules may be used as detection means in order to identify the presence of a AA4RP protein in a sample, preferably a biological sample.

A method for the screening of a candidate substance comprises the following steps:

a) providing a polypeptide comprising, consisting essentially of, or consisting of a AA4RP protein or a fragment comprising a contiguous span of at least 6 amino acids, preferably at least 8 to 10 amino acids, more preferably at least 12, 15, 20, 25, 30, 40, 50, or 100 amino acids of SEQ ID No 3:

b) obtaining a candidate substance;

c) bringing into contact said polypeptide with said candidate substance;

d) detecting the complexes formed between said polypeptide and said candidate substance.

The invention further concerns a kit for the screening of a candidate substance interacting with the AA4RP polypeptide, wherein said kit comprises:

a) a AA4RP protein having an amino acid sequence selected from the group consisting of the amino acid sequences of SEQ ID No 3 or a peptide fragment comprising a contiguous span of at least 6 amino acids, preferably at least 8 to 10 amino acids, more preferably at least 12, 15, 20, 25, 30, 40, 50, or 100 amino acids of SEQ ID No 3;

b) optionally means useful to detect the complex formed between the AA4RP protein or a peptide fragment or a variant thereof and the candidate substance.

In a preferred embodiment of the kit described above, the detection means comprises a monoclonal or polyclonal antibodies directed against the AA4RP protein or a peptide fragment or a variant thereof.

Various candidate substances or molecules can be assayed for interaction with a AA4RP polypeptide. These substances or molecules include, without being limited to, natural or synthetic organic compounds or molecules of biological origin such as polypeptides. When the candidate substance or molecule comprises a polypeptide, this polypeptide may be the resulting expression product of a phage clone belonging to a phage-based random peptide library, or alternatively the polypeptide may be the resulting expression product of a cDNA library cloned in a vector suitable for performing a two-hybrid screening assay.

The invention also pertains to kits useful for performing the hereinbefore described screening method. Preferably, such kits comprise a AA4RP polypeptide or a fragment or a variant thereof, and optionally means useful to detect the complex formed between the AA4RP polypeptide or its fragment or variant and the candidate substance. In a preferred embodiment the detection means comprise a monoclonal or polyclonal antibodies directed against the corresponding AA4RP polypeptide or a fragment or a variant thereof.

A. Candidate Ligands Obtained from Random Peptide Libraries

In a particular embodiment of the screening method, the putative ligand is the expression product of a DNA insert contained in a phage vector (Parmley and Smith, 1988). Specifically, random peptide phages libraries are used. The random DNA inserts encode for peptides of 8 to 20 amino acids in length (Oldenburg K. R. et al., 1992; Valadon P., et al., 1996; Lucas A. H., 1994; Westerink M. A. J., 1995; Felici F. et al., 1991). According to this particular embodiment, the recombinant phages expressing a protein that binds to the immobilized AA4RP protein is retained and the complex formed between the AA4RP protein and the recombinant phage may be subsequently immunoprecipitated by a polyclonal or a monoclonal antibody directed against the AA4RP protein.

Once the ligand library in recombinant phages has been constructed, the phage population is brought into contact with the immobilized AA4RP protein. Then the preparation of complexes is washed in order to remove the non-specifically bound recombinant phages. The phages that bind specifically to the AA4RP protein are then eluted by a buffer (acid pH) or immunoprecipitated by the monoclonal antibody produced by the hybridoma anti-AA4RP, and this phage population is subsequently amplified by an over-infection of bacteria (for example E. coli). The selection step may be repeated several times, preferably 2-4 times, in order to select the more specific recombinant phage clones. The last step comprises characterizing the peptide produced by the selected recombinant phage clones either by expression in infected bacteria and isolation, expressing the phage insert in another host-vector system, or sequencing the insert contained in the selected recombinant phages.

B. Candidate Ligands Obtained by Competition Experiments

Alternatively, peptides, drugs or small molecules which bind to the AA4RP protein, or a fragment comprising a contiguous span of at least 6 amino acids, preferably at least 8 to 10 amino acids, more preferably at least 12, 15, 20, 25, 30, 40, 50, or 100 amino acids of SEQ ID No 3, may be identified in competition experiments. In such assays, the AA4RP protein, or a fragment thereof, is immobilized to a surface, such as a plastic plate. Increasing amounts of the peptides, drugs or small molecules are placed in contact with the immobilized AA4RP protein, or a fragment thereof, in the presence of a detectable labeled known AA4RP protein ligand. For example, the AA4RP ligand may be detectably labeled with a fluorescent, radioactive, or enzymatic tag. The ability of the test molecule to bind the AA4RP protein, or a fragment thereof, is determined by measuring the amount of detectably labeled known ligand bound in the presence of the test molecule. A decrease in the amount of known ligand bound to the AA4RP protein, or a fragment thereof, when the test molecule is present indicated that the test molecule is able to bind to the AA4RP protein, or a fragment thereof.

C. Candidate Ligands Obtained by Affinity Chromatography

Proteins or other molecules interacting with the AA4RP protein, or a fragment comprising a contiguous span of at least 6 amino acids, preferably at least 8 to 10 amino acids, more preferably at least 12, 15, 20, 25, 30, 40, 50, or 100 amino acids of SEQ ID No 3, can also be found using affinity columns which contain the AA4RP protein, or a fragment thereof. The AA4RP protein, or a fragment thereof, may be attached to the column using conventional techniques including chemical coupling to a suitable column matrix such as agarose, Affi Gel®, or other matrices familiar to those of skill in art. In some embodiments of this method, the affinity column contains chimeric proteins in which the AA4RP protein, or a fragment thereof, is fused to glutathion S transferase (GST). A mixture of cellular proteins or pool of expressed proteins as described above is applied to the affinity column. Proteins or other molecules interacting with the AA4RP protein, or a fragment thereof, attached to the column can then be isolated and analyzed on 2-D electrophoresis gel as described in Ramunsen et al. (1997), the disclosure of which is incorporated by reference. Alternatively, the proteins retained on the affinity column can be purified by electrophoresis based methods and sequenced. The same method can be used to isolate antibodies, to screen phage display products, or to screen phage display human antibodies.

D. Candidate Ligands Obtained by Optical Biosensor Methods

Proteins interacting with the AA4RP protein, or a fragment comprising a contiguous span of at least 6 amino acids, preferably at least 8 to 10 amino acids, more preferably at least 12, 15, 20, 25, 30, 40, 50, or 100 amino acids of SEQ ID No 3, can also be screened by using an Optical Biosensor as described in Edwards and Leatherbarrow (1997) and also in Szabo et al. (1995), the disclosure of which is incorporated by reference. This technique permits the detection of interactions between molecules in real time, without the need of labeled molecules. This technique is based on the surface plasmon resonance (SPR) phenomenon. Briefly, the candidate ligand molecule to be tested is attached to a surface (such as a carboxymethyl dextran matrix). A light beam is directed towards the side of the surface that does not contain the sample to be tested and is reflected by said surface. The SPR phenomenon causes a decrease in the intensity of the reflected light with a specific association of angle and wavelength. The binding of candidate ligand molecules cause a change in the refraction index on the surface, which change is detected as a change in the SPR signal. For screening of candidate ligand molecules or substances that are able to interact with the AA4RP protein, or a fragment thereof, the AA4RP protein, or a fragment thereof, is immobilized onto a surface. This surface comprises one side of a cell through which flows the candidate molecule to be assayed. The binding of the candidate molecule on the AA4RP protein, or a fragment thereof, is detected as a change of the SPR signal. The candidate molecules tested may be proteins, peptides, carbohydrates, lipids, or small molecules generated by combinatorial chemistry. This technique may also be performed by immobilizing eukaryotic or prokaryotic cells or lipid vesicles exhibiting an endogenous or a recombinantly expressed AA4RP protein at their surface.

The main advantage of the method is that it allows the determination of the association rate between the AA4RP protein and molecules interacting with the AA4RP protein. It is thus possible to select specifically ligand molecules interacting with the AA4RP protein, or a fragment thereof, through strong or conversely weak association constants.

E. Candidate Ligands Obtained Through a Two-Hybrid Screening Assay

The yeast two-hybrid system is designed to study protein-protein interactions in vivo (Fields and Song, 1989), and relies upon the fusion of a bait protein to the DNA binding domain of the yeast Gal4 protein. This technique is also described in the U.S. Pat. Nos. 5,667,973 and the 5,283,173 (Fields et al.) the technical teachings of both patents being herein incorporated by reference.

The general procedure of library screening by the two-hybrid assay may be performed as described by Harper et al. (1993) or as described by Cho et al. (1998) or also Fromont-Racine et al. (1997).

The bait protein or polypeptide comprises, consists essentially of, or consists of a AA4RP polypeptide or a fragment comprising a contiguous span of at least 6 amino acids, preferably at least 8 to 10 amino acids, more preferably at least 12, 15, 20, 25, 30, 40, 50, or 100 amino acids of SEQ ID No 3.

More precisely, the nucleotide sequence encoding the AA4RP polypeptide or a fragment or variant thereof is fused to a polynucleotide encoding the DNA binding domain of the GAL4 protein, the fused nucleotide sequence being inserted in a suitable expression vector, for example pAS2 or pM3.

Then, a human cDNA library is constructed in a specially designed vector, such that the human cDNA insert is fused to a nucleotide sequence in the vector that encodes the transcriptional domain of the GAL4 protein. Preferably, the vector used is the pACT vector. The polypeptides encoded by the nucleotide inserts of the human cDNA library are termed “pray” polypeptides.

A third vector contains a detectable marker gene, such as beta galactosidase gene or CAT gene that is placed under the control of a regulation sequence that is responsive to the binding of a complete Gal4 protein containing both the transcriptional activation domain and the DNA binding domain. For example, the vector pGSEC may be used.

Two different yeast strains are also used. As an illustrative but non-limiting example the two different yeast strains may be selected from the following:

Y190, the phenotype of which is (MATa, Leu2-3, 112 ura3-12, trp1-901, his3-D200, ade2-101, gal4Dgal180D URA3 GAL-LacZ, LYS GAL-HIS3, cyh′; Y187, the phenotype of which is (MATagal4gal80 his3 trp1-901 ade2-101 ura3-52 leu2-3, -112 URA3 GAL-lacZmet⁻), which is the opposite mating type of Y190.

Briefly, 20 μg of pAS2/AA4RP and 20 μg of pACT-cDNA library are co-transformed into yeast strain Y190. The transformants are selected for growth on minimal media lacking histidine, leucine and tryptophan, but containing the histidine synthesis inhibitor 3-AT (50 mM). Positive colonies are screened for beta galactosidase by filter lift assay. The double positive colonies (His+, beta-gal+) are then grown on plates lacking histidine, leucine, but containing tryptophan and cycloheximide (10 mg/ml) to select for loss of pAS2/AA4RP plasmids bu retention of pACT-cDNA library plasmids. The resulting Y 190 strains are mated with Y187 strains expressing AA4RP or non-related control proteins; such as cyclophilin B, lamin, or SNF1, as Gal4 fusions as described by Harper et al. (1993) and by Bram et al. (Bram R J et al., 1993), and screened for beta galactosidase by filter lift assay. Yeast clones that are beta gal-after mating with the control Gal4 fusions are considered false positives.

In another embodiment of the two-hybrid method according to the invention, interaction between the AA4RP or a fragment or variant thereof with cellular proteins may be assessed using the Matchmaker Two Hybrid System 2 (Catalog No. K1604-1, Clontech). As described in the manual accompanying the Matchmaker Two Hybrid System 2 (Catalog No. K1604-1, Clontech), the disclosure of which is incorporated herein by reference, nucleic acids encoding the AA4RP protein or a portion thereof, are inserted into an expression vector such that they are in frame with DNA encoding the DNA binding domain of the yeast transcriptional activator GAL4. A desired cDNA, preferably human cDNA, is inserted into a second expression vector such that they are in frame with DNA encoding the activation domain of GAL4. The two expression plasmids are transformed into yeast and the yeast are plated on selection medium which selects for expression of selectable markers on each of the expression vectors as well as GAL4 dependent expression of the HIS3 gene. Transformants capable of growing on medium lacking histidine are screened for GAL4 dependent lacZ expression. Those cells which are positive in both the histidine selection and the lacZ assay contain interaction between AA4RP and the protein or peptide encoded by the initially selected cDNA insert.

XVI. Methods for Screening Substances Interacting with the Regulatory Sequences of the AA4RP Gene

The present invention also concerns a method for screening substances or molecules that are able to interact with the regulatory sequences of the AA4RP gene, such as for example promoter or enhancer sequences.

Nucleic acids encoding proteins which are able to interact with the regulatory sequences of the AA4RP gene, more particularly a nucleotide sequence selected from the group consisting of the polynucleotides of the 5′ and 3′ regulatory region or a fragment or variant thereof, and preferably a variant comprising one of the biallelic markers of the invention, may be identified by using a one-hybrid system, such as that described in the booklet enclosed in the Matchmaker One-Hybrid System kit from Clontech (Catalog Ref. no K1603-1), the technical teachings of which are herein incorporated by reference. Briefly, the target nucleotide sequence is cloned upstream of a selectable reporter sequence and the resulting DNA construct is integrated in the yeast genome (Saccharomyces cerevisiae). The yeast cells containing the reporter sequence in their genome are then transformed with a library comprising fusion molecules between cDNAs encoding candidate proteins for binding onto the regulatory sequences of the AA4RP gene and sequences encoding the activator domain of a yeast transcription factor such as GAL4. The recombinant yeast cells are plated in a culture broth for selecting cells expressing the reporter sequence. The recombinant yeast cells thus selected contain a fusion protein that is able to bind onto the target regulatory sequence of the AA4RP gene. Then, the cDNAs encoding the fusion proteins are sequenced and may be cloned into expression or transcription vectors in vitro. The binding of the encoded polypeptides to the target regulatory sequences of the AA4RP gene may be confirmed by techniques familiar to the one skilled in the art, such as gel retardation assays or DNAse protection assays.

Gel retardation assays may also be performed independently in order to screen candidate molecules that are able to interact with the regulatory sequences of the AA4RP gene, such as described by Fried and Crothers (1981), Garner and Revzin (1981) and Dent and Latchman (1993), the teachings of these publications being herein incorporated by reference. These techniques are based on the principle according to which a DNA fragment which is bound to a protein migrates slower than the same unbound DNA fragment. Briefly, the target nucleotide sequence is labeled. Then the labeled target nucleotide sequence is brought into contact with either a total nuclear extract from cells containing transcription factors, or with different candidate molecules to be tested. The interaction between the target regulatory sequence of the AA4RP gene and the candidate molecule or the transcription factor is detected after gel or capillary electrophoresis through a retardation in the migration.

XVII. Method for Screening Ligands That Modulate the Expression of the AA4RP Gene

Another subject of the present invention is a method for screening molecules that modulate the expression of the AA4RP protein. Such a screening method comprises the steps of:

a) cultivating a prokaryotic or an eukaryotic cell that has been transfected with a nucleotide sequence encoding the AA4RP protein or a variant or a fragment thereof, placed under the control of its own promoter;

b) bringing into contact the cultivated cell with a molecule to be tested;

c) quantifying the expression of the AA4RP protein or a variant or a fragment thereof.

In an embodiment, the nucleotide sequence encoding the AA4RP protein or a variant or a fragment thereof consists of an allele of at least one of the biallelic markers 20-828-311, 17-42-319, 17-41-250, 20-841-149, 20-842-115, and 20-853-415, and the complements thereof.

Using DNA recombination techniques well known by the one skill in the art, the AA4RP protein encoding DNA sequence is inserted into an expression vector, downstream from its promoter sequence. As an illustrative example, the promoter sequence of the AA4RP gene is contained in the nucleic acid of the 5′ regulatory region.

The quantification of the expression of the AA4RP protein may be realized either at the mRNA level or at the protein level. In the latter case, polyclonal or monoclonal antibodies may be used to quantify the amounts of the AA4RP protein that have been produced, for example in an ELISA or a RIA assay.

In a preferred embodiment, the quantification of the AA4RP mRNA is realized by a quantitative PCR amplification of the cDNA obtained by a reverse transcription of the total mRNA of the cultivated AA4RP-transfected host cell, using a pair of primers specific for AA4RP.

The present invention also concerns a method for screening substances or molecules that are able to increase, or in contrast to decrease, the level of expression of the AA4RP gene. Such a method may allow the one skilled in the art to select substances exerting a regulating effect on the expression level of the AA4RP gene and which may be useful as active ingredients included in pharmaceutical compositions for treating patients suffering from lipid metabolism related disorders.

Thus, also part of the present invention is a method for screening of a candidate substance or molecule that modulated the expression of the AA4RP gene, this method comprises the following steps:

a) providing a recombinant cell host containing a nucleic acid, wherein said nucleic acid comprises a nucleotide sequence of the 5′ regulatory region or a biologically active fragment or variant thereof located upstream a polynucleotide encoding a detectable protein;

b) obtaining a candidate substance; and

c) determining the ability of the candidate substance to modulate the expression levels of the polynucleotide encoding the detectable protein.

In a further embodiment, the nucleic acid comprising the nucleotide sequence of the 5′ regulatory region or a biologically active fragment or variant thereof also includes a 5 ′UTR region of the AA4RP cDNA of SEQ ID No 2, or one of its biologically active fragments or variants thereof.

Among the preferred polynucleotides encoding a detectable protein, there may be cited polynucleotides encoding beta galactosidase, green fluorescent protein (GFP) and chloramphenicol acetyl transferase (CAT).

The invention also pertains to kits useful for performing the herein described screening method. Preferably, such kits comprise a recombinant vector that allows the expression of a nucleotide sequence of the 5′ regulatory region or a biologically active fragment or variant thereof located upstream and operably linked to a polynucleotide encoding a detectable protein or the AA4RP protein or a fragment or a variant thereof.

In another embodiment of a method for the screening of a candidate substance or molecule that modulates the expression of the AA4RP gene, wherein said method comprises the following steps:

a) providing a recombinant host cell containing a nucleic acid, wherein said nucleic acid comprises a 5′UTR sequence of the AA4RP cDNA of SEQ ID No 2, or one of its biologically active fragments or variants, the 5′UTR sequence or its biologically active fragment or variant being operably linked to a polynucleotide encoding a detectable protein;

b) obtaining a candidate substance; and

c) determining the ability of the candidate substance to modulate the expression levels of the polynucleotide encoding the detectable protein.

In a specific embodiment of the above screening method, the nucleic acid that comprises a nucleotide sequence selected from the group consisting of the 5′UTR sequence of the AA4RP cDNA of SEQ ID No 2 or one of its biologically active fragments or variants, includes a promoter sequence which is endogenous with respect to the AA4RP 5′UTR sequence.

In another specific embodiment of the above screening method, the nucleic acid that comprises a nucleotide sequence selected from the group consisting of the 5′UTR sequence of the AA4RP cDNA of SEQ ID No 2 or one of its biologically active fragments or variants, includes a promoter sequence which is exogenous with respect to the AA4RP 5′UTR sequence defined therein.

In a further preferred embodiment, the nucleic acid comprising the 5′-UTR sequence of the AA4RP cDNA or SEQ ID No 2 or the biologically active fragments thereof includes a biallelic marker selected from the group consisting of 20-828-311, 17-42-319, 17-41-250, 20-841-149, 20-842-115, and 20-853-415, and the complements thereof.

The invention further comprises with a kit for the screening of a candidate substance modulating the expression of the AA4RP gene, wherein said kit comprises a recombinant vector that comprises a nucleic acid including a 5′UTR sequence of the AA4RP cDNA of SEQ ID No 2, or one of their biologically active fragments or variants, the 5′UTR sequence or its biologically active fragment or variant being operably linked to a polynucleotide encoding a detectable protein.

For the design of suitable recombinant vectors useful for performing the screening methods described above, it will be referred to the section of the present specification wherein the preferred recombinant vectors of the invention are detailed.

Expression levels and patterns of AA4RP may be analyzed by solution hybridization with long probes as described in International Patent Application No. WO 97/05277, the entire contents of which are incorporated herein by reference. Briefly, the AA4RP cDNA or the AA4RP genomic DNA described above, or fragments thereof, is inserted at a cloning site immediately downstream of a bacteriophage (T3, T7 or SP6) RNA polymerase promoter to produce antisense RNA. Preferably, the AA4RP insert comprises at least 100 or more consecutive nucleotides of the genomic DNA sequence or the cDNA sequences. The plasmid is linearized and transcribed in the presence of ribonucleotides comprising modified ribonucleotides (i.e. biotin-UTP and DIG-UTP). An excess of this doubly labeled RNA is hybridized in solution with mRNA isolated from cells or tissues of interest. The hybridization is performed under standard stringent conditions (40-50° C. for 16 hours in an 80% formamide, 0.4 M NaCl buffer, pH 7-8). The unhybridized probe is removed by digestion with ribonucleases specific for single-stranded RNA (i.e. RNases CL3, T1, Phy M, U2 or A). The presence of the biotin-UTP modification enables capture of the hybrid on a microtitration plate coated with streptavidin. The presence of the DIG modification enables the hybrid to be detected and quantified by ELISA using an anti-DIG antibody coupled to alkaline phosphatase.

Quantitative analysis of AA4RP gene expression may also be performed using arrays. As used herein, the term array means a one dimensional, two dimensional, or multidimensional arrangement of a plurality of nucleic acids of sufficient length to permit specific detection of expression of mRNAs capable of hybridizing thereto. For example, the arrays may contain a plurality of nucleic acids derived from genes whose expression levels are to be assessed. The arrays may include the AA4RP genomic DNA, the AA4RP cDNA sequences or the sequences complementary thereto or fragments thereof, particularly those comprising at least one of the biallelic markers according the present invention, preferably at least one of the biallelic markers 20-828-311, 17-42-319, 17-41-250, 20-841-149, 20-842-115, and 20-853-415. Preferably, the fragments are at least 15 nucleotides in length. In other embodiments, the fragments are at least 25 nucleotides in length. In some embodiments, the fragments are at least 50 nucleotides in length. More preferably, the fragments are at least 100 nucleotides in length. In another preferred embodiment, the fragments are more than 100 nucleotides in length. In some embodiments the fragments may be more than 500 nucleotides in length.

For example, quantitative analysis of AA4RP gene expression may be performed with a complementary DNA microarray as described by Schena et al.(1995 and 1996). Full length AA4RP cDNAs or fragments thereof are amplified by PCR and arrayed from a 96-well microtiter plate onto silylated microscope slides using high-speed robotics. Printed arrays are incubated in a humid chamber to allow rehydration of the array elements and rinsed, once in 0.2% SDS for 1 min, twice in water for 1 min and once for 5 min in sodium borohydride solution. The arrays are submerged in water for 2 min at 95° C., transferred into 0.2% SDS for 1 min, rinsed twice with water, air dried and stored in the dark at 25° C.

Cell or tissue mRNA is isolated or commercially obtained and probes are prepared by a single round of reverse transcription. Probes are hybridized to 1 cm² microarrays under a 14×14 mm glass coverslip for 6-12 hours at 60° C. Arrays are washed for 5 min at 25° C. in low stringency wash buffer (1× SSC/0.2% SDS), then for 10 min at room temperature in high stringency wash buffer (0.1×SSC/0.2% SDS). Arrays are scanned in 0.1×SSC using a fluorescence laser scanning device fitted with a custom filter set. Accurate differential expression measurements are obtained by taking the average of the ratios of two independent hybridizations.

Quantitative analysis of AA4RP gene expression may also be performed with full length AA4RP cDNAs or fragments thereof in complementary DNA arrays as described by Pietu et al.(1996). The full length AA4RP cDNA or fragments thereof is PCR amplified and spotted on membranes. Then, mRNAs originating from various tissues or cells are labeled with radioactive nucleotides. After hybridization and washing in controlled conditions, the hybridized mRNAs are detected by phospho-imaging or autoradiography. Duplicate experiments are performed and a quantitative analysis of differentially expressed mRNAs is then performed.

Alternatively, expression analysis using the AA4RP genomic DNA, the AA4RP cDNA, or fragments thereof can be done through high density nucleotide arrays as described by Lockhart et al.(1996) and Sosnowsky et al.(1997). Oligonucleotides of 15-50 nucleotides from the sequences of the AA4RP genomic DNA, the AA4RP cDNA sequences particularly those comprising at least one of biallelic markers according the present invention, preferably at least one biallelic marker selected from the group consisting of 20-828-311, 17-42-319, 17-41-250, 20-841-149, 20-842-115, and 20-853-415, or the sequences complementary thereto, are synthesized directly on the chip (Lockhart et al., supra) or synthesized and then addressed to the chip (Sosnowski et al., supra). Preferably, the oligonucleotides are about 20 nucleotides in length.

AA4RP cDNA probes labeled with an appropriate compound, such as biotin, digoxigenin or fluorescent dye, are synthesized from the appropriate mRNA population and then randomly fragmented to an average size of 50 to 100 nucleotides. The said probes are then hybridized to the chip. After washing as described in Lockhart et al., supra and application of different electric fields (Sosnowsky et al., 1997)., the dyes or labeling compounds are detected and quantified. Duplicate hybridizations are performed. Comparative analysis of the intensity of the signal originating from cDNA probes on the same target oligonucleotide in different cDNA samples indicates a differential expression of AA4RP mRNA.

XVIII. Methods for Inhibiting the Expression of a AA4RP Gene

Other therapeutic compositions according to the present invention comprise advantageously an oligonucleotide fragment of the nucleic sequence of AA4RP as an antisense tool or a triple helix tool that inhibits the expression of the corresponding AA4RP gene. A preferred fragment of the nucleic sequence of AA4RP comprises an allele of at least one of the biallelic markers 20-828-311, 17-42-319, 17-41-250, 20-841-149, 20-842-115, and 20-853-415.

A. Antisense Approach

Preferred methods using antisense polynucleotide according to the present invention are the procedures described by Sczakiel et al.(11995).

Preferably, the antisense tools are chosen among the polynucleotides (15-200 bp long) that are complementary to the 5′end of the AA4RP mRNA. In another embodiment, a combination of different antisense polynucleotides complementary to different parts of the desired targeted gene are used.

Preferred antisense polynucleotides according to the present invention are complementary to a sequence of the mRNAs of AA4RP that contains either the translation initiation codon ATG or a splicing donor or acceptor site.

The antisense nucleic acids should have a length and melting temperature sufficient to permit formation of an intracellular duplex having sufficient stability to inhibit the expression of the AA4RP mRNA in the duplex. Strategies for designing antisense nucleic acids suitable for use in gene therapy are disclosed in Green et al., (1986) and Izant and Weintraub, (1984), the disclosures of which are incorporated herein by reference.

In some strategies, antisense molecules are obtained by reversing the orientation of the AA4RP coding region with respect to a promoter so as to transcribe the opposite strand from that which is normally transcribed in the cell. The antisense molecules may be transcribed using in vitro transcription systems such as those which employ T7 or SP6 polymerase to generate the transcript. Another approach involves transcription of AA4RP antisense nucleic acids in vivo by operably linking DNA containing the antisense sequence to a promoter in a suitable expression vector.

Alternatively, suitable antisense strategies are those described by Rossi et al.(11991), in the International Applications Nos. WO 94/23026, WO 95/04141, WO 92/18522 and in the European Patent Application No. EP 0 572 287 A2

An alternative to the antisense technology that is used according to the present invention comprises using ribozymes that will bind to a target sequence via their complementary polynucleotide tail and that will cleave the corresponding RNA by hydrolyzing its target site (namely “hammerhead ribozymes”). Briefly, the simplified cycle of a hammerhead ribozyme comprises (1) sequence specific binding to the target RNA via complementary antisense sequences; (2) site-specific hydrolysis of the cleavable motif of the target strand; and (3) release of cleavage products, which gives rise to another catalytic cycle. Indeed, the use of long-chain antisense polynucleotide (at least 30 bases long) or ribozymes with long antisense arms are advantageous. A preferred delivery system for antisense ribozyme is achieved by covalently linking these antisense ribozymes to lipophilic groups or to use liposomes as a convenient vector. Preferred antisense ribozymes according to the present invention are prepared as described by Sczakiel et al.(1995), the specific preparation procedures being referred to in said article being herein incorporated by reference.

B. Triple Helix Approach

The AA4RP genomic DNA may also be used to inhibit the expression of the AA4RP gene based on intracellular triple helix formation.

Triple helix oligonucleotides are used to inhibit transcription from a genome. They are particularly useful for studying alterations in cell activity when it is associated with a particular gene.

Similarly, a portion of the AA4RP genomic DNA can be used to study the effect of inhibiting AA4RP transcription within a cell. Traditionally, homopurine sequences were considered the most useful for triple helix strategies. However, homopyrimidine sequences can also inhibit gene expression. Such homopyrimidine oligonucleotides bind to the major groove at homopurine:homopyrimidine sequences. Thus, both types of sequences from the AA4RP genomic DNA are contemplated within the scope of this invention.

To carry out gene therapy strategies using the triple helix approach, the sequences of the AA4RP genomic DNA are first scanned to identify 10-mer to 20-mer homopyrimidine or homopurine stretches which could be used in triple-helix based strategies for inhibiting AA4RP expression. Following identification of candidate homopyrimidine or homopurine stretches, their efficiency in inhibiting AA4RP expression is assessed by introducing varying amounts of oligonucleotides containing the candidate sequences into tissue culture cells which express the AA4RP gene.

The oligonucleotides can be introduced into the cells using a variety of methods known to those skilled in the art, including but not limited to calcium phosphate precipitation, DEAE-Dextran, electroporation, liposome-mediated transfection or native uptake.

Treated cells are monitored for altered cell function or reduced AA4RP expression using techniques such as Northern blotting, RNase protection assays, or PCR based strategies to monitor the transcription levels of the AA4RP gene in cells which have been treated with the oligonucleotide.

The oligonucleotides which are effective in inhibiting gene expression in tissue culture cells may then be introduced in vivo using the techniques described above in the antisense approach at a dosage calculated based on the in vitro results, as described in antisense approach.

In some embodiments, the natural (beta) anomers of the oligonucleotide units can be replaced with alpha anomers to render the oligonucleotide more resistant to nucleases. Further, an intercalating agent such as ethidium bromide, or the like, can be attached to the 3′ end of the alpha oligonucleotide to stabilize the triple helix. For information on the generation of oligonucleotides suitable for triple helix formation see Griffin et al.(11989), which is hereby incorporated by this reference.

XIX. Pharmaceutical Compositions of the Invention

The AA4RP polypeptides of the invention can be administered to a mammal, including a human patient, alone or in pharmaceutical compositions where they are mixed with suitable carriers or excipient(s). The pharmaceutical composition is then provided at a therapeutically effective dose. A therapeutically effective dose refers to that amount of AA4RP sufficient to result in amelioration of symptoms of a disease related to lipid metabolism as determined by the methods described herein. A therapeutically effective dose can also refer to the amount of AA4RP necessary for a reduction in weight or a prevention of an increase in weight in persons desiring this affect for aesthetic reasons alone. A therapeutically effective dosage of a AA4RP polypeptide of the invention is that dosage that is adequate to promote weight loss or weight gain with continued periodic use or administration. Techniques for formulation and administration of AA4RP may be found in “Remington's Pharmaceutical Sciences,” Mack Publishing Co., Easton, Pa., latest edition.

Other diseases or disorders that AA4RP could be used to treat or prevent include, but are not limited to, obesity-related atherosclerosis, obesity-related insulin resistance, obesity-related hypertension, microangiopathic lesions resulting from obesity-related Type II diabetes, ocular lesions caused by microangiopathy in obese individuals with Type II diabetes, renal lesions caused by microangiopathy in obese individuals with Type II diabetes, atherosclerosis, cardiovascular disorders such as coronary heart disease, neurodegenerative disorders such as Alzheimer's disease or dementia, coronary artery disease, mitochondriocytopathies, hyperlipidemia, familial combined hyperlipidemia (FCHL) and hypercholesterolemia.

A. Apo A-IV and Related Proteins as a Pharmaceutical Composition

Apo A-IV circulates in the blood, and is therefore easily amenable to therapeutic intervention, by direct administration into the blood of synthetic peptide analogs that mimic its activity or function as competitive antagonists (dominant negatives). Since this protein is involved in fat transport and in cholesterol trafficking within the body and mediates the changes in blood cholesterol in response to dietary changes, interventions targeted at this protein will be useful for cholesterol lowering and anti-atherosclerosis therapeutics, and in the control of diabetes and obesity.

Apolipoprotein A-IV peptides, namely the amino terminal portion of apo A-IV and related proteins, have eating suppressant properties when administered centrally or peripherally. The peptides may be used in compositions and methods for suppressing the appetite and controlling food intake (U.S. Pat. No. 5,840,688).

Apolipoprotein A-IV may serve as a therapeutic agent in the treatment of septic shock, a morbid condition frequently induced by a toxin, the introduction or accumulation of which is most commonly caused by infection or trauma. Among the well described bacterial toxins are the endotoxins or lipopolysaccharides (LPS) of the gram-negative bacteria. A composition of homogeneous particles comprising phospholipids, a lipid exchange protein, and a apolipoprotein such as apo A-IV or a related protein serve as an effective pharmaceutical agent for neutralizing gram-negative endtoxin to prevent or alleviate symptoms of sepsis and septic shock (U.S. Pat. No. 5,932,536).

A therapeutic lipoprotein particle comprising lecithin phospholipids with low phase transition temperatures and human apolipoproteins such as apo A-IV or a related protein may also serve as a therapeutic agent in the treatment of disease conditions associated with elevated serum Lipoprotein(a) levels, as well as hypertension and acute renal failure (U.S. Pat. No. 5, 948, 756).

Means of lowering the plasma levels of cholesterol and low density lipoprotein (LDL) have proved to be effective in the prevention of the vascular coronary pathologies and in the treatment of atheromatous plaques (Steinberg D. (1985)). This risk is a function of both the LDL plasma concentration and LDL qualitative characteristics; the possible modifications of LDL structure and composition can in fact lead to increased formation of atheromatous plaques (Steinberg D. (1989)). Such LDL modifications are the result of oxidative agents present in the plasma and endothelial cells of the arterial wall (Esterbauer H. et al. (1993)). Peptides derived from apo A-IV possess lipid oxidation supressant properties as well as hypolipidaemic properties, in particular they show the capability to prevent and/or delay the oxidative modification of LDL. Therefore, apo A-IV and its derivatives when administered orally or intravenously represent a viable means for treating atherosclerosis and other oxidative disorders (PCT/US99/06580).

B. Routes of Administration

Suitable routes of administration include oral, rectal, transmucosal, or intestinal administration, parenteral delivery, including intramuscular, subcutaneous, intramedullary injections, as well as intrathecal, direct intraventricular, intravenous, intraperitoneal, intranasal or intraocular injections. A particularly useful method of administering compounds for promoting weight loss involves surgical implantation, for example into the abdominal cavity of the recipient, of a device for delivering AA4RP over an extended period of time. Sustained release formulations of the invented medicaments particularly are contemplated.

C. Composition/Formulation

Pharmaceutical compositions and medicaments for use in accordance with the present invention may be formulated in a conventional manner using one or more physiologically acceptable carriers comprising excipients and auxiliaries. Proper formulation is dependent upon the route of administration chosen.

Certain of the medicaments described herein will include a pharmaceutically acceptable carrier and at least one polypeptide that is a AA4RP polypeptide of the invention. For injection, the agents of the invention may be formulated in aqueous solutions, preferably in physiologically compatible buffers such as Hanks's solution, Ringer's solution, or physiological saline buffer such as a phosphate or bicarbonate buffer. For transmucosal administration, penetrants appropriate to the barrier to be permeated are used in the formulation. Such penetrants are generally known in the art.

Pharmaceutical preparations that can be taken orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable gaseous propellant, e.g., carbon dioxide. In the case of a pressurized aerosol the dosage unit may be determined by providing a valve to deliver a metered amount. Capsules and cartridges of, e.g., gelatin, for use in an inhaler or insufflator, may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in aqueous vehicles, and may contain formulatory agents such as suspending, stabilizing and/or dispersing agents.

Pharmaceutical formulations for parenteral administration include aqueous solutions of the active compounds in water-soluble form. Aqueous suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder or lyophilized form for constitution with a suitable vehicle, such as sterile pyrogen-free water, before use.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days.

Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

D. Effective Dosage

Pharmaceutical compositions suitable for use in the present invention include compositions wherein the active ingredients are contained in an effective amount to achieve their intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

For any compound used in the method of the invention, the therapeutically effective dose can be estimated initially from cell culture assays. For example, a dose can be formulated in animal models to achieve a circulating concentration range that includes or encompasses a concentration point or range shown to increase leptin or lipoprotein uptake or binding in an in vitro system. Such information can be used to more accurately determine useful doses in humans.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the LD50, (the dose lethal to 50% of the test population) and the ED50 (the dose therapeutically effective in 50% of the population). The dose ratio between toxic and therapeutic effects is the therapeutic index and it can be expressed as the ratio between LD50 and ED50. Compounds that exhibit high therapeutic indices are preferred.

The data obtained from these cell culture assays and animal studies can be used in formulating a range of dosage for use in human. The dosage of such compounds lies preferably within a range of circulating concentrations that include the ED50, with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, in “The Pharmacological Basis of Therapeutics”, Ch. 1).

Dosage amount and interval may be adjusted individually to provide plasma levels of the active compound which are sufficient to maintain the weight loss or prevention of weight gain effects. Dosages necessary to achieve these effects will depend on individual characteristics and route of administration.

Dosage intervals can also be determined using the value for the minimum effective concentration. Compounds should be administered using a regimen that maintains plasma levels above the minimum effective concentration for 10-90% of the time, preferably between 30-90%; and most preferably between 50-90%. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

A preferred dosage range for the amount of a AA4RP polypeptide of the invention, that can be administered on a daily or regular basis to achieve desired results, including a reduction in levels of circulating plasma triglyceride-rich lipoproteins, range from 0.1-50 mg/kg body mass. A more preferred dosage range is from 0.2-25 mg/kg. A still more preferred dosage range is from 1.0-20 mg/kg, while the most preferred range is from 2.0-10 mg/kg. Of course, these daily dosages can be delivered or administered in small amounts periodically during the course of a day.

XX. Administering a Drug or Treatment Related to the Invention

An embodiment of the present invention is a method of administering a drug or a treatment comprising the steps of: a) obtaining a nucleic acid sample from an individual; b) determining the identity of the polymorphic base of at least one AA4RP-related biallelic marker which is associated with a positive response to the treatment or the drug; or at least one biallelic AA4RP-related biallelic marker which is associated with a negative response to the treatment or the drug; and c) administering the treatment or the drug to the individual if the nucleic acid sample contains said biallelic marker associated with a positive response to the treatment or the drug or if the nucleic acid sample lacks said biallelic marker associated with a negative response to the treatment or the drug. In addition, the methods of the present invention for administering a drug or a treatment encompass methods with any further limitation described in this disclosure, or those following, specified alone or in any combination: optionally, said AA4RP-related biallelic marker may be in a sequence selected individually or in any combination from the group consisting of SEQ ID Nos. 1, 2 and 4, and the complements thereof; or optionally, the administering step comprises administering the drug or the treatment to the individual if the nucleic acid sample contains said biallelic marker associated with a positive response to the treatment or the drug and the nucleic acid sample lacks said biallelic marker associated with a negative response to the treatment or the drug.

An embodiment of the present invention is a method of selecting an individual for inclusion in a clinical trial of a treatment or drug comprising the steps of: a) obtaining a nucleic acid sample from an individual; b) determining the identity of the polymorphic base of at least one AA4RP-related biallelic marker which is associated with a positive response to the treatment or the drug, or at least one AA4RP-related biallelic marker which is associated with a negative response to the treatment or the drug in the nucleic acid sample, and c) including the individual in the clinical trial if the nucleic acid sample contains said AA4RP-related biallelic marker associated with a positive response to the treatment or the drug or if the nucleic acid sample lacks said biallelic marker associated with a negative response to the treatment or the drug. In addition, the methods of the present invention for selecting an individual for inclusion in a clinical trial of a treatment or drug encompass methods with any further limitation described in this disclosure, or those following, specified alone or in any combination: Optionally, said AA4RP-related biallelic marker may be in a sequence selected individually or in any combination from the group consisting of SEQ ID Nos. 1, 2 and 4, and the complements thereof; optionally, the including step comprises administering the drug or the treatment to the individual if the nucleic acid sample contains said biallelic marker associated with a positive response to the treatment or the drug and the nucleic acid sample lacks said biallelic marker associated with a negative response to the treatment or the drug.

XXI. Computer-Related Embodiments

As used herein the term “nucleic acid codes of the invention” encompass the nucleotide sequences comprising, consisting essentially of, or consisting of any one of the following: a) a contiguous span of at least 12, 15, 18, 20, 25, 30, 35, 40, 50, 60, 70, 80, 90, 100, 150, 200, 500, or 1000 nucleotides of SEQ ID No 1 or 4, wherein said contiguous span comprises at least 1, 2, 3, 5, or 10 of the following nucleotide positions of SEQ ID No 1:739-1739; 10946-12958; 13470-13526; 13641-13752; 14271-17969;41718-42718;44942-45942; and 76558-77558; or wherein said contiguous span comprises at least 1, 2, 3, 5, or 10 of the following nucleotide positions of SEQ ID No 4:1-1498; 1613-1724; 2243-3940; and 3941-5381; b) a contiguous span of at least 12, 15, 18, 20, 25, 30, 35, 40, 50, 60, 70, 80, 90, 100, 150, 200, 500, or 1000 nucleotides of SEQ ID No 4 or the complements thereof, wherein said contiguous span comprises one or more of the nucleotides at positions 1241 and 1447; c) a contiguous span of at least 12, 15, 18, 20, 25, 30, 35, 40, 50, 60, 70, 80, 90, 100, 150, 200, 500, or 1000 nucleotides of SEQ ID No 1 or the complements thereof, wherein said contiguous span comprises a T at position 1239, a T at position 12347, a T at position 15241, a G at position 42218, an A at 45442, and a T at 77058; d) a contiguous span of at least 12, 15, 18, 20, 25, 30, 35, 40, 50, 60, 70, 80, 90, 100, 150, 200, 500, or 1000 nucleotides of SEQ ID No 4 or the complements thereof, wherein said contiguous span comprises a T at position 319 and a T at position 3213; e) a contiguous span of at least 12, 15, 18, 20, 25, 30, 35, 40, 50, 60, 70, 80, 90, 100, 150, 200, 500, or 1000 nucleotides of SEQ ID No 2 or the complements thereof, wherein said contiguous span comprises at least 1, 2, 3, 5, or 10 of the following nucleotide positions of SEQ ID No 2:1-1879; f) a contiguous span of at least 12, 15, 18, 20, 25, 30, 35, 40, 50, 60, 70, 80, 90, 100, 150, 200, 500, or 1000 nucleotides of SEQ ID No 2 or the complements thereof, wherein said contiguous span comprises a T at position 1153; and, g) a nucleotide sequence complementary to any one of the preceding nucleotide sequences.

The “nucleic acid codes of the invention” further encompass nucleotide sequences homologous to: a) a contiguous span of at least 12, 15, 18, 20, 25, 30, 35, 40, 50, 60, 70, 80, 90, 100, 150, 200, 500, or 1000 nucleotides of SEQ ID No 1 or 4, wherein said contiguous span comprises at least 1, 2, 3, 5, or 10 of the following nucleotide positions of SEQ ID No 1: 739-1739; 10946-12958; 13470-13526; 13641-13752; 14271-17969; 41718-42718; 44942-45942; and 76558-77558; or wherein said contiguous span comprises at least 1, 2, 3, 5, or 10 of the following nucleotide positions of SEQ ID No 4:1-1498; 1613-1724; 2243-3940; and 3941-5381; b) a contiguous span of at least 12, 15, 18, 20, 25, 30, 35, 40, 50, 60, 70, 80, 90, 100, 150, 200, 500, or 1000 nucleotides of SEQ ID No 2 or the complements thereof, wherein said contiguous span comprises at least 1, 2, 3, 5, or 10 of the following nucleotide positions of SEQ ID No 2:1-1879; and, c) sequences complementary to all of the preceding sequences. Homologous sequences refer to a sequence having at least 99%, 98%, 97%, 96%, 95%, 90%, 85%, 80%, or 75% homology to these contiguous spans. Homology may be determined using any method described herein, including BLAST2N with the default parameters or with any modified parameters. Homologous sequences also may include RNA sequences in which uridines replace the thymines in the nucleic acid codes of the invention. It will be appreciated that the nucleic acid codes of the invention can be represented in the traditional single character format (See the inside back cover of Stryer, Lubert. Biochemistry, 3^{rd} edition. W. H Freeman & Co., New York.) or in any other format or code which records the identity of the nucleotides in a sequence.

As used herein the term “polypeptide codes of the invention” encompass the polypeptide sequences comprising a contiguous span of at least 6, 8, 10, 12, 15, 20, 25, 30, 40, 50, or 100 amino acids of SEQ ID No 3. It will be appreciated that the polypeptide codes of the invention can be represented in the traditional single character format or three letter format (See the inside back cover of Stryer, Lubert. Biochemistry, 3^{rd} edition. W. H Freeman & Co., New York.) or in any other format or code which records the identity of the polypeptides in a sequence.

It will be appreciated by those skilled in the art that the nucleic acid codes of the invention and polypeptide codes of the invention can be stored, recorded, and manipulated on any medium which can be read and accessed by a computer. As used herein, the words “recorded” and “stored” refer to a process for storing information on a computer medium. A skilled artisan can readily adopt any of the presently known methods for recording information on a computer readable medium to generate manufactures comprising one or more of the nucleic acid codes of the invention, or one or more of the polypeptide codes of the invention. Another aspect of the present invention is a computer readable medium having recorded thereon at least 2, 5, 10, 15, 20, 25, 30, or 50 nucleic acid codes of the invention. Another aspect of the present invention is a computer readable medium having recorded thereon at least 2, 5, 10, 15, 20, 25, 30, or 50 polypeptide codes of the invention.

Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of other media known to those skilled in the art.

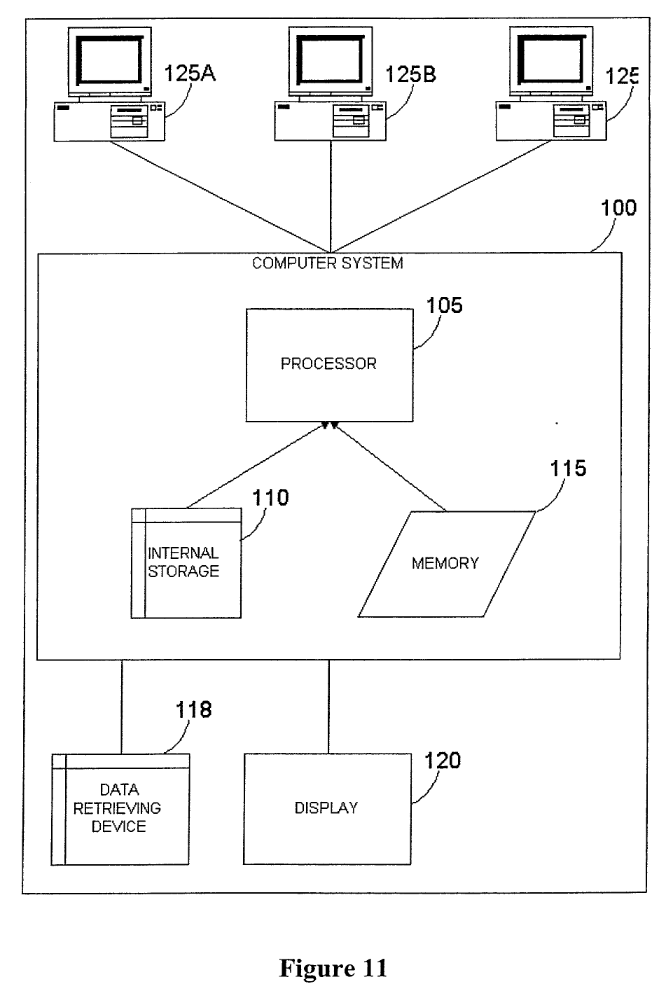

Embodiments of the present invention include systems, particularly computer systems which store and manipulate the sequence information described herein. One example of a computer system 100 is illustrated in block diagram form in FIG. 11. As used herein, “a computer system” refers to the hardware components, software components, and data storage components used to analyze the nucleotide sequences of the nucleic acid codes of the invention or the amino acid sequences of the polypeptide codes of the invention. In one embodiment, the computer system 100 is a Sun Enterprise 1000 server (Sun Microsystems, Palo Alto, Calif.). The computer system 100 preferably includes a processor for processing, accessing and manipulating the sequence data. The processor 105 can be any well-known type of central processing unit, such as the Pentium III from Intel Corporation, or similar processor from Sun, Motorola, Compaq or International Business Machines.

Preferably, the computer system 100 is a general purpose system that comprises the processor 105 and one or more internal data storage components 110 for storing data, and one or more data retrieving devices for retrieving the data stored on the data storage components. A skilled artisan can readily appreciate that any one of the currently available computer systems are suitable.

In one particular embodiment, the computer system 100 includes a processor 105 connected to a bus which is connected to a main memory 115 (preferably implemented as RAM) and one or more internal data storage devices 110, such as a hard drive and/or other computer readable media having data recorded thereon. In some embodiments, the computer system 100 further includes one or more data retrieving device 118 for reading the data stored on the internal data storage devices 110.

The data retrieving device 118 may represent, for example, a floppy disk drive, a compact disk drive, a magnetic tape drive, etc. In some embodiments, the internal data storage device 110 is a removable computer readable medium such as a floppy disk, a compact disk, a magnetic tape, etc. containing control logic and/or data recorded thereon. The computer system 100 may advantageously include or be programmed by appropriate software for reading the control logic and/or the data from the data storage component once inserted in the data retrieving device.

The computer system 100 includes a display 120 which is used to display output to a computer user. It should also be noted that the computer system 100 can be linked to other computer systems 125a-c in a network or wide area network to provide centralized access to the computer system 100.

Software for accessing and processing the nucleotide sequences of the nucleic acid codes of the invention or the amino acid sequences of the polypeptide codes of the invention (such as search tools, compare tools, and modeling tools etc.) may reside in main memory 115 during execution.

In some embodiments, the computer system 100 may further comprise a sequence comparer for comparing the above-described nucleic acid codes of the invention or the polypeptide codes of the invention stored on a computer readable medium to reference nucleotide or polypeptide sequences stored on a computer readable medium. A “sequence comparer” refers to one or more programs which are implemented on the computer system 100 to compare a nucleotide or polypeptide sequence with other nucleotide or polypeptide sequences and/or compounds including but not limited to peptides, peptidomimetics, and chemicals stored within the data storage means. For example, the sequence comparer may compare the nucleotide sequences of nucleic acid codes of the invention or the amino acid sequences of the polypeptide codes of the invention stored on a computer readable medium to reference sequences stored on a computer readable medium to identify homologies, motifs implicated in biological function, or structural motifs. The various sequence comparer programs identified elsewhere in this patent specification are particularly contemplated for use in this aspect of the invention.

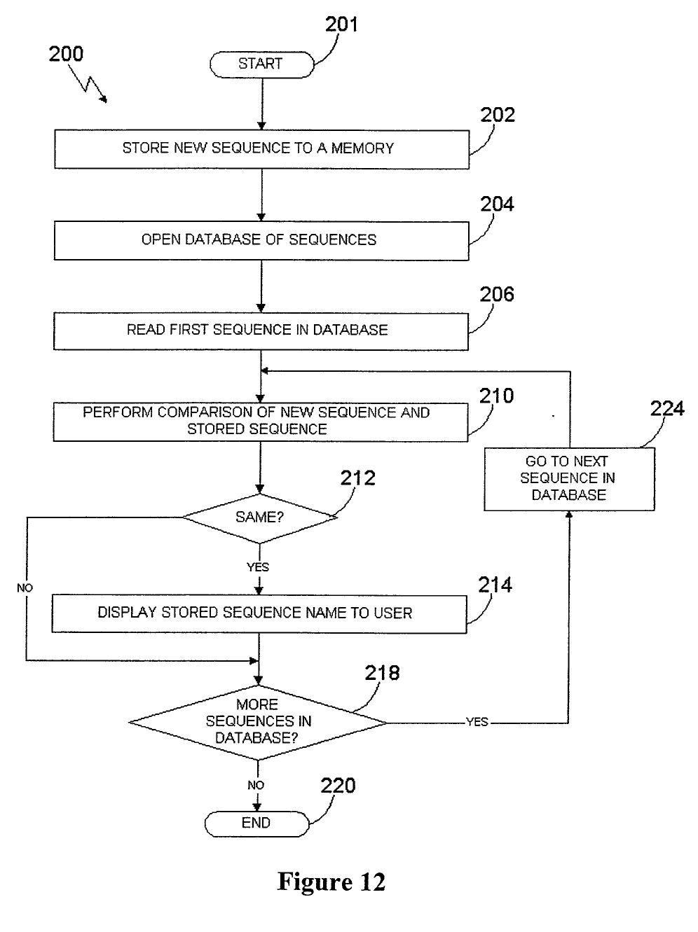

FIG. 12 is a flow diagram illustrating one embodiment of a process 200 for comparing a new nucleotide or protein sequence with a database of sequences in order to determine the homology levels between the new sequence and the sequences in the database. The database of sequences can be a private database stored within the computer system 100, or a public database such as GENBANK, PIR OR SWISSPROT that is available through the Internet.

The process 200 begins at a start state 201 and then moves to a state 202 wherein the new sequence to be compared is stored to a memory in a computer system 100. As discussed above, the memory could be any type of memory, including RAM or an internal storage device.

The process 200 then moves to a state 204 wherein a database of sequences is opened for analysis and comparison. The process 200 then moves to a state 206 wherein the first sequence stored in the database is read into a memory on the computer. A comparison is then performed at a state 210 to determine if the first sequence is the same as the second sequence. It is important to note that this step is not limited to performing an exact comparison between the new sequence and the first sequence in the database. Well-known methods are known to those of skill in the art for comparing two nucleotide or protein sequences, even if they are not identical. For example, gaps can be introduced into one sequence in order to raise the homology level between the two tested sequences. The parameters that control whether gaps or other features are introduced into a sequence during comparison are normally entered by the user of the computer system.

Once a comparison of the two sequences has been performed at the state 210, a determination is made at a decision state 210 whether the two sequences are the same. Of course, the term “same” is not limited to sequences that are absolutely identical. Sequences that are within the homology parameters entered by the user will be marked as “same” in the process 200.

If a determination is made that the two sequences are the same, the process 200 moves to a state 214 wherein the name of the sequence from the database is displayed to the user. This state notifies the user that the sequence with the displayed name fulfills the homology constraints that were entered. Once the name of the stored sequence is displayed to the user, the process 200 moves to a decision state 218 wherein a determination is made whether more sequences exist in the database. If no more sequences exist in the database, then the process 200 terminates at an end state 220. However, if more sequences do exist in the database, then the process 200 moves to a state 224 wherein a pointer is moved to the next sequence in the database so that it can be compared to the new sequence. In this manner, the new sequence is aligned and compared with every sequence in the database.

It should be noted that if a determination had been made at the decision state 212 that the sequences were not homologous, then the process 200 would move immediately to the decision state 218 in order to determine if any other sequences were available in the database for comparison.

Accordingly, one aspect of the present invention is a computer system comprising a processor, a data storage device having stored thereon a nucleic acid code of the invention or a polypeptide code of the invention, a data storage device having retrievably stored thereon reference nucleotide sequences or polypeptide sequences to be compared to the nucleic acid code of the invention or polypeptide code of the invention and a sequence comparer for conducting the comparison. The sequence comparer may indicate a homology level between the sequences compared or identify structural motifs in the nucleic acid code of the invention and polypeptide codes of the invention or it may identify structural motifs in sequences which are compared to these nucleic acid codes and polypeptide codes. In some embodiments, the data storage device may have stored thereon the sequences of at least 2, 5, 10, 15, 20, 25, 30, or 50 of the nucleic acid codes of the invention or polypeptide codes of the invention.

Another aspect of the present invention is a method for determining the level of homology between a nucleic acid code of the invention and a reference nucleotide sequence, comprising the steps of reading the nucleic acid code and the reference nucleotide sequence through the use of a computer program which determines homology levels and determining homology between the nucleic acid code and the reference nucleotide sequence with the computer program. The computer program may be any of a number of computer programs for determining homology levels, including those specifically enumerated herein, including BLAST2N with the default parameters or with any modified parameters. The method may be implemented using the computer systems described above. The method may also be performed by reading 2, 5, 10, 15, 20,25, 30, or 50 of the above described nucleic acid codes of the invention through the use of the computer program and determining homology between the nucleic acid codes and reference nucleotide sequences.

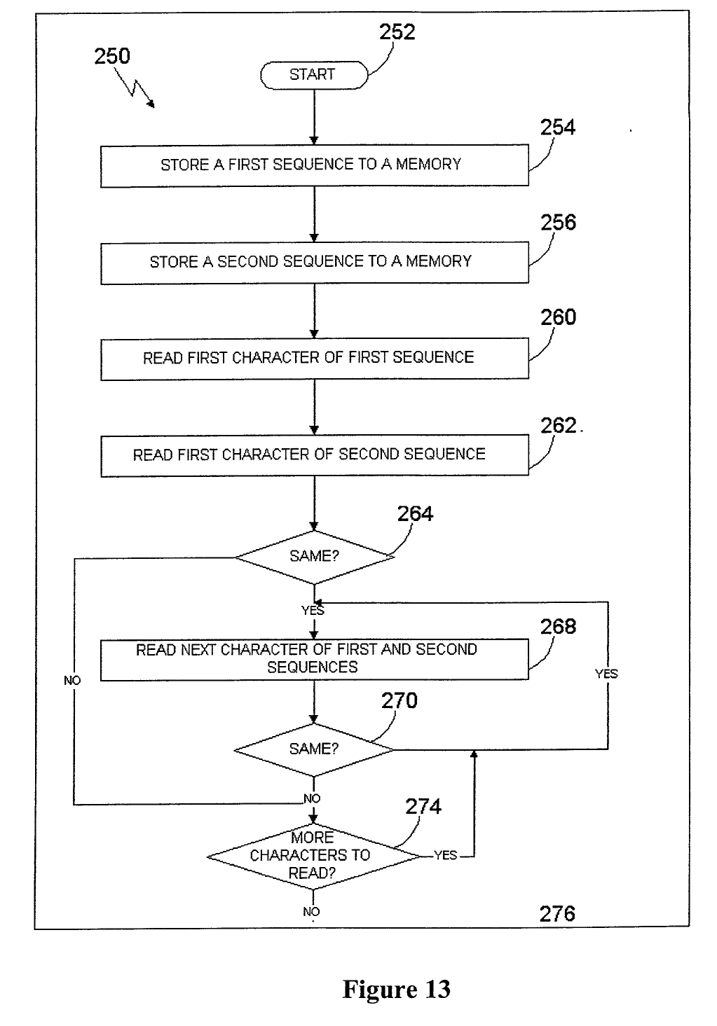

FIG. 13 is a flow diagram illustrating one embodiment of a process 250 in a computer for determining whether two sequences are homologous. The process 250 begins at a start state 252 and then moves to a state 254 wherein a first sequence to be compared is stored to a memory. The second sequence to be compared is then stored to a memory at a state 256. The process 250 then moves to a state 260 wherein the first character in the first sequence is read and then to a state 262 wherein the first character of the second sequence is read. It should be understood that if the sequence is a nucleotide sequence, then the character would normally be either A, T, C, G or U. If the sequence is a protein sequence, then it should be in the single letter amino acid code so that the first and sequence sequences can be easily compared.

A determination is then made at a decision state 264 whether the two characters are the same. If they are the same, then the process 250 moves to a state 268 wherein the next characters in the first and second sequences are read. A determination is then made whether the next characters are the same. If they are, then the process 250 continues this loop until two characters are not the same. If a determination is made that the next two characters are not the same, the process 250 moves to a decision state 274 to determine whether there are any more characters either sequence to read.

If there aren't any more characters to read, then the process 250 moves to a state 276 wherein the level of homology between the first and second sequences is displayed to the user. The level of homology is determined by calculating the proportion of characters between the sequences that were the same out of the total number of sequences in the first sequence. Thus, if every character in a first 100 nucleotide sequence aligned with a every character in a second sequence, the homology level would be 100%.

Alternatively, the computer program may be a computer program which compares the nucleotide sequences of the nucleic acid codes of the present invention, to reference nucleotide sequences in order to determine whether the nucleic acid code of the invention differs from a reference nucleic acid sequence at one or more positions. Optionally such a program records the length and identity of inserted, deleted or substituted nucleotides with respect to the sequence of either the reference polynucleotide or the nucleic acid code of the invention. In one embodiment, the computer program may be a program which determines whether the nucleotide sequences of the nucleic acid codes of the invention contain one or more single nucleotide polymorphisms (SNP) with respect to a reference nucleotide sequence. These single nucleotide polymorphisms may each comprise a single base substitution, insertion, or deletion.

Another aspect of the present invention is a method for determining the level of homology between a polypeptide code of the invention and a reference polypeptide sequence, comprising the steps of reading the polypeptide code of the invention and the reference polypeptide sequence through use of a computer program which determines homology levels and determining homology between the polypeptide code and the reference polypeptide sequence using the computer program.

Accordingly, another aspect of the present invention is a method for determining whether a nucleic acid code of the invention differs at one or more nucleotides from a reference nucleotide sequence comprising the steps of reading the nucleic acid code and the reference nucleotide sequence through use of a computer program which identifies differences between nucleic acid sequences and identifying differences between the nucleic acid code and the reference nucleotide sequence with the computer program. In some embodiments, the computer program is a program which identifies single nucleotide polymorphisms The method may be implemented by the computer systems described above and the method illustrated in FIG. 13. The method may also be performed by reading at least 2, 5, 10, 15, 20, 25, 30, or 50 of the nucleic acid codes of the invention and the reference nucleotide sequences through the use of the computer program and identifying differences between the nucleic acid codes and the reference nucleotide sequences with the computer program.

In other embodiments the computer based system may further comprise an identifier for identifying features within the nucleotide sequences of the nucleic acid codes of the invention or the amino acid sequences of the polypeptide codes of the invention.

An “identifier” refers to one or more programs which identifies certain features within the above-described nucleotide sequences of the nucleic acid codes of the invention or the amino acid sequences of the polypeptide codes of the invention. In one embodiment, the identifier may comprise a program which identifies an open reading frame in the cDNAs codes of the invention.

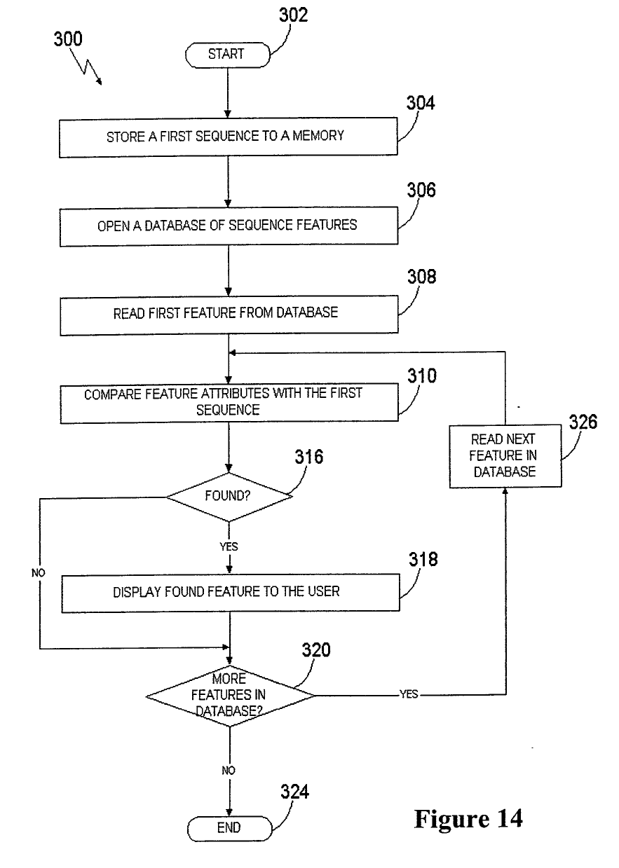

FIG. 14 is a flow diagram illustrating one embodiment of an identifier process 300 for detecting the presence of a feature in a sequence. The process 300 begins at a start state 302 and then moves to a state 304 wherein a first sequence that is to be checked for features is stored to a memory 115 in the computer system 100. The process 300 then moves to a state 306 wherein a database of sequence features is opened. Such a database would include a list of each feature's attributes along with the name of the feature. For example, a feature name could be “Initiation Codon” and the attribute would be “ATG”. Another example would be the feature name “TAATAA Box” and the feature attribute would be “TAATAA”. An example of such a database is produced by the University of Wisconsin Genetics Computer Group (www.gcg.com).

Once the database of features is opened at the state 306, the process 300 moves to a state 308 wherein the first feature is read from the database. A comparison of the attribute of the first feature with the first sequence is then made at a state 310. A determination is then made at a decision state 316 whether the attribute of the feature was found in the first sequence. If the attribute was found, then the process 300 moves to a state 318 wherein the name of the found feature is displayed to the user.

The process 300 then moves to a decision state 320 wherein a determination is made whether move features exist in the database. If no more features do exist, then the process 300 terminates at an end state 324. However, if more features do exist in the database, then the process 300 reads the next sequence feature at a state 326 and loops back to the state 310 wherein the attribute of the next feature is compared against the first sequence.

It should be noted, that if the feature attribute is not found in the first sequence at the decision state 316, the process 300 moves directly to the decision state 320 in order to determine if any more features exist in the database.

In another embodiment, the identifier may comprise a molecular modeling program which determines the 3-dimensional structure of the polypeptides codes of the invention. In some embodiments, the molecular modeling program identifies target sequences that are most compatible with profiles representing the structural environments of the residues in known three-dimensional protein structures. (See, e.g., Eisenberg et al., U.S. Pat. No. 5,436,850 issued Jul. 25, 1995). In another technique, the known three-dimensional structures of proteins in a given family are superimposed to define the structurally conserved regions in that family. This protein modeling technique also uses the known three-dimensional structure of a homologous protein to approximate the structure of the polypeptide codes of the invention. (See e.g., Srinivasan, et al., U.S. Pat. No. 5,557,535 issued Sep. 17, 1996). Conventional homology modeling techniques have been used routinely to build models of proteases and antibodies. (Sowdhamini et al., (1997)). Comparative approaches can also be used to develop three-dimensional protein models when the protein of interest has poor sequence identity to template proteins. In some cases, proteins fold into similar three-dimensional structures despite having very weak sequence identities. For example, the three-dimensional structures of a number of helical cytokines fold in similar three-dimensional topology in spite of weak sequence homology.

The recent development of threading methods now enables the identification of likely folding patterns in a number of situations where the structural relatedness between target and template(s) is not detectable at the sequence level. Hybrid methods, in which fold recognition is performed using Multiple Sequence Threading (MST), structural equivalencies are deduced from the threading output using a distance geometry program DRAGON to construct a low resolution model, and a full-atom representation is constructed using a molecular modeling package such as QUANTA.

According to this 3-step approach, candidate templates are first identified by using the novel fold recognition algorithm MST, which is capable of performing simultaneous threading of multiple aligned sequences onto one or more 3-D structures. In a second step, the structural equivalencies obtained from the MST output are converted into interresidue distance restraints and fed into the distance geometry program DRAGON, together with auxiliary information obtained from secondary structure predictions. The program combines the restraints in an unbiased manner and rapidly generates a large number of low resolution model confirmations. In a third step, these low resolution model confirmations are converted into full-atom models and subjected to energy minimization using the molecular modeling package QUANTA. (See e.g., Aszódi et al., (1997)).

The results of the molecular modeling analysis may then be used in rational drug design techniques to identify agents which modulate the activity of the polypeptide codes of the invention. Accordingly, another aspect of the present invention is a method of identifying a feature within the nucleic acid codes of the invention or the polypeptide codes of the invention comprising reading the nucleic acid code(s) or the polypeptide code(s) through the use of a computer program which identifies features therein and identifying features within the nucleic acid code(s) or polypeptide code(s) with the computer program. In one embodiment, computer program comprises a computer program which identifies open reading frames. In a further embodiment, the computer program identifies structural motifs in a polypeptide sequence. In another embodiment, the computer program comprises a molecular modeling program. The method may be performed by reading a single sequence or at least 2, 5, 10, 15, 20, 25, 30, or 50 of the nucleic acid codes of the invention or the polypeptide codes of the invention through the use of the computer program and identifying features within the nucleic acid codes or polypeptide codes with the computer program.

The nucleic acid codes of the invention or the polypeptide codes of the invention may be stored and manipulated in a variety of data processor programs in a variety of formats. For example, they may be stored as text in a word processing file, such as MicrosoftWORD or WORDPERFECT or as an ASCII file in a variety of database programs familiar to those of skill in the art, such as DB2, SYBASE, or ORACLE. In addition, many computer programs and databases may be used as sequence comparers, identifiers, or sources of reference nucleotide or polypeptide sequences to be compared to the nucleic acid codes of the invention or the polypeptide codes of the invention. The following list is intended not to limit the invention but to provide guidance to programs and databases which are useful with the nucleic acid codes of the invention or the polypeptide codes of the invention. The programs and databases which may be used include, but are not limited to: MacPattern (EMBL), DiscoveryBase (Molecular Applications Group), GeneMine (Molecular Applications Group), Look (Molecular Applications Group), MacLook (Molecular Applications Group), BLAST and BLAST2 (NCBI), BLASTN and BLASTX (Altschul et al, 1990), FASTA (Pearson and Lipman, 1988), FASTDB (Brutlag et al., 1990), Catalyst (Molecular Simulations Inc.), Catalyst/SHAPE (Molecular Simulations Inc.), Cerius².DBAccess (Molecular Simulations Inc.), HypoGen (Molecular Simulations Inc.), Insight II, (Molecular Simulations Inc.), Discover (Molecular Simulations Inc.), CHARMm (Molecular Simulations Inc.), Felix (Molecular Simulations Inc.), DelPhi, (Molecular Simulations Inc.), QuanteMM, (Molecular Simulations Inc.), Homology (Molecular Simulations Inc.), Modeler (Molecular Simulations Inc.), ISIS (Molecular Simulations Inc.), Quanta/Protein Design (Molecular Simulations Inc.), WebLab (Molecular Simulations Inc.), WebLab Diversity Explorer (Molecular Simulations Inc.), Gene Explorer (Molecular Simulations Inc.), SeqFold (Molecular Simulations Inc.), the EMBL/Swissprotein database, the MDL Available Chemicals Directory database, the MDL Drug Data Report data base, the Comprehensive Medicinal Chemistry database, Derwents's World Drug Index database, the BioByteMasterFile database, the Genbank database, and the Genseqn database. Many other programs and data bases would be apparent to one of skill in the art given the present disclosure.

Motifs which may be detected using the above programs include sequences encoding leucine zippers, helix-turn-helix motifs, glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites.

Throughout this application, various publications, patents and published patent applications are cited. The disclosures of these publications, patents and published patent specification referenced in this application are hereby incorporated by reference into the present disclosure to more fully describe the sate of the art to which this invention pertains.

### EXAMPLES

### Example 1

### De Novo Identification of Biallelic Markers

The biallelic markers set forth in this application were isolated from human genomic sequences. To identify biallelic markers, genomic fragments were amplified, sequenced and compared in a plurality of individuals.

DNA Samples

Donors were unrelated and healthy. They represented a sufficient diversity for being representative of a French heterogeneous population. The DNA from 100 individuals was extracted and tested for the de novo identification of biallelic markers.

DNA samples were prepared from peripheral venous blood as follows. Thirty ml of peripheral venous blood were taken from each donor in the presence of EDTA. Cells (pellet) were collected after centrifugation for 10 minutes at 2000 rpm. Red cells were lysed in a lysis solution (50 ml final volume: 10 mM Tris pH 7.6; 5 mM MgCl₂; 10 mM NaCl). The solution was centrifuged (10 minutes, 2000 rpm) as many times as necessary to eliminate the residual red cells present in the supernatant, after resuspension of the pellet in the lysis solution. The pellet of white cells was lysed overnight at 42° C. with 3.7 ml of lysis solution composed of: (a) 3 ml TE 10-2 (Tris-HCl 10 mM, EDTA 2 mM)/NaCl 0.4 M; (b) 200 μl SDS 10%; and (c) 500 μl K-proteinase (2 mg K-proteinase in TE 10-2/NaCl 0.4 M).

For the extraction of proteins, 1 ml saturated NaCl (6M) (1/3.5 v/v) was added. After vigorous agitation, the solution was centrifuged for 20 minutes at 10000 rpm. For the precipitation of DNA, 2 to 3 volumes of 100% ethanol were added to the previous supernatant, and the solution was centrifuged for 30 minutes at 2000 rpm. The DNA solution was rinsed three times with 70% ethanol to eliminate salts, and centrifuged for 20 minutes at 2000 rpm. The pellet was dried at 37° C., and resuspended in 1 ml TE 10-1 or 1 ml water. The DNA concentration was evaluated by measuring the OD at 260 nm (1 unit OD=50 pg/ml DNA). To determine the presence of proteins in the DNA solution, the OD 260/OD 280 ratio was determined. Only DNA preparations having a OD 260/OD 280 ratio between 1.8 and 2 were used in the subsequent examples described below. DNA pools were constituted by mixing equivalent quantities of DNA from each individual.

Amplification of Genomic DNA by PCR

Amplification of specific genomic sequences was carried out on pooled DNA samples obtained as described above.

Amplification Primers

The primers used for the amplification of human genomic DNA fragments were defined with the OSP software (Hillier & Green, 1991). Preferably, primers included, upstream of the specific bases targeted for amplification, a common oligonucleotide tail useful for sequencing. Primers PU contain the following additional PU 5′ sequence: TGTAAAACGACGGCCAGT; primers RP contain the following RP 5′ sequence: CAGGAAACAGCTATGACC. Primers are listed in FIG. 5.

Amplification

PCR assays were performed using the following protocol:

| | | |
|---|---|---|
| Final volume | 25 | μl |
| DNA | 2 | ng/μl |
| MgCl₂ | 2 | mM |
| dNTP (each) | 200 | μM |
| primer (each) | 2.9 | ng/μl |
| Ampli Taq Gold DNA polymerase | 0.05 | unit/μl |
| PCR buffer (10x = 0.1 M TrisHCl | | |
| pH 8.3 0.5 M KCl) 1x | | |

DNA amplification was performed on a Genius II thermocycler. After heating at 94° C. for 10 min, 40 cycles were performed. Cycling times and temperatures were: 30 sec at 94° C., 55° C. for 1 min and 30 sec at 72° C. Holding for 7 min at 72° C. allowed final elongation. The quantities of the amplification products obtained were determined on 96-well microtiter plates, using a fluorometer and Picogreen as intercalant agent (Molecular Probes).

Sequencing of Amplified Genomic DNA and Identification of Biallelic Polymorphisms

Sequencing of the amplified DNA was carried out on ABI 377 sequencers. The sequences of the amplification products were determined using automated dideoxy terminator sequencing reactions with a dye terminator cycle sequencing protocol. The products of the sequencing reactions were run on sequencing gels and the sequences were determined using gel image analysis (ABI Prism DNA Sequencing Analysis software 2.1.2 version).

The sequence data were further evaluated to detect the presence of biallelic markers within the amplified fragments. The polymorphism search was based on the presence of superimposed peaks in the electrophoresis pattern resulting from different bases occurring at the same position. However, the presence of two peaks can be an artifact due to background noise. To exclude such an artifact, the two DNA strands were sequenced and a comparison between the two strands was carried out. In order to be registered as a polymorphic sequence, the polymorphism had to be detected on both strands. Further, biallelic single nucleotide polymorphisms were confirmed by microsequencing as described below.

Biallelic markers were identified in the analyzed fragments and are shown in FIG. 1.

### Example 2

### Genotyping of Biallelic Markers

The biallelic markers identified as described above were further confirmed and their respective frequencies were determined through microsequencing. Microsequencing was carried out on individual DNA samples obtained as described herein.

Microsequencing Primers

Amplification of genomic DNA fragments from individual DNA samples was performed as described in Example 1 using the same set of PCR primers. Microsequencing was carried out on the amplified fragments using specific primers. The preferred primers for use in microsequencing were between 19 and 21 nucleotides in length and hybridized just upstream of the considered polymorphic base. Preferred microsequencing primers are shown in FIG. 4.

The microsequencing reactions were performed as follows: 5 μl of PCR products were added to 5 μl purification mix [2U SAP (Shrimp alkaline phosphate) (Amersham E70092X)); 2U Exonuclease I (Amersham E70073Z); and 1 μl SAP buffer (200 mM Tris-HCl pH 8, 100 mM MgCl₂) in a microtiter plate. The reaction mixture was incubated 30 minutes at 37° C., and denatured 10 minutes at 94° C. afterwards. Twenty μl of microsequencing reaction mixture was added to each well. The microsequencing reaction mixture contained 10 pmol microsequencing oligonucleotide (19mers, GENSET, crude synthesis, 5 OD), 1 U Thermosequenase (Amersham E79000G), 1.25 μl Thermosequenase buffer (260 mM Tris HCl pH 9.5, 65 mM MgCl₂), and the two appropriate fluorescent ddNTPs complementary to the nucleotides at the polymorphic site corresponding to both polymorphic bases (11.25 nM TAMRA-ddTTP; 16.25 nM ROX-ddCTP; 1.675 nM REG-ddATP; 1.25 nM RHO-ddGTP; Perkin Elmer, Dye Terminator Set 401095). After 4 minutes at 94° C., 20 PCR cycles of 15 sec at 55° C., 5 sec at 72° C., and 10 sec at 94° C. were carried out in a

Tetrad PTC-225 thermocycler (MJ Research). The microtiter plate was centrifuged 10 sec at 1500 rpm. The unincorporated dye terminators were removed by precipitation with 19 μl MgCl₂ 2 mM and 55 μl 100% ethanol. After 15 minute incubation at room temperature, the microtiter plate was centrifuged at 3300 rpm 15 minutes at 4° C. After discarding the supernatants, the microplate was evaporated to dryness under 5 reduced pressure (Speed Vac). Samples were resuspended in 2.5 μl formamide EDTA loading buffer and heated for 2 min at 95° C. 0.8 μl microsequencing reaction were loaded on a 10% (19:1) polyacrylamide sequencing gel. The data were collected by an ABI PRISM 377 DNA sequencer and processed using the GENESCAN software (Perkin Elmer).

### Example 3

### Preparation of Antibody Compositions to the AA4RP protein

Substantially pure protein or polypeptide is isolated from transfected or transformed cells containing an expression vector encoding the AA4RP protein or a portion thereof. The concentration of protein in the final preparation is adjusted, for example, by concentration on an Amicon filter device, to the level of a few micrograms/ml. Monoclonal or polyclonal antibody to the protein can then be prepared as follows:

Monoclonal Antibody Production by Hybridoma Fusion

Monoclonal antibody to epitopes in the AA4RP protein or a portion thereof can be prepared from murine hybridomas according to the classical method of Kohler, G. and Milstein, C., (1975) or derivative methods thereof. Also see Harlow, E., and D. Lane. 1988.

Briefly, a mouse is repetitively inoculated with a few micrograms of the AA4RP protein or a portion thereof over a period of a few weeks. The mouse is then sacrificed, and the antibody producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall, (1980), and derivative methods thereof. Selected positive clones can be expanded and their monoclonal antibody product harvested for use. Detailed procedures for monoclonal antibody production are described in Davis, L. et al. Basic Methods in Molecular Biology Elsevier, New York. Section 21-2.

Polyclonal Antibody Production by Immunization

Polyclonal antiserum containing antibodies to heterogeneous epitopes in the AA4RP protein or a portion thereof can be prepared by immunizing suitable non-human animal with the AA4RP protein or a portion thereof, which can be unmodified or modified to enhance immunogenicity. A suitable non-human animal is preferably a non-human mammal is selected, usually a mouse, rat, rabbit, goat, or horse. Alternatively, a crude preparation which has been enriched for AA4RP concentration can be used to generate antibodies. Such proteins, fragments or preparations are introduced into the non-human mammal in the presence of an appropriate adjuvant (e.g. aluminum hydroxide, RIBI, etc.) which is known in the art. In addition the protein, fragment or preparation can be pretreated with an agent which will increase antigenicity, such agents are known in the art and include, for example, methylated bovine serum albumin (mBSA), bovine serum albumin (BSA), Hepatitis B surface antigen, and keyhole limpet hemocyanin (KLH). Serum from the immunized animal is collected, treated and tested according to known procedures. If the serum contains polyclonal antibodies to undesired epitopes, the polyclonal antibodies can be purified by immunoaffinity chromatography.

Effective polyclonal antibody production is affected by many factors related both to the antigen and the host species. Also, host animals vary in response to site of inoculations and dose, with both inadequate or excessive doses of antigen resulting in low titer antisera. Small doses (ng level) of antigen administered at multiple intradermal sites appears to be most reliable. Techniques for producing and processing polyclonal antisera are known in the art, see for example, Mayer and Walker (1987). An effective immunization protocol for rabbits can be found in Vaitukaitis, J. et al. (1971).

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony, 0. et al., (1973). Plateau concentration of antibody is usually in the range of 0.1 to 0.2 mg/ml of serum (about 12 μM). Affinity of the antisera for the antigen is determined by preparing competitive binding curves, as described, for example, by Fisher, D., (1980).

Antibody preparations prepared according to either the monoclonal or the polyclonal protocol are useful in quantitative immunoassays which determine concentrations of antigen-bearing substances in biological samples; they are also used semi-quantitatively or qualitatively to identify the presence of antigen in a biological sample. The antibodies may also be used in therapeutic compositions for killing cells expressing the protein or reducing the levels of the protein in the body.

### Example 4

### Generation of AA4RP by Recombinant Methodology

PCR Cloning

Another approach is to PCR the region of interest from the intact sequence (if cDNA is available) using primers with restriction sites on the end so that PCR products can be directly cloned into vectors of interest. Alternatively, AA4RP can also be generated using RT-PCR to isolate it from tissue RNA.

E. coli Vector

For example, the coding sequence of the aApo A-IV-related DNA can be cloned into pTrcHisB, by putting a Bam HI site on the sense oligo and a Xho I site on the antisense oligo. This allows isolation of the PCR product, digestion of that product, and ligation into the pTrcHisB vector that has also been digested with Bam HI and Xho I. The vector, pTrcHisB, has an N-terminal 6-Histidine tag, that allows purification of the over expressed protein from the lysate using a Nickel resin column. The pTrcHisB vector is used for over-expression of proteins in E. coli.

The following are exemplary PCR conditions.

Final concentrations in the reaction are:

1× PE Biosystems buffer A

1.5 mM MgCl₂

200 uM of each dNTP (dATP, dCTP, dGTP, dTTP)

2.5 Units of Amplitaq Gold from PE Biosystems

0.4uM of each primer (sense and antisense)

10 ng of plasmid template

Cycling parameters:

95C 10 min—1 cycle

95C 30 sec

56C 30 sec

72C 30 sec

repeat above 3 steps for 30 cycles

72C 7 min—1 cycle.

BAC Vector

The coding sequence of the apo A-IV-related DNA can also be over expressed in a Baculovirus system using the 6× His Baculovirus kit (Pharmingen), for example. The coding sequence of the apo A-IV-related DNA is cloned into the appropriate vector using enzymes available in the multiple cloning site. This allows over-expression of the protein in a eukaryotic system which has some advantages over the E. coli system, including: Multiple gene expression, Signal peptide cleavage, Intron splicing, Nuclear transport, Functional protein, Phosphorylation, Glycosylation, and Acylation.

The coding sequence of the apo A-IV-related DNA was amplified by PCR using oligos containing restriction sites for EcoRI or PstI. The resulting DNA product was digested with EcoRI and PstI and subcloned into the baculovirus expression vector pAcHLT (which carries a 6× His tag sequence). The expression vector containing the apo A-IV-related DNA was transfected into Sf9 insect cells by standard procedures (Pharmingen). Recombinant virus was collected, amplified, and used to infect Sf9 cells at a MOI <1. Recombinant protein was recovered and purified over a Ni resin using standard procedures (Pharmingen).

The following are exemplary PCR conditions.

Final concentrations in the reaction are:

1× PE Biosystems buffer A

1.5 mM MgCl₂

200 uM of each dNTP (dATP, dCTP, dGTP, dTTP)

2.5 Units of Amplitaq Gold from PE Biosystems

0.4 uM of each primer (sense and antisense)

10 ng of plasmid template

Cycling parameters:

95C 10 min—1 cycle

95C 30 sec

60C 30 sec

72C 30 sec

repeat above 3 steps for 30 cycles

72C 7 min—1 cycle.

Mammalian Vector

The coding sequence of the apo A-IV-related DNA can also be cloned into a mammalian expression vector and expressed in and purified from mammalian cells. AA4RP is then generated in an environment very close to its endogenous environment. However, this is not necessarily the most efficient way to make protein.

### Example 5

### In Vitro Tests of AA4RP Activity

The activity of various preparations and various sequence variants of AA4RP are assessed using various in vitro assays including those provided below. The system described below invloves the lipolysis stimulated receptor, which has been shown to be important/involved in obesity and diabetes. These assays are also exemplary of those that can be used to develop AA4RP antagonists and agonists. To do that, the effect of AA4RP on lipid metabolism and/or liver regeneration in the presence of the candidate molecules would be compared with the effect of AA4RP on lipid metabolism and/or liver regeneration in the absence of the candidate molecules. Since the inventors found AA4RP is differentially expressed in obese mouse models: up regulated in mice fed a high fat diet (cafeteria diet) and in naturally obese mice (NZO), while it was not differentially expressed in either mice lacking the gene for leptin (ob/ob) or in mice lacking the gene for the leptin receptor (db/db), suggesting AA4RP is regulated by diet, these assays serve to identify candidate treatments for reducing (or increasing) body weight. Specifically, inhibitors of gene expression and antagonists of protein activity that decrease the concentration of AA4RP should serve as important therapeutic compounds in the treatment of lipid metabolism related disorders, while up-regulators of the gene and protein agonists could serve as a means of weight gain for patients.

FACs Analysis of LSR Expression

Tests of the effect of AA4RP on LSR (lipolysis stimulated receptor) can be done using liver cell lines, including for example, PLC, HepG2, Hep3B (human), BPRCL (mouse), or MCA-RH777, MCA-RH8994 (rat).

The effect of AA4RP on LSR can be assessed by measuring the level of LSR expression at the cell surface by flow surface cytometry, using anti-LSR antibodies and fluorescent secondary antibodies. This is a high through-put assay that could be easily adapted to screen AA4RP variants as well as putative agonists or antagonists of AA4RP. An exemplary assay is provided below. The antibody, cell-line and AA4RP analog would vary depending on the experiment, but a human cell-line, human anti-LSR antibody and AA4RP could be used to screen for variants, agonists, and antagonists to be used to treat humans.

Cells are pretreated with AA4RP (or untreated) before harvesting and analysis by FACS. Cells are harvested using non-enzymatic dissociation solution (Sigma), and then are incubated for 1 h at 4° C. with a 1:200 dilution of anti-LSR 81B or an irrelevant anti-serum in PBS containing 1% (w/v) BSA. After washing twice with the same buffer, goat anti-rabbit FITC-conjugated antibody (Rockland, Gilbertsville, Pa.) is added to the cells, followed by a further incubation for 30 min at 4° C. After washing, the cells are fixed in 2% formalin. Flow cytometry analysis is done on a FACSCalibur cytometer (Becton-Dickinson, Franklin Lakes, N.J.).

Effect on LSR as a Lipoprotein Receptor

The effect of AA4RP on the lipoprotein binding, internalizing and degrading activity of LSR can also be tested. Measurement of LSR as lipoprotein receptor is described in Bihain & Yen, 1992 (hereby incorporated herein in its entirety including any drawings, tables, or figures). The effect of AA4RP on the lipoprotein binding, internalizing and degrading activity of LSR (or other receptors) can be assessed using untreated cells as a control. This assay can also be used to screen for active and inhibitory variants of AA4RP, as well as agonists and antagonists of AA4RP activity.

Human liver PLC cells (ATCC Repository) are plated at a density of 300,000 cells/well in 6-well plates (day 0) in DMEM (high glucose) containing glutamine and penicillin-streptomycin (Bihain & Yen, 1992). Media is changed on day 2. On day 3, the confluent monolayers are washed once with phoshphate-buffered saline (PBS, pH 7.4) (2 mL/well). Cells are incubated at 37° C. for 30 min with 10 ng/mL human recombinant leptin in DMEM containing 0.2% (w/v) BSA, 5 mM Hepes, 2 mM CaCl₂, 3.7 g/L sodium bicarbonate, pH 7.5, followed by another 30 min incubation at 37° C. with increasing concentrations of AA4RP. Incubations are continued for 2 h at 37° C. after addition of 0.8 mM oleate and 20 μg/mL ¹²⁵1-LDL. Monolayers are washed 2 times consecutively with PBS containing 0.2% BSA, followed by 1 wash with PBS/BSA, and then 2 times consecutively with PBS. The amounts of oleate-induced binding, uptake and degradation of ¹²⁵I-LDL are measured as previously described (Bihain & Yen, 1992).

This assay could be used to determine the efficiency of the compound (or agonists or antagonists) to increase or decrease LSR activity (or lipoprotein uptake, binding and degradation through other receptors), 5 and thus affect the rate of clearance of triglyceride-rich lipoproteins.

### Example 6

### Effect of AA4RP on Mice Fed a High-Fat Diet

Experiments are performed using approximately 6 week old C57B1/6 mice (8 per group). All mice are housed individually. The mice are maintained on a high fat diet throughout each experiment. The high fat diet (cafeteria diet; D12331 from Research Diets, Inc.) has the following composition: protein kcal % 16, sucrose kcal % 26, and fat kcal % 58. The fat is primarily composed of coconut oil, hydrogenated.

After the mice have been fed a high fat diet for 6 days, micro-osmotic pumps are inserted using isoflurane anesthesia, and are used to provide AA4RP, saline, and an irrelevant peptide to the mice subcutaneously (s.c.) for 18 days. AA4RP is provided at doses of 50, 25, and 2.5 μg/day; and the irrelevant peptide is provided at 10 μg/day. Body weight is measured on the first, third and fifth day of the high fat diet, and then daily after the start of treatment. Final blood samples are taken by cardiac puncture and used to determine triglyceride (TG), total cholesterol (TC), glucose, leptin, and insulin levels. The amount of food consumed per day is also determined for each group.

### Example 7

### Effect of AA4RP on plasma Free Fatty Acid in Mice

The effect of AA4RP on postprandial lipemia (PPL) in normal mice can be tested. The AA4RP used is generated by recombinant methodology as described previously in Example 4.

The mice used in this experiment are fasted for 2 hours prior to the experiment after which a baseline blood sample is taken. All blood samples are taken from the tail using EDTA coated capillary tubes (50 μL each time point). At time 0 (8:30 AM), a standard high fat meal (6 g butter, 6 g sunflower oil, 10 g nonfat dry milk, 10 g sucrose, 12 ml distilled water prepared fresh following Nb#6, JF, pg.1) is given by gavage (vol.=1% of body weight) to all animals.

Immediately following the high fat meal, 25 μg AA4RP is injected i.p. in 100 μL saline. The same dose (25 μg/mL in 100 μL) is again injected at 45 min and at 1 hr 45 min (treated group, n=8). Control animals (n=8) are injected with saline (3×100 μL). Untreated and treated animals are handled in an alternating mode.

Blood samples are taken in hourly intervals, and are immediately put on ice. Plasma is prepared by centrifugation following each time point. Plasma is kept at −20° C. and free fatty acids (FFA), triglycerides (TG) and glucose are determined within 24 hours using standard test kits (Sigma and Wako). If limited amount of plasma is available, glucose is determined in duplicate using pooled samples. For each time point, equal volumes of plasma from all 8 animals per treatment group are pooled.

### Example 8

### Effect of AA4RP on Plasma Leptin and Insulin in Mice

The effect of AA4RP on plasma leptin and insulin levels during postprandial lipemia (PPL) in normal mice can be tested. The experimental procedure is the same as that described in Example 7, except that blood is drawn only at 0, 2 and 4 hours to allow for greater blood samples needed for the determination of leptin and insulin by RIA.

Briefly, 16 mice are fasted for 2 hours prior to the experiment after which a baseline blood sample is taken. All blood samples are taken from the tail using EDTA coated capillary tubes (100 μL each time point). At time 0 (9:00 AM), a standard high fat meal (see Example 7) is given by gavage (vol.=1% of body weight) to all animals. Immediately following the high fat meal, 25 μg AA4RP is injected i.p. in 100 μL saline. The same dose (25 μg in 100 μL) is again injected at 45 min and at 1 hr 45 min (treated group, n=8). Control animals (n=8) are injected with saline (3×100 μL). Untreated and treated animals are handled in an alternating mode.

Blood samples are immediately put on ice and plasma is prepared by centrifugation following each time point. Plasma is kept at −20° C. and free fatty acids (FFA) are determined within 24 hours using a standard test kit (Wako). Leptin and insulin are determined by RIA (ML-82K and SRI-13K, LINCO Research, Inc., St. Charles, Mo.) following the manufacturer's protocol. However, only 20 μL plasma is used. Each determination is done in duplicate. If limited amount of plasma is available, leptin and insulin are determined in 4 pools of 2 animals each in both treatment groups.

While the preferred embodiment of the invention has been illustrated and described, it will be appreciated that various changes can be made therein by the one skilled in the art without departing from the spirit and scope of the invention.

### REFERENCES

Abbondanzo S J et al., 1993, Methods in Enzymology, Academic Press, New York, pp 803-823

Ajioka R. S. et al., Am. J Hum. Genet., 60:1439-1447, 1997

Altschul et al., 1990, J. Mol. Biol. 215(3):403-410

Altschul et al., 1993, Nature Genetics 3:266-272

Altschul et al., 1997, Nuc. Acids Res. 25:3389-3402

Ames, R. S. et al. (1995) J. Immunol. Methods 184:177-186.

Anton M. et al., 1995, J. Virol., 69: 4600-4606

Araki Ketal. (1995) Proc. Natl. Acad. Sci USA. 92(1):160-4.

Ashkenazi, A. et al. (1991) PNAS 88:10535-10539.

Aszódi et al., Proteins:Structure, Function, and Genetics, Supplement 1:38-42 (1997)

Ausubel et al. (1989)Current Protocols in Molecular Biology, Green Publishing Associates and Wiley Interscience, N.Y.

Bartunek, P. et al. (1996) Cytokine 8(1):14-20.

Baubonis W. (1993) Nucleic Acids Res. 21 (9):2025-9.

Beaucage et al., Tetrahedron Lett 1981, 22: 1859-1862

Better, M. et al. (1988) Science 240:1041-1043.

Bradley A., 1987, Production and analysis of chimaeric mice. In: E. J. Robertson (Ed.), Teratocarcinomas and embryonic stem cells: A practical approach. IRL Press, Oxford, pp. 113.

Bram R J et al., 1993, Mol. Cell Biol., 13: 4760-4769

Brinkman U. et al. (1995) J. Immunol. Methods 182:41-50.

Brown E L, Belagaje R, Ryan M J, Khorana H G, Methods Enzymol 1979;68:109-151.

Brutlag et al. Comp. App. Biosci. 6:237-245, 1990.

Burton, D. R. et al. (1994) Advances in Immunology 57:191-280.

Bush et al., 1997, J. Chromatogr., 777: 311-328.

Claros and von Heijne, CABIOS applic. Notes, 10:685-686 (1994).

Carlson, N. G. et al. (1997) J. Biol. Chem. 272(17): 11295-11301.

Chai H. et al. (1993) Biotechnol. Appl. Biochem.18:259-273.

Chee et al. (1996) Science. 274:610-614.

Chen and Kwok Nucleic Acids Research 25:347-353 1997.

Chen et al. (1987) Mol. Cell. Biol. 7:2745-2752.

Chen et al. Proc. Natl. Acad. Sci. USA 94/20 10756-10761,1997.

Chen, Z. et al. (1998) Cancer Res. 58(16):3668-3678.

Cho R J et al., 1998, Proc. Natl. Acad. Sci. USA, 95(7): 3752-3757.

Chou J. Y., 1989, Mol. Endocrinol., 3: 1511-1514.

Clark A. G. (1990) Mol. Biol. Evol. 7:111-122.

Coles R, Caswell R, Rubinsztein D C, Hum Mol Genet 1998;7:791-800.

Compton J. (1991) Nature. 350(6313):91-92.

Davis L. G., M. D. Dibner, and J. F. Battey, Basic Methods in Molecular Biology, ed., Elsevier Press, NY, 1986.

Dempster et al., (1977) J. R. Stat. Soc., 39B:1-38.

Deng, B. et al. (1998) Blood 92(6):1981-1988.

Dent D S & Latchman D S (1993) The DNA mobility shift assay. In: Transcription Factors: A Practical Approach (Latchman D S, ed.) pp 1-26. Oxford: IRL Press

Duverger, N. et al. (1996) Science. 273, 966-968.

Eckner R. et al. (1991) EMBO J. 10:3513-3522.

Edwards et Leatherbarrow, Analytical Biochemistry, 246, 1-6 (1997)

Engvall, E., Meth. Enzymol. 70:419 (1980).

Esterbauer H. et al., Brit. Med. Bull., 49: 566-576, 1993.

Excoffier L. and Slatkin M. (1995) Mol. Biol. Evol., 12(5): 921-927.

Feldman and Steg, 1996, Medecine/Sciences, synthese, 12:47-55.

Felici F., 1991, J. Mol. Biol., Vol. 222:301-310.

Fell, H. P. et al. (1991) J. Immunol. 146:2446-2452.

Fields and Song, 1989, Nature, 340: 245-246.

Fisher, D., Chap. 42 in: Manual of Clinical Immunology, 2d Ed. (Rose and Friedman, Eds.) Amer. Soc. For Microbiol., Washington, D.C. (1980).

Flotte et al. (1992) Am. J. Respir. Cell Mol. Biol. 7:349-356.

Fodor et al. (1991) Science 251:767-777.

Fraley et al. (1979) Proc. Natl. Acad. Sci. USA. 76:3348-3352.

Fried M, Crothers D M, Nucleic Acids Res 1981;9:6505-6525

Fromont-Racine M. et al., 1997, Nature Genetics, 16(3): 277-282.

Fujimoto et al., Am. J Physiol. 262: G1002-G-1006, 1992.

Fuller S. A. et al. (1996) Immunology in Current Protocols in Molecular Biology, Ausubel et al. Eds, John Wiley & Sons, Inc., USA.

Furth P. A. et al. (1994) Proc. Natl. Acad. Sci USA. 91:9302-9306.

Garner M M, Revzin A, Nucleic Acids Res 1981;9:3047-3060

Geysen H. Mario et al. 1984. Proc. Natl. Acad. Sci. U.S.A. 81:3998-4002

Ghosh and Bacchawat, 1991, Targeting of liposomes to hepatocytes, IN: Liver Diseases, Targeted diagnosis and therapy using specific rceptors and ligands. Wu et al. Eds., Marcel Dekeker, New York, pp. 87-104.

Gillies, S. D. et al. (1989) J. Immunol. Methods 125:191-202.

Gillies, S. O. et al. (1992) PNAS 89:1428-1432.

Gonnet et al., 1992, Science 256:1443-1445.

Gopal (1985) Mol. Cell. Biol., 5:1188-1190.

Gordon et al., J. Biol. Chem., 257: 8418-8423, 1982.

Gordon et al., Biochem., 259: 468-474, 1984.

Gossen M. et al. (1992) Proc. Natl. Acad. Sci. USA. 89:5547-5551.

Gossen M. et al. (1995) Science. 268:1766-1769.

Graham et al. (1973) Virology 52:456-457.

Green et al., Ann. Rev. Biochem. 55:569-597 (1986).

Greenspan and Bona, FASEB J. 7(5):437-444 (1989).

Griffm et al. Science 245:967-971 (1989).

Grompe, M. (1993) Nature Genetics. 5:111-117.

Grompe, M. et al. (1989) Proc. Natl. Acad. Sci. U.S.A. 86:5855-5892.

Gu H. et al. (1993) Cell 73:1155-1164.

Gu H. et al. (1994) Science 265:103-106.

Guatelli J C et al. Proc. Natl. Acad. Sci. USA. 35:273-286.

Hacia J G, Brody L C, Chee M S, Fodor S P, Collins F S, Nat Genet 1996;14(4):441-447.

Haff L. A. and Smirnov I. P. (1997) Genome Research, 7:378-388.

Hames B. D. and Higgins S. J. (1985) Nucleic Acid Hybridization: A Practical Approach. Hames and Higgins Ed., IRL Press, Oxford.

Hammerling, et al., in: Monoclonal Antibodies and T-Cell Hybridomas. 563-681 (Elsevier, N.Y., 1981

Harju L, Weber T, Alexandrova L, Lukin M, Ranki M, Jalanko A, Clin Chem 1993;39(11Pt 1):2282-2287.

Harland et al. (1985) J. Cell. Biol. 101:1094-1095.

Harlow, E., and D. Lane. 1988. Antibodies A Laboratory Manual. Cold Spring Harbor Laboratory. pp. 53-242.

Harper J W et al., 1993, Cell, 75: 805-816

Harrop, J. A. et al. (1998) J. Immunol. 161(4):1786-1794.

Hawley M. E. et al. (1994) Am. J. Phys. Anthropol. 18:104.

Hayashi et al., L. Lipid Res., 31: 1613-1625, 1990.

Henikoff and Henikoff, 1993, Proteins 17:49-61

Higgins et al., 1996, Methods Enzymol. 266:383-402

Hillier L. and Green P. Methods Appl., 1991, 1: 124-8.

Hoess et al. (1986) Nucleic Acids Res. 14:2287-2300.

Huston et al. (1991) Methods in Enzymology 203:46-88.

Huang L. et al. (1996) Cancer Res 56(5):1137-1141.

Huygen et al. (1996) Nature Medicine. 2(8):893-898.

Izant J G, Weintraub H, Cell 1984 Apr;36(4):1007-15

Julan et al. (1992) J Gen. Virol. 73:3251-3255.

Kanegae Y. et al., Nuc. Acids Res. 23:3816-3821 (1995).

Karlin and Altschul, 1990, Proc. Natl. Acad. Sci. USA 87:2267-2268

Kettleborough, C. A. et al. (1994) Eur. J. Immunol. 24:952-958.

Khoury J. et al., Fundamentals of Genetic Epidemiology, Oxford University Press, NY, 1993.

Kim U -J. et al. (1996) Genomics 34:213-218.

Klein et al. (1987) Nature. 327:70-73.

Kohler, G. and Milstein, C., Nature 256:495 (1975).

Koller et al., Proc. Natl. Acad. Sci. USA 86:8932-8935 (1989).

Koller et al. (1992) Annu. Rev. Immunol. 10:705-730.

Kostelny, S. A. et al. (1992) J. Immunol. 148:1547-1553.

Kozal M J, Shah N, Shen N, Yang R, Fucini R, Merigan T C, Richman D D, Morris D, Hubbell E, Chee M, Gingeras T R, Nat Med 1996;2(7):753-759.

Lander and Schork, Science, 265, 2037-2048, 1994

Landegren U. et al. (1998) Genome Research, 8:769-776.

Lange K. (1997) Mathematical and Statistical Methods for Genetic Analysis. Springer, New York.

Lenhard T. et al. (1996) Gene. 169:187-190.

Liautard, J. et al. (1997) Cytokinde 9(4):233-241.

Linton M. F. et al. (1993) J. Clin. Invest. 92:3029-3037.

Liu Z. et al. (1994) Proc. Natl. Acad. Sci. USA. 91: 4528-4262.

Livak et al., Nature Genetics, 9:341-342, 1995

Livak K J, Hainer J W, Hum Mutat 1994;3(4):3 79-385

Lockhart et al. Nature Biotechnology 14: 1675-1680, 1996

Lucas A. H., 1994, In: Development and Clinical Uses of Haempophilus b Conjugate.

Mansour S. L. et al. (1988) Nature. 336:348-352.

Marshall R. L. et al. (1994) PCR Methods and Applications. 4:80-84.

McCormick et al. (1994) Genet. Anal. Tech. Appl. 11: 158-164.

McLaughlin B. A. et al. (1996) Am. J Hum. Genet. 59:561-569.

Morrison, Science 229:1202 (1985).

Morton N. E., Am. J Hum. Genet., 7:277-318, 1955

Muller, Y. A. et al. (1998) Structure 6(9): 1153-1167;

Mullinax, R. L. et al. (1992) BioTechniques 12(6):864-869.

Muzyczka et al. (1992) Curr. Topics in Micro. and Immunol. 158:97-129.

Nada S. et al. (1993) Cell 73:1125-1135.

Nagy A. et al., 1993, Proc. Natl. Acad. Sci. USA, 90: 8424-8428.

Naramura, M. et al. (1994) Immunol. Lett. 39:91-99.

Narang S A, Hsiung H M, Brousseau R, Methods Enzymol 1979;68:90-98

Neda et al. (1991) J. Biol. Chem. 266:14143-14146.

Newton et al. (1989) Nucleic Acids Res. 17:2503-2516.

Nickerson D. A. et al. (1990) Proc. Natl. Acad. Sci U.S.A. 87:8923-8927.

Nicolau C. et al., 1987, Methods Enzymol., 149:157-76.

Nicolau et al. (1982) Biochim. Biophys. Acta. 721:185-190.

Nissinoff, J. Immunol. 147(8):2429-243 8 (1991).

Noma, A., et al., Atherosclerosis 49:1, 1983; Illingworth, D. and Conner, W., Endocrinology & Metabolism, McGraw-Hill, New York 1987.

Nyren P, Pettersson B, Uhlen M, Anal Biochem 1993;208(1):171-175

O'Reilly et al. (1992) Baculovirus Expression Vectors: A Laboratory Manual. W. H. Freeman and Co., New York.

Ochoa A, Bovard-Houppermans S, Zakin M M. Biochim Biophys Acta. 1993 Dec 2;1210(1):41-7.

Ohno et al. (1994) Science. 265:781-784.

Oi et al., BioTechniques 4:214 (1986).

Oldenburg K. R. et al., 1992, Proc. Natl. Acad. Sci., 89:5393-5397.

Orita et al. (1989) Proc. Natl. Acad. Sci. U.S.A. 86:2776-2770.

Ott J., Analysis of Human Genetic Linkage, John Hopkins University Press, Baltimore, 1991

Ouchterlony, O. et al., Chap. 19 in: Handbook of Experimental Immunology D. Wier (ed) Blackwell (1973)

Padlan E. A., (1991) Molecular Immunology 28(4/5):489-498.

Parmley and Smith, Gene, 1988, 73:305-318

Pastinen et al., Genome Research 1997; 7:606-614

Pearson and Lipman, 1988, Proc. Natl. Acad. Sci. USA 85(8):2444-2448

Pease S. ans William R. S., 1990, Exp. Cell. Res., 190: 209-211.

Perlin et al. (1994) Am. J Hum. Genet. 55:777-787.

Persic, L. et al. (1997) Gene 187 9-18.

Peterson et al., 1993, Proc. Natl. Acad. Sci. USA, 90: 7593-7597.

Pietu et al. Genome Research 6:492-503, 1996

Pitard, V. et al. (1997) J. Immunol. Methods 205(2):177-190.

Potter et al. (1984) Proc. Natl. Acad. Sci U.S.A. 81(22):7161-7165.

Prat, M. et al. (1998) J. Cell. Sci. 111 (Pt2):237-247.

Ramunsen et al., 1997, Electrophoresis, 18: 588-598.

Reid L. H. et al. (1990) Proc. Natl. Acad. Sci. U.S.A. 87:4299-4303.

Rewers M. et al., (1994) Diabetes 43 (12):1485-1489.

Risch, N. and Merikangas, K. (Science, 273:1516-1517, 1996

Robertson E., 1987, Embryo-derived stem cell lines. In: E. J. Robertson Ed. Teratocarcinomas and embrionic stem cells: a practical approach. IRL Press, Oxford, pp. 71.

Roguska M. A. et al. (1994) PNAS 91:969-973).

Rossi et al., Pharmacol. Ther. 50:245-254, (1991)

Roth J. A. et al. (1996) Nature Medicine. 2(9):985-991.

Roux et al. (1989) Proc. Natl. Acad. Sci. U.S.A. 86:9079-9083.

Ruano et al. (1990) Proc. Natl. Acad. Sci. U.S.A. 87:6296-6300.

Sambrook, J., Fritsch, E. F., and T. Maniatis. (1989) Molecular Cloning: A Laboratory Manual. 2ed. Cold Spring Harbor Laboratory, Cold Spring Harbor, N.Y.

Samson M, et al. (1996) Nature, 382(6593):722-725.

Samulskietal. (1989) J. Virol. 63:3822-3828.

Sanchez-PescadorR. (1988) J. Clin. Microbiol. 26(10):1934-1938.

Sarkar, G. and Sommer S. S. (1991) Biotechniques.

Sauer B. et al. (1988) Proc. Natl. Acad. Sci. U.S.A. 85:5166-5170.

Sawai, H. et al. (1995) AJRI 34:26-34.

Schaid D. J. et al., Genet. Epidemiol.,13:423-450, 1996

Schedl A. et al., 1993a, Nature, 362: 258-261.

Schedl et al., 1993b, Nucleic Acids Res., 21: 4783-4787.

Schena et al. Science 270:467-470, 1995

Schena et al., 1996, Proc Natl Acad Sci U S A,.93(20):10614-10619.

Schneider et al.(1997) Arlequin: A Software For Population Genetics Data Analysis. University of Geneva.

Schwartz and Dayhoff, eds., 1978, Matrices for Detecting Distance Relationships: Atlas of Protein Sequence and Structure, Washington: National Biomedical Research Foundation

Sczakiel G. et al. (1995) Trends Microbiol. 3(6):213-217.

Shay J. W. et al., 1991, Biochem. Biophys. Acta, 1072:1-7.

Sheffield, V. C. et al. (1991) Proc. Natl. Acad. Sci. U.S.A. 49:699-706.

Shizuya et al. (1992) Proc. Natl. Acad. Sci. U.S.A. 89:8794-8797.

Shoemaker D D, et al., Nat Genet 1996;14(4):450-456 Shu, L. et al. (1993) PNAS 90:7995-7999.

Simonet W. S. et al., 1993, Ochoa A. et al., 1993, Elshourbagy N. A. et al., 1987.

Skerra, A. et al. (1988) Science 240:1038-1040.

Smith (1957) Ann. Hum. Genet. 21:254-276.

Smith et al. (1983) Mol. Cell. Biol. 3:2156-2165.

Sosnowski R G, et al., Proc Natl Acad Sci USA 1997;94:1119-1123

Sowdhamini et al., Protein Engineering 10:207, 215 (1997)

Spielmann S. and Ewens W. J., Am. J Hum. Genet., 62:450-458, 1998

Spielmann S. et al., Am. J Hum. Genet., 52:506-516, 1993

Steinberg D., J.A.M.A., 253: 2080-2086, 1985.

Steinberg D., New Eng. J. Med., 320: 915-924, 1989.

Sternberg N. L. (1992) Trends Genet. 8:1-16.

Stemberg N. L. (1994) Mamm. Genome. 5:397-404.

Stryer, L., Biochemistry, 4th edition, 1995

Studnicka G. M. et al. (1994) Protein Engineering 7(6):805-814.

Swaney et al., Biochemistry 6: 271-279, 1977.

Swaney et al., Biochemistry 6: 271-279, 1977; Sherman et al., Gastroenterology 95: 394-401, 1988

Syvanen A C, Clin Chim Acta 1994;226(2):225-236

Szabo A. et al. Curr Opin Struct Biol 5, 699-705 (1995)

Tacson et al. (1996) Nature Medicine. 2(8):888-892.

Taryman, R. E. et al. (1995) Neuron 14(4):755-762.

Te Riele et al. (1990) Nature. 348:649-651.

Terwilliger J. D. and Ott J., Handbook of Human Genetic Linkage, John Hopkins University Press, London, 1994

Thomas K. R. et al. (1986) Cell. 44:419-428.

Thomas K. R. et al. (1987) Cell. 51:503-512.

Thompson et al., 1994, Nucleic Acids Res. 22(2):4673-4680

Tur-Kaspa et al. (1986) Mol. Cell. Biol. 6:716-718.

Tutt, A. et al. (1991) J. Immunol. 147:60-69

Tyagi et al. (1998) Nature Biotechnology. 16:49-53.

Urdea M. S. (1988) Nucleic Acids Research. 11:4937-4957.

Urdea M. S. et al.(1991)Nucleic Acids Symp. Ser. 24:197-200.

Vaitukaitis, J. et al. J. Clin. Endocrinol. Metab. 33:988-991(1971)

Valadon P., et al., 1996, J. Mol. Biol., 261:11-22.

Van der Lugt et al. (1991) Gene. 105:263-267.

Verges B. (1995) Diabete Metab 21 (2): 99-105.

Vil, H. et al. (1992) PNAS 89:11337-11341.

Vlasak R. et al. (1983) Eur. J. Biochem. 135:123-126.

Wabiko et al. (1986) DNA.5(4):305-314.

Walker et al. (1996) Clin. Chem. 42:9-13.

Wang et al., 1997, Chromatographia, 44: 205-208.

Wang Z. et al., 1995, Chung Hua Ping Li Hsueh Tsa Chih 24(1):8-10.

Weir, B. S. (1996) Genetic data Analysis II: Methods for Discrete population genetic Data, Sinauer Assoc., Inc., Sunderland, Mass., U.S.A.

Westerink M. A. J., 1995, Proc. Natl. Acad. Sci., 92:4021-4025

White, M. B. et al. (1992) Genomics. 12:301-306.

White, M. B. et al. (1997) Genomics. 12:301-306.

Wong et al. (1980) Gene. 10:87-94.

Wood S. A. et al., 1993, Proc. Natl. Acad. Sci. USA, 90: 4582-4585.

Wu and Wu (1987) J. Biol. Chem. 262:4429-4432.

Wu and Wu (1988) Biochemistry. 27:887-892.

Wu et al. (1989) Proc. Natl. Acad. Sci. U.S.A. 86:2757.

Wu et al. (1997) Biochem. And Biophys. Res. Comm. 232:817-821.

Yagi T. et al. (1990) Proc. Natl. Acad. Sci. U.S.A. 87:9918-9922.

Yoon, D. Y. et al. (1998) J. Immunol. 160(7):3170-3179.

Zhao et al., Am. J. Hum. Genet., 63:225-240, 1998.

Zheng, X. X. et al. (1995) J. Immunol. 154:5590-5600.

Zhu, Z. et al. (1998) Cancer Res. 58(15):3209-3214.

Zou Y. R. et al. (1994) Curr. Biol. 4:1099-1103.

What is claimed is:

1. An isolated, purified, or recombinant polynucleotide comprising a contiguous span of 8 to 50 nucleotides of any one of SEQ ID Nos 1, 2, 4 or the complement thereof, wherein said span includes a AA4RP-related biallelic marker in said sequence.

2. The isolated, purified, or recombinant polynucleotide of claim 1, wherein said polynucleotide is selected from the group consisting of:
(a) an isolated, purified, or recombinant polynucleotide comprising a contiguous span of at least 12 nucleotides of SEQ ID No 1 or the complements thereof, wherein said contiguous span comprises at least one of the nucleotide positions of SEQ ID No 1 selected from the group consisting of a T at position 1239, a T at position 12347, a C at position 13269, an A at position 13475, a T at position 15241, a G at position 42218, an A at position 45442, and a T at position 77058;
(b) an isolated, purified, or recombinant polynucleotide comprising a contiguous span of at least 12 nucleotides of SEQ ID No 1 or the complements thereof, wherein said contiguous span comprises at least 10 consecutive nucleotides of at least one of the nucleotide positions of SEQ ID No 1, wherein said positions are selected from the group consisting of 12947 to 12958, 13470 to 13526, 13641 to 13752, and 14271 to 15968;
(c) an isolated, purified, or recombinant polynucleotide comprising a contiguous span of at least 12 nucleotides of SEQ ID No 4 or the complements thereof, wherein said contiguous span comprises at least one of the nucleotide positions of SEQ ID No 4 selected from the group consisting of a T at position 319, a C at position 1241, an A at position 1447, and a T at position 3213;
(d) an isolated, purified, or recombinant polynucleotide comprising a contiguous span of at least 12 nucleotides of SEQ ID No 4 or the complements thereof, wherein said contiguous span comprises at least 10 consecutive nucleotides of at least one of the nucleotide positions of SEQ ID No 4, wherein said positions are selected from the group consisting of 919 to 930, 1442 to 1498, 1613 to 1724 and 2243 to 3940;
(e) an isolated, purified, or recombinant polynucleotide comprising a contiguous span of at least 12 nucleotides of SEQ ID No 2 or the complements thereof;
(f) a polynucleotide according to (e), wherein said contiguous span comprises a T at position 1153;
(g) a polynucleotide according to (f) wherein said contiguous span comprises at least 10 consecutive nucleotides selected within positions 21-1121;
(h) an isolated, purified, or recombinant polynucleotide wherein said contiguous span is 18 to 35 nucleotides in length and said biallelic marker is within 4 nucleotides of the center of said polynucleotide;
(i) a polynucleotide according to (h), wherein said polynucleotide consists of said contiguous span and said contiguous span is 25 nucleotides in length and said biallelic marker is at the center of said polynucleotide; and
(j) an isolated, purified, or recombinant polynucleotide, wherein the 3′ end of said contiguous span is located at the 3′ end of said polynucleotide and said biallelic marker is present at the 3′ end of said polynucleotide.

3. A recombinant vector comprising a polynucleotide of claim 1.

4. A host cell comprising a recombinant vector according to claim 3.

5. A non-human host animal or mammal comprising a recombinant vector according to claim 4.

6. A non-human host animal or mammal comprising an AA4RP gene disrupted by homologous recombination with a knock out vector, wherein said vector comprises a polynucleotide of claim 1.

7. A method of genotyping comprising determining the identity of a nucleotide at a AA4RP-related biallelic marker or the complement thereof in a biological sample.

8. A method according to claim 7, further comprising amplifying a portion of said sequence comprising the biallelic marker prior to said determining step.

9. A method according to claim 7, wherein said determining is performed by an assay selected from the group consisting of a hybridization assay, a sequencing assay, a microsequencing assay, and an enzyme-based mismatch detection assay.

10. A method of estimating the frequency of at least one allele of at least one AA4RP-related biallelic marker in at least one population comprising:
a) genotyping individuals from said population for said biallelc marker according to the method of claim 7; and
b) determining the proportional representation of said biallelic marker in said at least one population.

11. A method of detecting an association between a genotype and a trait, comprising the steps of:
(a) determining the frequency of at least one AA4RP-related biallelic marker in trait positive population according to the method of claim 10;
(b) determining the frequency of at least one AA4RP-related biallelic marker in a control population according to the method of claim 10; and
(c) determining whether a statistically significant association exists between said genotype and said trait.

12. A method of estimating the frequency of a haplotype for a set of biallelic markers in at least one population, comprising:
(a) genotyping at least one AA4RP-related biallelic marker according to claim 7 for each individual in said at least one population;
(b) genotyping a second biallelic marker by determining the identity of the nucleotides at said second biallelic marker for both copies of said second biallelic marker present in the genome of each individual in said at least one population; and
(c) applying a haplotype determination method to the identities of the nucleotides determined in steps (a) and (b) to obtain an estimate of said frequency.

13. A method according to claim 12, wherein said haplotype determination method is selected from the group consisting of asymmetric PCR amplification, double PCR amplification of specific alleles, the Clark algorithm, or an expectation-maximization algorithm.

14. A method of detecting an association between a haplotype and a trait, comprising the steps of:
(a) estimating the frequency of at least one haplotype in a trait positive population according to the method of claim 13;
(b) estimating said frequency of said haplotype in said control population according to the method of claim 13; and
(c) determining whether a statistically significant association exists between said haplotype and said trait.

15. An isolated, purified, or recombinant polynucleotide that encodes a polypeptide comprising a contiguous span of at least 6 amino acids in SEQ ID No 3.

16. An isolated, purified, or recombinant polypeptide comprising a contiguous span of at least 6 amino acids of SEQ ID No 3.

17. An isolated or purified antibody composition that selectively binds to an epitope-containing fragment of a polypeptide of claim 16.
